(19) United States
(12) Patent Application Publication
(10) Pub. No.: US 2024/0140986 A1
(43) Pub. Date: May 2, 2024

(54) SYNTHETIC URSOLIC ACID DERIVATIVES AND METHODS OF USE THEREOF

(71) Applicant: REATA PHARMACEUTICALS, INC., Irving, TX (US)

(72) Inventors: Xin JIANG, Coppell, TX (US); Ha DO, Irving, TX (US); Haizhou SUN, Irving, TX (US); Melean VISNICK, Irving, TX (US); Robert M. KRAL, JR., Irving, TX (US)

(21) Appl. No.: 18/261,732

(22) PCT Filed: Jan. 18, 2022
(86) PCT No.: PCT/US2022/012831

§ 371 Date: Jul. 17, 2023

Related U.S. Application Data
(60) Provisional application No. 63/199,694, filed on Jan. 18, 2021.

(51) Int. Cl. C07J 63/00; A61P 37/02

(52) U.S. Cl. CPC C07J 63/008; A61P 37/02

(57) ABSTRACT

Disclosed herein are compounds of Formula (I-A), wherein the vari-ables are defined herein. Also provided are pharmaceutical compositions thereof. In some aspects, the compounds and compositions provided herein may be used as antioxidant inflammation modulators. In some aspects, the present disclosure provides methods wherein the compounds and composition described herein are used for the treatment of diseases and disorders associated with inflammation and cancer.

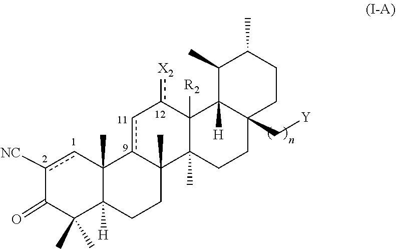

### REFERENCE TO RELATED APPLICATIONS

This application claims the benefit of priority to U.S. Provisional Application No. 63/199,694, filed on Jan. 18, 2021, the entire contents of which is hereby incorporated by reference.

Pursuant to 37 C.F.R. 1.821(c), a sequence listing is submitted herewith as an ASCII compliant text file named “REATP0119WO_ST25.txt”, created on Jan. 17, 2022 and having a size of ˜1 kilobytes. The content of the aforementioned file is hereby incorporated by reference in its entirety.

### BACKGROUND OF THE INVENTION

### I. Field of the Invention

The present invention relates generally to the fields of biology, chemistry, and medicine. More particularly, it concerns compounds, compositions and methods for the treatment and prevention of diseases and disorders such as those associated with oxidative stress and inflammation.

### II. Description of Related Art

The anti-inflammatory and anti-proliferative activity of naturally occurring triterpenoids, including ursolic acid and oleanolic acid, has been improved by chemical modifications. For example, 2-cyano-3,12-dioxoursan-1,9(11)-dien-28-oic acid (CDDU), 2-cyano-3,12-dioxooleana-1,9(11)-dien-28-oic acid (CDDO), and related compounds have been developed (Fu et al., 2014; Honda et al., 1997; Honda et al., 1998; Honda et al., 1999; Honda et al., 2000a; Honda et al., 2000b; Honda, et al., 2002; Suh et al. 1998; Suh et al., 1999; Place et al., 2003; Liby et al., 2005; and U.S. Pat. Nos. 7,915,402; 7,943,778; 8,071,632; 8,124,799; 8,129,429; 8,338,618, 8,993,640, 9,701,709, 9,512,094, and 9,889,143). The methyl ester, bardoxolone methyl (CDDO-Me), has been evaluated clinically, including for the treatment of cancer and chronic kidney disease (Pergola et al., 2011; Hong et al., 2012).

Synthetic triterpenoid analogs of oleanolic acid have also been shown to be inhibitors of cellular inflammatory processes, such as the induction by IFN-γ of inducible nitric oxide synthase (iNOS) and of COX-2 in mouse macrophages. See Honda et al. (2000a); Honda et al. (2000b), and Honda et al. (2002). Synthetic derivatives of another triterpenoid, betulinic acid, have also been shown to inhibit cellular inflammatory processes, although these compounds have been less extensively characterized (Honda et al., 2006). The pharmacology of these synthetic triterpenoid molecules is complex. Compounds derived from oleanolic acid have been shown to affect the function of multiple protein targets and thereby modulate the activity of several important cellular signaling pathways related to oxidative stress, cell cycle control, and inflammation (e.g., Dinkova-Kostova et al., 2005; Ahmad et al., 2006; Ahmad et al., 2008; Liby et al., 2007a). Derivatives of betulinic acid, though they have shown comparable anti-inflammatory properties, also appear to have significant differences in their pharmacology compared to oleanolic acid derived compounds (Liby et al., 2007b). Given that the biological activity profiles of known triterpenoid derivatives vary, and in view of the wide variety of diseases that may be treated or prevented with compounds having potent antioxidant and anti-inflammatory effects, and the high degree of unmet medical need represented within this variety of diseases, it is desirable to synthesize new compounds, including those sourced from different natural products, with diverse structures that may have improved biological activity profiles for the treatment of one or more indications.

### SUMMARY OF THE INVENTION

The present disclosure provides novel synthetic triterpenoid derivatives with anti-inflammatory and/or antioxidant properties, pharmaceutical compositions, and methods for their manufacture, and methods for their use.

In one aspect, there are provided compounds of the formula:

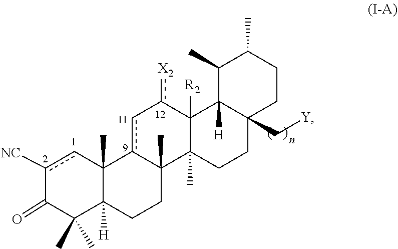

wherein:
the bond between atom 1 and atom 2 is a single bond, a double bond, or an epoxidized double bond;
the bond between atom 9 and atom 11 is a single bond or a double bond;
the bond between atom 12 and X₂ is a single bond or a double bond;
n is 0-6;
X₂ is oxo, or X₂ is taken together with Y as defined below, provided that when X₂ is oxo, then the bond between atom 12 and X₂ is a double bond, and when X₂ is taken together with Y as defined below, that the bond between atom 12 and X₂ is a single bond;
R₂ is hydrogen or hydroxy; or R₂ is taken together with Y as defined below; and
Y is hydrogen, hydroxy, halo, or amino; or
   alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups; or
   —arenediyl_{(C≤12)}—R₃, substituted -arenediyl_{(C≤12)}—R₃, -heteroarenediyl_{(C≤12)}—R₃, or substituted -heteroarenediyl_{(C≤12)}—R₃, wherein:
      R₃ is alkyl_{(C≤12)}, cycloalkyl_{(C≤12)}, heterocycloalkyl_{(C≤12)}, or a substituted version of any of these groups; or
         alkanediyl_{(C≤12)}—R₄ or substituted -alkanediyl_{(C≤12)}—R₄, wherein:
         R₄ is alkoxy_{(C≤12)}, alkylamino_{(C≤12)}, dialkylamino_{(C≤12)}, or a substituted version of any of these groups; or
   alkanediyl_{(C≤12)}—C(O)R₅ or substituted -alkanediyl_{(C≤12)}—C(O)R₅, wherein:
      R₅ is hydroxy or amino; or alkoxy_{(C≤12)}, alkylamino_{(C≤12)}, dialkylamino(c₅i2), cycloalkyl_{(C≤12)}, cycloalkoxy_{(C≤12)}, cycloalkylamino_{(C≤12)}, heterocycloalkyl_{(C≤12)}, or a substituted version of any of these groups; or
   —C(O)R₇, wherein:
      R₇ is hydrogen, heterocycloalkyl_{(C≤12)}, substituted heterocycloalkyl_{(C≤12)}, cycloalkylamino_{(C≤12)}, substituted cycloalkylamino_{(C≤12)}, —NHC(NH)-alkyl_{(C≤12)}, or —NHOR₁₃, wherein:
         R₁₃ is hydrogen, alkyl_{(C≤12)}, or substituted alkyl_{(C≤12)}; or
   NR₈R₉, wherein:
      R₈ is hydrogen, alkyl_{(C≤12)}, or substituted alkyl_{(C≤12)}; and
         R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)}, or
         CO₂R₁₀, wherein:
         R₁₀ is hydrogen, alkyl_{(C≤12)}, cycloalkyl_{(C≤12)}, or a substituted version of any of these groups; or
         —C(O)R₁₂, wherein: R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups; or
   CH═NOR₁₁, wherein:
      R₁₁ is hydrogen, alkyl_{(C≤12)}, or substituted alkyl_{(C≤12)}; or
Y is taken together with R₂ and is —(CH₂)ₘX₁—, wherein:
   m is 0-6; and
   X₁ is —O—; or
Y is taken together with X₂ and is —(CH₂)ₒC(O)—, wherein:
   is 0-6; and
   X₂ is —O—;
      or a pharmaceutically acceptable salt thereof.

In some embodiments, the compound is further defined as:

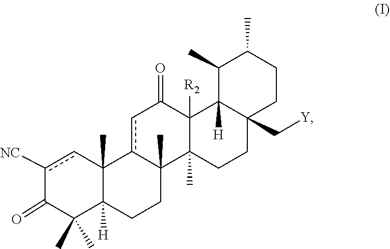

wherein:
the bond between atom 1 and atom 2 is a single bond, a double bond, or an epoxidized double bond;
the bond between atom 9 and atom 11 is a single bond or a double bond;
n is 0-6;
R₂ is hydrogen or hydroxy; or R₂ is taken together with Y as defined below; and
Y is hydrogen, hydroxy, halo, or amino; or
   alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups; or
   —arenediyl_{(C≤12)}—R₃, substituted -arenediyl_{(C≤12)}—R₃, -heteroarenediyl_{(C≤12)}—R₃, or substituted -heteroarenediyl_{(C≤12)}—R₃, wherein:
      R₃ is alkyl_{(C≤12)}, cycloalkyl_{(C≤12)}, heterocycloalkyl_{(C≤12)}, or a substituted version of any of these groups; or
         alkanediyl_{(C≤12)}—R₄ or substituted -alkanediyl_{(C≤12)}—R₄, wherein:
         R₄ is alkoxy_{(C≤12)}, alkylamino_{(C≤12)}, dialkylamino_{(C≤12)}, or a substituted version of any of these groups; or —alkanediyl_{(C≤12)}C(O)—R₅ or substituted -alkanediyl_{(C≤12)}—C(O)R₅, wherein:
      R₅ is hydroxy or amino; or
         alkoxy_{(C≤12)}, alkylamino(₅12), dialkylamino_{(C≤12)}, cycloalkyl_{(C≤12)}, cycloalkoxy_{(C≤12)}, cycloalkylamino_{(C≤12)}, heterocycloalkyl_{(C≤12)}, or a substituted version of any of these groups; or
   —C(O)R₇, wherein:
      R₇ is hydrogen, heterocycloalkyl_{(C≤12)}, substituted heterocycloalkyl_{(C≤12)}, cycloalkylamino_{(C≤12)}, substituted cycloalkylamino_{(C≤12)}, —NHC(NH)-alkyl_{(C≤12)}, or —NHOR₁₃, wherein:
         R₁₃ is hydrogen, alkyl_{(C≤12)}, or substituted alkyl_{(C≤12)}; or
   NR₈R₉, wherein:
      R₈ is hydrogen, alkyl_{(C≤12)}, or substituted alkyl_{(C≤12)}; and
         R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)}, or
         CO₂R₁₀, wherein: R₁₀ is hydrogen, alkyl_{(C≤12)}, cycloalkyl_{(C≤12)}, or a substituted version of any of these groups; or
         —C(O)R₁₂, wherein:
         R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups; or
   CH═NOR₁₁, wherein:
      R₁₁ is hydrogen, alkyl_{(C≤12)}, or substituted alkyl_{(C≤12)}; or
Y is taken together with R₂ and is —(CH₂)ₘX₁—, wherein:
   m is 0-6; and
   X₁ is —O—;
      or a pharmaceutically acceptable salt thereof.

In further embodiments, the compound is further defined as:

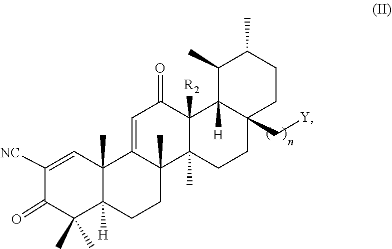

wherein:
n is 0-6;
R₂ is hydrogen or hydroxy; or R₂ is taken together with Y as defined below; and
Y is hydrogen, hydroxy, halo, or amino; or
   alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups; or
   —arenediyl_{(C≤12)}—R₃, substituted -arenediyl_{(C≤12)}—R₃, -heteroarenediyl_{(C≤12)}—R₃, or substituted -heteroarenediyl_{(C≤12)}—R₃, wherein:
      R₃ is alkyl_{(C≤12)}, cycloalkyl_{(C≤12)}, heterocycloalkyl_{(C≤12)}, or a substituted version of any of these groups; or —alkanediyl_{(C≤12)}—R₄ or substituted -alkanediyl_{(C≤12)}—R₄, wherein:
         R₄ is alkoxy_{(C≤12)}, alkylamino_{(C≤12)}, dialkylamino_{(C≤12)}, or a substituted version of any of these groups; or —alkanediyl_{(C≤12)}—C(O)R₅ or substituted -alkanediyl_{(C≤12)}—C(O)R₃, wherein:
      R₅ is hydroxy or amino; or
         alkoxy_{(C≤12)}, alkylamino_{(C≤12)}, dialkylamino_{(C≤12)}, cycloalkyl_{(C≤12)}, cycloalkoxy_{(C≤12)}, cycloalkylamino_{(C≤12)}, heterocycloalkyl_{(C≤12)}, or a substituted version of any of these groups;
   —C(O)R₇, wherein:
      R₇ is hydrogen, heterocycloalkyl_{(C≤12)}, substituted heterocycloalkyl_{(C≤12)}, cycloalkylamino_{(C≤12)}, substituted cycloalkylamino_{(C≤12)}, —NHC(NH)-alkyl_{(C≤12)}, or —NHOR₁₃, wherein:
      R₁₃ is hydrogen, alkyl_{(C≤12)}, or substituted alkyl_{(C≤12)};
   NR₈R₉, wherein:
      R₈ is hydrogen, alkyl_{(C≤12)}, or substituted alkyl_{(C≤12)}; and
         R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)}, or
         CO₂R₁₀, wherein:
         R₁₀ is hydrogen, alkyl_{(C≤12)}, cycloalkyl_{(C≤12)}, or a substituted version of any of these groups; or
         —C(O)R₁₂, wherein:
         R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups; or
   CH═NOR₁₁, wherein:
      R₁₁ is hydrogen, alkyl_{(C≤12)}, or substituted alkyl_{(C≤12)}; or
Y is taken together with R₂ and is —(CH₂)ₘX₁—, wherein:
   m is 0-6;
   X₁ is —O—;
      or a pharmaceutically acceptable salt thereof.

In still further embodiments, the compound is further defined as:

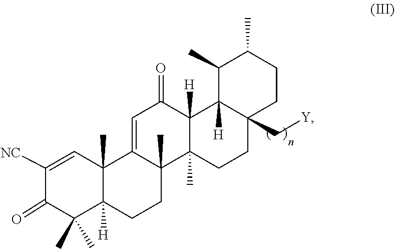

wherein:
n is 0-6;
Y is hydrogen, hydroxy, halo, or amino; or
   alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups; or
   —arenediyl_{(C≤12)}—R₃, substituted -arenediyl_{(C≤12)}—R₃, -heteroarenediyl_{(C≤12)}—R₃, or substituted -heteroarenediyl_{(C≤12)}—R₃, wherein:
      R₃ is alkyl_{(C≤12)}, cycloalkyl_{(C≤12)}, heterocycloalkyl_{(C≤12)}, or a substituted version of any of these groups; or
      —alkanediyl_{(C≤12)}—R₄ or substituted -alkanediyl_{(C≤12)}—R₄, wherein:
         R₄ is alkoxy_{(C≤12)}, alkylamino_{(C≤12)}, dialkylamino_{(C≤12)}, or a substituted version of any of these groups; or
   alkanediyl_{(C≤12)}—C(O)R₅ or substituted -alkanediyl_{(C≤12)}—C(O)R₅, wherein:
      R₅ is hydroxy or amino; or
      alkoxy_{(C≤12)}, alkylamino_{(C≤12)}, dialkylamino_{(C≤12)}, cycloalkyl_{(C≤12)}, heterocycloalkyl_{(C≤12)}, or a substituted version of any of these groups; or
         —C(O)R₇, wherein:
      R₇ is hydrogen, heterocycloalkyl_{(C≤12)}, substituted heterocycloalkyl_{(C≤12)}, cycloalkylamino_{(C≤12)}, substituted cycloalkylamino_{(C≤12)}, —NHC(NH)-alkyl_{(C≤12)}, or —NHOR₁₃, wherein:
      R₁₃ is hydrogen, alkyl_{(C≤12)}, or substituted alkyl_{(C≤12)};
   —NR₈R₉, wherein:
      R₈ is hydrogen, alkyl_{(C≤12)}, or substituted alkyl_{(C≤12)}; and
         R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)}, or
         CO₂R₁₀, wherein:
         R₁₀ is hydrogen, alkyl_{(C≤12)}, cycloalkyl_{(C≤12)}, or a substituted version of any of these groups; or
         —C(O)R₁₂, wherein:
         R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups; or
   CH═NOR₁₁, wherein:
      Rn is hydrogen, alkyl_{(C≤12)}, or substituted alkyl_{(C≤12)};
or a pharmaceutically acceptable salt thereof. In some embodiments, the bond between atom 1 and atom 2 is a double bond or an epoxidized double bond. In some embodiments, the bond between atom 1 and atom 2 is a double bond. In other embodiments, the bond between atom 1 and atom 2 is an epoxidized double bond. In some embodiments, the bond between atom 9 and atom 11 is a single bond. In other embodiments, the bond between atom 9 and atom 11 is a double bond.

In some embodiments, X₂ is oxo. In some embodiments, n is 0, 1, or 2. In some embodiments, n is 0. In other embodiments, n is 1. In still other embodiments, n is 2.

In some embodiments, Y is hydrogen, hydroxy, halo, or amino; or
alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups; or —arenediyl_{(C≤12)}—R₃, substituted -arenediyl_{(C≤12)}—R₃, -heteroarenediyl_{(C≤12)}—R₃, or substituted —heteroarenediyl_{(C≤12)}—R₃, wherein:
R₃ is alkyl_{(C≤12)}, cycloalkyl_{(C≤12)}, heterocycloalkyl_{(C≤12)}, or a substituted version of any of these groups; or
   —alkanediyl_{(C≤12)}—R₄ or substituted -alkanediyl_{(C≤12)}—R₄, wherein:
      R₄ is alkoxy_{(C≤12)}, alkylamino_{(C≤12)}, dialkylamino_{(C≤12)}, or a substituted version of any of these groups; or —alkanediyl_{(C≤12)}C(O)—R₅ or substituted -alkanediyl_{(C≤12)}—R₅, wherein:
R₅ is hydroxy or amino; or alkoxy_{(C≤12)}, alkylamino_{(C≤12)}, dialkylamino_{(C≤12)}, cycloalkyl_{(C≤12)}, heterocycloalkyl_{(C≤12)}, or a substituted version of any of these groups.

In some embodiments, Y is amino; or alkyl_{(C≤12)}, amido_{(C≤12)}, heterocycloalkyl_{(C≤12)}, heteroaryl_{(C≤12)}, or a substituted version of any of these groups; or —heteroarenediyl_{(C≤12)}—R₃ or substituted -heteroarenediyl_{(C≤12)}—R₃, wherein:
R₃ is alkyl_{(C≤12)}, cycloalkyl_{(C≤12)}, heterocycloalkyl_{(C≤12)}, or a substituted version of any of these groups); or —alkanediyl_{(C≤12)}—R₄ or substituted -alkanediyl_{(C≤12)}—R₄, wherein:
R₄ is alkoxy_{(C≤12)} or substituted alkoxy_{(C≤12)}.

In some embodiments, Y is amino. In other embodiments, Y is alkyl_{(C≤12)} or substituted alkyl_{(C≤12)}. In further embodiments, Y is substituted alkyl_{(C≤12)}, such as hydroxymethyl or methylaminomethyl. In still other embodiments, Y is amido_{(C≤12)} or substituted amido_{(C≤12)}. In further embodiments, Y is amido_{(C≤12)}, such as acetamido or propionamido. In other embodiments, Y is substituted amido_{(C≤12)}, such as 2,2-difluoropropionamido or 2,2-difluoroacetamido. In yet other embodiments, Y is heterocycloalkyl_{(C≤12)} or substituted heterocycloalkyl_{(C≤12)}. In some embodiments, Y is heterocycloalkyl_{(C≤12)}, such as oxazolidin-3-ylmethyl or azetidin-1-yl. In other embodiments, Y is substituted heterocycloalkyl_{(C≤12)}, such as 2-oxooxazolidin-3-yl, 3-methyl-2-oxoimidazolidin-1-yl, 2-oxoimidazolidin-1-yl, 2,5-dioxopyrrolidin-1-yl, methyl 3-oxopyrazolidine-1-carboxylate, 5-oxopyrazolidin-1-yl, 2-oxoazetidin-1-yl and 2-oxopyrrolidin-1-yl. In other embodiments, Y is heteroaryl_{(C≤12)} or substituted heteroaryl_{(C≤12)}. In further embodiments, Y is heteroaryl_{(C≤12)}, such as 3-methyl-1,2,4-oxadiazol-5-yl, 3-ethyl-1,2,4-oxadiazol-5-yl, 1H-pyrazol-1-yl, 1H-1,2,4-triazol-1-yl, 4-methyl-1H-1,2,3-triazol-1-yl, 1H-tetrazol-1-yl, 1H-1,2,3-triazol-1-yl, 1H-imidazol-1-yl, 5-methyl-1,3,4-oxadiazol-2-yl, or 5-methyl-1,2,4-oxadiazol-3-yl. In other embodiments, Y is substituted heteroaryl_{(C≤12)}, such as 4-bromo-1H-pyrazol-1-yl, 3-(2-methoxyethyl)-1,2,4-oxadiazol-5-yl, 3-(methoxymethyl)-1,2,4-oxadiazol-5-yl, 3-(2-hydroxyethyl)-1,2,4-oxadiazol-5-yl, 3-(hydroxymethyl)-1,2,4-oxadiazol-5-yl, 3-((dimethylamino)methyl)-1,2,4-oxadiazol-5-yl, 3-(1-methoxyethyl)-1,2,4-oxadiazol-5-yl, or 3-(fluoromethyl)-1,2,4-oxadiazol-5-yl.

In still other embodiments, Y is -heteroarenediyl_{(C≤12)}—R₃ or substituted —heteroarenediyl_{(C≤12)}—R₃. In further embodiments, Y is -heteroarenediyl_{(C≤12)}—R₃. In still further embodiments, Y is a group of the formula:

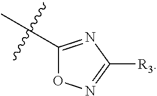

In other embodiments, Y is a group of the formula:

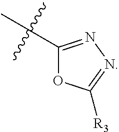

In still other embodiments, Y is a group of the formula:

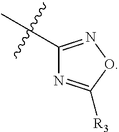

In some embodiments, R₃ is alkyl_{(C≤12)} or substituted alkyl_{(C≤12)}. In further embodiments, R₃ is alkyl_{(C≤12)}, such as methyl or ethyl. In other embodiments, R₃ is substituted alkyl_{(C≤12)}, such as 2-methoxyethyl, methoxymethyl, 2-hydroxyethyl, hydroxymethyl, (dimethylamino)methyl, 1-methoxyethyl, or fluoromethyl. In some embodiments, R₃ is polar-substituted alkyl_{(C≤12)}. In further embodiments, R₃ is monopolar-substituted alkyl_{(C≤12)}. In still further embodiments, monoaminoalkyl_{(C≤12)}, monofluoroalkyl_{(C≤12)}, or monohydroxyalkyl_{(C≤12)}. In further embodiments, R₃ is monofluoroalkyl_{(C≤12)} or monohydroxyalkyl_{(C≤12)}. In still further embodiments, R₃ is monohydroxyalkyl_{(C≤12)}, such as 2-hydroxyethyl or hydroxymethyl. In other embodiments, R₃ is monofluoroalkyl_{(C≤12)}, such as fluoromethyl. In other embodiments, R₃ is cycloalkyl_{(C≤12)} or substituted cycloalkyl_{(C≤12)}. In further embodiments, R₃ is cycloalkyl_{(C≤12)}, such as cyclopropyl. In still other embodiments, R₃ is -alkanediyl_{(C≤12)}—R₄ or substituted -alkanediyl_{(C≤12)}—R₄. In further embodiments, R₃ is -methanediyl-R₄. In some embodiments, R₄ is alkoxy_{(C≤12)}, such as t-butoxy.

In some embodiments, Y is -alkanediyl_{(C≤12)}—C(O)R₅ or substituted —alkanediyl_{(C≤12)}—C(O)R₅, wherein: R₅ is hydroxy or amino; or alkoxy_{(C≤12)}, alkylamino_{(C≤12)}, dialkylamino_{(C≤12)}, cycloalkyl_{(C≤12)}, cycloalkoxy_{(C≤12)}, cycloalkylamino_{(C≤12)}, heterocycloalkyl_{(C≤12)}, or a substituted version of any of these groups.

In some embodiments, R₅ is hydroxy. In other embodiments, R₅ is amino. In still other embodiments, R₅ is alkoxy_{(C≤12)}, such as methoxy. In some embodiments, R₅ is alkylamino_{(C≤12)}, such as methylamino or ethylamino. In other embodiments, R₅ is substituted alkylamino_{(C≤12)}, such as 2,2-difluoroethan-1-amino. In some embodiments, R₅ is cycloalkyl_{(C≤12)} or substituted cycloalkyl_{(C≤12)}. In further embodiments, R₅ is cycloalkyl_{(C≤12)}, such as cyclopropyl. In some embodiments, R₅ is cycloalkylamino_{(C≤12)}, or substituted cycloalkylamino_{(C≤12)}. In further embodiments, R₅ is cycloalkylamino_{(C≤12)}, such as cyclopropylamino. In some embodiments, R₅ is heterocycloalkyl_{(C≤12)} or substituted heterocycloalkyl_{(C≤12)}. In further embodiments, R₅ is heterocycloalkyl_{(C≤12)}, such as azetidine or pyrrolidine.

In some embodiments, Y is —C(O)R₇, wherein: R₇ is hydrogen, heterocycloalkyl_{(C≤12)}, cycloalkylamino_{(C≤12)}, or substituted cycloalkylamino_{(C≤12)}, —NHC(NH)-alkyl_{(C≤12)}, or —NHOR_{13(C≤12)}, wherein: R₁₃ is hydrogen, alkyl, or substituted alkyl; or —NR₈R₉, wherein: R₈ is hydrogen, alkyl_{(C≤12)}, or substituted alkyl_{(C≤12)}; and R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)}, or —CO₂R₁₀, wherein: R₁₀ is hydrogen, alkyl_{(C≤12)}, or a substituted version of any of these groups; or —C(O)R₁₂, wherein: R₁₂ is hydrogen, amino, alkylamino_{(C≤12)}, dialkylamino_{(C≤12)}, cycloalkylamino_{(C≤12)}, heterocycloalkyl_{(C≤12)} or a substituted version of any of these groups; or —CH═NOR₁₁, wherein: R₁₁ is hydrogen, alkyl_{(C≤12)}, or substituted alkyl_{(C≤12)}.

In further embodiments, Y is —C(O)R₇, wherein: R₇ is hydrogen, heterocycloalkyl_{(C≤12)}, cycloalkylamino_{(C≤12)} or substituted cycloalkylamino_{(C≤12)}, —NHC(NH)-alkyl_{(C≤12)}, or —NHOR_{13(C≤12)}, wherein: R₁₃ is hydrogen, alkyl, or substituted alkyl.

In some embodiments, R₇ is hydrogen. In other embodiments, R₇ is cycloalkylamino_{(C≤12)} or substituted cycloalkylamino_{(C≤12)}, In further embodiments, R₇ is cycloalkylamino_{(C≤12)}, such as cyclopropylamino. In some embodiments, R₇ is heterocycloalkyl_{(C≤12)} or substituted heterocycloalkyl_{(C≤12)}. In further embodiments, R₇ is heterocycloalkyl_{(C≤12)}, such as azetidine or pyrrolidine. In some embodiments, R₇ is —NHC(NH)-alkyl_{(C≤12)}, such as —NHC(NH)CH₃. In some embodiments, R₇ is —NHOR_{13(C≤12)}, wherein: R₁₃ is hydrogen, alkyl_{(C≤12)}, or substituted alkyl_{(C≤12)}.

In some embodiments, R₁₃ is hydrogen. In other embodiments, R₁₃ is alkyl_{(C≤12)}, such as methyl.

In some embodiments, Y is-NR₈R₉, wherein: R₈ is hydrogen, alkyl_{(C≤12)}, or substituted alkyl_{(C≤12)}; and R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)}; or —CO₂R₁₀, wherein R₁₀ is hydrogen, alkyl_{(C≤12)}, or substituted alkyl_{(C≤12)}, or a substituted version of any of these groups; or —C(O)R₁₂, wherein: R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups.

In some embodiments, R₈ is hydrogen. In other embodiments, R₈ is alkyl_{(C≤12)} or substituted alkyl_{(C≤12)}. In further embodiments, R₈ is alkyl_{(C≤12)}, such as methyl. In some embodiments, R₉ is acyl_{(C≤12)} or substituted acyl_{(C≤12)}. In further embodiments, R₉ is acyl_{(C≤12)}, such as acetyl, methylacetyl, cyclopropylcarboxyl, or cyclobutylcarboxyl. In other embodiments, R₉ is substituted acyl_{(C≤12)}, such as methylaminocarbonyl, difluoroacetyl, or difluoromethylacetyl. In some embodiments, R₉ is alkylsulfonyl_{(C≤12)}, substituted alkylsulfonyl_{(C≤12)}, cycloalkylsulfonyl_{(C≤12)}, or substituted cycloalkylsulfonyl_{(C≤12)}. In further embodiments, R₉ is alkylsulfonyl_{(C≤12)}, such as is methylsulfonyl or ethylsulfonyl.

In some embodiments, R₉ is cycloalkylsulfonyl_{(C≤12)}, such as cyclopropylsulfonyl_{(C≤12)}.

In some embodiments, R₉ is —CO₂R₁₀, wherein R₁₀ is hydrogen, alkyl_{(C≤12)}, or substituted alkyl_{(C≤12)}, or a substituted version of any of these groups. In some embodiments, R₁₀ is alkyl_{(C≤12)} or substituted alkyl_{(C≤12)}. In further embodiments, R₁₀ is alkyl_{(C≤12)}, such as methyl or t-butyl. In other embodiments, R₉ is —C(O)R₁₂, wherein: R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups.

In some embodiments, R₁₂ is hydrogen. In other embodiments, R₁₂ is amino. In still other embodiments, R₁₂ is alkylamino_{(C≤12)} or substituted alkylamino_{(C≤12)}. In further embodiments, R₁₂ is alkylamino_{(C≤12)}, such as methylamino or ethylamino. In some embodiments, R₁₂ is dialkylamino_{(C≤12)} or substituted dialkylamino_{(C≤12)}. In other embodiments, R₁₂ is cycloalkylamino_{(C≤12)} or substituted cycloalkylamino_{(C≤12)}. In further embodiments, R₁₂ is cycloalkylamino_{(C≤12)}, such as cyclopropylamino. In some embodiments, R₁₂ is heterocycloalkyl_{(C≤12)} or substituted heterocycloalkyl_{(C≤12)}. In further embodiments, R₁₂ is heterocycloalkyl_{(C≤12)}, such as azetidine.

In some embodiments, Y is: —CH═NOR₁₁, wherein: Rn is hydrogen, alkyl_{(C≤12)}, or substituted alkyl_{(C≤12)}.

In some embodiments, Rn is hydrogen. In other embodiments, R₁₁ is alkyl_{(C≤12)} or substituted alkyl_{(C≤12)}. In further embodiments, R₁₁ is alkyl_{(C≤12)}, such as methyl.

In some embodiments, Y is taken together with R₂ and is —(CH₂)ₘX₁—, wherein:
m is 0-6 and X₁ is —O—. In some embodiments, m is 1. In other embodiments, m is 2.

In some embodiments, Y is taken together with X₂ and is —(CH₂)ₒC(O)—, wherein: o is 0-6. In further embodiments, o is 1.

In some embodiments, the compound is further defined as:

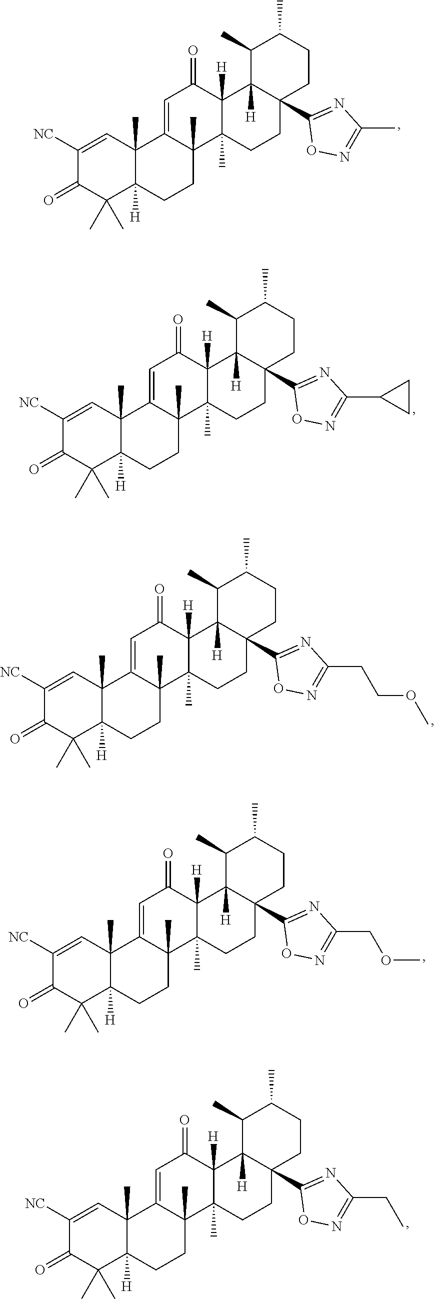

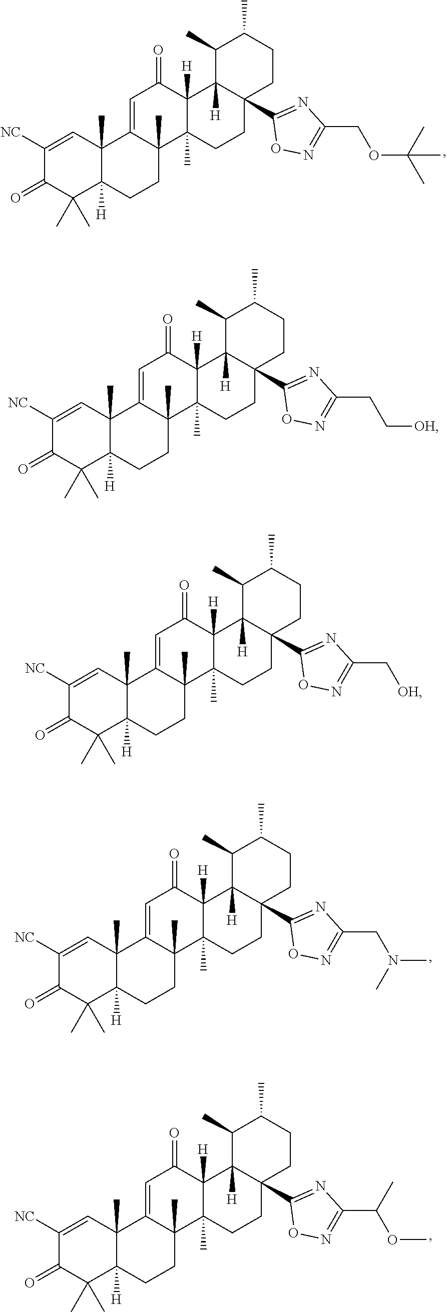

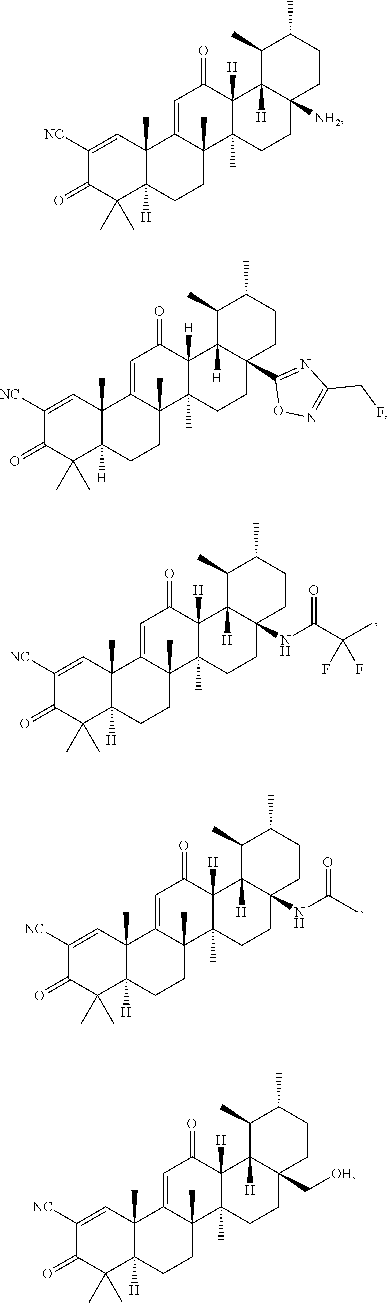

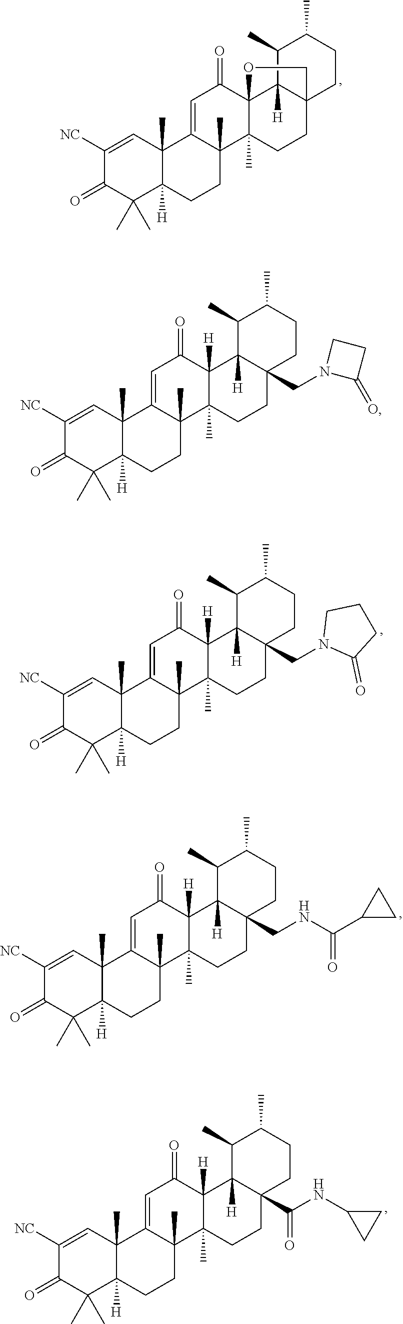

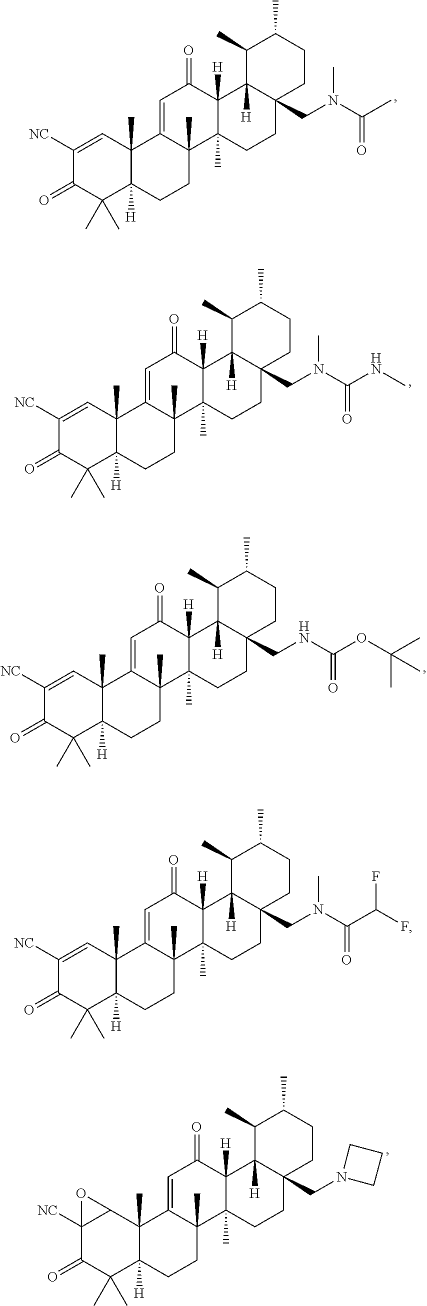

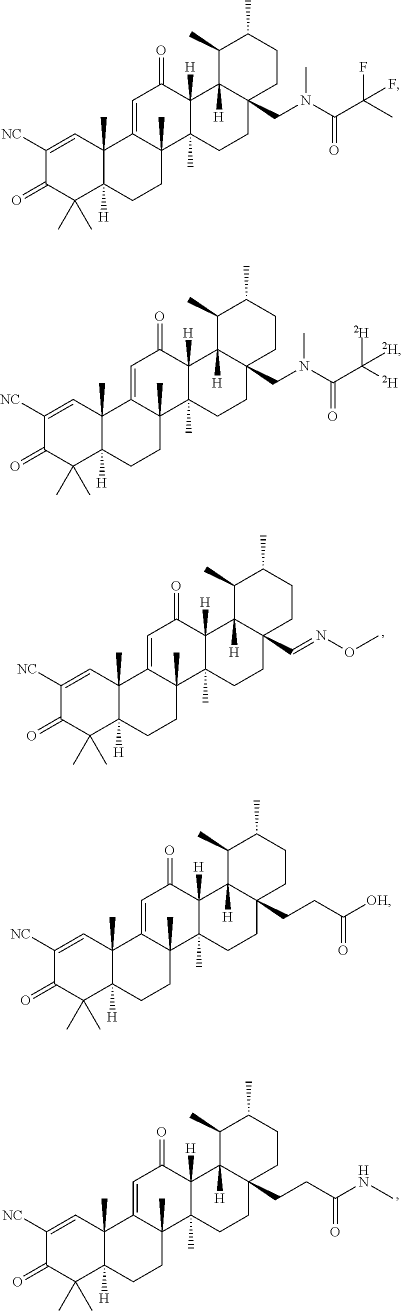

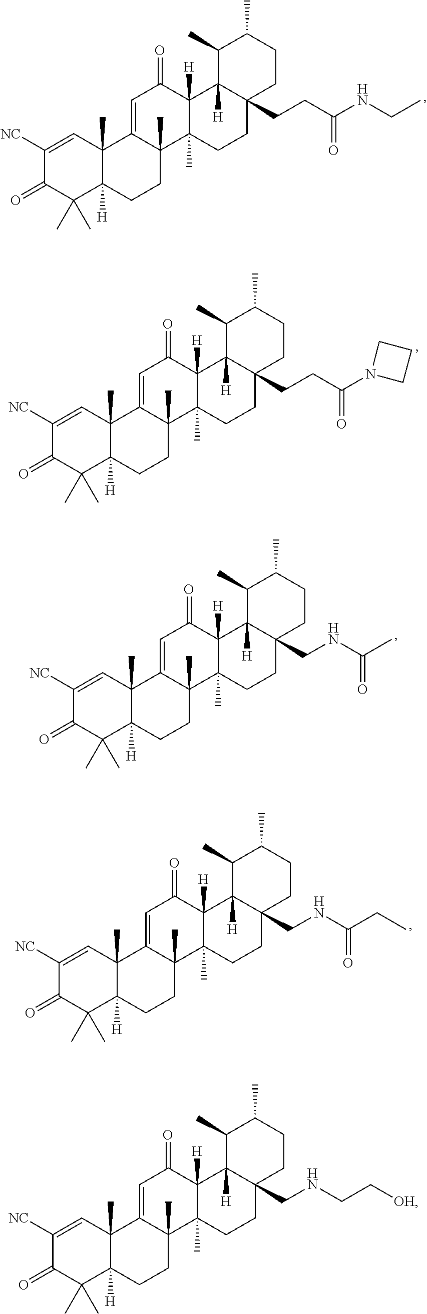

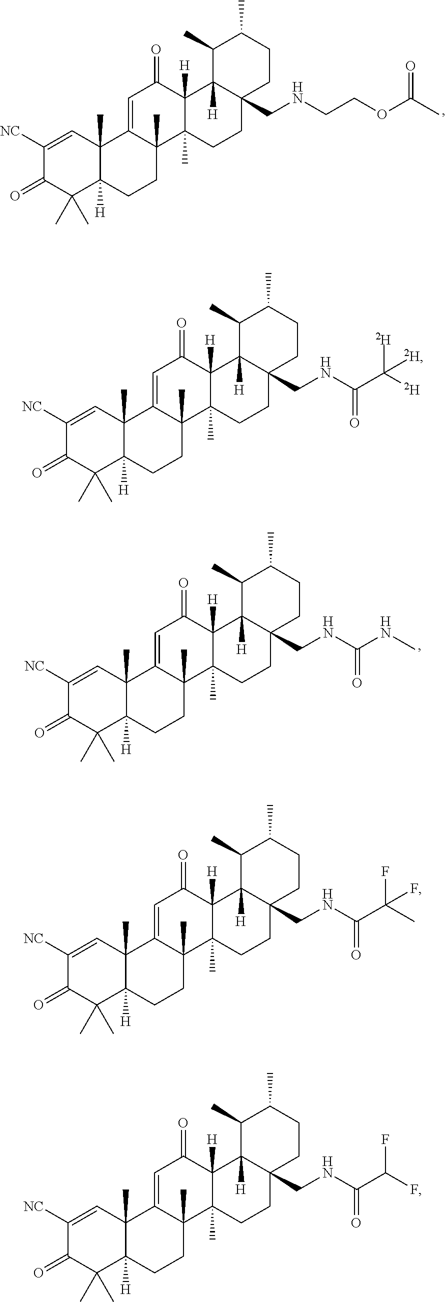

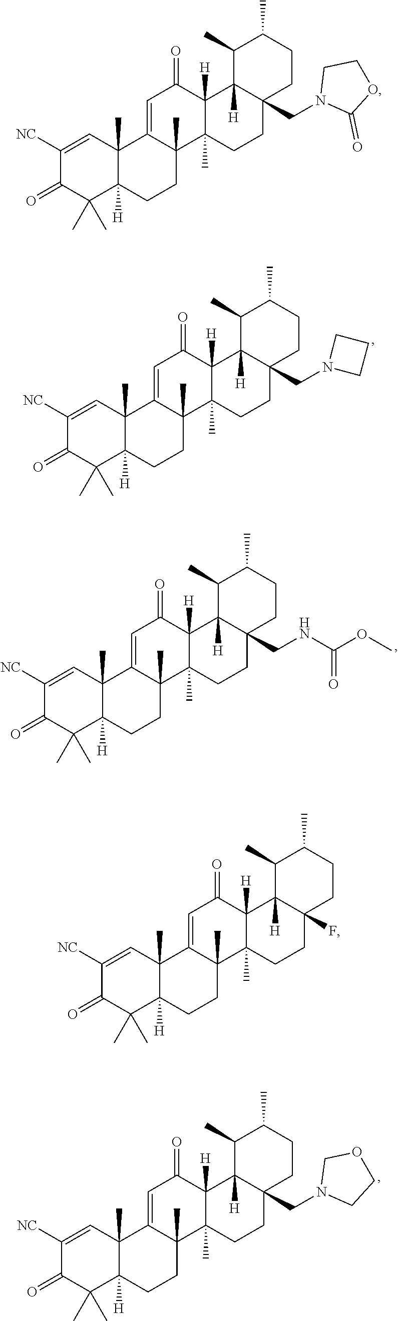

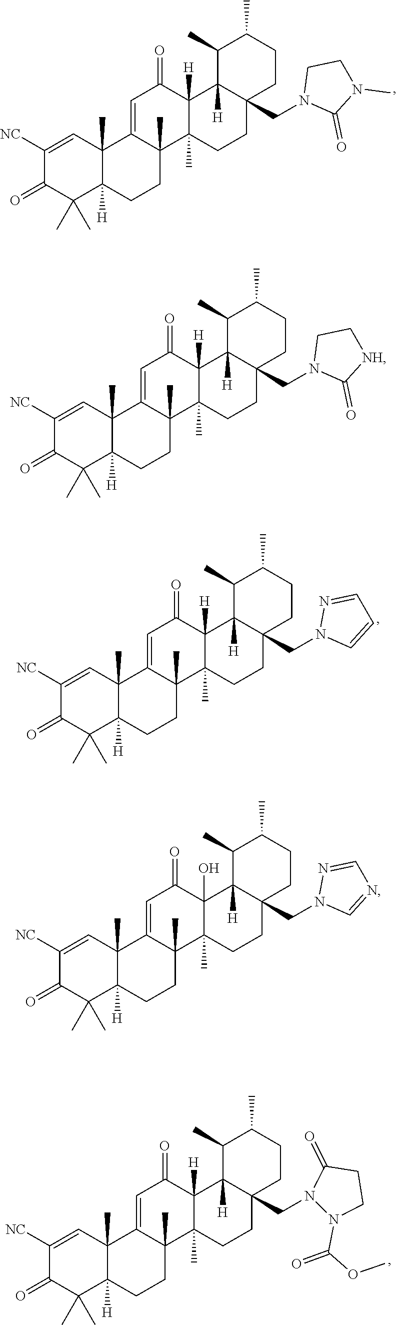

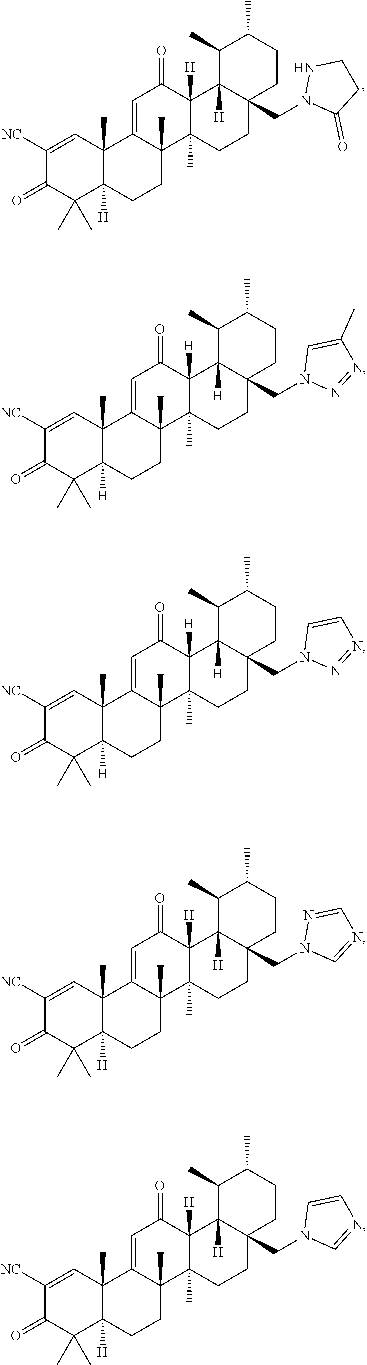

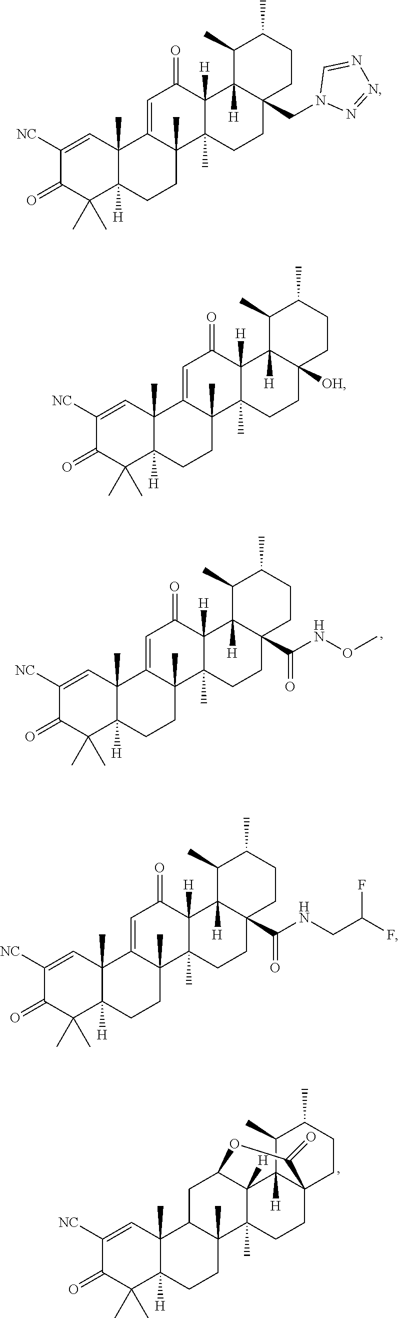

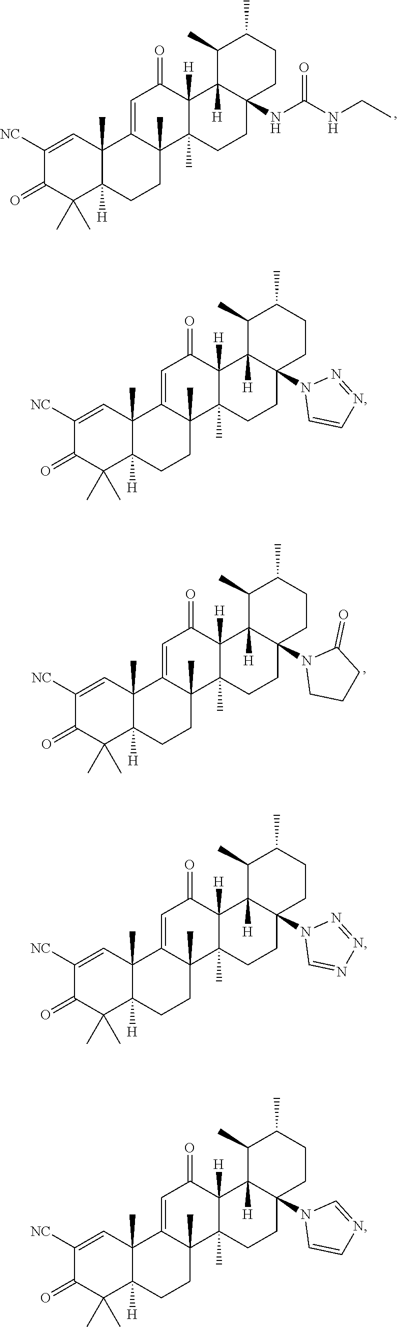

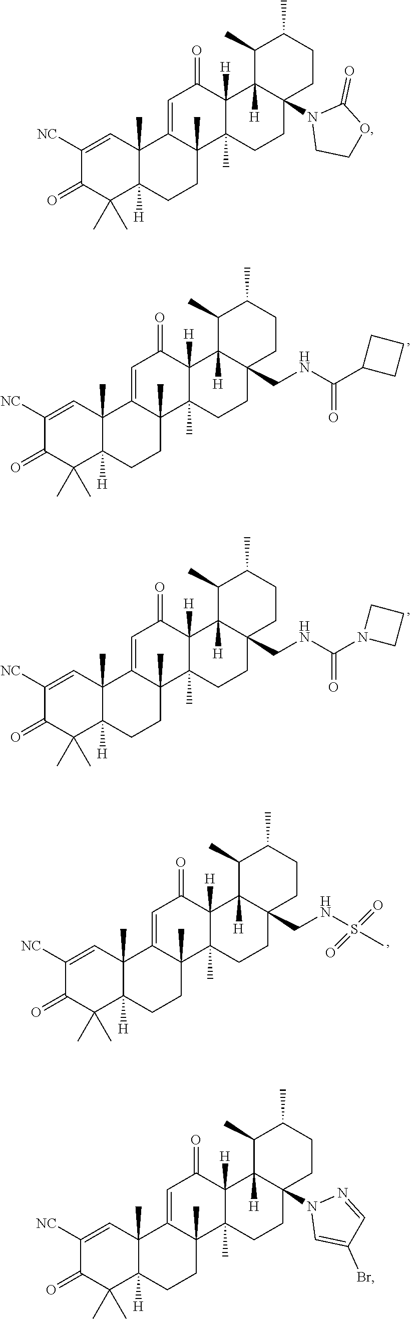

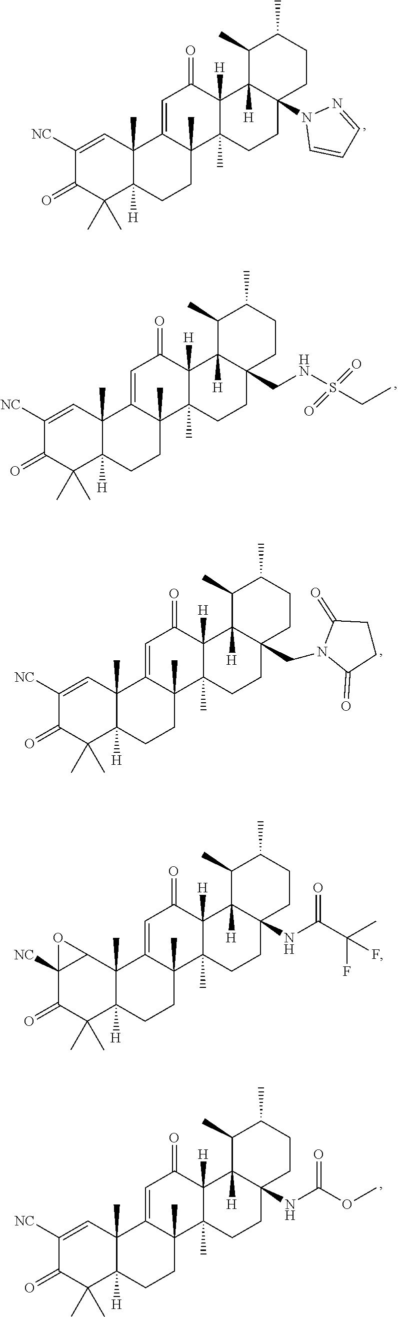

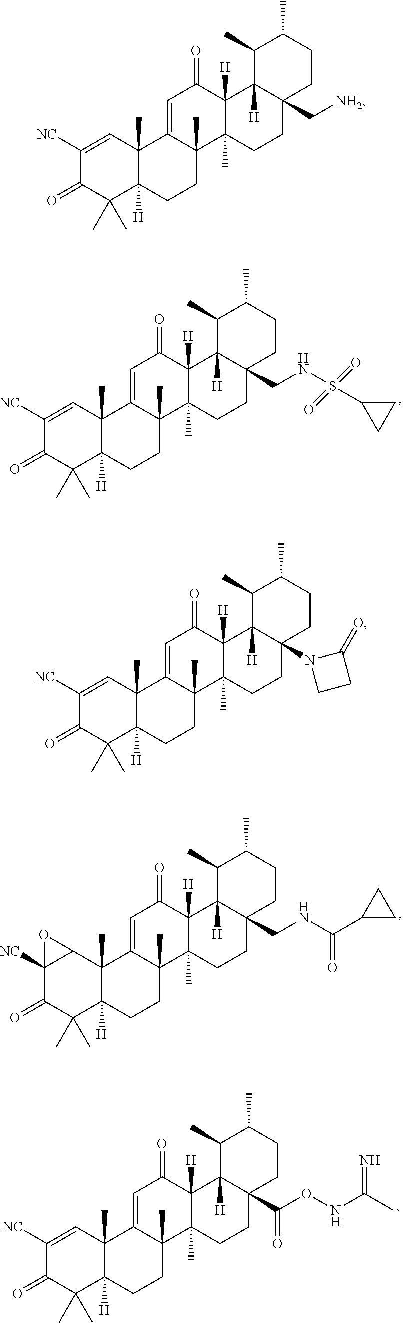

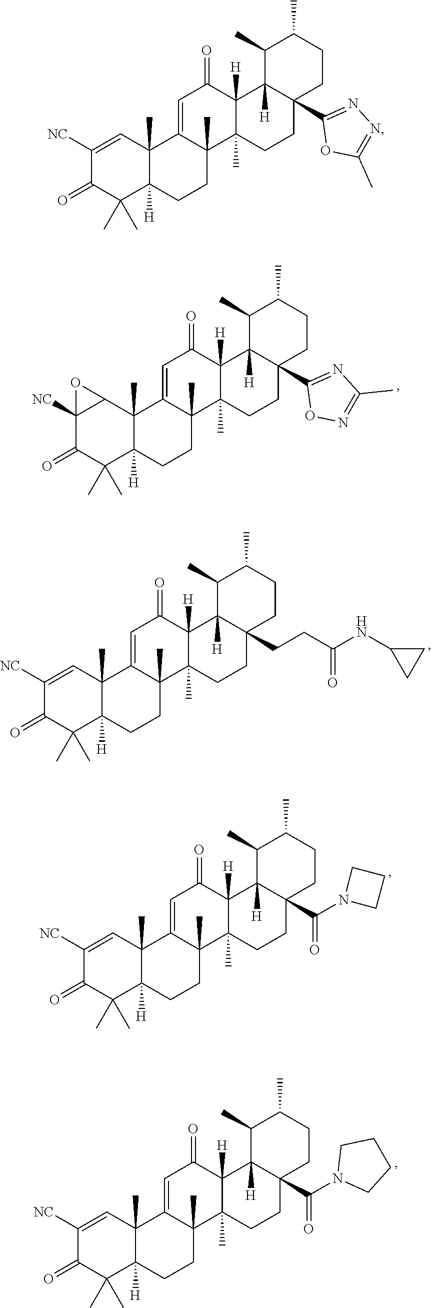

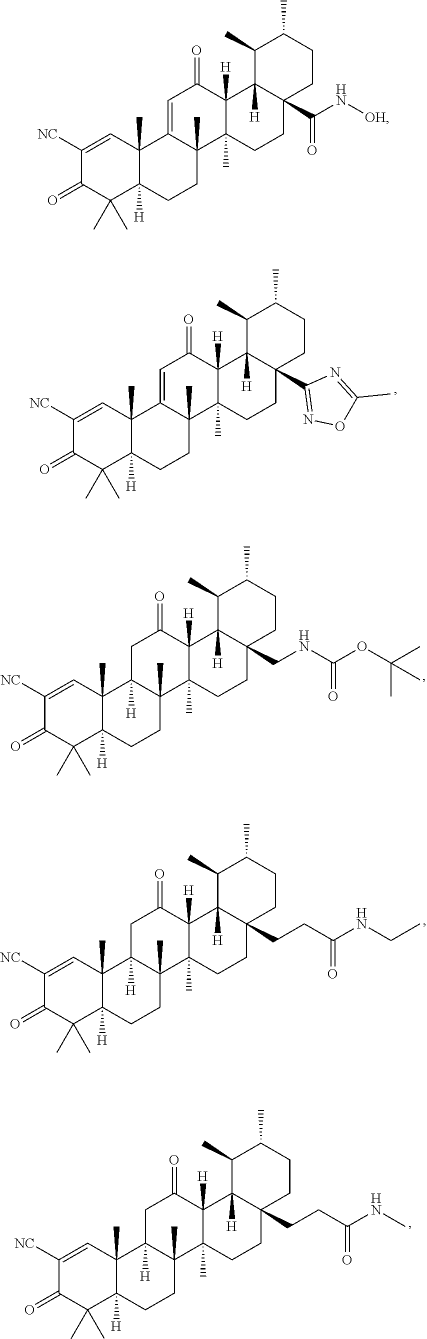

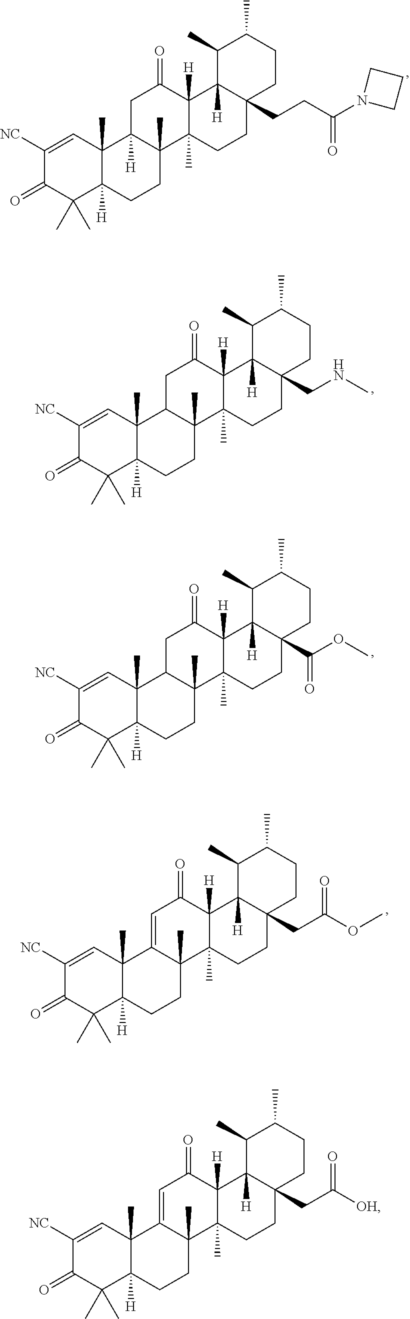

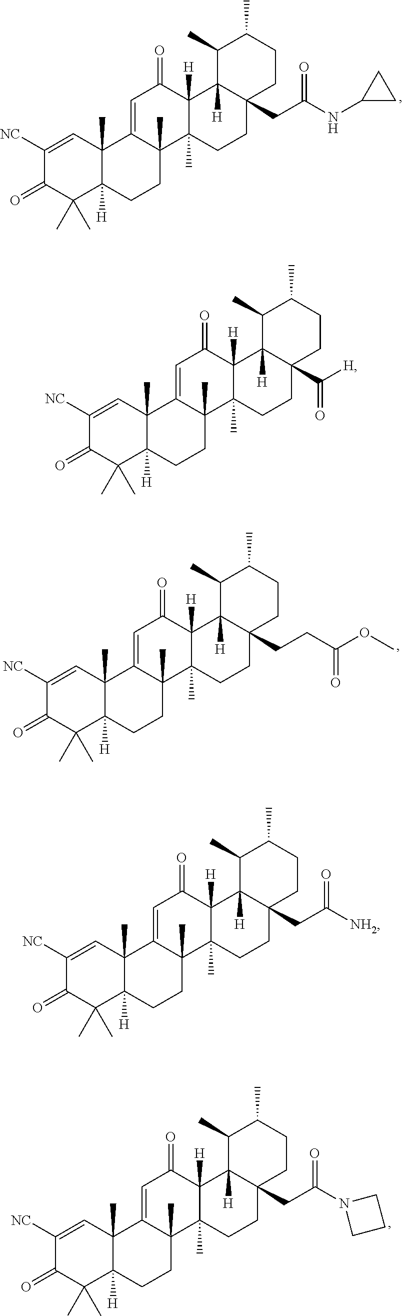

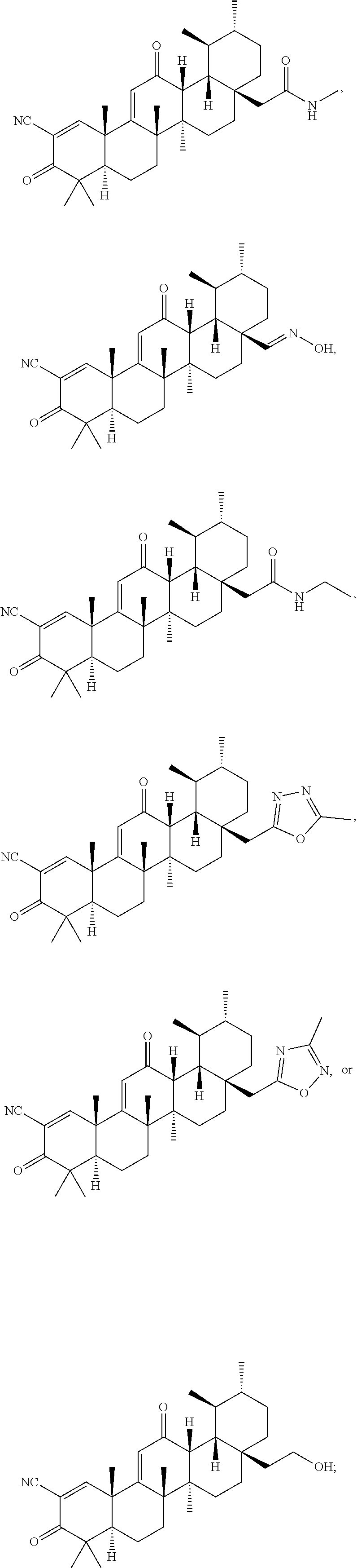

or a pharmaceutically acceptable salt thereof.

In further embodiments, the compound is further defined as:

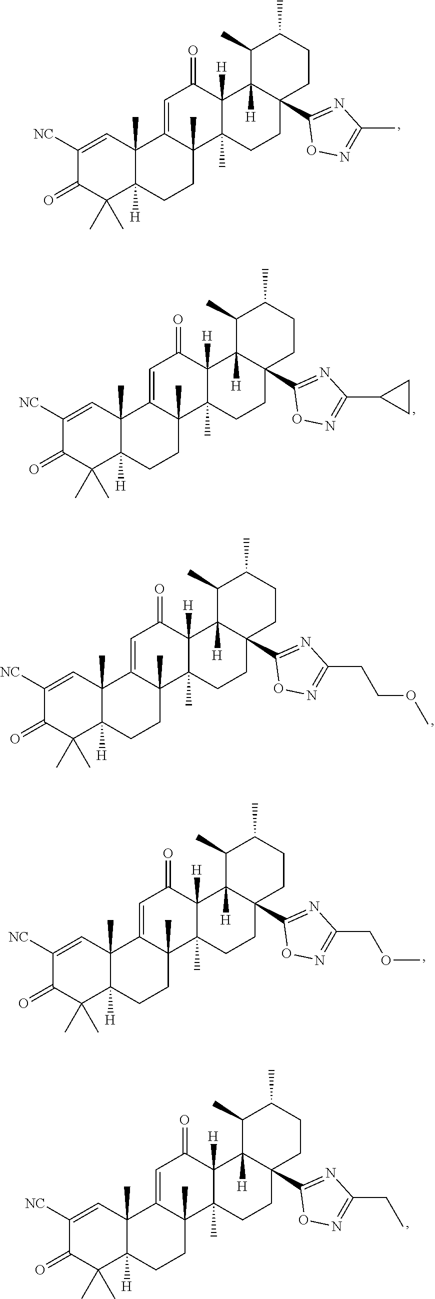

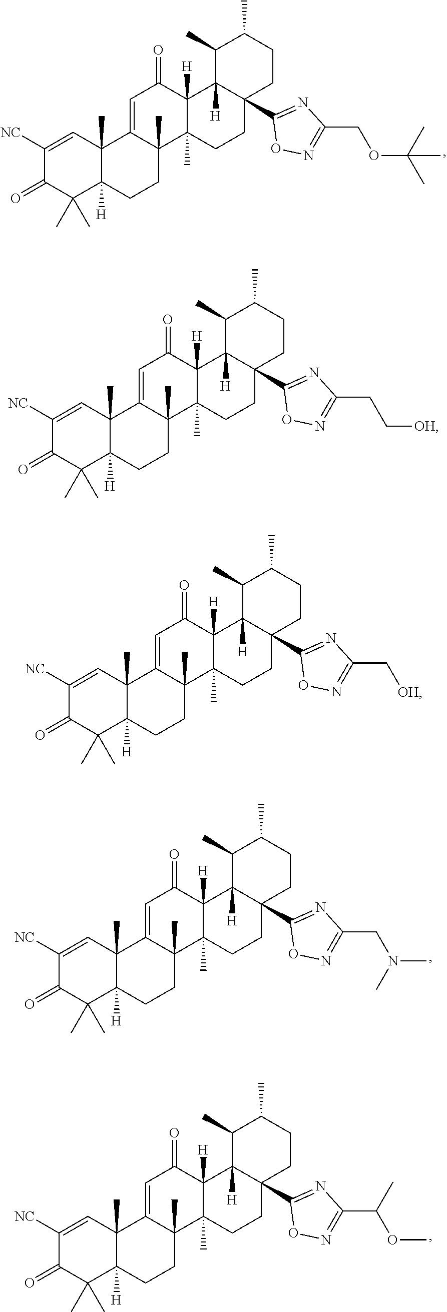

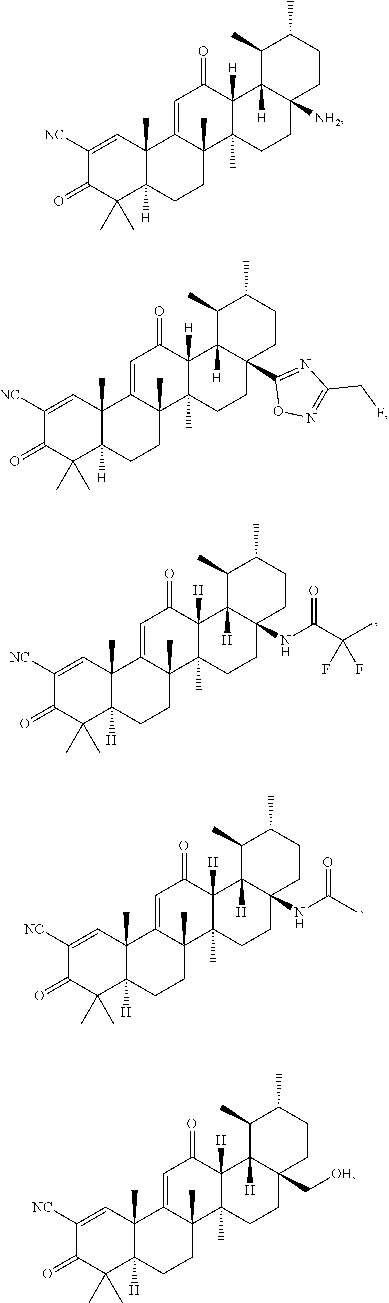

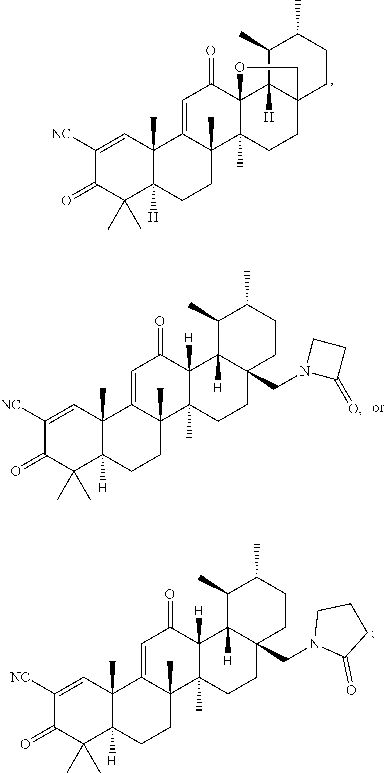

or a pharmaceutically acceptable salt thereof.

In further embodiments, the compound is further defined as:

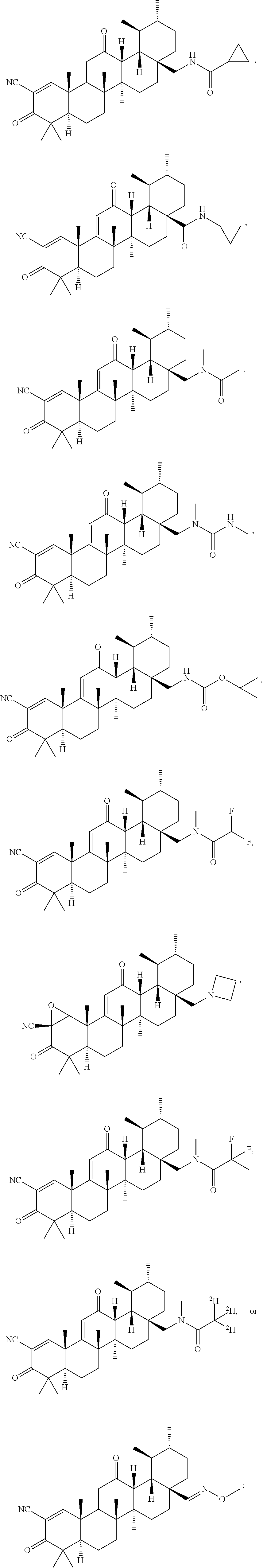

or a pharmaceutically acceptable salt thereof.

In some embodiments, the compound is further defined as:
(4aR,6aS,6bR,8aS,11 IR,12S,12aS,12bR,14bS)-4,4,6a,6b,11,12,14b-heptamiethyl-8a-(3-methyl-1,2,4-oxadiazol-5-yl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-cyclopropyl-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(2-methoxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(methoxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-ethyl-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(tert-butoxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(2-hydroxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(hydroxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-((dimethylamino)methyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(1-methoxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-amino-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(fluoromethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-2,2-difluoropropanamide;
N-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetamide;
(4aR,6aS,6bR,8aS,11R,12S,12aS,12bR,14bS)-8a-(hydroxymethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aR,6bS,11R,12S,12aR,12bS,14bS)-4,4,6a,6b,11,12,14b-heptamethyl-3,13-dioxo-4,4a,5,6,6a,6b,7,8,10,11,12,12a,13,14b-tetradecahydro-3H,9H-12b,8a-(epoxymethano)picene-2-carbonitrile;
(4aR,6aS,6bR,8aS,11R,12S,12aS,12bR,14bS)-4,4,6a,6b,11,12,14b-heptamethyl-3,13-dioxo-8a-((2-oxoazetidin-1-yl)methyl)-3,4,4a,5,6,6a,6b,7,8,8a,9,10,11,12,12a,12b,13,14b-octadecahydropicene-2-carbonitrile; or
(4aR,6aS,6bR,8aS,11R,12S,12aS,12bR,14bS)-4,4,6a,6b,11,12,14b-heptamethyl-3,13-dioxo-8a-((2-oxopyrrolidin-1-yl)methyl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)cyclopropanecarboxamide;
(1S,2R,4aS,6aR,6bS,8aR,12aS,14aR,14bS)-11-cyano-N-cyclopropyl-1,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxamide;
(4aR,6aS,6bR,8aS,11R,12S,12aS,12bR,14bS)-8a-hydroxy-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-fluoro-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-N-methylacetamide;
1-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-1,3-dimethylurea;
tert-butyl (((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)carbamate;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)acetamide;
1-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-3-methylurea;
(4aR,6aS,6bR,8aS,11R,12S,12aS,12bR,14bS)-8a-((1H-imidazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-((1H-tetrazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-((1H-1,2,3-triazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-((2-oxooxazolidin-3-yl)methyl)-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-((1H-pyrazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-((1H-1,2,4-triazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-8a-((3-methyl-2-oxoimidazolidin-1-yl)methyl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(((2-hydroxyethyl)amino)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-8a-(oxazolidin-3-ylmethyl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-8a-((4-methyl-1H-1,2,3-triazol-1-yl)methyl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(azetidin-1-ylmethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)acetamide-2,2,2-d3;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)propionamide;
(4aR,6aS,6bR,8aS,11R,12S,12aS,12bR,14bS)-4,4,6a,6b,11,12,14b-heptamethyl-3,13-dioxo-8a-((5-oxopyrazolidin-1-yl)methyl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-2,2-difluoropropanamide;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-2,2-difluoroacetamide;
(4aR,6aS,6bR,8aS,11R,12S,12aS,12bR,14bS)-4,4,6a,6b,11,12,14b-heptamethyl-3,13-dioxo-8a-((2-oxoimidazolidin-1-yl)methyl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-2,2-difluoro-N-methylacetamide;
2-((((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)amino)ethyl acetate;
(3aR,5aS,5bR,7aS,10R,11S,11aS,11bR,13bS)-7a-(azetidin-1-ylmethyl)-3,3,5a,5b,10,11,13b-heptamethyl-2,12-dioxo-3,3a,4,5,5a,5b,6,7,7a,8,9,10,11,11a,1 b,12,13b,13c-octadecahydropiceno[1,2-b]oxirene-1a(2H)-carbonitrile;
methyl (((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)carbamate;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-2,2-difluoro-N-methylpropanamide;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-N-methylacetamide-d3;
(4aR,6aR,6bS,8aS,11R,12S,12aR,14bS)-8a-((1H-1,2,4-triazol-1-yl)methyl)-12b-hydroxy-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
methyl 2-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-3-oxopyrazolidine-1-carboxylate;
(4aR,6aS,6bR,8aS,11R,12S,12aS,12bR,14bS)-8a-((E)-(methoxyimino)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(1S,2R,4aS,6aR,6bS,8aR,12aS,14aR,14bS)-11-cyano-N-methoxy-1,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxamide;
(1S,2R,4aS,6aR,6bS,8aR,12aS,14aR,14bS)-11-cyano-N-(2,2-difluoroethyl)-1,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxamide;
(4aR,6aR,6bR,8aS,11R,12S,12aS,12bR,13R,14bR)-4,4,6a,6b,11,12,14b-heptamethyl-3,16-dioxo-4,4a,5,6,6a,6b,7,8,9,10,11,12,12a,12b,13,14,14a,14b-octadecahydro-3H-13,8a-(epoxymethano)picene-2-carbonitrile; 1-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-3-ethylurea;
(4aR,6aS,6bR,8aS,11R,12S,12aS,12bR,14bS)-4,4,6a,6b,11,12,14b-heptamethyl-3,13-dioxo-8a-(1H-1,2,3-triazol-1-yl)-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-(2-oxopyrrolidin-¹-yl)-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-(1H-tetrazol-1-yl)-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(1H-imidazol-1-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-(2-oxooxazolidin-3-yl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)cyclobutanecarboxamide;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)azetidine-1-carboxamide;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)methanesulfonamide;
(4aR,6aS,6bR,8aS,11R,12S,12aS,12bR,14bS)-8a-(4-bromo-1H-pyrazol-1-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-(1H-pyrazol-1-yl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)ethanesulfonamide;
(4aR,6aS,6bR,8aS,11R,12S,12aS,12bR,14bS)-8a-((2,5-dioxopyrrolidin-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile; N-((1aR,3aR,5aS,5bR,7aS,10R,11S,11aS,11bR,13bS)-1a-cyano-3,3,5a,5b,10,11,13b-heptamethyl-2,12-dioxo-1a,3,3a,4,5,5a,5b,6,7,8,9,10,11,11a,11b,12,13b,13c-octadecahydropiceno[1,2-b]oxiren-7a(2H)-yl)-2,2-difluoropropanamide;
methyl ((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)carbamate;
(4aR,6aS,6bR,8aS,11R,12S,12aS,12bR,14bS)-8a-(aminomethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)cyclopropanesulfonamide;
(4aR,6aS,6bR,8aS,11R,12S,12aS,12bR,14bS)-4,4,6a,6b,11,12,14b-heptamethyl-3,13-dioxo-8a-(2-oxoazetidin-1-yl)-3,4,4a,5,6,6a,6b,7,8,8a,9,10,11,12,12a,12b,13,14b-octadecahydropicene-2-carbonitrile;
N-(((1aR,3aR,5aS,5bR,7aS,10R,11S,11aS,11bR,13bS)-1a-cyano-3,3,5a,5b,10,11,13b-heptamethyl-2,12-dioxo-1a,3,3a,4,5,5a,5b,6,7,8,9,10,11,11a,11b,12,13b,13c-octadecahydropiceno[1,2-b]oxiren-7a(2H)-yl)methyl)cyclopropanecarboxamide;
N-(((1S,2R,4aS,6aR,6bS,8aR,12aS,14aR,14bS)-11-cyano-1,2,6a,6b,9,9,12a-heptamethyl-10,14-dioxo-1,2,3,4,4a,5,6,6a,6b,7,8,8a,9,10,12a,14,14a,14b-octadecahydropicene-4a-carbonyl)oxy)acetimidamide;
(4aR,6aS,6bR,8aS,11R,12S,12aS,12bR,14bS)-4,4,6a,6b,11,12,14b-heptamethyl-8a-(5-methyl-1,3,4-oxadiazol-2-yl)-3,13-dioxo-3,4,4a,5,6,6a,6b,7,8,8a,9,10,11,12,12a,12b,13,14b-octadecahydropicene-2-carbonitrile;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)propanoic acid;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-methylpropanamide;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-ethylpropanamide;
(4aR,6aS,6bR,8aR,11R,12S,12aS,12bR,14bS)-8a-(3-(azetidine-¹-yl)-³-oxopropyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile; (1aR,3aR,5aS,5bR,7aS,10R,11S,11aS,11bR,13bS)-3,3,5a,5b,10,11,13b-heptamethyl-7a-(3-methyl-1,2,4-oxadiazol-5-yl)-2,12-dioxo-3,3a,4,5,5a,5b,6,7,7a,8,9,10,11,11a,11b,12,13b,13c-octadecahydropiceno[1,2-b]oxirene-1a(2H)-carbonitrile;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-cyclopropylpropanamide;
(4aR,6aS,6bR,8aS,11R,12S,12aS,12bR,14bS)-8a-(azetidine-1-carbonyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-(pyrrolidine-1-carbonyl)-3,4,4a,5,6,6a,6b,7,8,8a,9,10,11,12,12a,12b,13,14b-octadecahydropicene-2-carbonitrile;
(1S,2R,4aS,6aR,6bS,8aR,12aS,14aR,14bS)-11-cyano-N-hydroxy-1,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxamide;
(4aR,6aS,6bR,8aS,11R,12S,12aS,12bR,14bS)-4,4,6a,6b,11,12,14b-heptamethyl-8a-(5-methyl-1,2,4-oxadiazol-3-yl)-3,13-dioxo-3,4,4a,5,6,6a,6b,7,8,8a,9,10,11,12,12a,12b,13,14b-octadecahydropicene-2-carbonitrile;
tert-butyl (((1S,2R,4aS,6aR,6bR,8aR,12aR,14aR,14bS)-11-cyano-1,2,6a,6b,9,9,12a-heptamethyl-10,14-dioxo-1,3,4,5,6,6a,6b,7,8,8a,9,10,12a,12b,13,14,14a,14b-octadecahydropicen-4a(2H)-yl)methyl)carbamate;
3-((1S,2R,4aR,6aR,6bR,8aR,12aR,14aR,14bS)-11-cyano-1,2,6a,6b,9,9,12a-heptamethyl-10,14-dioxo-1,3,4,5,6,6a,6b,7,8,8a,9,10,12a,12b,13,14,14a,14b-octadecahydropicen-4a(2H)-yl)-N-ethylpropanamide;
3-((1S,2R,4aR,6aR,6bR,8aR,12aR,14aR,14bS)-11-cyano-1,2,6a,6b,9,9,12a-heptamethyl-10,14-dioxo-1,3,4,5,6,6a,6b,7,8,8a,9,10,12a,12b,13,14,14a,14b-octadecahydropicen-4a(2H)-yl)-N-methylpropanamide;
(4aR,6aR,6bR,8aR,11R,12S,12aS,12bR,14bR)-8a-(3-(azetidine-1-yl)-3-oxopropyl)-4,4,6a,6b,11,12,14b-heptamethyl-3,13-dioxo-3,4,4a,5,6,6a,6b,7,8,8a,9,10,11,12,12a,12b,13,14,14a,14b-icosahydropicene-2-carbonitrile;
(4aR,6aS,6bR,8aS,11R,12S,12aS,12bR,14bS)-4,4,6a,6b,11,12,14b-heptamethyl-8a-((methylamino)methyl)-3,13-dioxo-3,4,4a,5,6,6a,6b,7,8,8a,9,10,11,12,12a,12b,13,14b-octadecahydropicene-2-carbonitrile;
methyl (1S,2R,4aS,6aR,6bR,8aR,12aR,14aR,14bS)-11-cyano-1,2,6a,6b,9,9,12a-heptamethyl-10,14-dioxo-1,3,4,5,6,6a,6b,7,8,8a,9,10,12a,12b,13,14,14a,14b-octadecahydropicene-4a(2H)-carboxylate;
methyl 2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetate;
2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetic acid;
2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-cyclopropylacetamide;
(4aR,6aS,6bR,8aS,11R,12S,12aS,12bR,14bS)-8a-formyl-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
methyl 3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)propanoate;
2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetamide;
(4aR,6aS,6bR,8aR,11R,12S,12aS,12bR,14bS)-8a-(2-(azetidine-1-yl)-2-oxoethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-methylacetamide;
(4aR,6aS,6bR,8aS,11R,12S,12aS,12bR,14bS)-8a-((E)-(hydroxyimino)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile
2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-ethylacetamide
(4aR,6aS,6bR,8aR,11R,12S,12aS,12bR,14bS)-4,4,6a,6b,11,12,14b-heptamethyl-8a-((5-methyl-1,3,4-oxadiazol-2-yl)methyl)-3,13-dioxo-3,4,4a,5,6,6a,6b,7,8,8a,9,10,11,12,12a,12b,13,14b-octadecahydropicene-2-carbonitrile
(4aR,6aS,6bR,8aR,11R,12S,12aS,12bR,14bS)-4,4,6a,6b,11,12,14b-heptamethyl-8a-((3-methyl-1,2,4-oxadiazol-5-yl)methyl)-3,13-dioxo-3,4,4a,5,6,6a,6b,7,8,8a,9,10,11,12,12a,12b,13,14b-octadecahydropicene-2-carbonitrile
(4aR,6aS,6bR,8aR,11R,12S,12aS,12bR,14bS)-8a-(2-hydroxyethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile
   or a pharmaceutically acceptable salt thereof.
      or a pharmaceutically acceptable salt thereof.

In some embodiments, the compound is further defined as:
(4aR,6aS,6bR,8aS,11R,12S,12aS,12bR,14bS)-4,4,6a,6b,11,12,14b-heptamethyl-8a-(3-methyl-1,2,4-oxadiazol-5-yl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-cyclopropyl-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(2-methoxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(methoxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-ethyl-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(tert-butoxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(2-hydroxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(hydroxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-((dimethylamino)methyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(1-methoxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-amino-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(fluoromethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-2,2-difluoropropanamide;
N-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetamide;
(4aR,6aS,6bR,8aS,11R,12S,12aS,12bR,14bS)-8a-(hydroxymethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-((2-oxoazetidin-1-yl)methyl)-3,4,4a,5,6,6a,6b,7,8,8a,9,10,11,12,12a,12b,13,14b-octadecahydropicene-2-carbonitrile; or
(4aR,6aS,6bR,8aS,11R,12S,12aS,12bR,14bS)-4,4,6a,6b,11,12,14b-heptamethyl-3,13-dioxo-8a-((2-oxopyrrolidin-1-yl)methyl)-3,4,4a,5,6,6a,6b,7,8,8a,9,10,11,12,12a,12b,13,14b-octadecahydropicene-2-carbonitrile;
or a pharmaceutically acceptable salt thereof.

In one embodiment, the compound is further defined as:

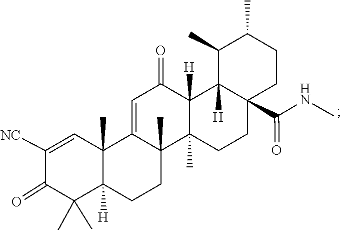

or a pharmaceutically acceptable salt thereof.

In a further embodiment, the compound is further defined as:
(1S,2R,4aS,6aR,6bS,8aR,12aS,14aR,14bS)-11-cyano-N,1,2,6a,6b,9,9,12a-octamethyl-10,14-dioxo-1,3,4,5,6,6a,6b,7,8,8a,9,10,12a,14,14a,14b-hexadecahydropicene-4a(2H)-carboxamide
or a pharmaceutically acceptable salt thereof.

In another aspect, the present disclosure provides pharmaceutical compositions comprising:
(A) a compound of the present disclosure; and
(B) an excipient.

In some embodiments, the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularily, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the pharmaceutical composition is formulated for oral administration. In some embodiments, the pharmaceutical composition is formulated for administration via injection. In some embodiments, the pharmaceutical composition is formulated for intraarterial administration, intramuscular administration, intraperitoneal administration, or intravenous administration. In some embodiments, the pharmaceutical composition is formulated for administration topically. In some embodiments, the pharmaceutical composition is formulated for topical administration to the skin or to the eye. In some embodiments, the pharmaceutical composition is formulated as a unit dose.

In still another aspect, the present disclosure provides methods of treating or preventing a disease or disorder in a patient in need thereof comprising administering to the patient a pharmaceutically effective amount of a compound or composition of the present disclosure. In some embodiments, the patient is a mammal, such as a human. In some embodiments, the disease or disorder is a condition associated with inflammation and/or oxidative stress. In some embodiments, the disease or disorder is cancer. In some embodiments, the disease or disorder is a cardiovascular disease, such as atherosclerosis. In some embodiments, the disease or disorder is an autoimmune disease, such as Crohn's disease, rheumatoid arthritis, lupus, or psoriasis. In some embodiments, the disease or disorder is a neurodegenerative disease, such as Alzheimer's disease, Parkinson's disease, amyotrophic lateral sclerosis, or Huntington's disease. In some embodiments, disease or disorder is chronic kidney disease, diabetes, mucositis, inflammatory bowel disease, dermatitis, sepsis, ischemia-reperfusion injury (including complications from sickle cell anemia), influenza, osteoarthritis, osteoporosis, pancreatitis, asthma, chronic obstructive pulmonary disease, cystic fibrosis, idiopathic pulmonary fibrosis, multiple sclerosis, muscular dystrophy, cachexia, or graft-versus-host disease. In some embodiments, the disease or disorder is an eye disease, such as uveitis, glaucoma, macular degeneration, or retinopathy. In some embodiments, the disease or disorder is neuropsychiatric, such as schizophrenia, depression, bipolar disorder, epilepsy, post-traumatic stress disorder, attention deficit disorder, autism, or anorexia nervosa. In some embodiments, the disease or disorder is associated with mitochondrial dysfunction, such as Friedreich's ataxia. In some embodiments, the disease or disorder is chronic pain. In some embodiments, the disease or disorder is neuropathic pain.

In another aspect, the present disclosure provides methods of inhibiting nitric oxide production comprising administering to a patient in need thereof an amount of a compound or composition of the present disclosure sufficient to cause inhibition of IFN-γ-induced nitric oxide production in one or more cells of the patient.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### DESCRIPTION OF ILLUSTRATIVE EMBODIMENTS

Disclosed herein are new compounds and compositions with antioxidant and/or anti-inflammatory properties, methods for their manufacture, and methods for their use, including for the treatment and/or prevention of disease.

### I. Compounds of the Present Invention

The compounds of the present invention (also referred to as “synthetic triterpenoid derivatives,” “compounds of the present disclosure” or “compounds disclosed herein”) are shown, for example, above, in the summary of the invention section, in the table below, in the Examples section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith. March 's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

TABLE 1
Examples of Synthetic Ursolic Acid Derivatives Provided Herein

| ID | Structural Formula |
|---|---|
| T1 | 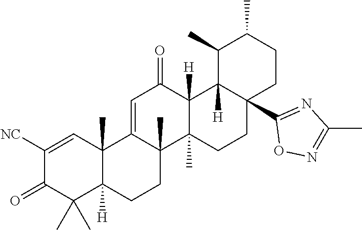 |
| T2 | 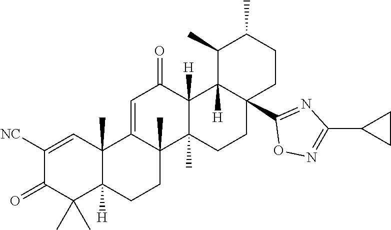 |
| T3 | 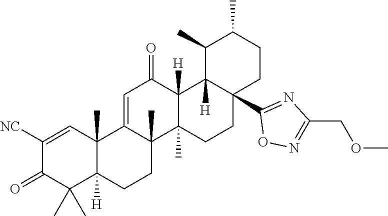 |
| T4 | 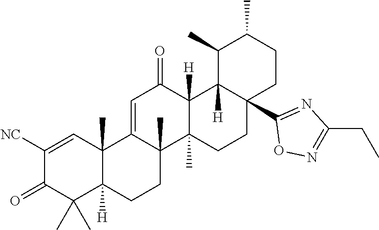 |
| T5 | 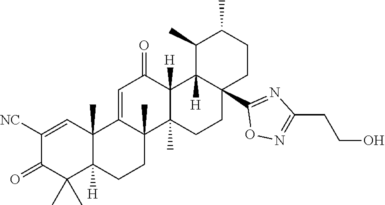 |
| T6 | 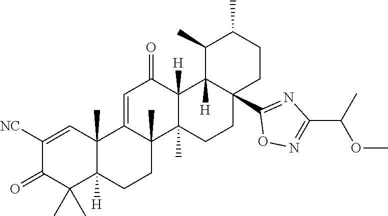 |
| T7 | 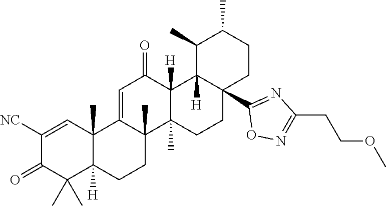 |
| T8 | 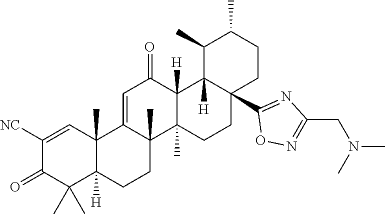 |
| T9 | 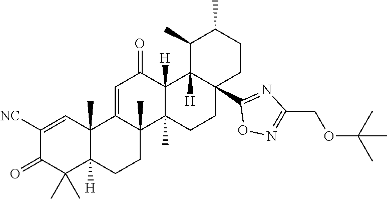 |
| T10 | 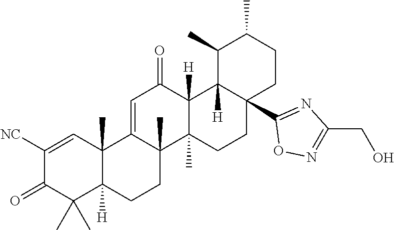 |
| T11 | 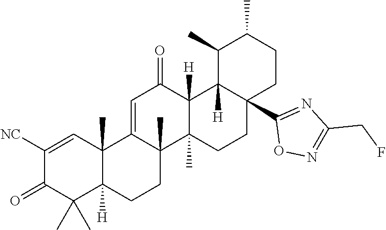 |
| T12 | 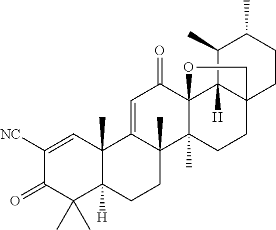 |
| T13 | 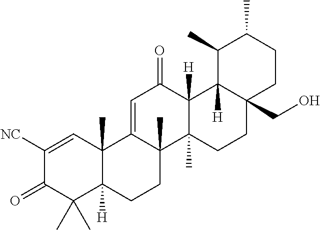 |
| T14 | 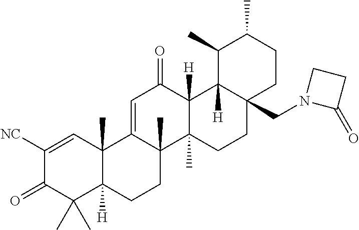 |
| T15 | 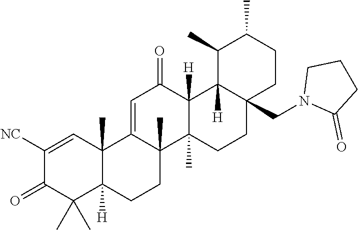 |
| T16 | 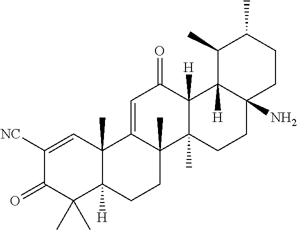 |
| T17 | 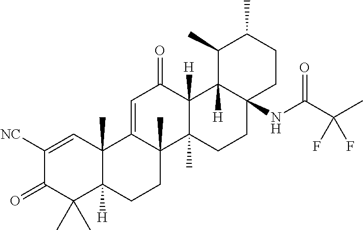 |
| T18 | 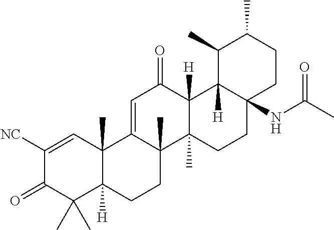 |
| T19 | 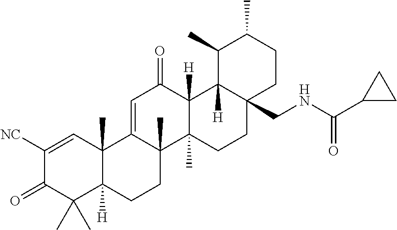 |
| T20 | 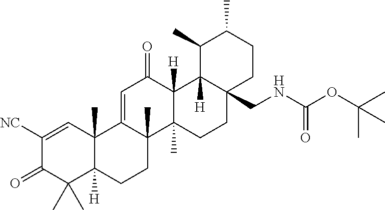 |
| T21 | 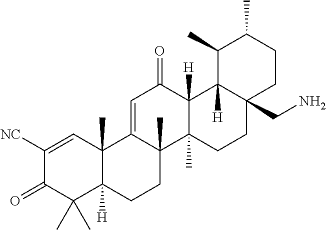 |
| T22 | 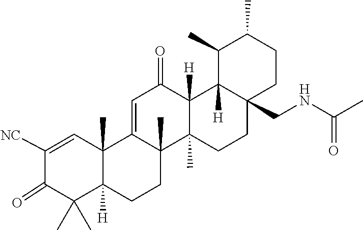 |
| T23 | 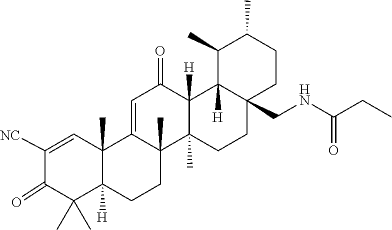 |
| T24 | 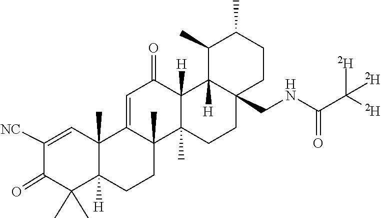 |
| T25 | 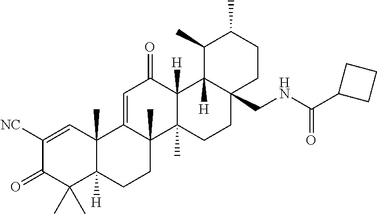 |
| T26 | 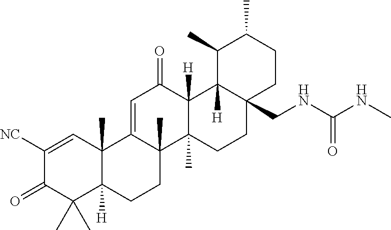 |
| T27 | 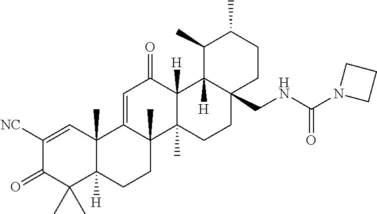 |
| T28 | 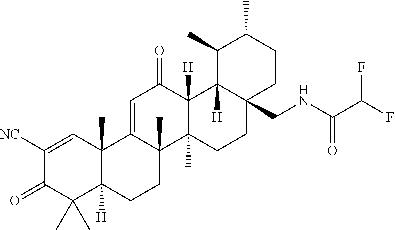 |
| T29 | 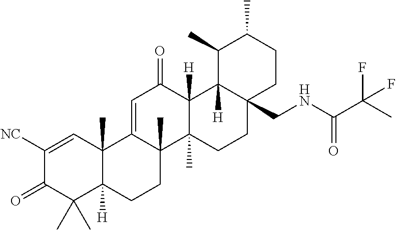 |
| T30 | 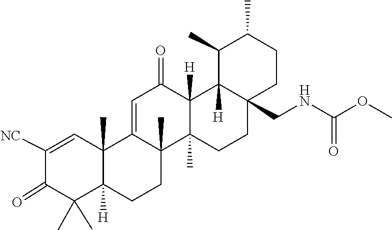 |
| T31 | 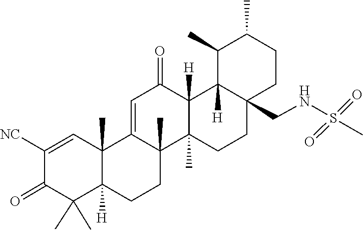 |
| T32 | 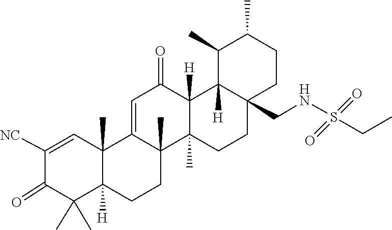 |
| T33 | 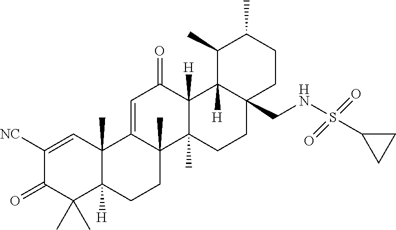 |
| T34 | 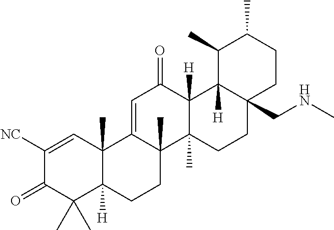 |
| T35 | 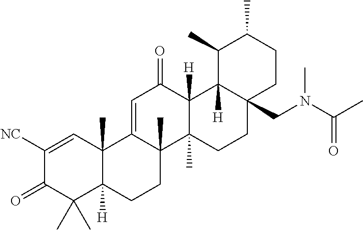 |
| T36 | 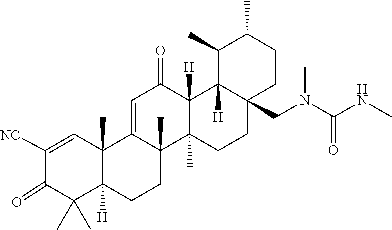 |
| T37 | 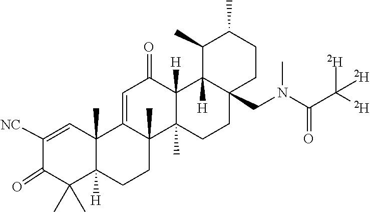 |
| T38 | 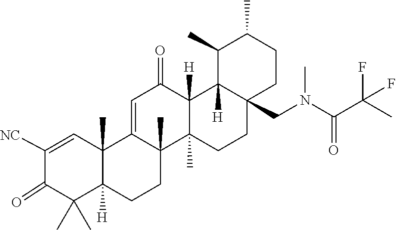 |
| T39 | 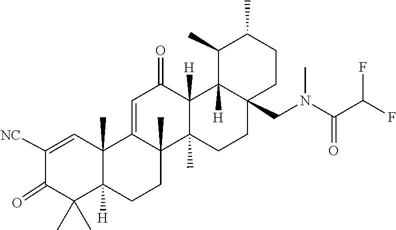 |
| T40 | 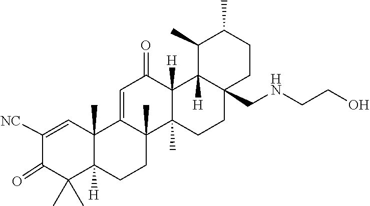 |
| T41 | 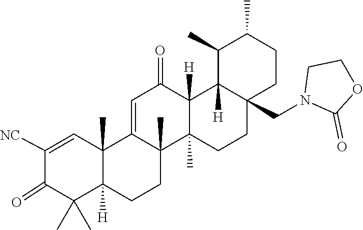 |
| T42 | 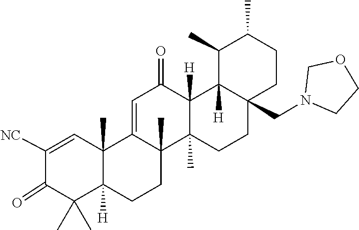 |
| T43 | 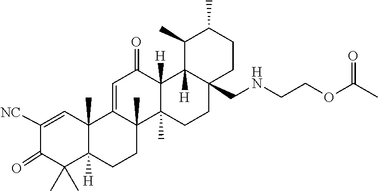 |
| T44 | 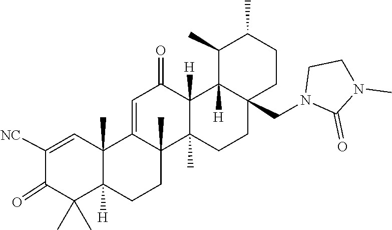 |
| T45 | 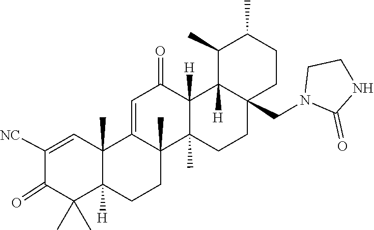 |
| T46 | 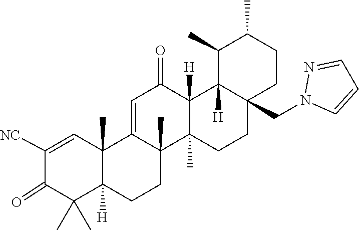 |
| T47 | 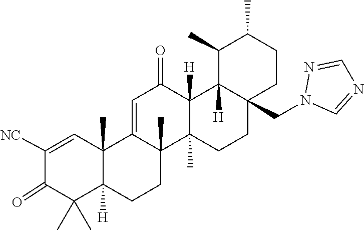 |
| T48 | 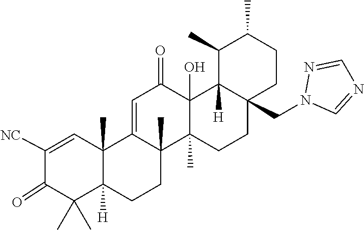 |
| T49 | 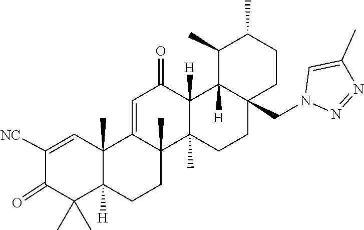 |
| T50 | 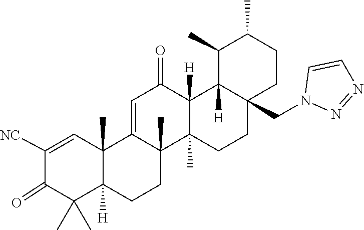 |
| T51 | 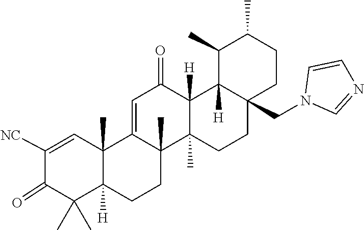 |
| T52 | 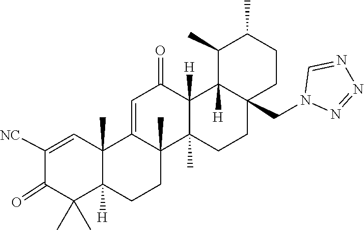 |
| T53 | 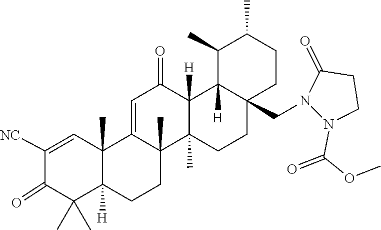 |
| T54 | 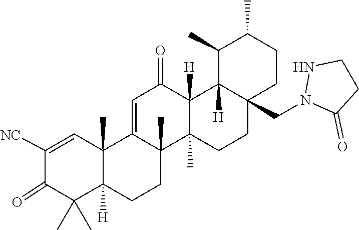 |
| T55 | 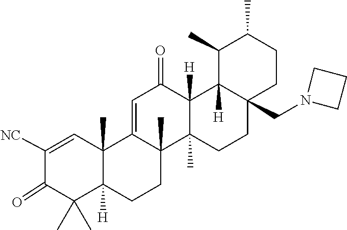 |
| T56 | 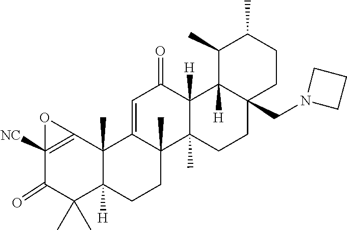 |
| T57 | 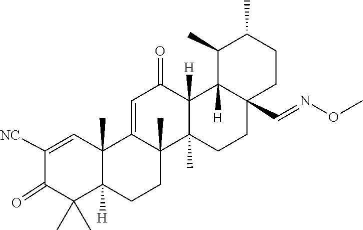 |
| T58 | 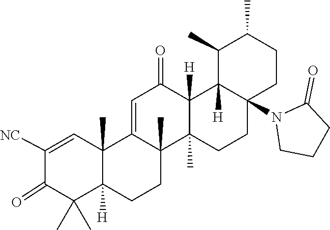 |
| T59 | 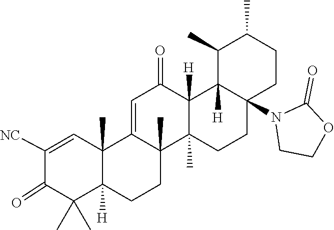 |
| T60 | 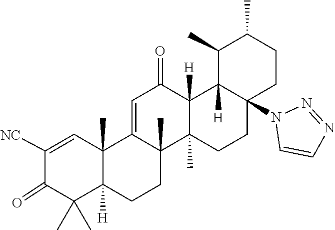 |
| T61 | 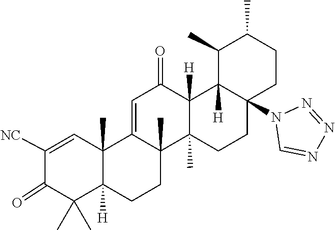 |
| T62 | 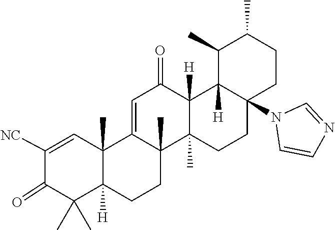 |
| T63 | 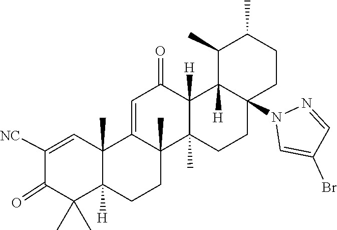 |
| T64 | 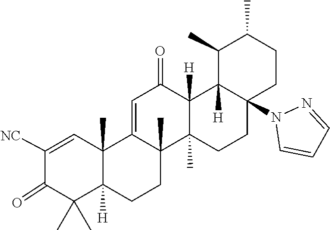 |
| T65 | 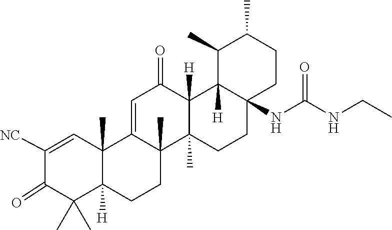 |
| T66 | 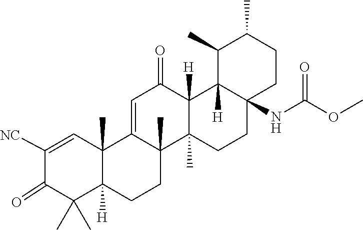 |
| T67 | 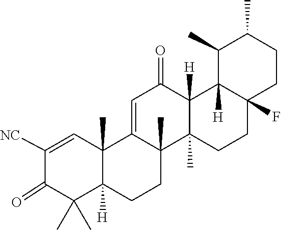 |
| T68 | 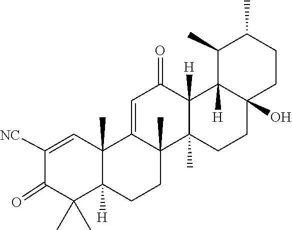 |
| T69 | 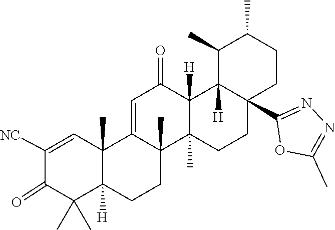 |
| T70 | 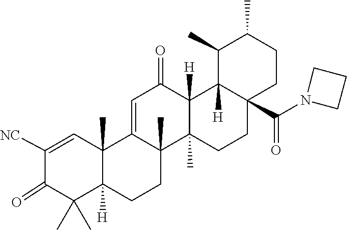 |
| T71 | 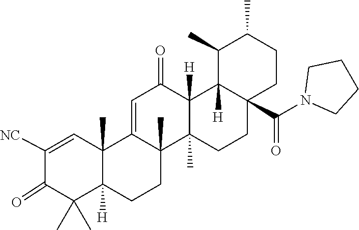 |
| T72 | 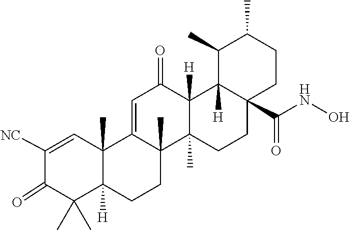 |
| T73 | 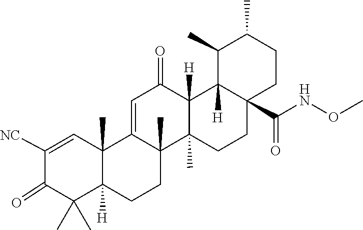 |
| T74 | 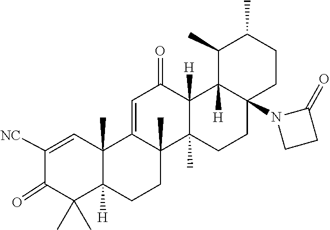 |
| T75 | 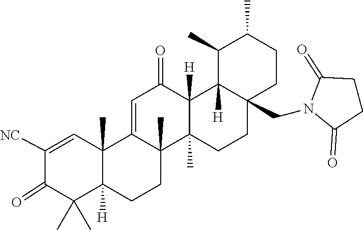 |
| T76 | 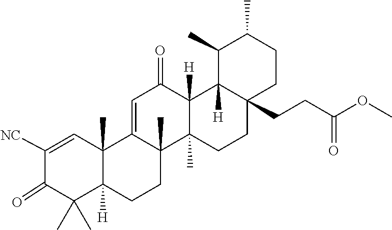 |
| T77 | 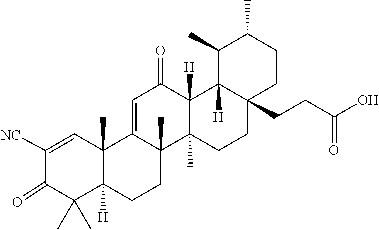 |
| T78 | 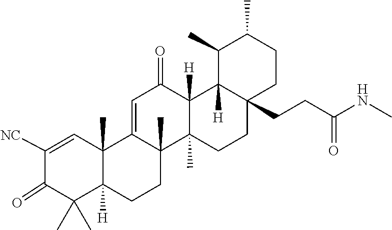 |
| T79 | 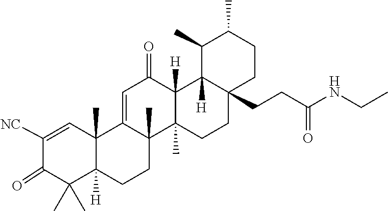 |
| T80 | 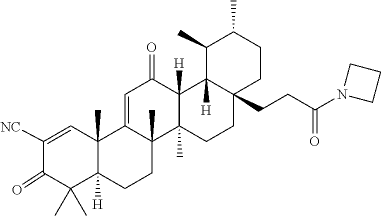 |
| T81 | 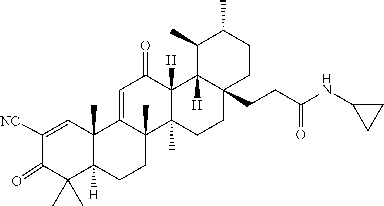 |
| T82 | 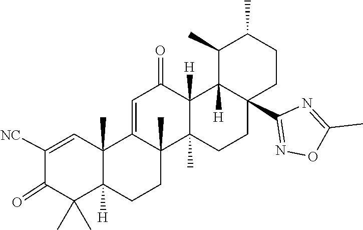 |
| T83 | 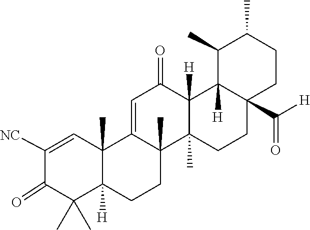 |
| T84 | 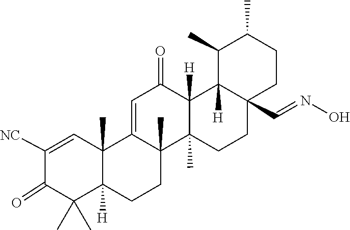 |
| T85 | 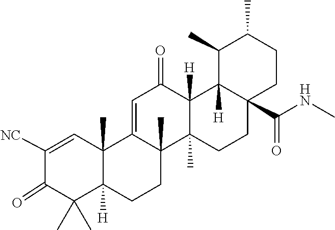 |
| T86 | 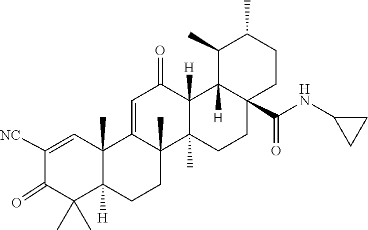 |
| T87 | 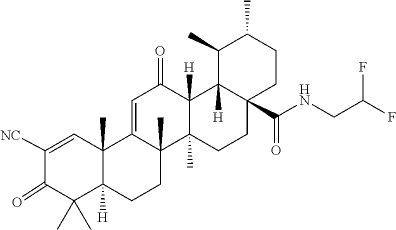 |
| T88 | 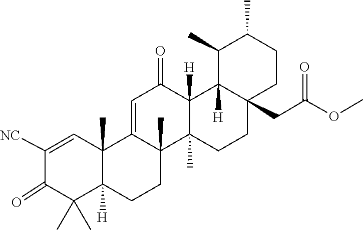 |
| T89 | 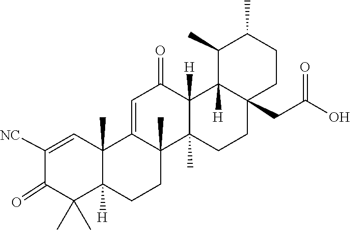 |
| T90 | 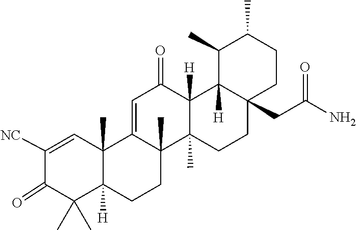 |
| T91 | 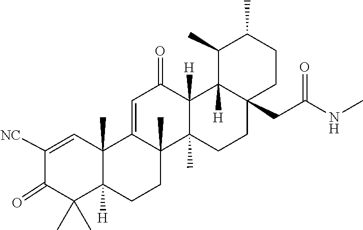 |
| T92 | 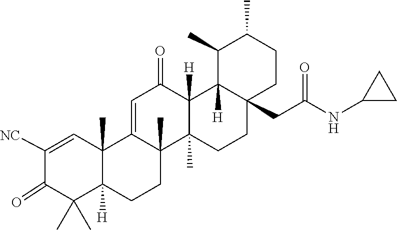 |
| T93 | 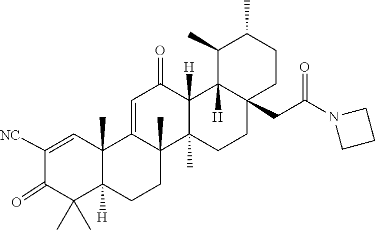 |
| T94 | 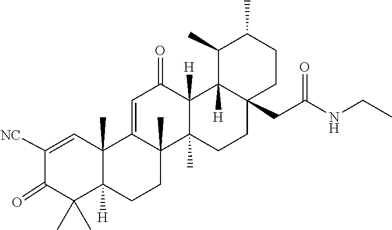 |
| T95 | 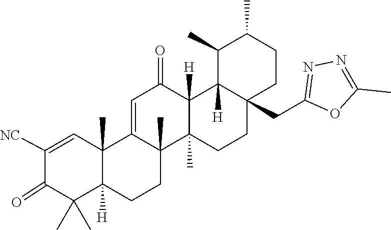 |
| T96 | 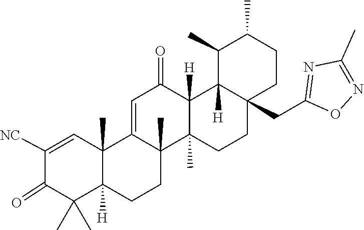 |
| T97 | 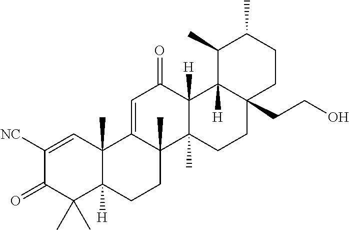 |
| T98 | 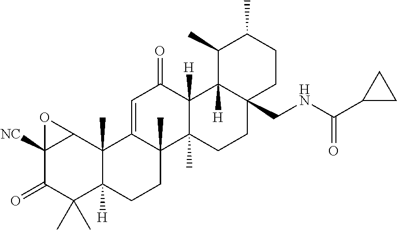 |
| T99 | 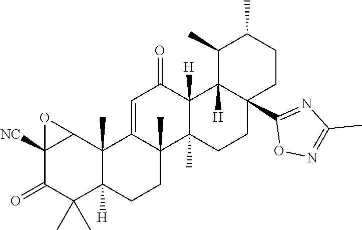 |
| T100 | 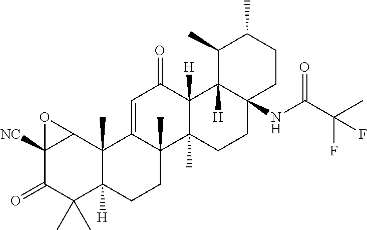 |
| T101 | 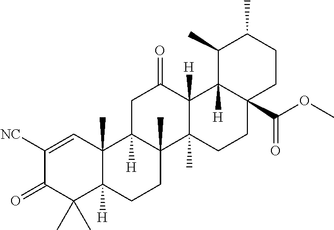 |
| T102 | 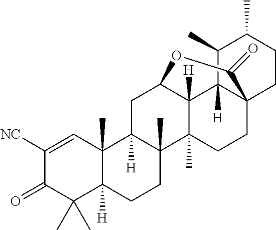 |
| T103 | 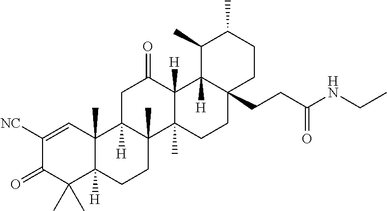 |
| T104 | 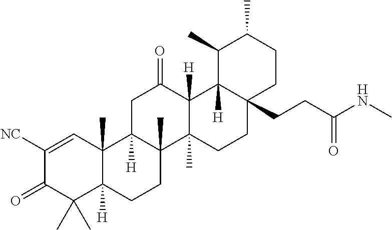 |
| T105 | 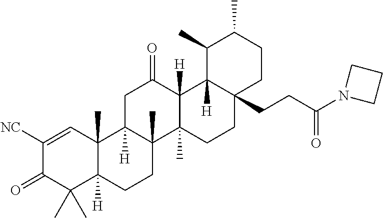 |
| T106 | 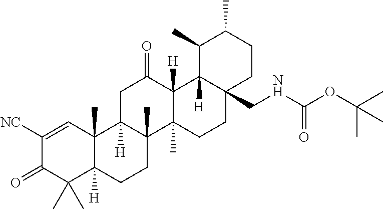 |
| T107 | 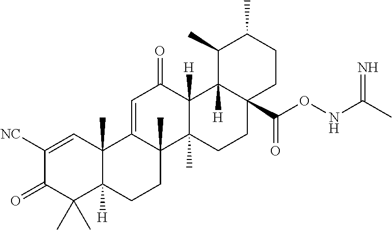 |

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may also be useful for the prevention and treatment of one or more diseases or disorders. Unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present invention have the advantage whether when compared in vivo, ex vivo, and/or in vitro, that they may be more efficacious than, be less toxic than, be longer acting than, be more potent than, produce fewer side effects than, be more easily absorbed than, be more metabolically stable than, be more lipophilic than, be more hydrophilic than, have better pharmacodynamics, and/or have a better pharmacokinetic profile (e.g., higher oral bioavailability and/or lower clearance) than compounds known in the prior art, whether for use in the indications discussed herein or otherwise. In some embodiments, the compounds of the present invention have the advantage that they have useful pharmacological, physical, and/or chemical properties over prior art compounds.

Compounds of the present invention may contain one or more asymmetrically substituted carbon, sulfur, or phosphorus atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### II. Biological Activity

The aberrant or excessive expression of iNOS has been implicated in the pathogenesis of many disease processes. For example, it is clear that NO is a potent mutagen (Tamir and Tannebaum, 1996), and that nitric oxide can also activate COX-2 (Salvemini et al., 1994). Furthermore, there is a marked increase in iNOS in rat colon tumors induced by the carcinogen, azoxymethane (Takahashi et al., 1997). A series of synthetic triterpenoid analogs of oleanolic acid have been shown to be powerful inhibitors of cellular inflammatory processes, such as the induction by IFN-γ of inducible nitric oxide synthase (iNOS) and of COX-2 in mouse macrophages. See Honda et al. (2000a); Honda et al. (2000b), and Honda et al. (2002), which are all incorporated herein by reference. Assay results for the suppression of IFNγ-induced NO production are shown for several of the compounds of the present invention in Tables 2 and 3 in Example 2. Details regarding this assay are also provided in the Example 2.In some embodiments, compounds disclosed herein are characterized by their ability to inhibit the production of nitric oxide in macrophage-derived RAW 264.7 cells induced by exposure to γ-interferon.

Nrf2 is a transcription factor that regulates cytoprotective genes that contain an antioxidant response element (ARE) in their promoters (Wu et al, 2006). Measurement of ARE-dependent luciferase activity allows quantitative assessment of Nrf2 induction. The AREc32 reported cell line has previously been used in studies characterizing different Nrf2 activators (Dinkova-Kostova & Wang, 2011; Roubalová et al., 2016; Roubalová et al, 2017; Wu et al, 2012). Assay results for the suppression of ARE-dependent luciferase activity are shown for several of the compounds of the present invention in Tables 2 and 3 in Example 2. Details regarding this assay are also provided in Example 2. In some embodiments, the compounds of the present invention may be used to activate the antioxidant/anti-inflammatory Nrf2 pathway(s) in cells, tissues, and patients in need thereof.

III. Diseases Associated with Inflammation and/or Oxidative Stress

Inflammation is a biological process that provides resistance to infectious or parasitic organisms and the repair of damaged tissue. Inflammation is commonly characterized by localized vasodilation, redness, swelling, and pain, the recruitment of leukocytes to the site of infection or injury, production of inflammatory cytokines such as TNF-α and IL-1, and production of reactive oxygen or nitrogen species such as hydrogen peroxide, superoxide and peroxynitrite. In later stages of inflammation, tissue remodeling, angiogenesis, and scar formation (fibrosis) may occur as part of the wound healing process. Under normal circumstances, the inflammatory response is regulated and temporary and is resolved in an orchestrated fashion once the infection or injury has been dealt with adequately. However, acute inflammation can become excessive and life-threatening if regulatory mechanisms fail. Alternatively, inflammation can become chronic and cause cumulative tissue damage or systemic complications. Based at least on the evidence presented herein, the compounds of this invention may be used in the treatment or prevention of inflammation or diseases or disorders associated with inflammation or oxidative stress.

Many serious and intractable human diseases involve dysregulation of inflammatory processes, including diseases such as cancer, atherosclerosis, and diabetes, which were not traditionally viewed as inflammatory conditions. In the case of cancer, the inflammatory processes are associated with tumor formation, progression, metastasis, and resistance to therapy. Atherosclerosis, long viewed as a disorder of lipid metabolism, is now understood to be primarily an inflammatory condition, with activated macrophages playing an important role in the formation and eventual rupture of atherosclerotic plaques. Activation of inflammatory signaling pathways has also been shown to play a role in the development of insulin resistance, as well as in the peripheral tissue damage associated with diabetic hyperglycemia. Excessive production of reactive oxygen species and reactive nitrogen species such as superoxide, hydrogen peroxide, nitric oxide, and peroxynitrite is a hallmark of inflammatory conditions. Evidence of dysregulated peroxynitrite production has been reported in a wide variety of diseases (Szabo et al., 2007; Schulz et al., 2008; Forstermann, 2006; Pall, 2007). In some embodiments, the compounds of the present invention may be used to reduce the excessive production of reactive oxygen species.

Autoimmune diseases such as rheumatoid arthritis, lupus, psoriasis, and multiple sclerosis involve inappropriate and chronic activation of inflammatory processes in affected tissues, arising from dysfunction of self vs. non-self recognition and response mechanisms in the immune system. In neurodegenerative diseases such as Alzheimer's and Parkinson's diseases, neural damage is correlated with activation of microglia and elevated levels of pro-inflammatory proteins such as inducible nitric oxide synthase (iNOS). In some embodiments, the compounds of the present invention may be used to reduce or prevent elevated levels of pro-inflammatory proteins. In some embodiments, the compounds of the present invention may be used to treat one or more autoimmune disease in patients in need thereof.

Chronic organ failure such as renal failure, heart failure, liver failure, and chronic obstructive pulmonary disease is closely associated with the presence of chronic oxidative stress and inflammation, leading to the development of fibrosis and eventual loss of organ function. Oxidative stress in vascular endothelial cells, which line major and minor blood vessels, can lead to endothelial dysfunction, and is believed to be an important contributing factor in the development of systemic cardiovascular disease, complications of diabetes, chronic kidney disease and other forms of organ failure, and a number of other aging-related diseases including degenerative diseases of the central nervous system and the retina. In some embodiments, the compounds of the present invention may be used to reduce or prevent oxidative stress and/or inflammation. In some embodiments, the compounds of the present invention may be used to treat or prevent chronic organic failure in patients in need thereof.

Many other disorders involve oxidative stress and inflammation in affected tissues, including inflammatory bowel disease; inflammatory skin diseases; mucositis related to radiation therapy and chemotherapy; eye diseases such as uveitis, glaucoma, macular degeneration, and various forms of retinopathy; transplant failure and rejection; ischemia-reperfusion injury; chronic pain; degenerative conditions of the bones and joints including osteoarthritis and osteoporosis; asthma and cystic fibrosis; seizure disorders; and neuropsychiatric conditions including schizophrenia, depression, bipolar disorder, post-traumatic stress disorder, attention deficit disorders, autism-spectrum disorders, and eating disorders such as anorexia nervosa. Dysregulation of inflammatory signaling pathways is believed to be a major factor in the pathology of muscle wasting diseases including muscular dystrophy and various forms of cachexia. In some embodiments, the compounds of the present invention may be used to treat or prevent disorders involving oxidative stress and inflammation in affected tissues in patients in need thereof.

A variety of life-threatening acute disorders also involve dysregulated inflammatory signaling, including acute organ failure involving the pancreas, kidneys, liver, or lungs, myocardial infarction or acute coronary syndrome, stroke, septic shock, trauma, severe burns, and anaphylaxis. In some embodiments, the compounds of the present invention may be used to treat or prevent dysregulated inflammatory signaling in patients in need thereof.

Many complications of infectious diseases also involve dysregulation of inflammatory responses. Although an inflammatory response can kill invading pathogens, an excessive inflammatory response can also be quite destructive and in some cases can be a primary source of damage in infected tissues. In some embodiments, the compounds of the present invention may be used to treat or prevent infectious diseases also involve dysregulation of inflammatory responses in patients in need thereof. Furthermore, an excessive inflammatory response can also lead to systemic complications due to overproduction of inflammatory cytokines such as TNF-α and IL-1. This is believed to be a factor in mortality arising from severe influenza, severe acute respiratory syndrome, and sepsis. In some embodiments, the compounds of the present invention may be used to reduce or prevent the overproduction of inflammatory cytokines in patients in need thereof.

In one aspect, compounds disclosed herein are characterized by their ability to inhibit the production of nitric oxide in macrophage-derived RAW 264.7 cells induced by exposure to y-interferon. In some embodiments, the compounds of the present invention are characterized by their ability to induce the expression of antioxidant proteins such as NQO1 and reduce the expression of pro-inflammatory proteins such as COX-2 and inducible nitric oxide synthase (iNOS). These properties are relevant to the treatment of a wide array of diseases and disorders involving oxidative stress and dysregulation of inflammatory processes including cancer, complications from localized or total-body exposure to ionizing radiation, mucositis resulting from radiation therapy or chemotherapy, autoimmune diseases, cardiovascular diseases including atherosclerosis, ischemia-reperfusion injury, acute and chronic organ failure including renal failure and heart failure, respiratory diseases, diabetes and complications of diabetes, severe allergies, transplant rejection, graft-versus-host disease, neurodegenerative diseases, diseases of the eye and retina, acute and chronic pain, degenerative bone diseases including osteoarthritis and osteoporosis, inflammatory bowel diseases, dermatitis and other skin diseases, sepsis, burns, seizure disorders, and neuropsychiatric disorders.

Without being bound by theory, the activation of the antioxidant/anti-inflammatory Keap1/Nrf2/ARE pathway is implicated in the anti-inflammatory and/or anti-carcinogenic properties of the compounds disclosed herein. In some embodiments, the compounds of the present invention may be used to activate the antioxidant/anti-inflammatory Keap1/Nrf2/ARE pathway. In some embodiments, the compounds of the present invention have anti-inflammatory and/or antioxidant properties.

In another aspect, compounds disclosed herein may be used for treating a subject having a condition caused by elevated levels of oxidative stress in one or more tissues. Oxidative stress results from abnormally high or prolonged levels of reactive oxygen species such as superoxide, hydrogen peroxide, nitric oxide, and peroxynitrite (formed by the reaction of nitric oxide and superoxide). The oxidative stress may be accompanied by either acute or chronic inflammation. The oxidative stress may be caused by mitochondrial dysfunction, by activation of immune cells such as macrophages and neutrophils, by acute exposure to an external agent such as ionizing radiation or a cytotoxic chemotherapy agent (e.g., doxorubicin), by trauma or other acute tissue injury, by ischemia/reperfusion, by poor circulation or anemia, by localized or systemic hypoxia or hyperoxia, by elevated levels of inflammatory cytokines and other inflammation-related proteins, and/or by other abnormal physiological states such as hyperglycemia or hypoglycemia.

In animal models of many such conditions, stimulating expression of inducible heme oxygenase (HO-1), a target gene of the Nrf2 pathway, has been shown to have a significant therapeutic effect including models of myocardial infarction, renal failure, transplant failure and rejection, stroke, cardiovascular disease, and autoimmune disease (e.g., Sacerdoti et al., 2005; Abraham & Kappas, 2005; Bach, 2006; Araujo et al., 2003; Liu et al., 2006; Ishikawa et al., 2001; Kruger et al., 2006; Satoh et al., 2006; Zhou et al., 2005; Morse and Choi, 2005; Morse and Choi, 2002). This enzyme breaks free heme down into iron, carbon monoxide (CO), and biliverdin (which is subsequently converted to the potent antioxidant molecule, bilirubin). In some embodiments, the compounds of the present invention may be used to stimulate expression of inducible heme oxygenase (HO-1).

In another aspect, compounds of this invention may be used in preventing or treating tissue damage or organ failure, acute and chronic, resulting from oxidative stress exacerbated by inflammation. Examples of diseases that fall in this category include heart failure, liver failure, transplant failure and rejection, renal failure, pancreatitis, fibrotic lung diseases (cystic fibrosis, COPD, and idiopathic pulmonary fibrosis, among others), diabetes (including complications), atherosclerosis, ischemia-reperfusion injury, glaucoma, stroke, autoimmune disease, autism, macular degeneration, and muscular dystrophy. For example, in the case of autism, studies suggest that increased oxidative stress in the central nervous system may contribute to the development of the disease (Chauhan and Chauhan, 2006).

Evidence also links oxidative stress and inflammation to the development and pathology of many other disorders of the central nervous system, including psychiatric disorders such as psychosis, major depression, and bipolar disorder; seizure disorders such as epilepsy; pain and sensory syndromes such as migraine, neuropathic pain or tinnitus; and behavioral syndromes such as the attention deficit disorders. See, e.g., Dickerson et al., 2007; Hanson et al., 2005; Kendall-Tackett, 2007; Lencz et al., 2007; Dudhgaonkar et al., 2006; Lee et al., 2007; Morris et al., 2002; Ruster et al., 2005; McIver et al., 2005; Sarchielli et al., 2006; Kawakami et al., 2006; Ross et al., 2003, which are all incorporated by reference herein. For example, elevated levels of inflammatory cytokines, including TNF, interferon-γ, and IL-6, are associated with major mental illness (Dickerson et al., 2007). Microglial activation has also been linked to major mental illness. Therefore, downregulating inflammatory cytokines and inhibiting excessive activation of microglia could be beneficial in patients with schizophrenia, major depression, bipolar disorder, autism-spectrum disorders, and other neuropsychiatric disorders. In some embodiments, the compounds of the present invention may be used to downregulate inflammatory cytokines and/or inhibit excessive activation of microglia.

Accordingly, in pathologies involving oxidative stress alone or oxidative stress exacerbated by inflammation, treatment may comprise administering to a subject a therapeutically effective amount of a compound of this invention, such as those described above or throughout this specification. In some embodiments, treatment may be administered preventively, in advance of a predictable state of oxidative stress (e.g., organ transplantation or the administration of radiation therapy to a cancer patient), or it may be administered therapeutically in settings involving established oxidative stress and inflammation.

In some embodiments, the compounds of the present invention may be used to treat inflammatory conditions, such as sepsis, dermatitis, autoimmune disease and osteoarthritis. In some embodiments, the compounds of the present invention may be used to treat inflammatory pain and/or neuropathic pain, for example, by inducing Nrf2 and/or inhibiting NF-κB.

In some embodiments, the compounds disclosed herein may be used in the treatment and prevention of diseases such as cancer, inflammation, Alzheimer's disease, Parkinson's disease, multiple sclerosis, autism, amyotrophic lateral sclerosis, Huntington's disease, autoimmune diseases such as rheumatoid arthritis, lupus, Crohn's disease and psoriasis, inflammatory bowel disease, all other diseases whose pathogenesis is believed to involve excessive production of either nitric oxide or prostaglandins, and pathologies involving oxidative stress alone or oxidative stress exacerbated by inflammation.

Another aspect of inflammation is the production of inflammatory prostaglandins such as prostaglandin E. These molecules promote vasodilation, plasma extravasation, localized pain, elevated temperature, and other symptoms of inflammation. The inducible form of the enzyme COX-2 is associated with their production, and high levels of COX-2 are found in inflamed tissues. Consequently, inhibition of COX-2 may relieve many symptoms of inflammation, and a number of important anti-inflammatory drugs (e.g., ibuprofen and celecoxib) act by inhibiting COX-2 activity. Recent research, however, has demonstrated that a class of cyclopentenone prostaglandins (cyPGs) (e.g., 15-deoxy prostaglandin J2, a.k.a. PGJ2) plays a role in stimulating the orchestrated resolution of inflammation (e.g., Rajakariar et al., 2007). COX-2 is also associated with the production of cyclopentenone prostaglandins. Consequently, inhibition of COX-2 may interfere with the full resolution of inflammation, potentially promoting the persistence of activated immune cells in tissues and leading to chronic, “smoldering” inflammation. This effect may be responsible for the increased incidence of cardiovascular disease in patients using selective COX-2 inhibitors for long periods of time. In some embodiments, the compounds of the present invention may be used to reduce or inhibit the production of COX-2.

In another aspect, the compounds disclosed herein may be used to control the production of pro-inflammatory cytokines within the cell by selectively activating regulatory cysteine residues (RCRs) on proteins that regulate the activity of redox-sensitive transcription factors. Activation of RCRs by cyPGs has been shown to initiate a pro-resolution program in which the activity of the antioxidant and cytoprotective transcription factor Nrf2 is potently induced and the activities of the pro-oxidant and pro-inflammatory transcription factors NF-κB and the STATs are suppressed. In some embodiments, this increases the production of antioxidant and reductive molecules (NQO1, HO-1, SOD1, γ-GCS) and decreases oxidative stress and the production of pro-oxidant and pro-inflammatory molecules (iNOS, COX-2, TNF-α). In some embodiments, the compounds of this invention may cause the cells that host the inflammatory event to revert to a non-inflammatory state by promoting the resolution of inflammation and limiting excessive tissue damage to the host.

### IV. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al. FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### V. Combination Therapy

In addition to being used as a monotherapy, the compounds of the present invention may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

Non-limiting examples of such combination therapy include combination of one or more compounds of the invention with another anti-inflammatory agent, a chemotherapeutic agent, radiation therapy, an antidepressant, an antipsychotic agent, an anticonvulsant, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders. Compounds of the invention may be combined with agents designed to improve a patient's immune response to cancer, including (but not limited to) cancer vaccines. See Lu et al. (2011), which is incorporated herein by reference.

### VI. Definitions

The definitions below supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

When used in the context of a chemical group: “hydrogen” means —H; “hydroxy” means —OH; “oxo” means=O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanyl” means —N═C═O; “azido” means —N₃; in a monovalent context “phosphate” means —OP(OXOH)₂ or a deprotonated form thereof; in a divalent context “phosphate” means —OP(OXOH)O— or a deprotonated form thereof; “mercapto” means —SH; and “thio” means=S; “thiocarbonyl” means —C(═S)—; “sulfonyl” means —S(O)₂—; and “sulfinyl” means —S(O)—.

In the context of chemical formulas, the symbol “−” represents a single bond, “═” represents a double bond; and “≡” represents triple bond. The symbol “————” represents an optional bond, which if present is either single or double. Unless indicated otherwise, the symbol “ [symbol] ” single bond or a double bond. For example, the symbol “ [symbol] ” can also represent a single bond, a double bond, or an “epoxidized double bond” when this is specifically provided for. An “epoxidized double bond” represents the group:

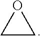

Furthermore, it is noted that the single bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

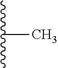

for methyl) indicates a point of attachment of the group. It is noted that the point of attachment is typically only identified in this manner for larger groups in order to assist the reader in unambiguously identifying a point of attachment. The symbol “ [symbol] ” represents a single bond where the group attached to the thick end of the wedge is “out of the page.” The symbol “ [symbol] ” represents a single bond where the group attached to the thick end of the wedge is “into the page”. The symbol “ [symbol] ” represents a single bond where the geometry around a double bond (e.g., either E or 2) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

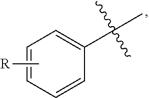

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

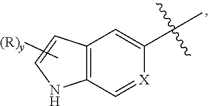

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic n system. An aromatic compound or chemical group may be depicted as a single resonance structure; however, depiction of one resonance structure is taken to also refer to any other resonance structure. For example:

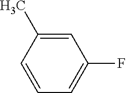

is also taken to refer to

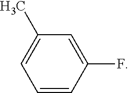

Aromatic compounds may also be depicted using a circle to represent the delocalized nature of the electrons in the fully conjugated cyclic n system, two non-limiting examples of which are shown below:

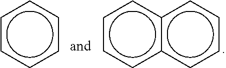

The term “alkyl” refers to a monovalent saturated aliphatic group with a carbon atom as the point of attachment, a linear or branched acyclic structure, and no atoms other than carbon and hydrogen. The groups —CH₃(Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

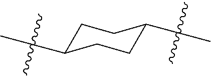 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

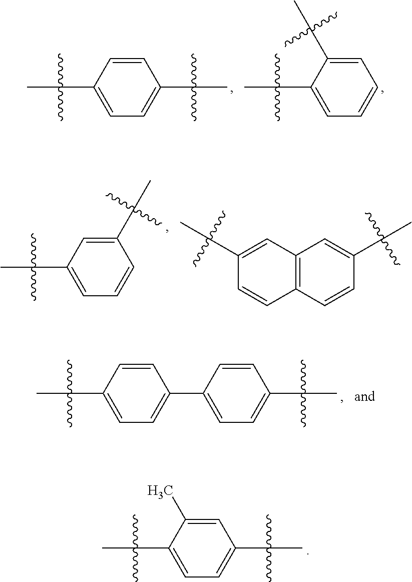

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenylethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

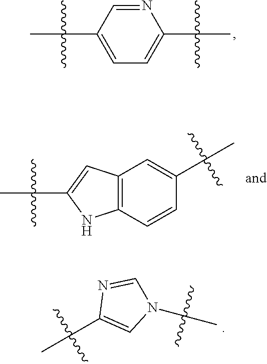 [SMILES: *c1ccc(*)nc1 ; *c1ccc2[nH]c(*)cc2c1 ; *c1cn(*)cn1]

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic.

Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. Non-limiting examples of N-heterocycloalkyl groups include N-pyrrolidinyl and

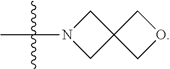 [SMILES: *N1CC2(COC2)C1]

When the term “heterocycloalkyl” is used with the “substituted” modifier, one or more hydrogen atom(s)has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂; or two or four more hydrogen atoms have been replaced with one or two oxo groups, respectively. For example, the following groups are non-limiting examples of substituted heterocycloalkyl groups (more specifically, substituted N-heterocycloalkyl groups):

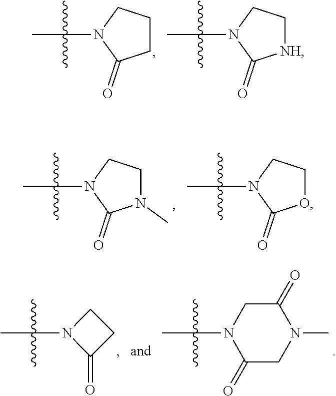

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C6H₃, and —C(O)C6H4CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “cycloalkylamino,” when used without the “substituted” modifier, refers to the group defined as —NHR, in which R is cycloalkyl. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

With the exception of the term “heterocycloalkyl”, when a chemical group is used with the “substituted” modifier, one or more hydrogen atom(s) of the group has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “hydroxyalkyl” is a subset of substituted alkyl, in which one or more hydrogen atom has been replaced with a hydroxy (i.e. —OH) group, such that no other atoms aside from carbon, hydrogen, and oxygen are present. The groups —CH₂OH, —CH₂CH₂OH, —CH(OH)CHOH, —CH₂CH(OH)CH₃, and —CH(OH)CH₂OH are non-limiting examples of hydroxyalkyl groups. The term “monohydroxyalkyl” is a subset of substituted alkyl, in which one hydrogen atom has been replaced with a hydroxy (i.e. —OH) group, such that no other atoms aside from carbon, hydrogen, and one oxygen are present. The groups —CH₂OH, —CH₂CH₂OH, and —CH₂CH(OH)CH₃ are non-limiting examples of monohydroxyalkyl groups. The term “fluoroalkyl” is a subset of substituted alkyl, in which one or more hydrogen atom has been replaced with a fluoro, such that no other atoms aside from carbon, hydrogen, and fluorine are present. The groups —CH₂F, —CHF₂, and —CF₃ are non-limiting examples of fluoroalkyl groups. The term “monofluoroalkyl” is a subset of substituted alkyl, in which one hydrogen atom has been replaced with a fluoro, such that no other atoms aside from carbon, hydrogen, and one fluorine are present. The groups —CH₂F, —CH₂CH₂F, and —CH₂CH(F)CH₃ are non-limiting examples of monofluoroalkyl groups. The term “aminoalkyl” is a subset of substituted alkyl, in which one or more hydrogen atom has been replaced with an amino (i.e. —NH₂) group, such that no other atoms aside from carbon, hydrogen, and nitrogen are present. The groups —CH₂NH₂, —CH(NH₂)CH₃, —CH₂CH₂NH₂, —CH₂CH(NH₂)CH₃ and —CH(NH₂)CH₂NH₂ are non-limiting examples of aminoalkyl groups. The term “monoaminoalkyl” is a subset of substituted alkyl, in which one hydrogen atom has been replaced with an amino (i.e. —NH₂) group, such that no other atoms aside from carbon, hydrogen, and one nitrogen are present. The groups —CH₂NH₂, —CH₂CH₂NH₂, and —CH₂CH(NH₂)CH₃ are non-limiting examples of monoaminoalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

When a chemical group is used with the “polar-substituted” modifier, one or more hydrogen atom(s) of the group has been replaced, independently at each instance, by one of the following polar substituents: —OH, —F, —NH₂, —CO₂H, —CO₂CH₃, —C(O)NH₂, —C(O)NHCH₃, —OC(O)CH₃, —NHC(O)CH₃, —NHC(O)OCH₃, —NHC(O)OCH₂CH₃, —NHC(O)NHCH₃, —NHC(O)NHCH₂CH₃, —S(O)₂OH, or —S(O)₂NH₂, provided that not every hydrogen is so replaced.

When a chemical group is used with the “monopolar-substituted” modifier, one and only one hydrogen atom of the group has been replaced by one of the following polar substituents: —OH, —F, —NH₂, —CO₂H, —CO₂CH₃, —C(O)NH₂, —C(O)NHCH₃, —OC(O)CH₃, —NHC(O)CH₃, —NHC(O)OCH₃, —NHC(O)OCH₂CH₃, —NHC(O)NHCH₃, —NHC(O)NHCH₂CH₃, —S(O)₂OH, or —S(O)₂NH₂. Non-limiting examples of monopolar-substituted alkyl groups include —CH₂F, —CH₂CH₂F, —CHFCH₃, —CH₂OH, —CH₂CH₂OH, —CH(OH)CH₂OH, —CH₂NH₂, —CH₂CH₂NH₂, and —CH(NH₂)CH₃.

Some of the abbreviations used herein are as follows: Ac indicates an acetyl group (—C(O)CH₃) Boc refers to tert-butyloxycarbonyl; COX-2, cyclooxygenase-2; cyPGs refers to cyclopentenone prostaglandins; DBDMH refers to 1,3-Dibromo-5,5-dimethylhydantoin; DIBAL-H is diisobutylaluminium hydride; DMAP refers to 4-dimethylaminopyridine; DMF is dimethylformamide; DMSO is dimethyl sulfoxide; EDC refers to 1-ethyl-3-(3-dimethylaminopropyl)carbodiimide; Et₂O, diethyl ether; HO-1 stands for inducible heme oxygenase IFNγ or IFN-γ stand for interferon-γ; IL-1β stands for interleukin-1β; iNOS stands for inducible nitric oxide synthase; NCS refers to N-Chlorosuccinimide; NMO refers to N-methylmorpholine N-oxide; NO stands for nitric oxide; Py stands for Pyridine; T3P refers to propylphosphonic anhydride; TFA is trifluoroacetic acid; THF is tetrahydrofuran; TNFα or TNF-α, tumor necrosis factor-α; TPAP is tetrapropylammonium perruthenate; Ts stands for tosyl; TsOH orp-TsOH is p-toluenesulfonic acid.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-p-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains <15%, more preferably <10%, even more preferably <5%, or most preferably <1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VII. Examples

### Example 1: Experimental Procedures and Characterization Data

### A. General Information

Unless otherwise stated, commercially reagents were used as received, and all reactions were run under nitrogen atmosphere. All solvents were of HPLC or ACS grade. Nuclear magnetic resonance (NMR) spectra were recorded on a Varian Inova-400 spectrometer at operating frequencies of 400 MHz (¹H NMR). Chemical shifts (δ) are given in ppm relative to residual solvent (usually chloroform δ 7.26 ppm for ¹H NMR) and coupling constants (J) in Hz. Multiplicity is tabulated as s for singlet, d for doublet, t for triplet, q for quadruplet, and m for multiplet. Mass spectra were recorded on Agilent 6120 mass spectrometer. The compounds of the present disclosure may be prepared according to the methods outlined in Example 1 as well as methods known to a skilled artisan, including those disclosed in WO 2009/129546, WO 2012/125488, and WO 2014/040056, which are incorporated by reference herein.

### B. Synthetic Routes to Compounds of the Present Disclosure

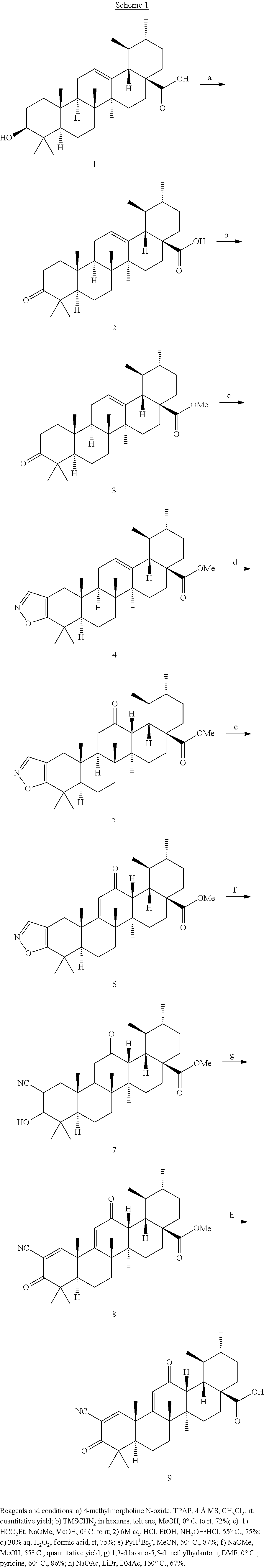

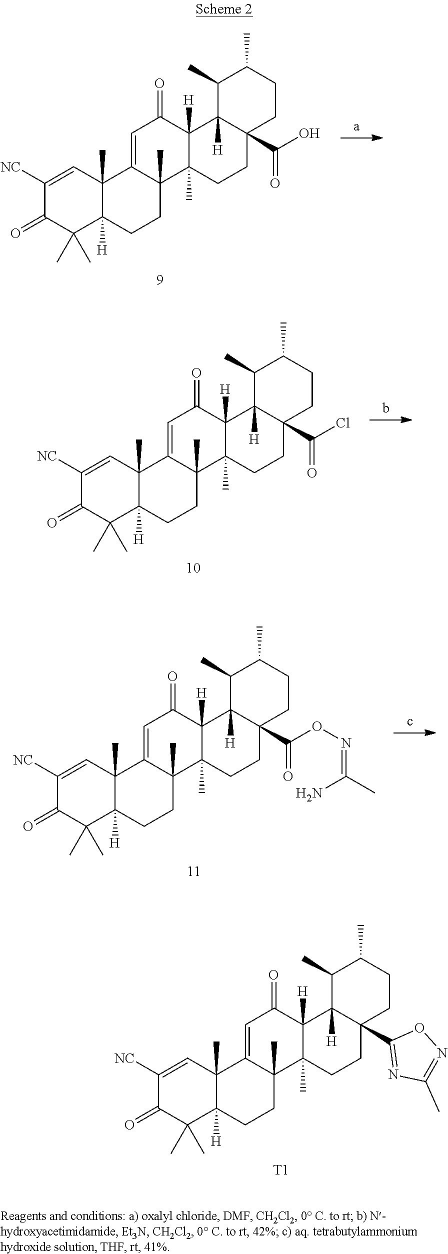

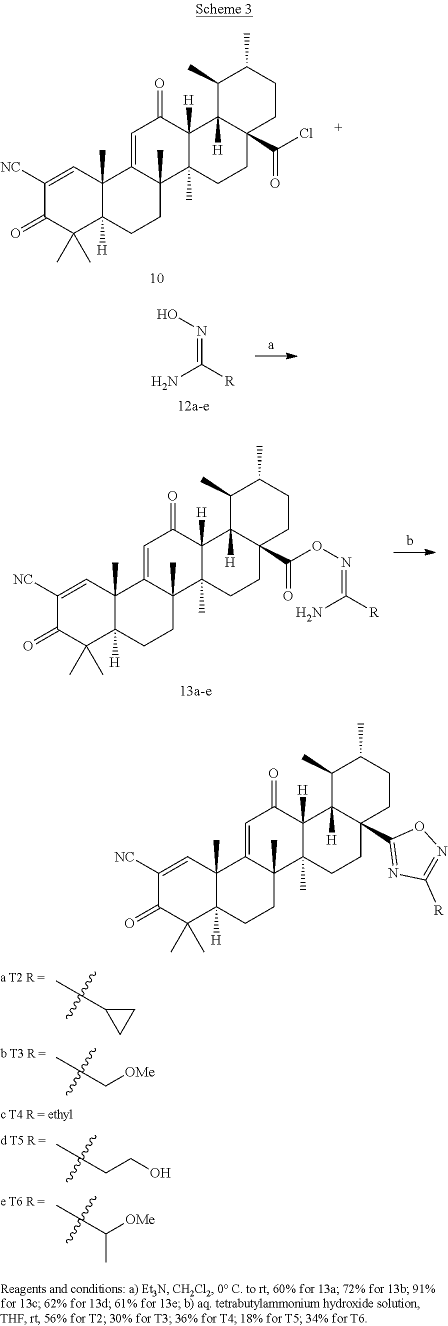

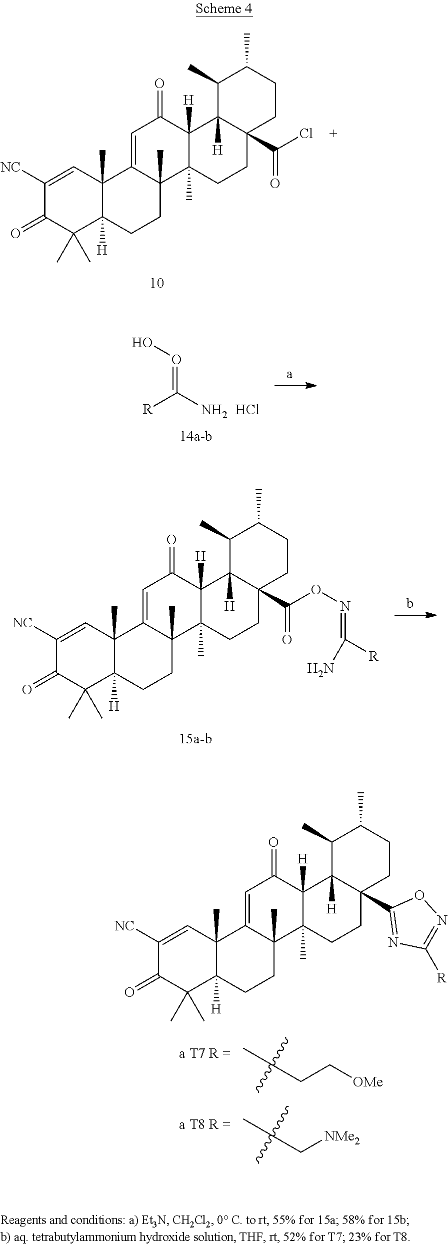

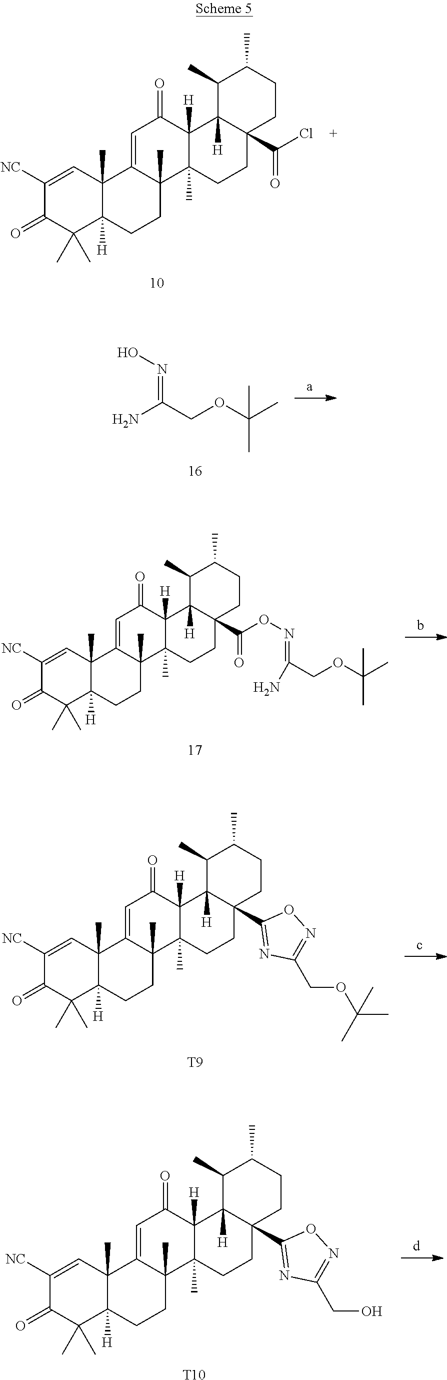

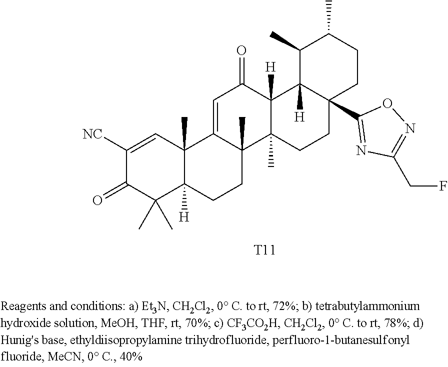

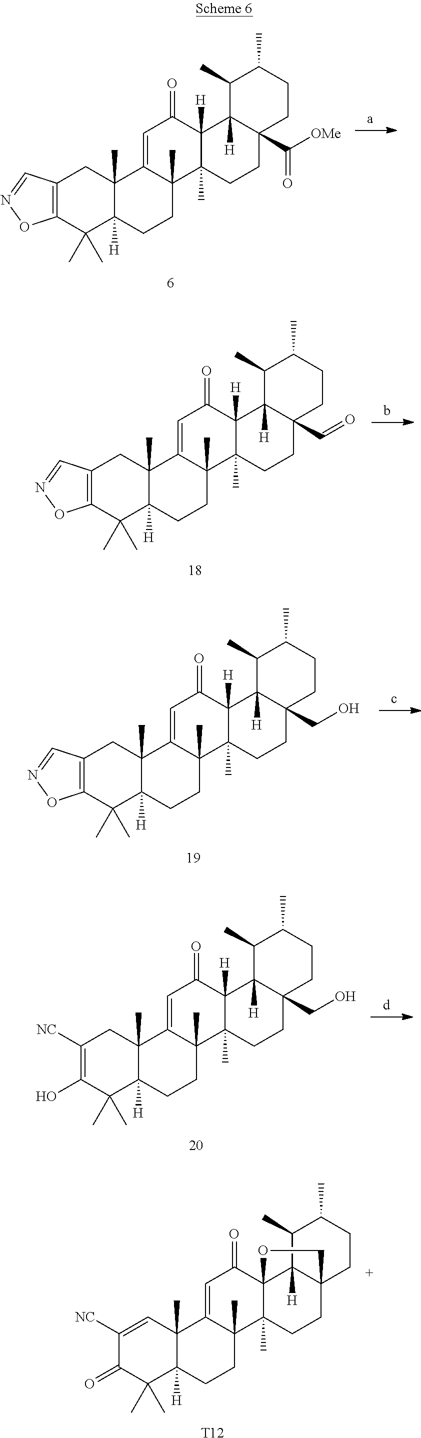

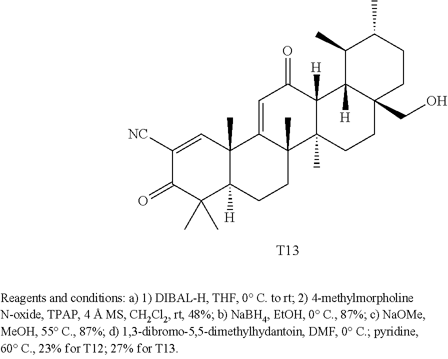

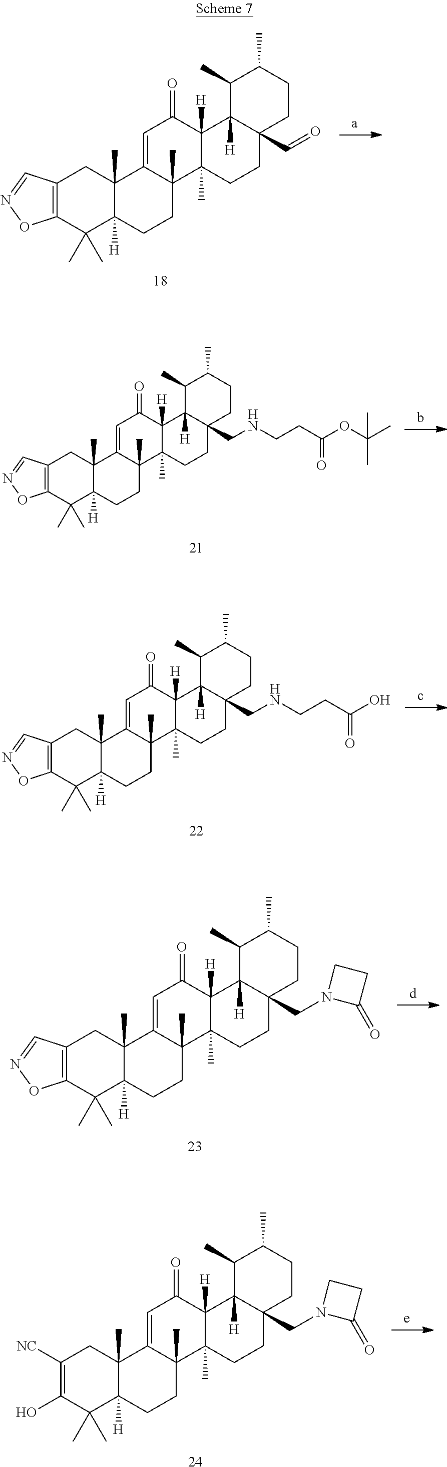

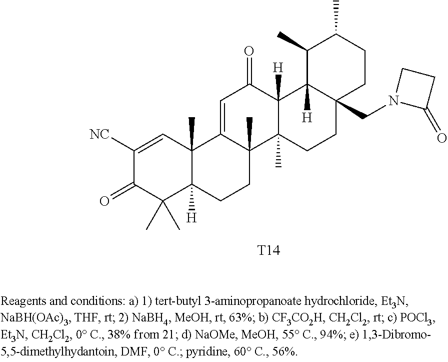

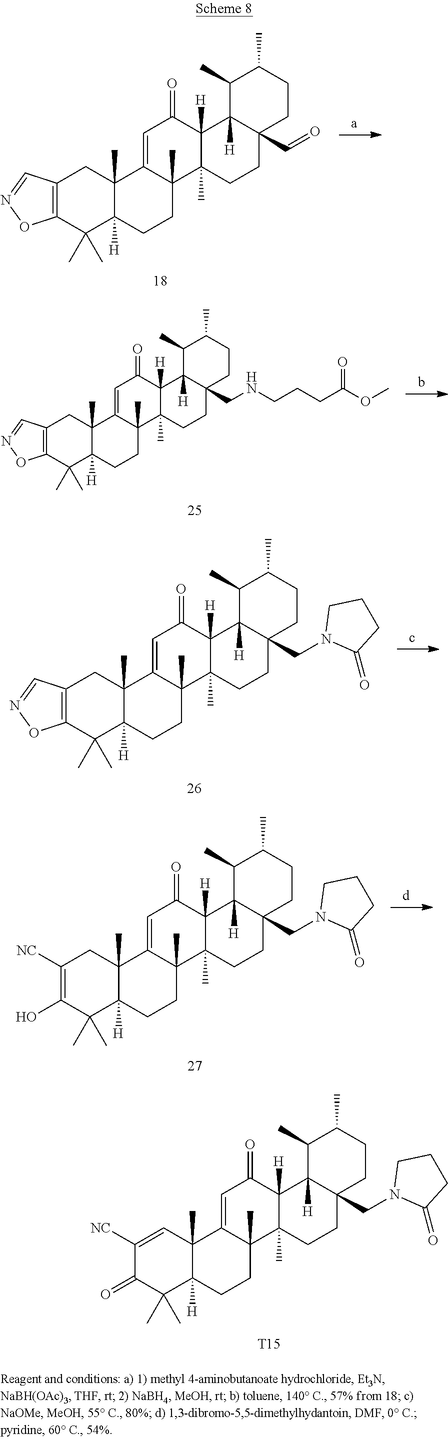

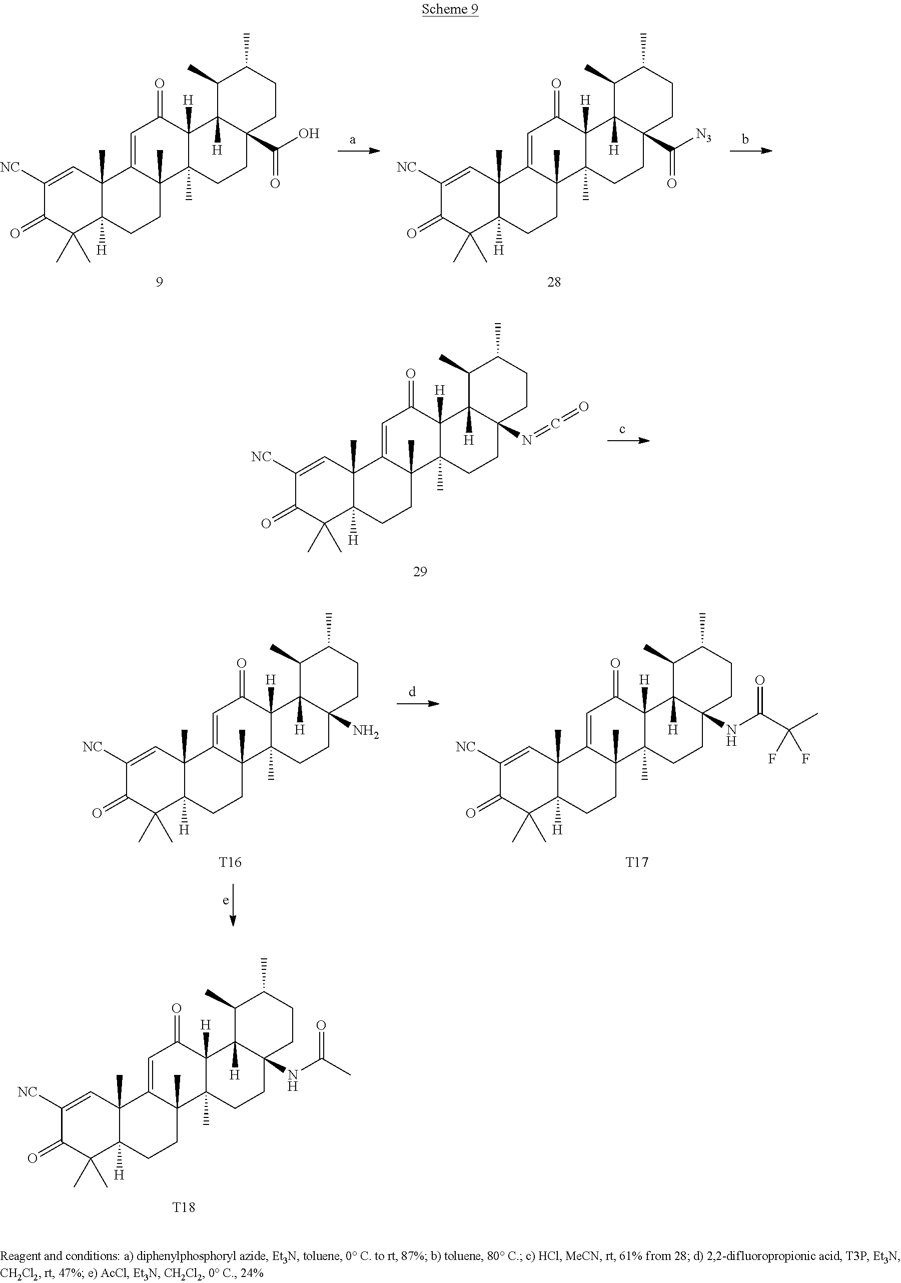

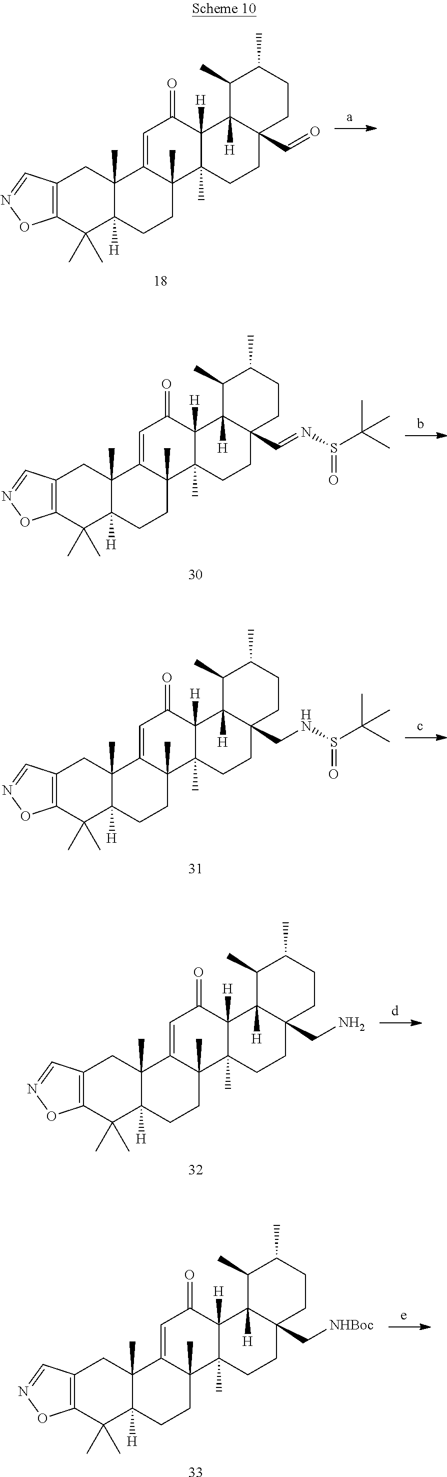

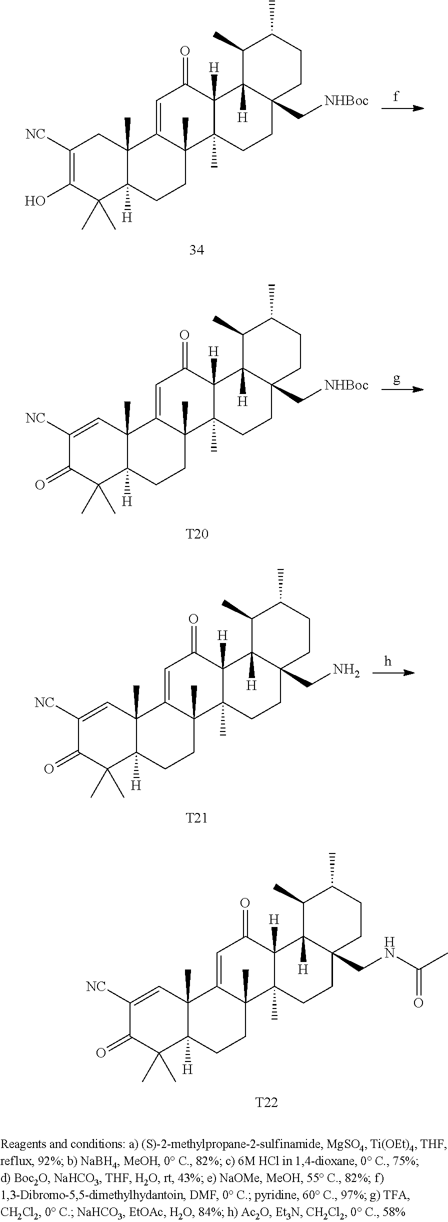

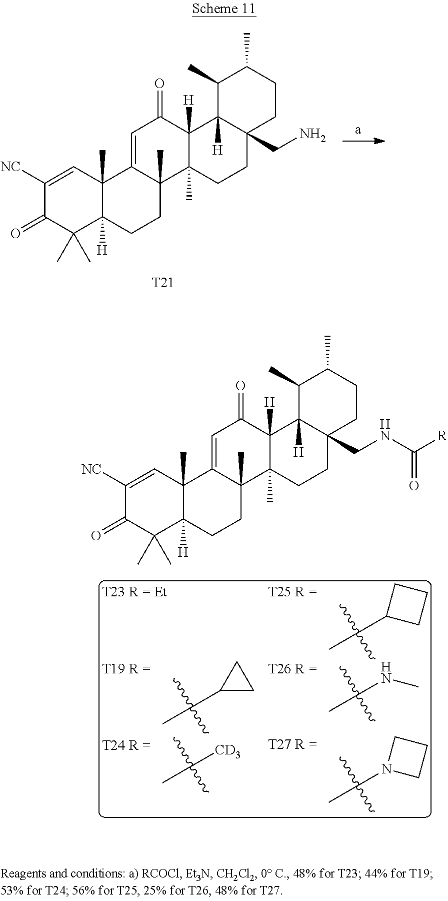

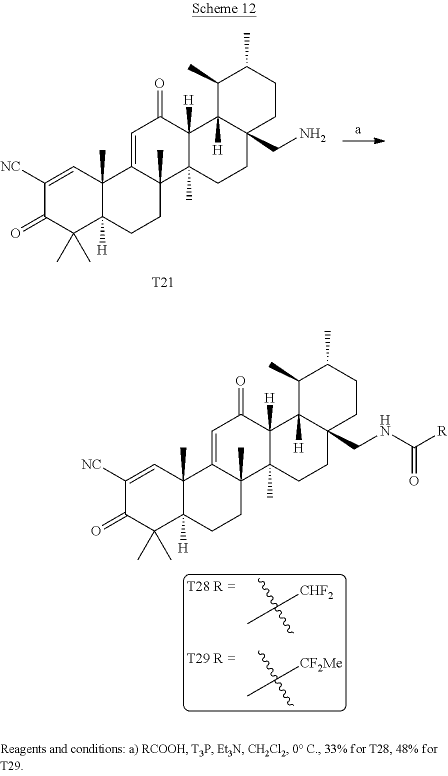

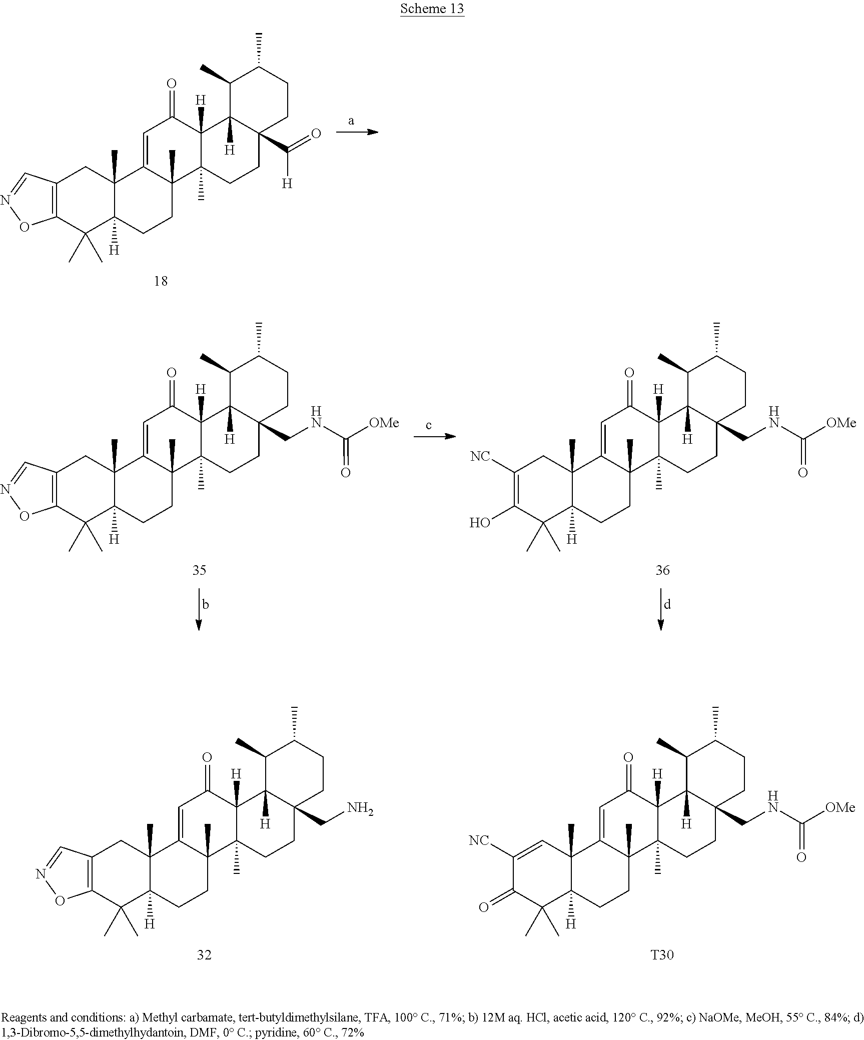

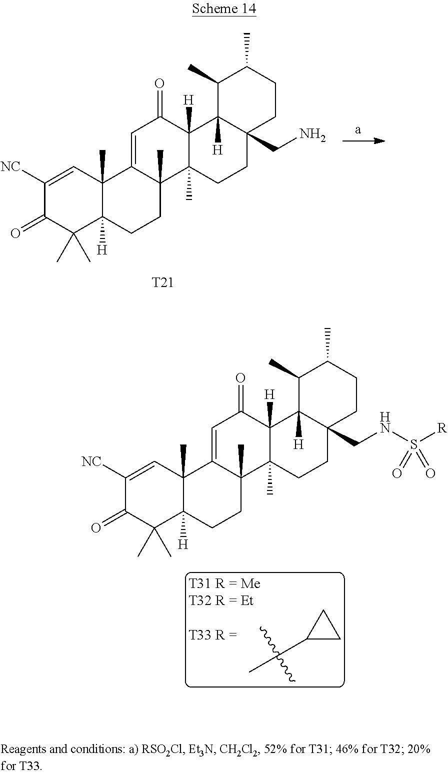

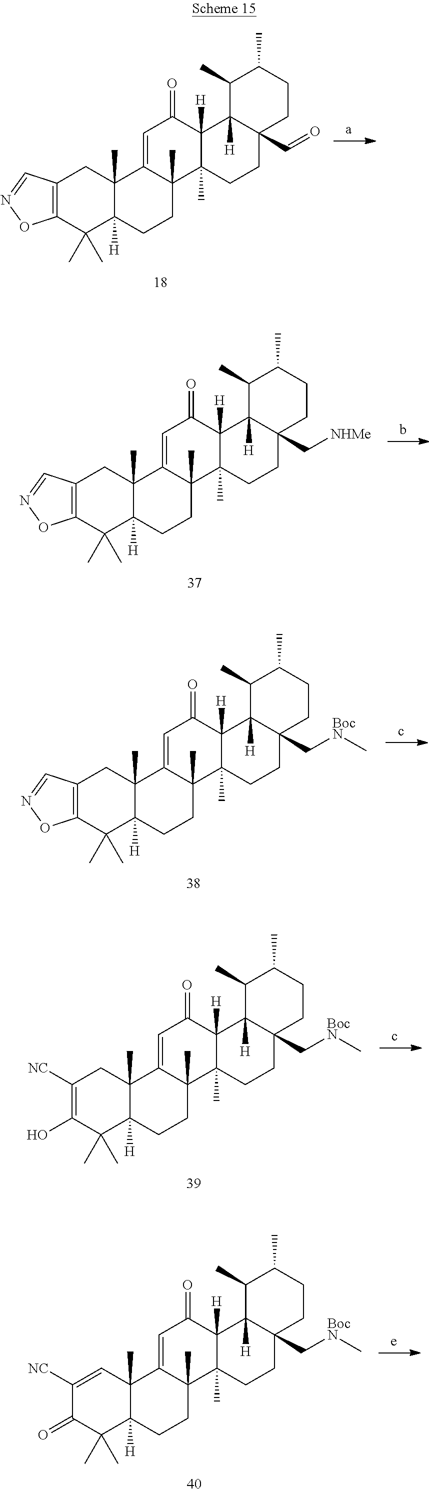

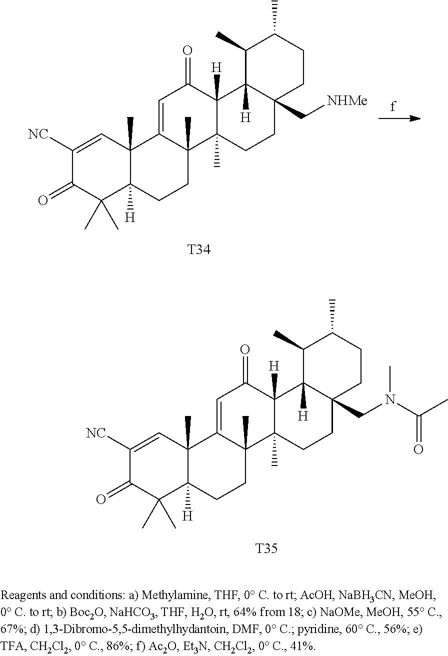

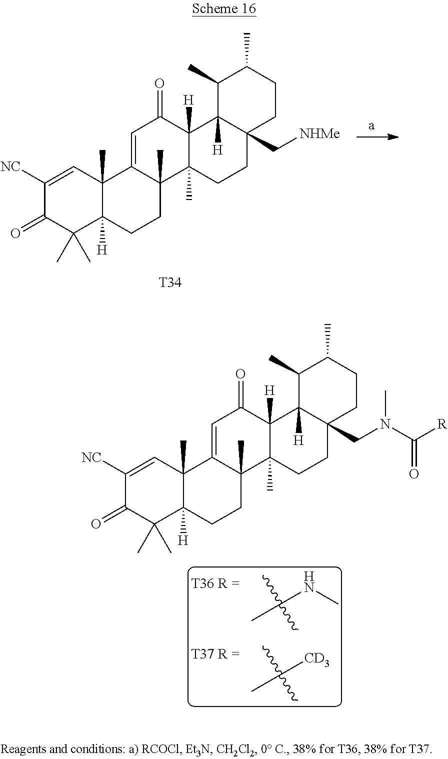

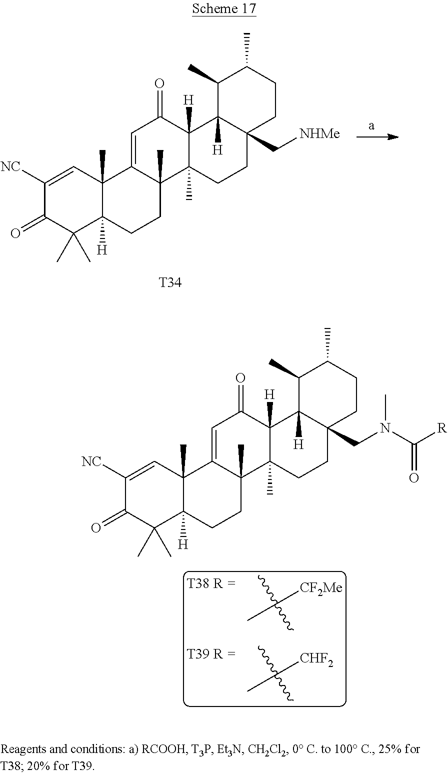

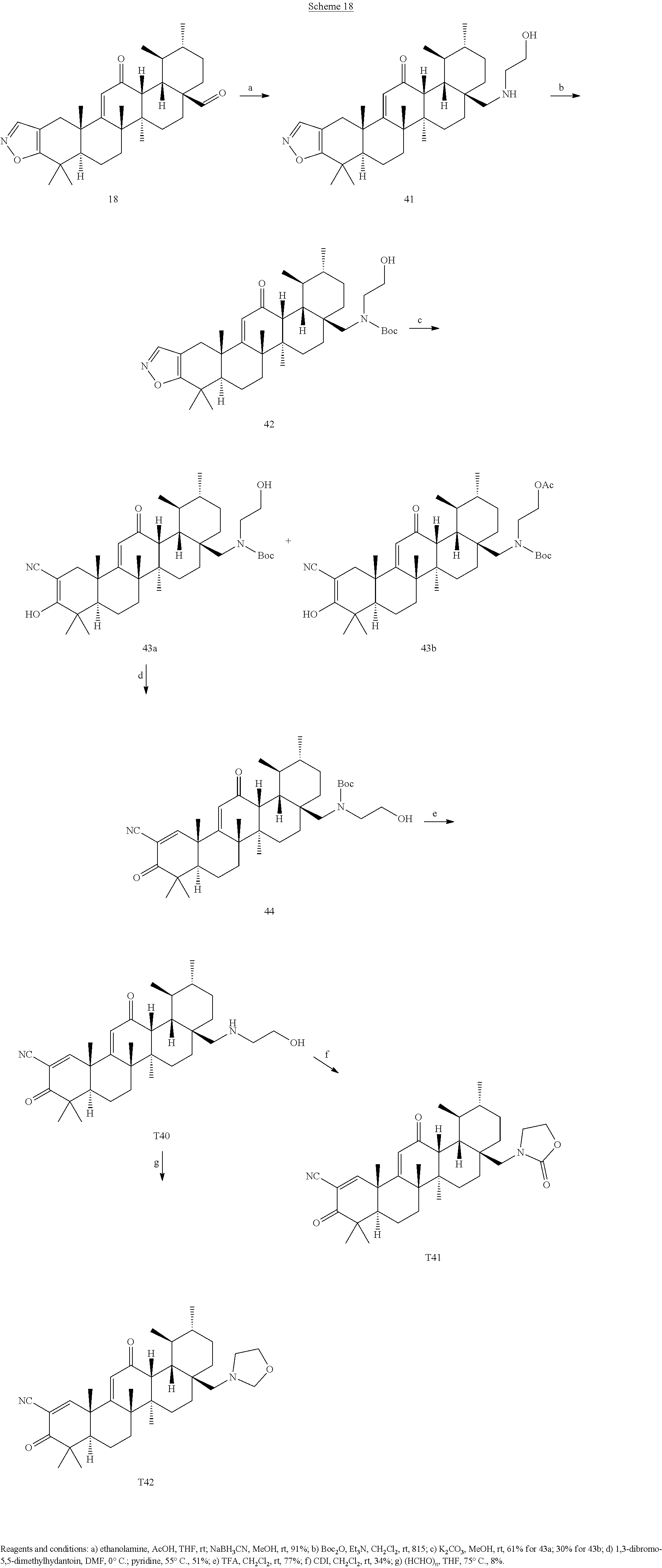

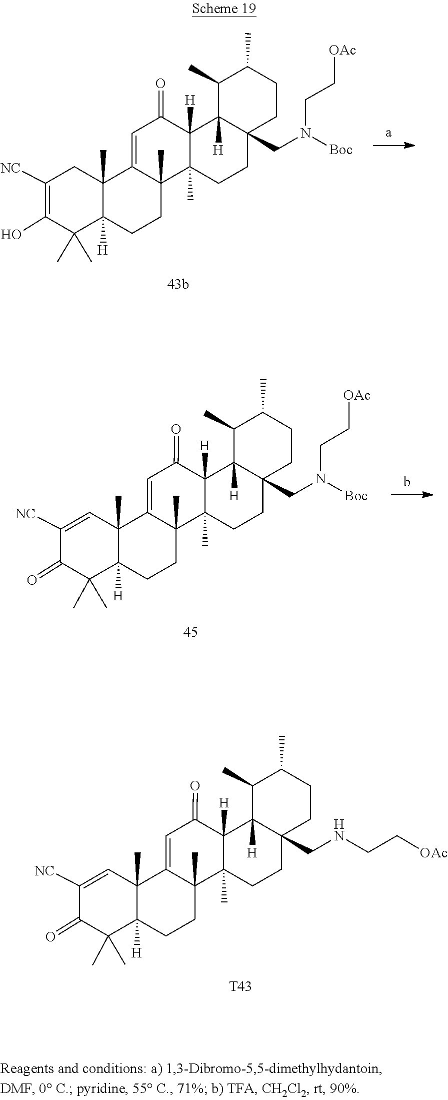

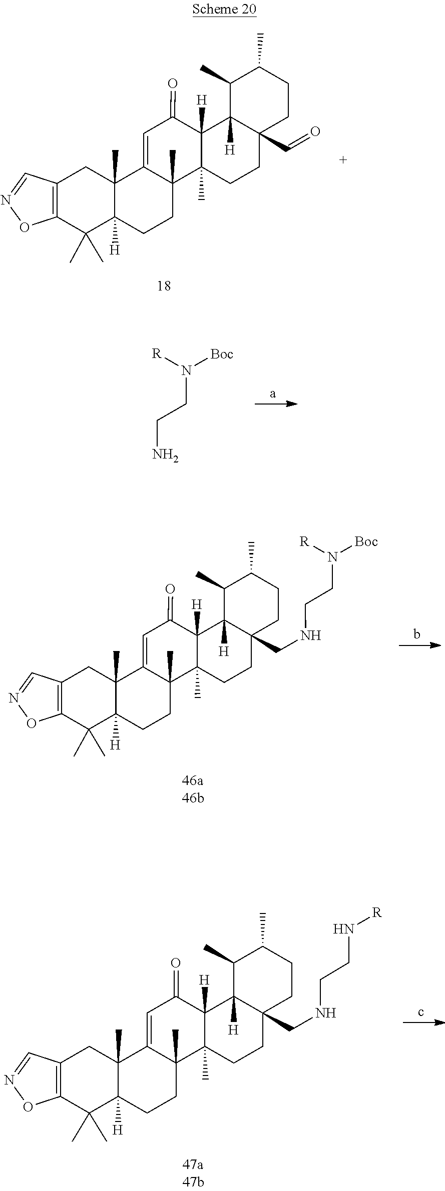

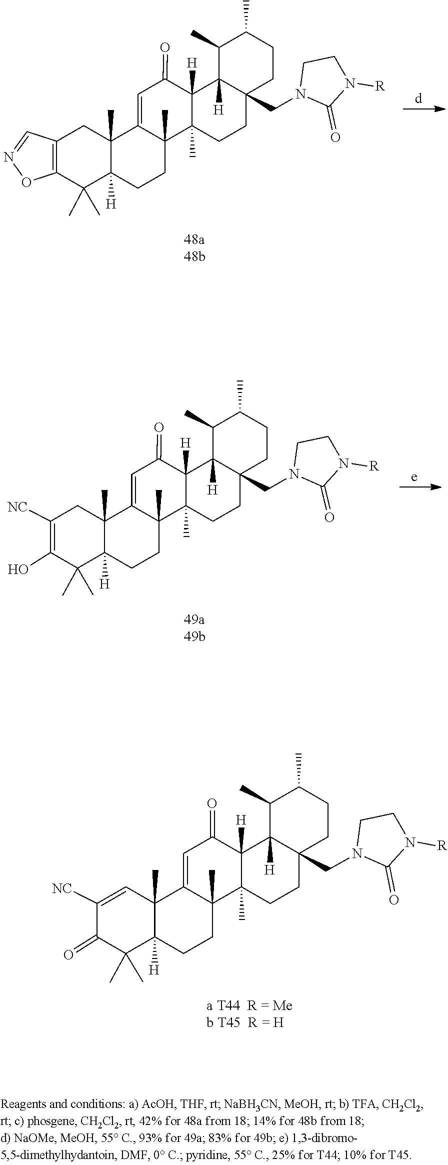

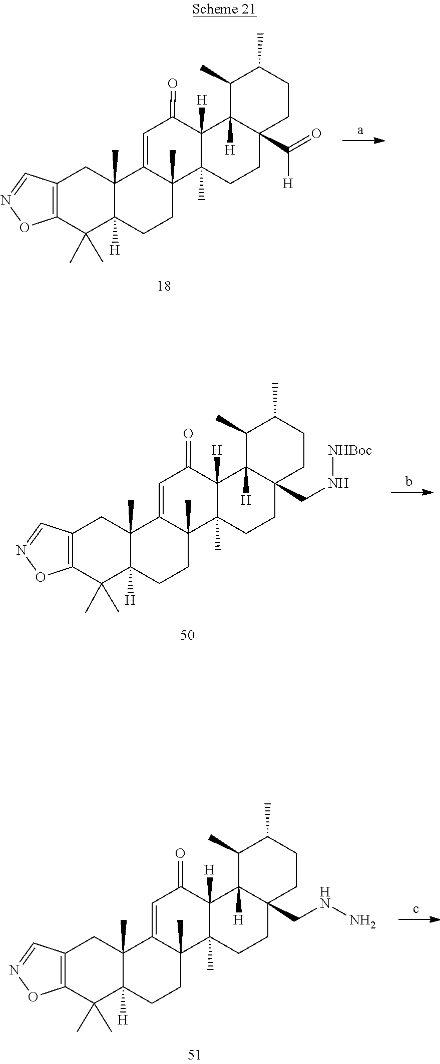

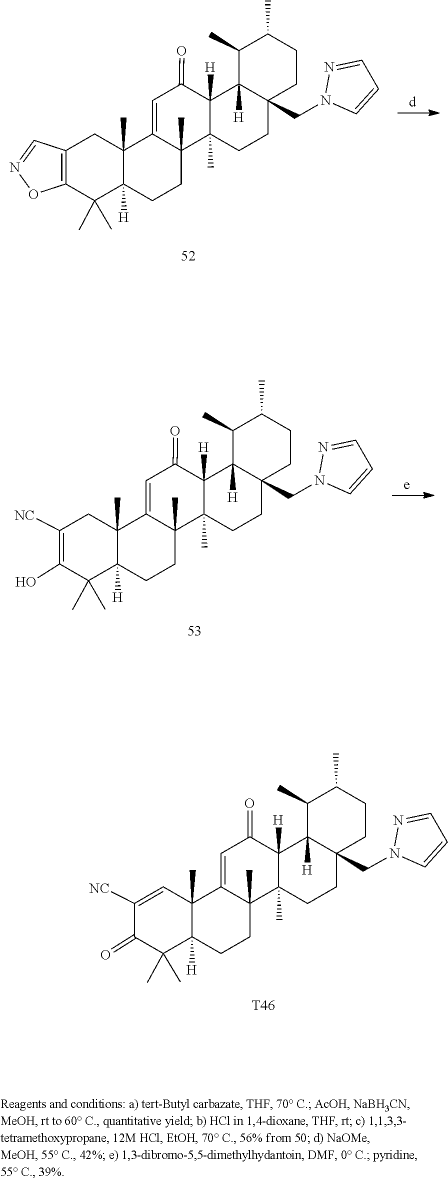

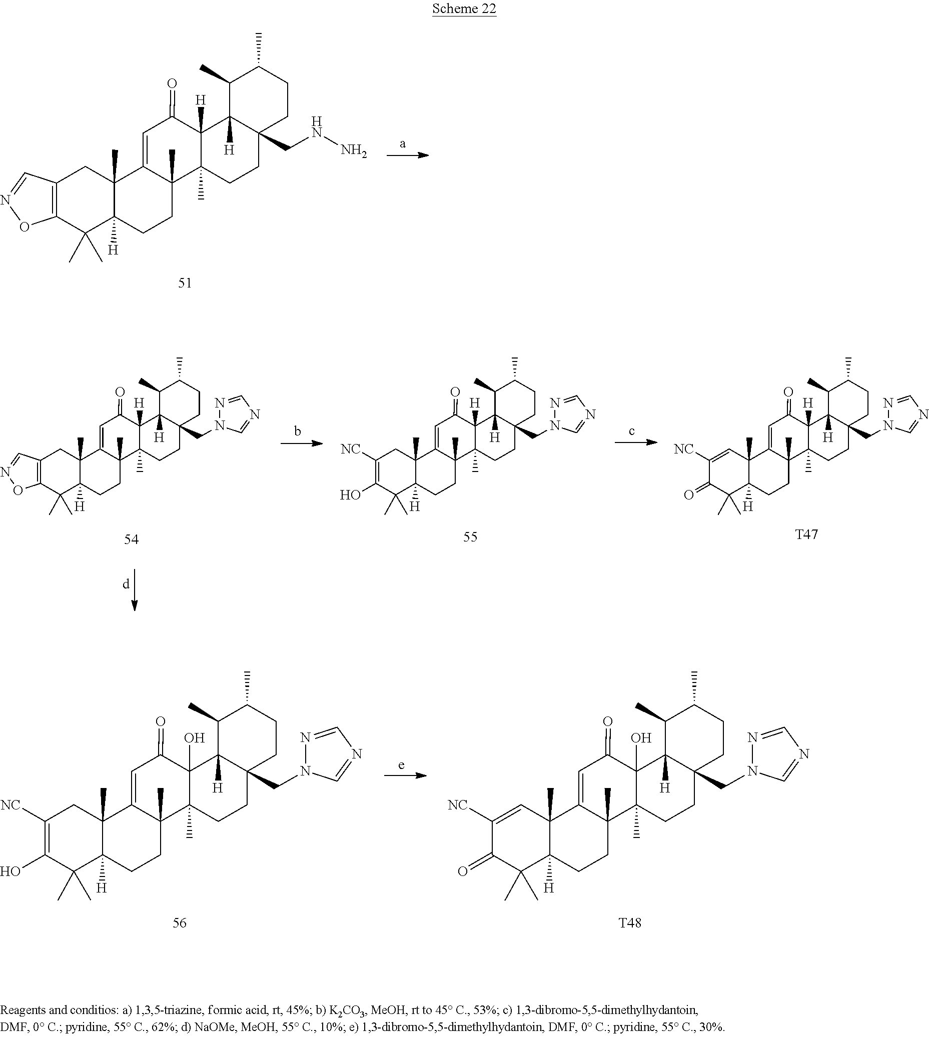

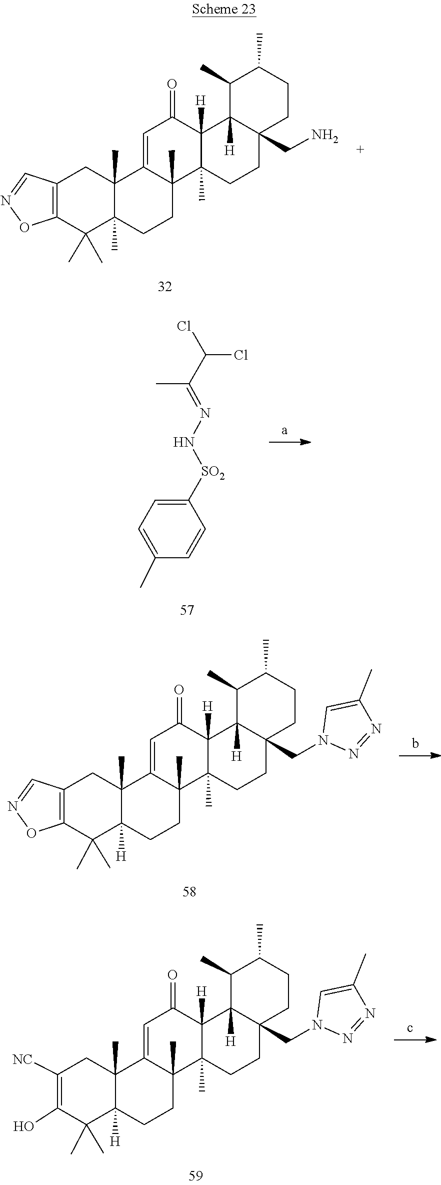

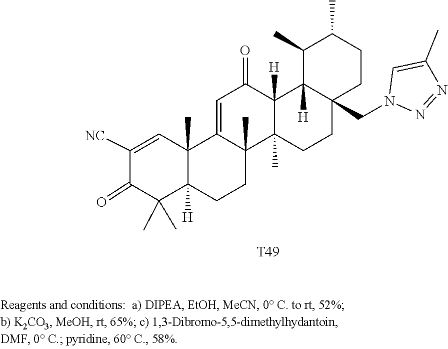

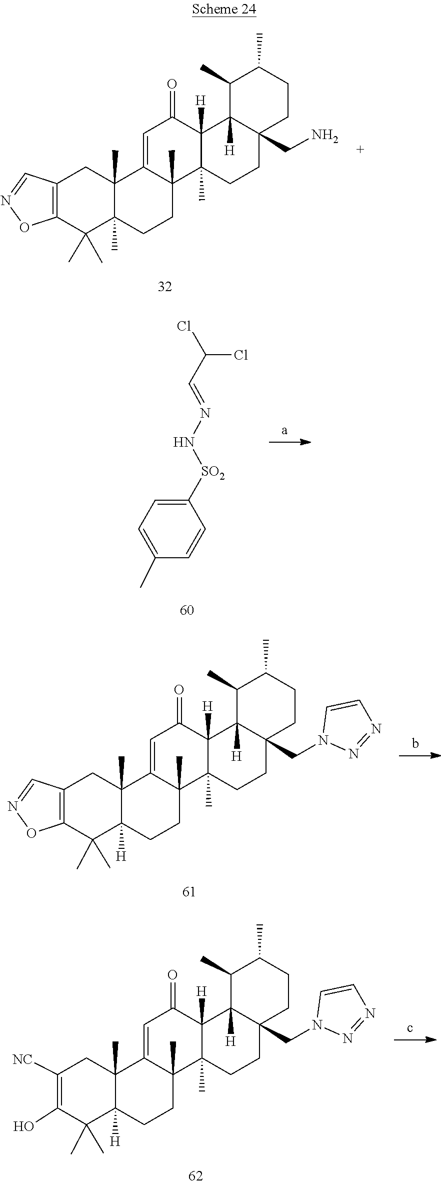

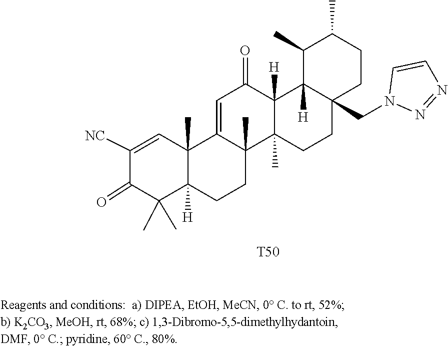

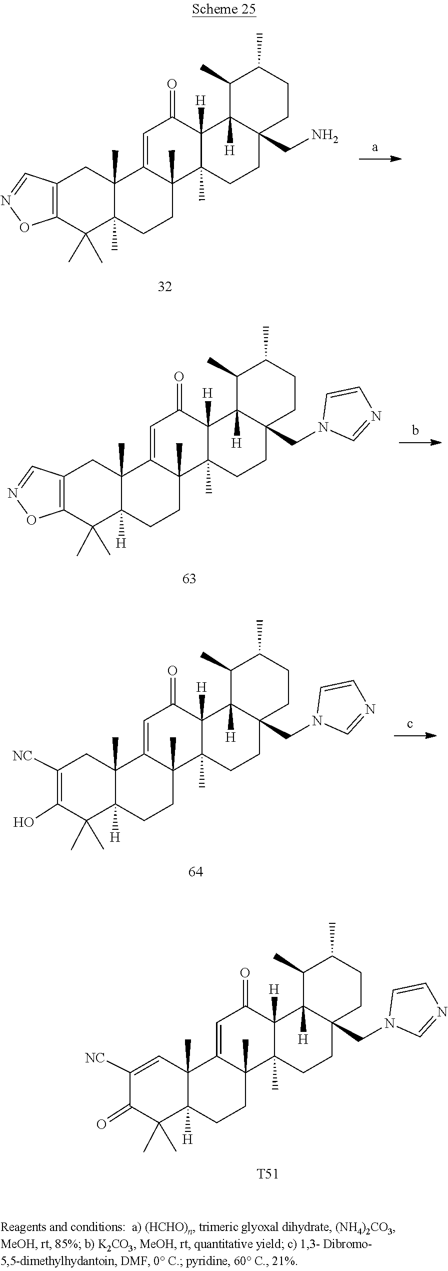

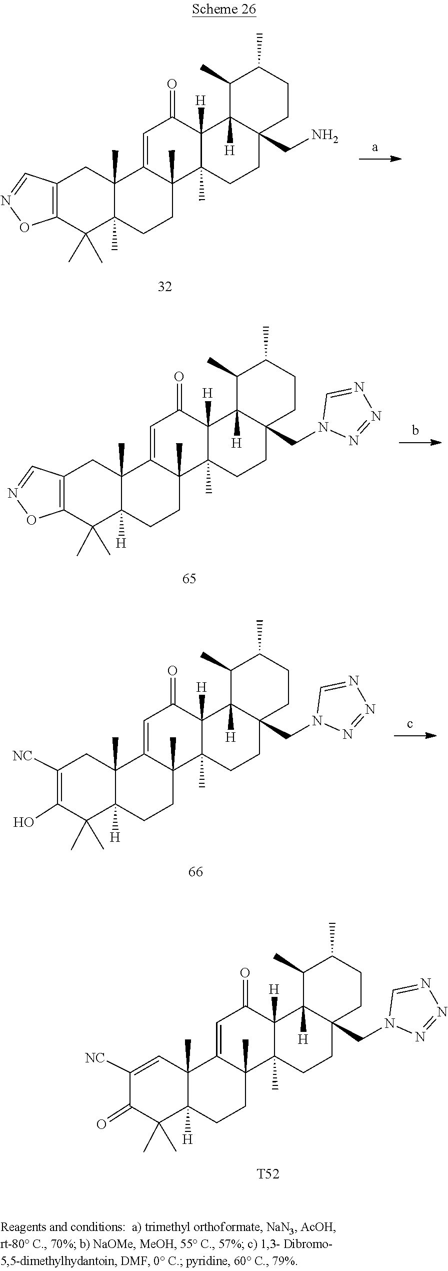

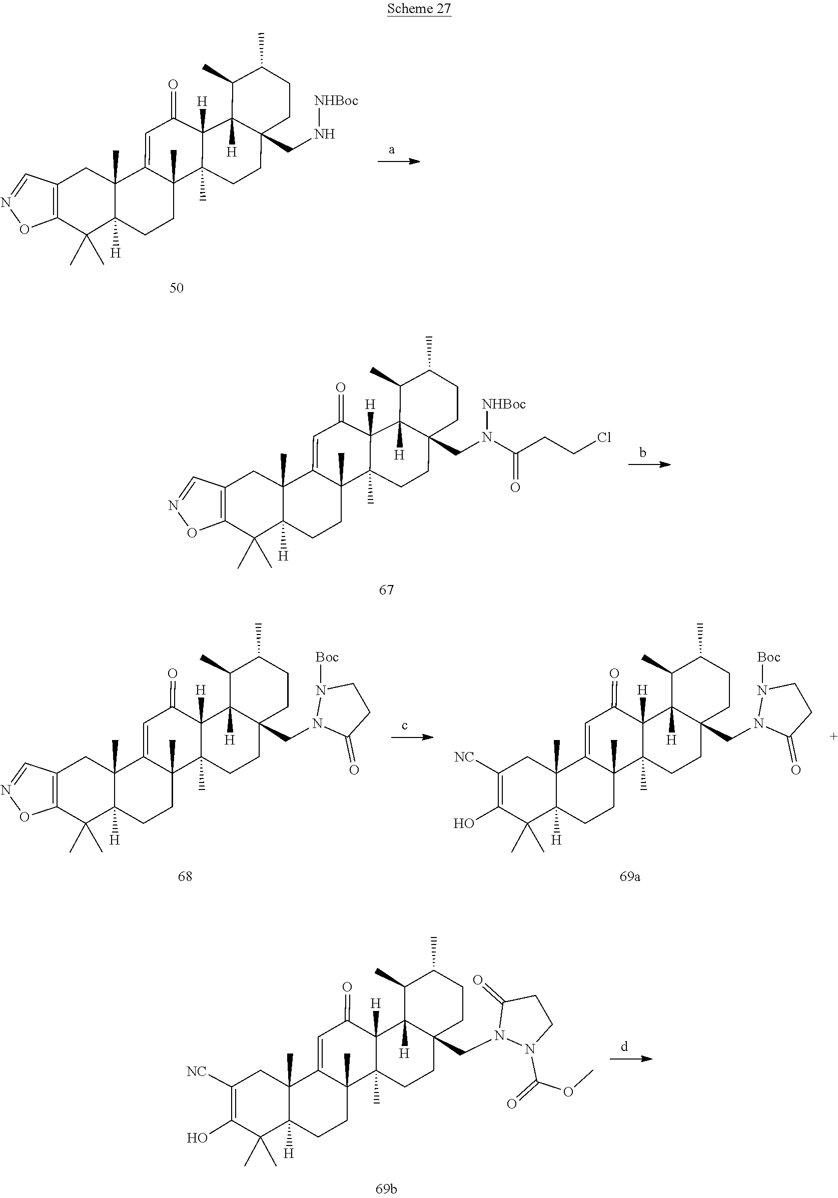

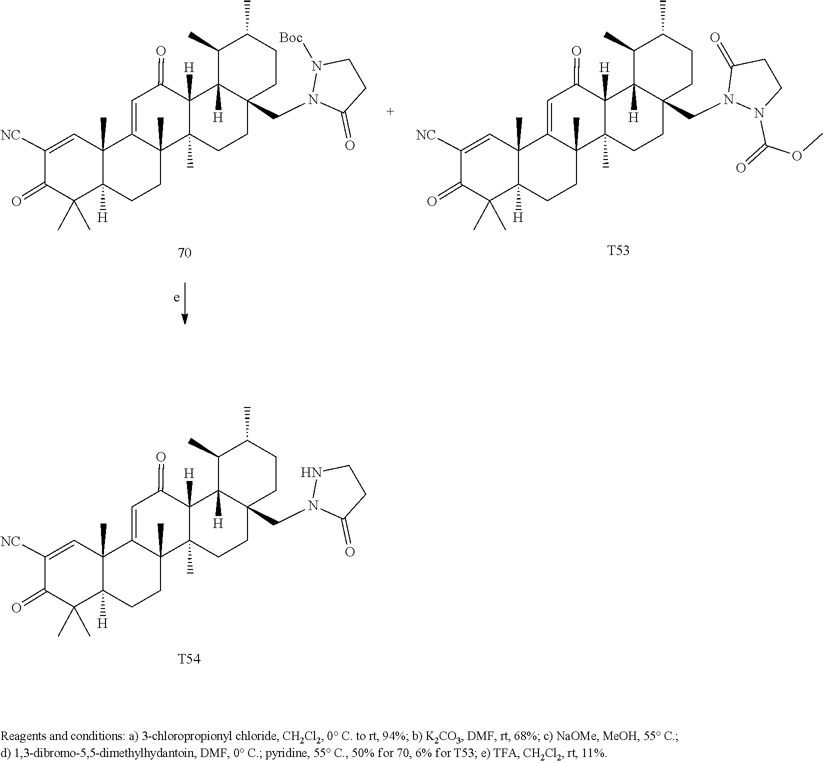

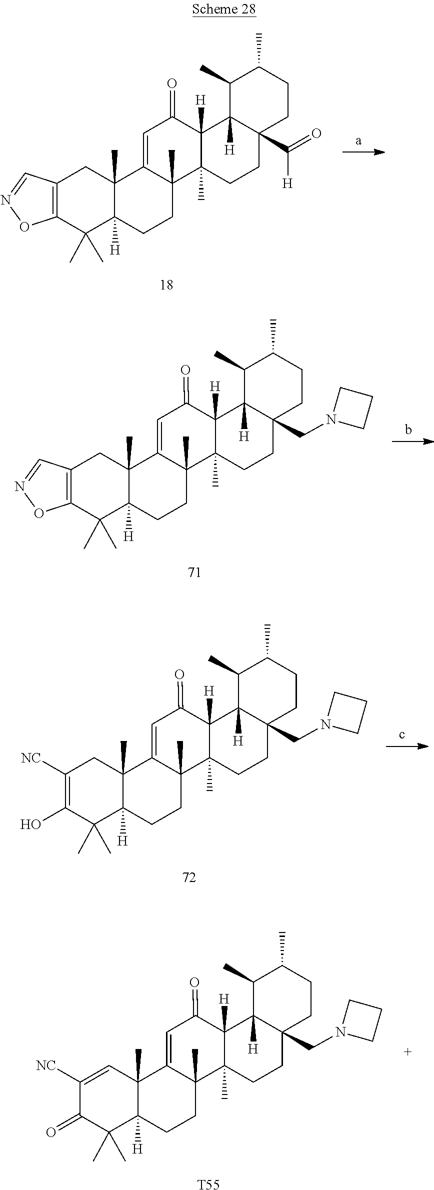

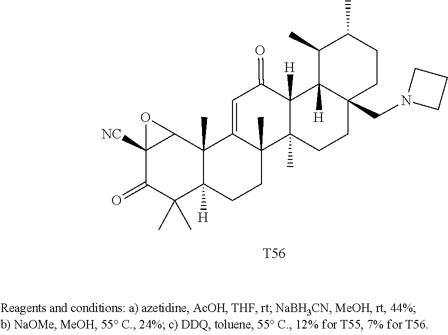

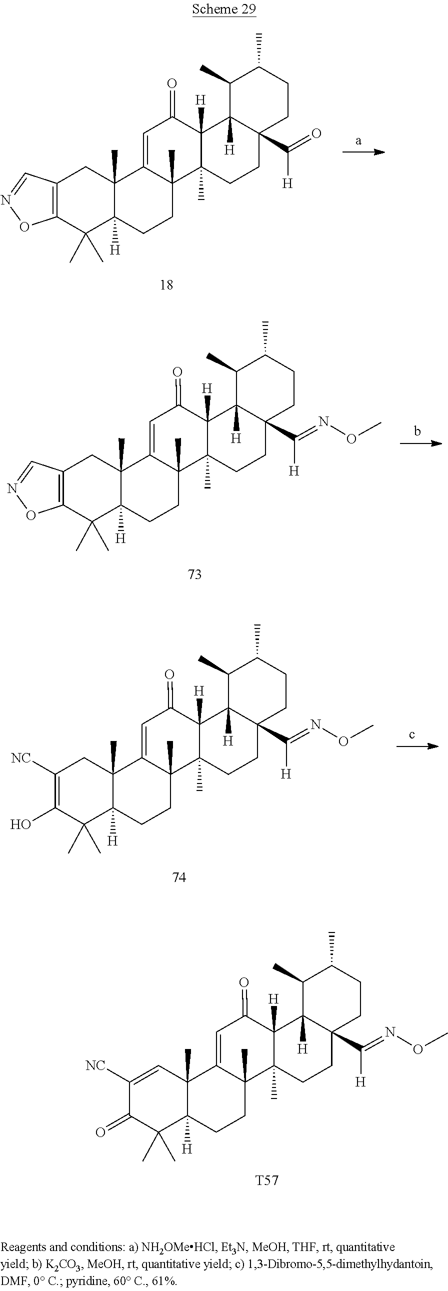

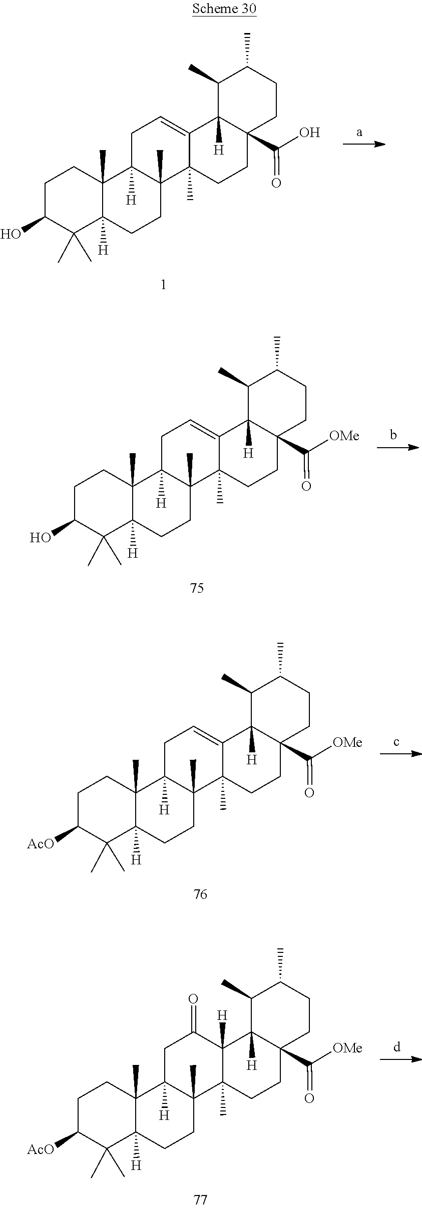

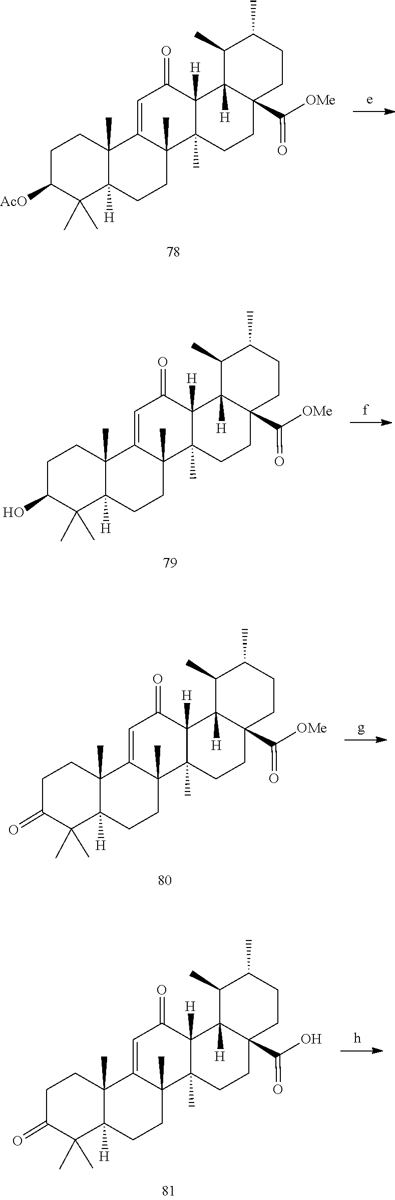

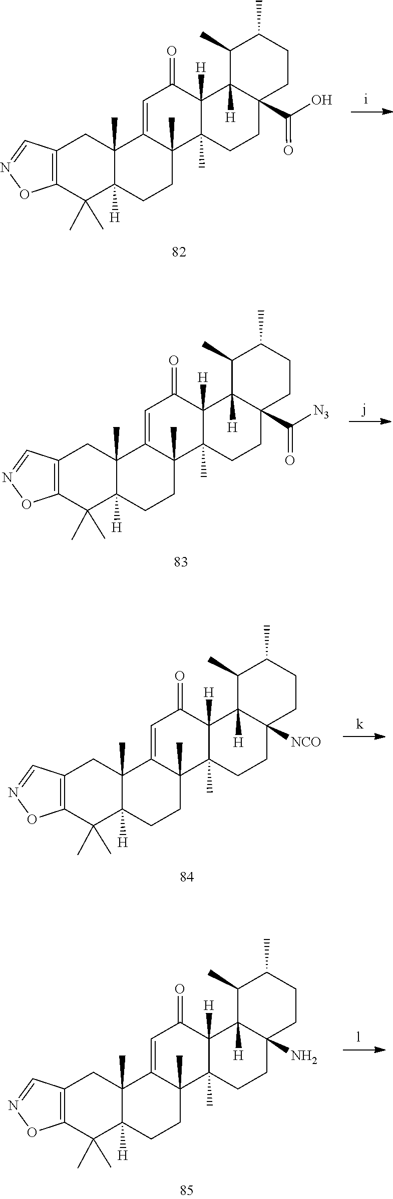

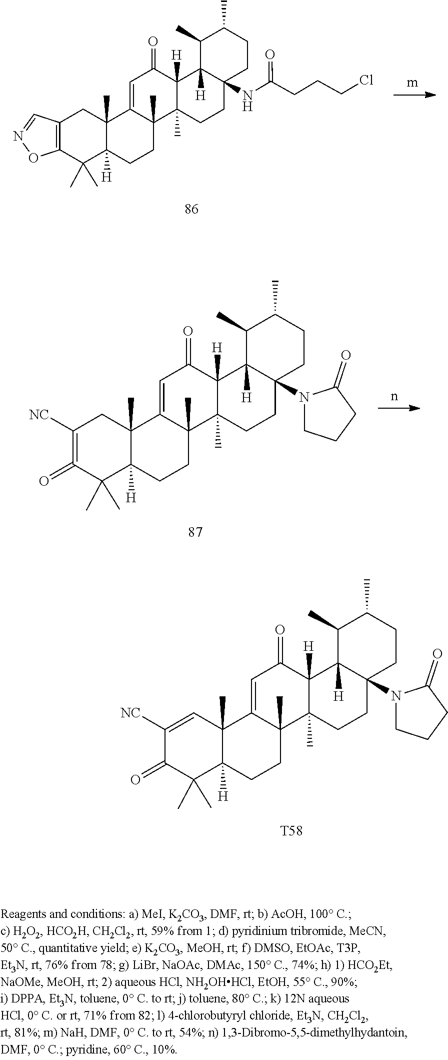

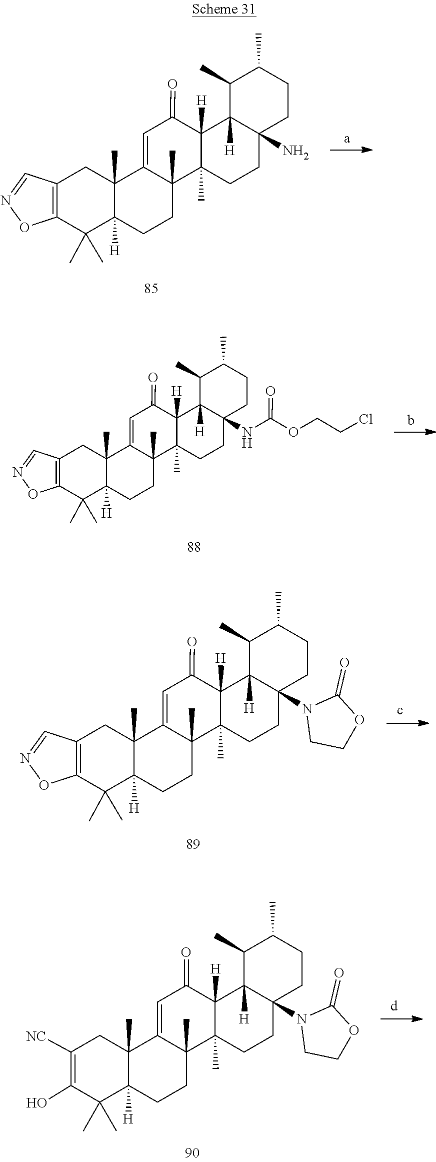

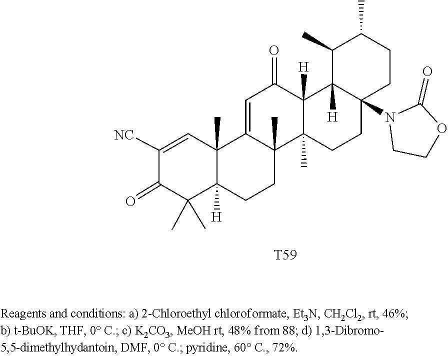

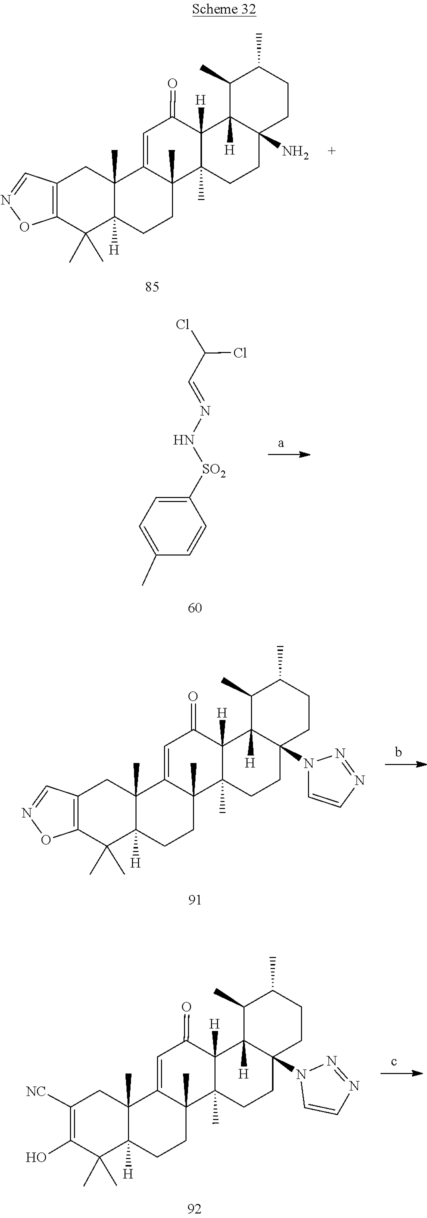

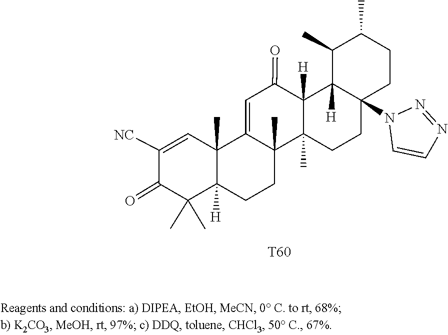

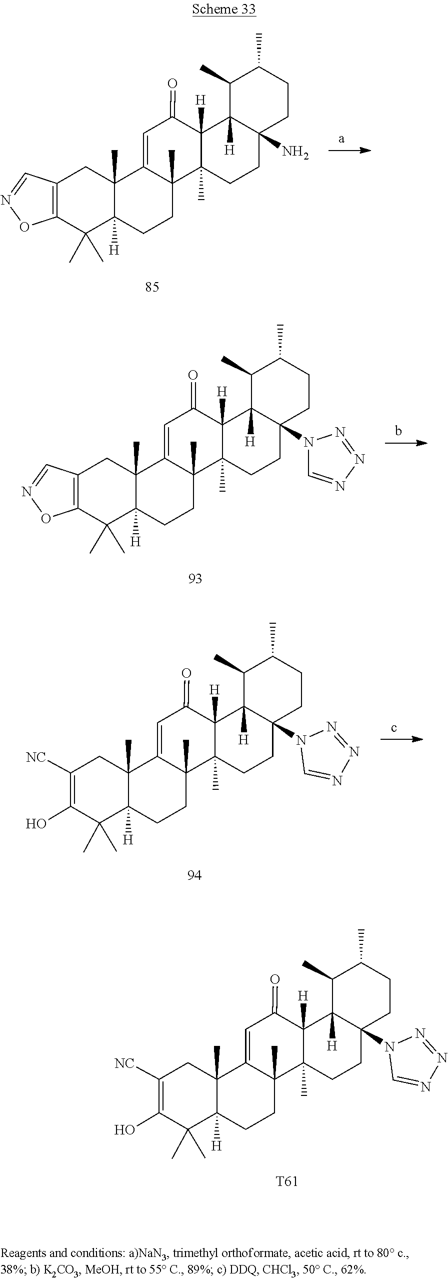

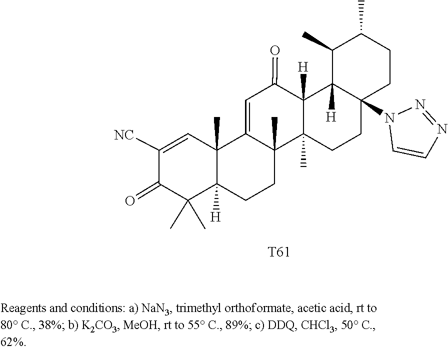

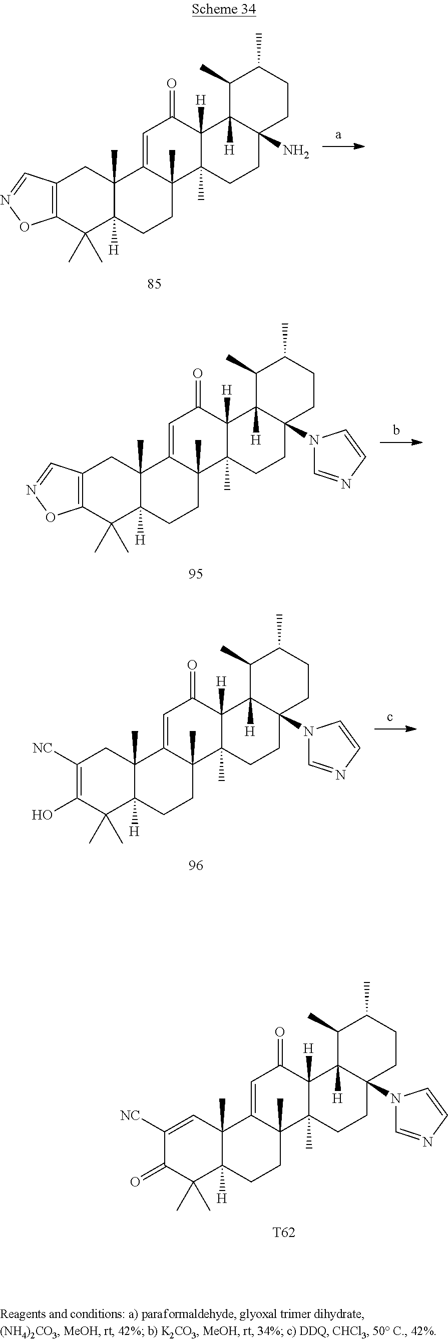

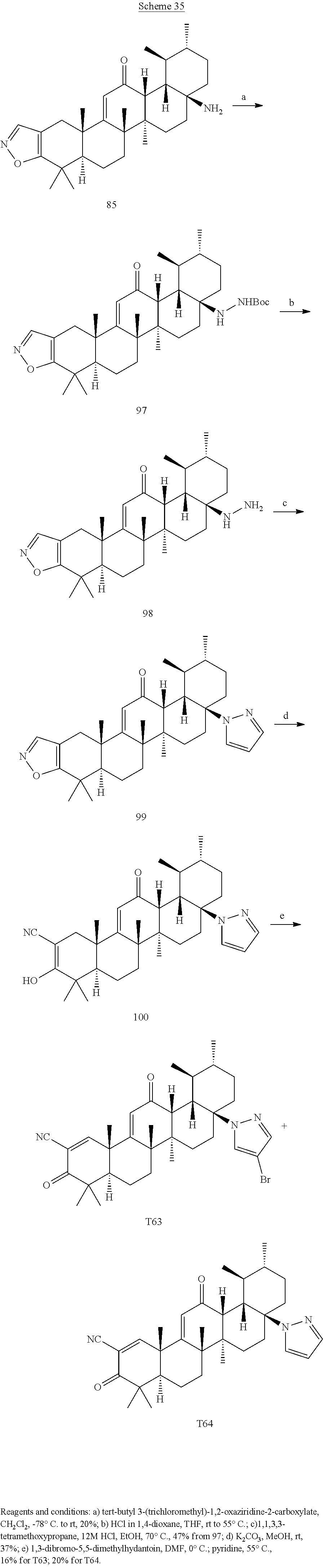

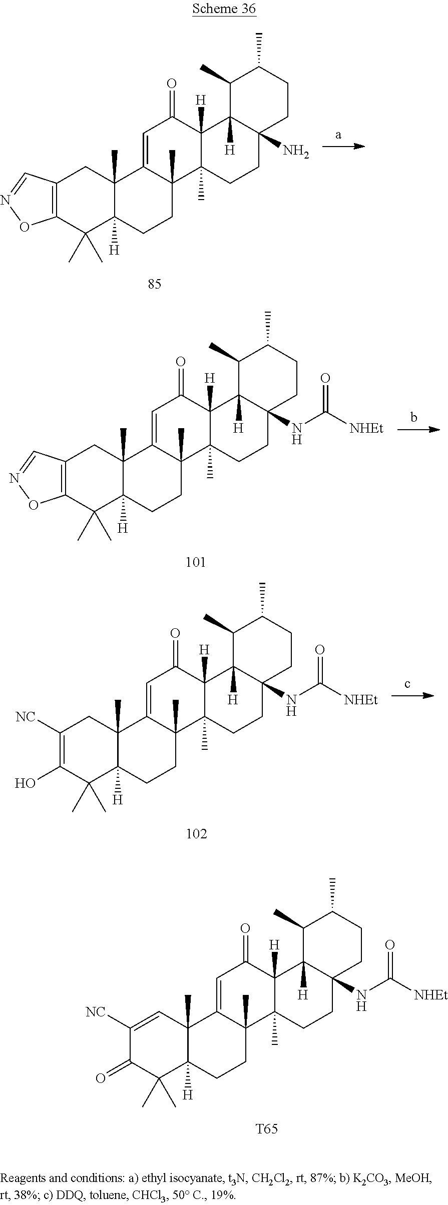

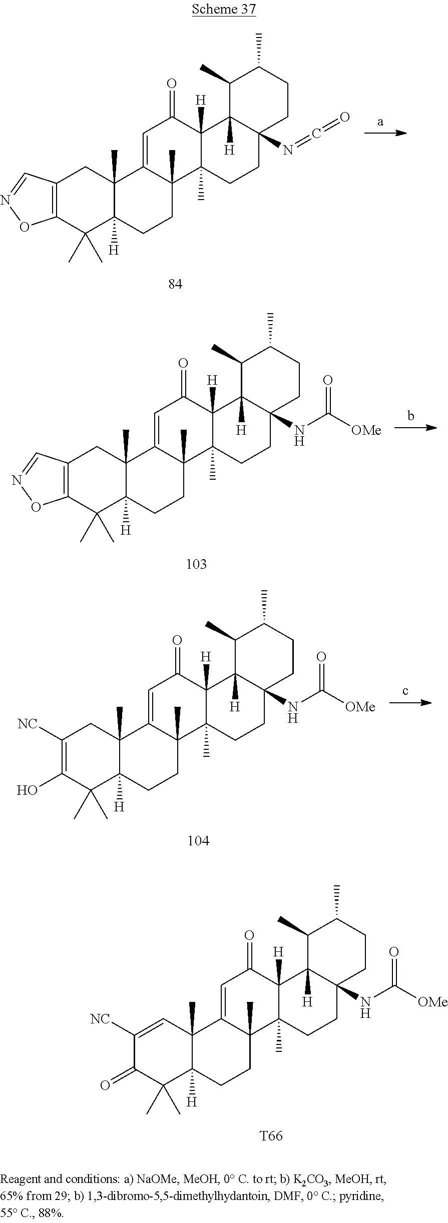

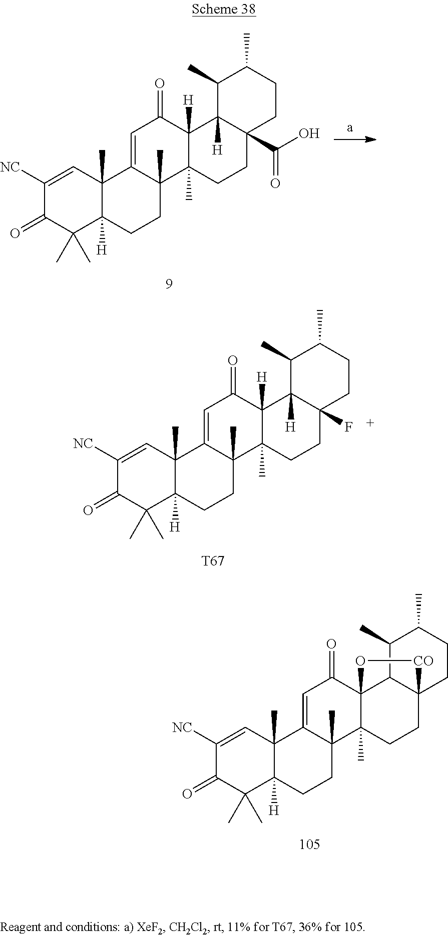

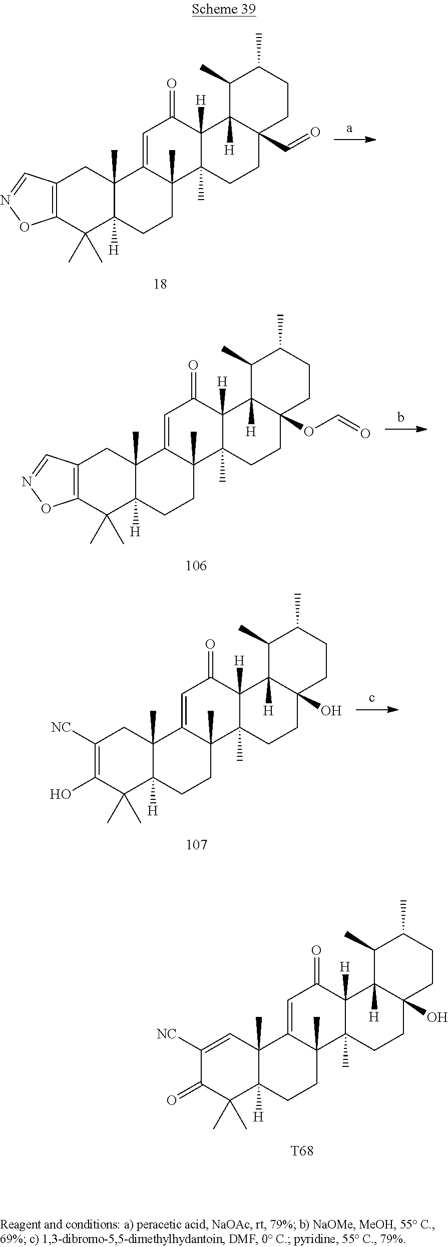

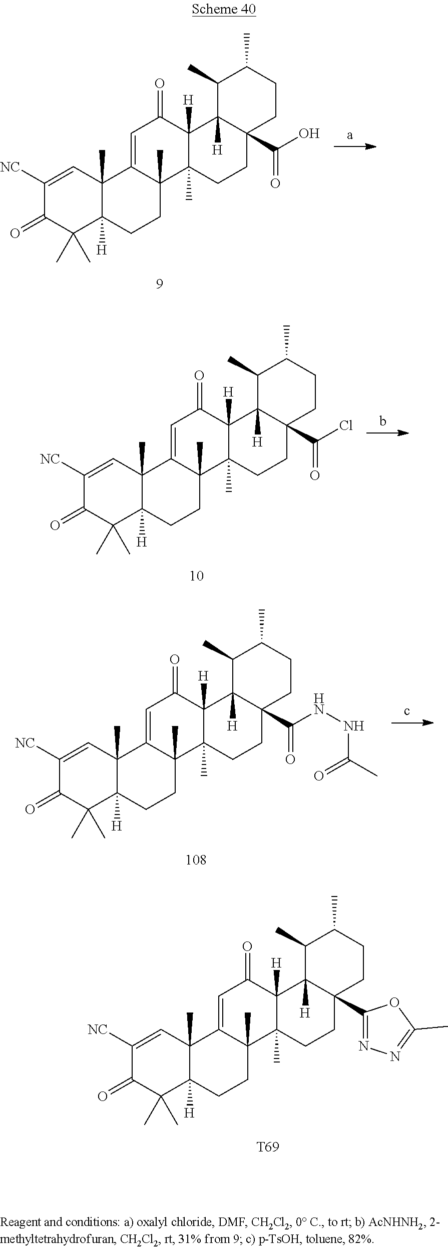

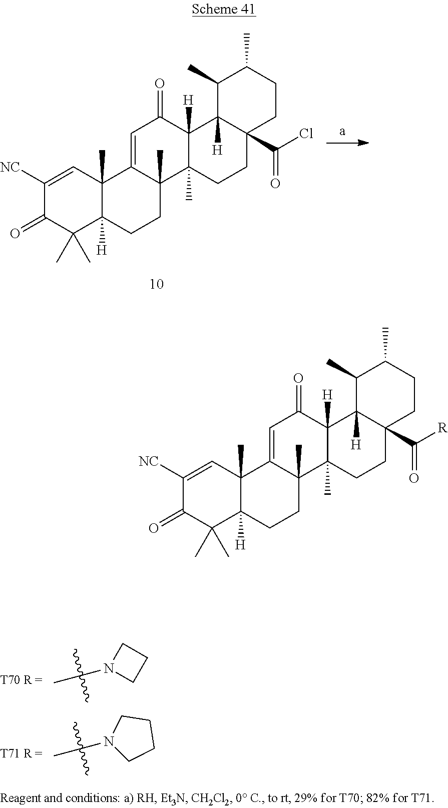

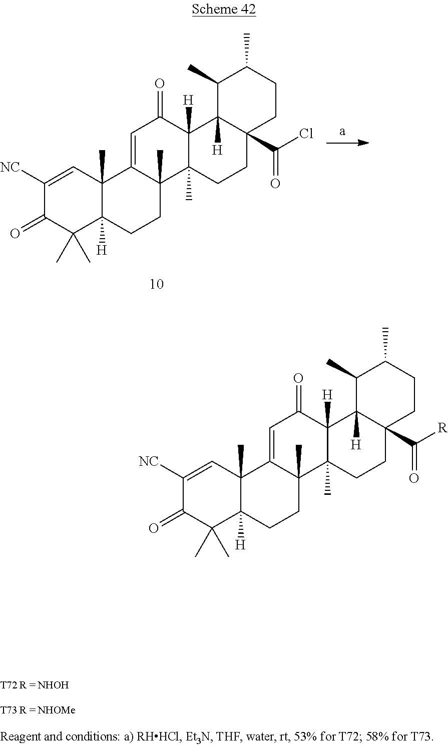

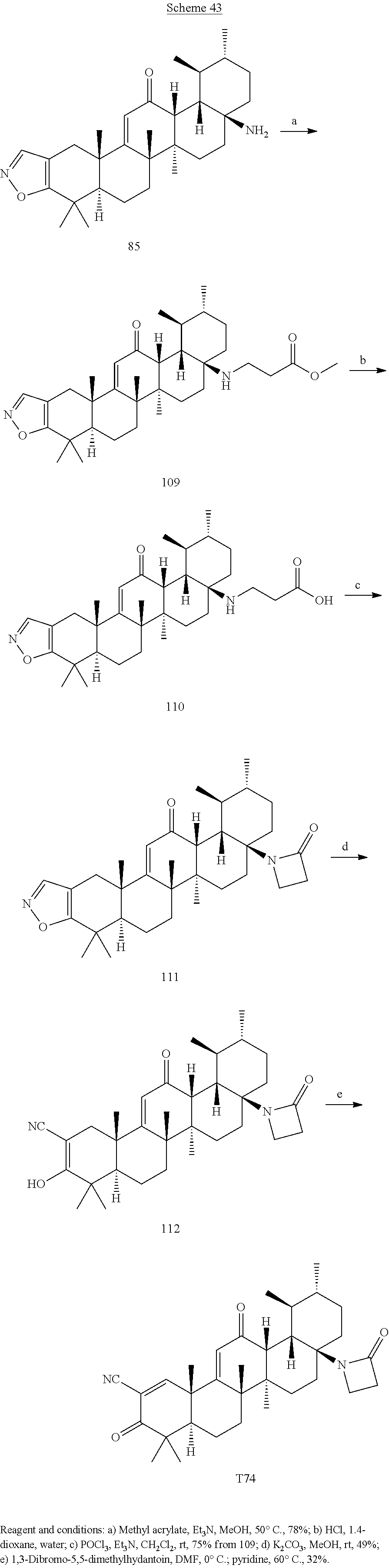

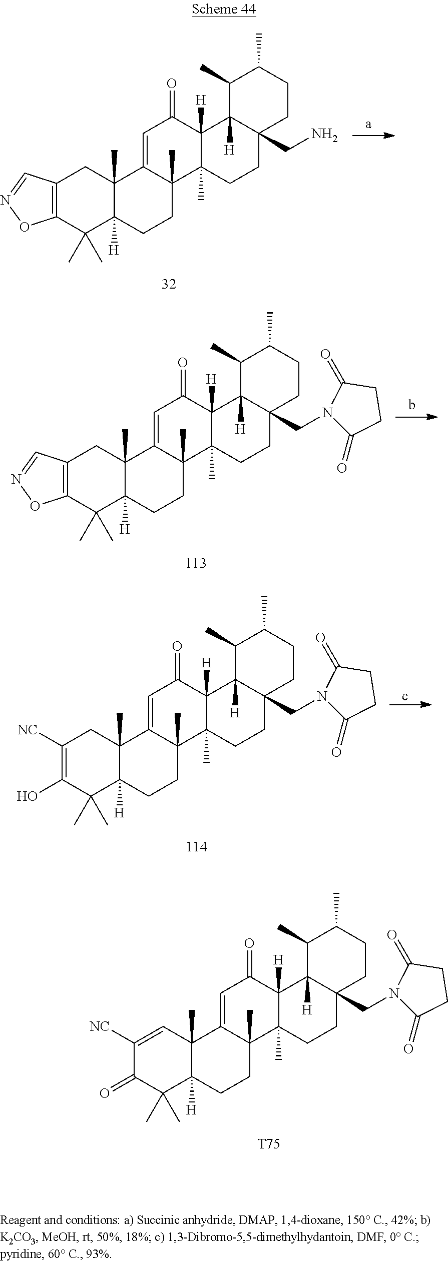

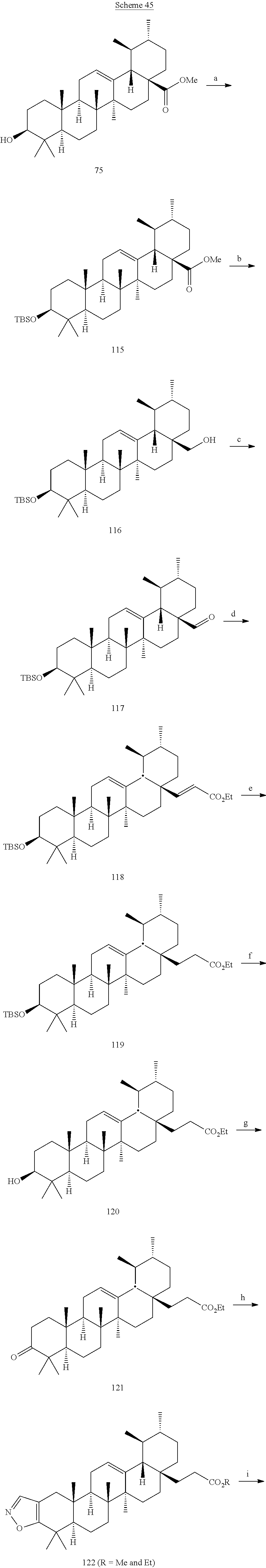

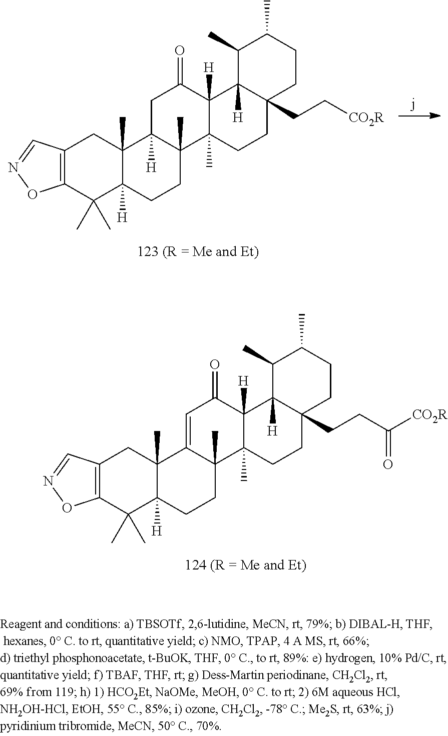

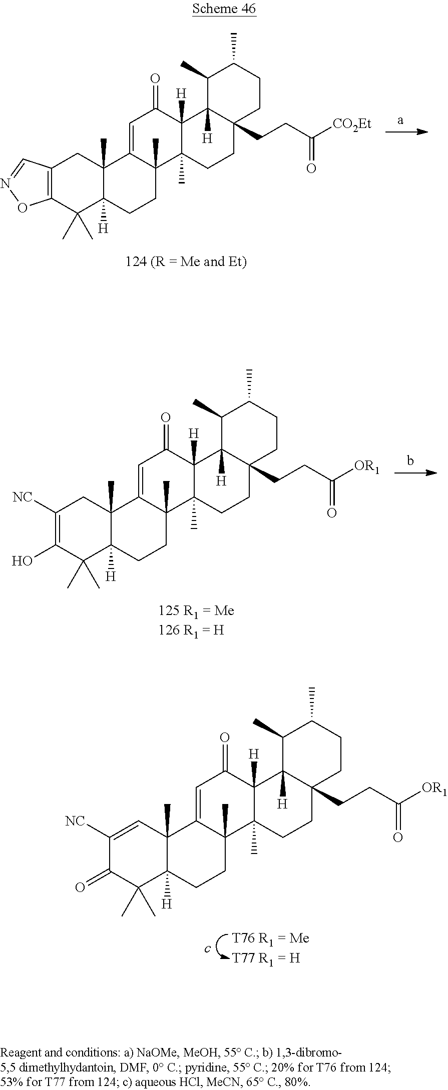

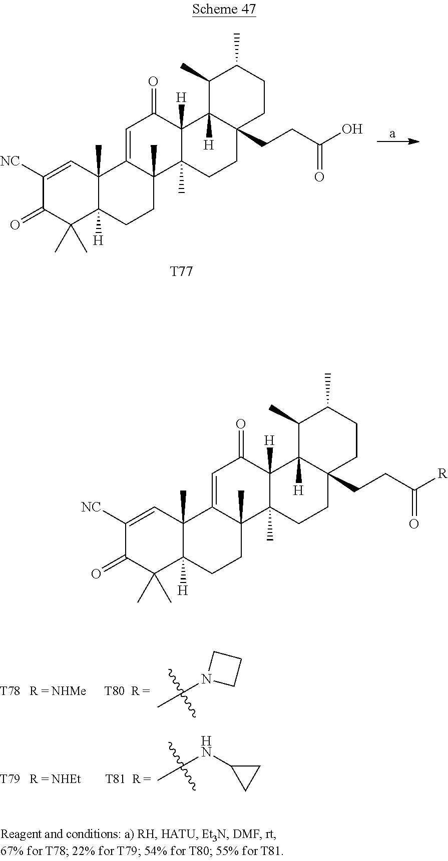

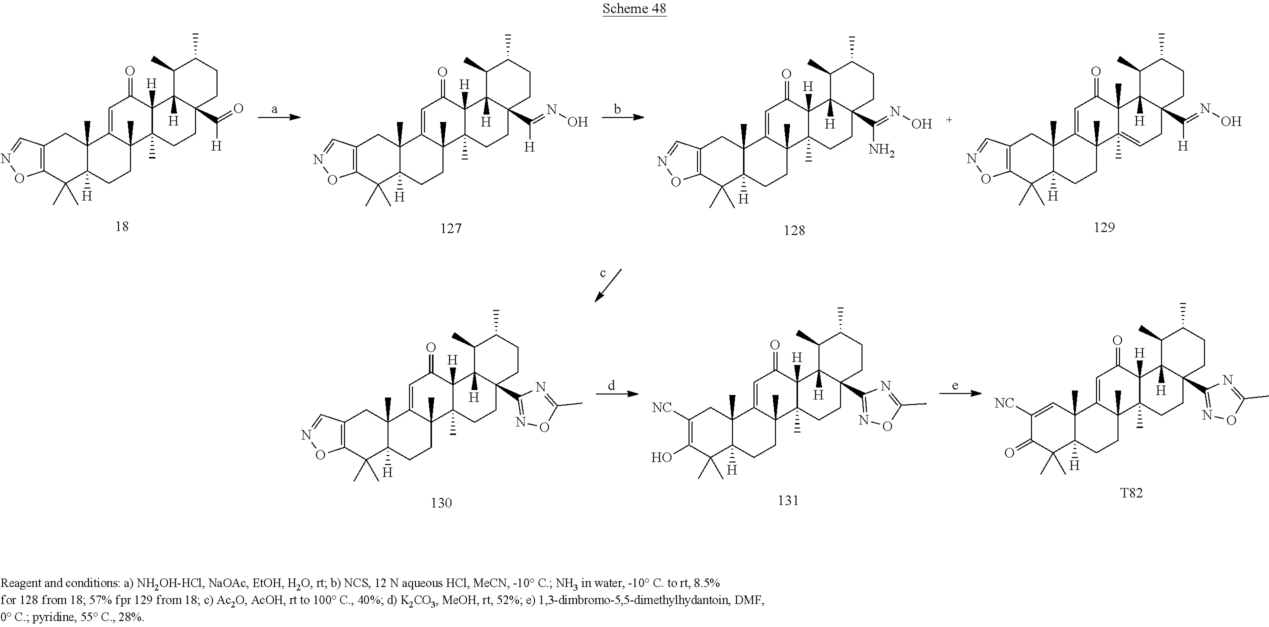

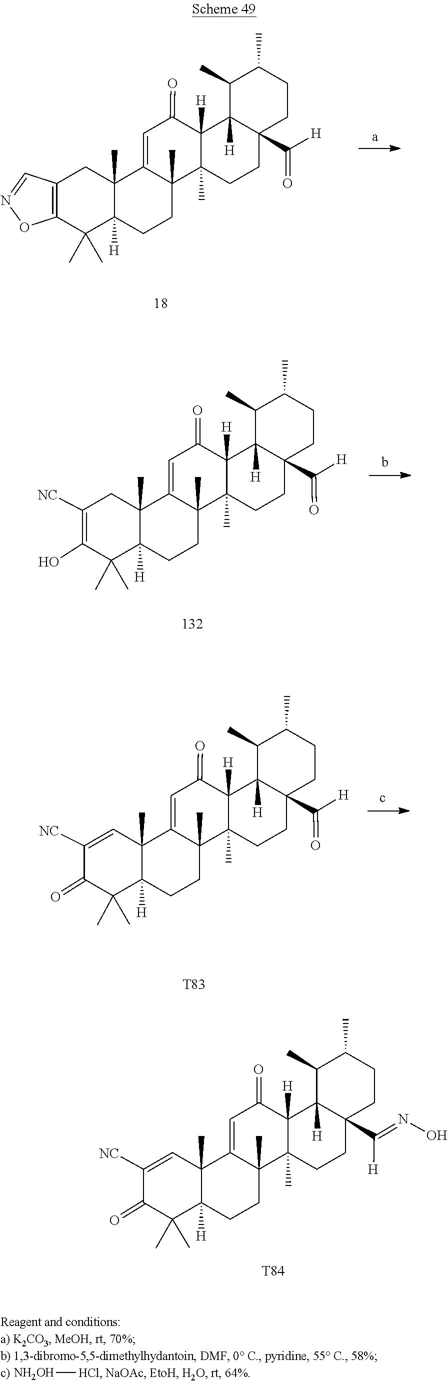

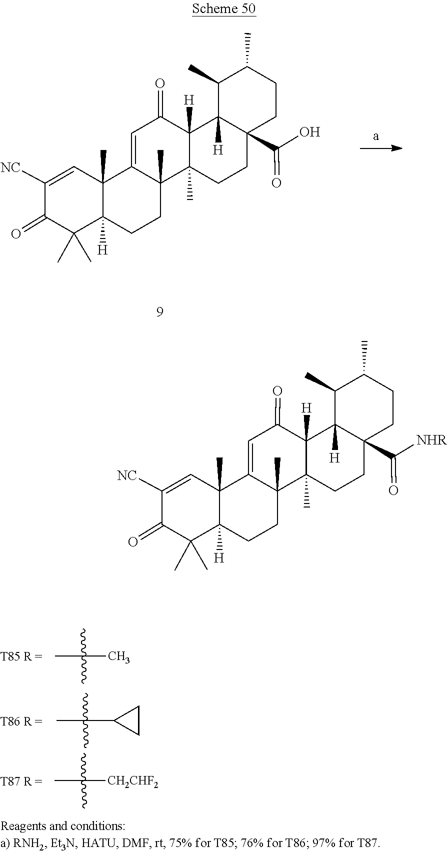

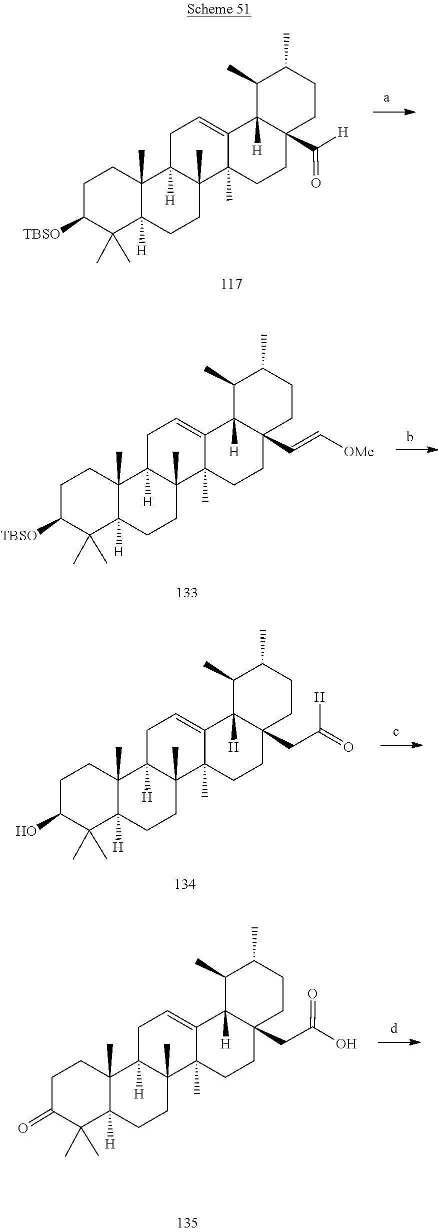

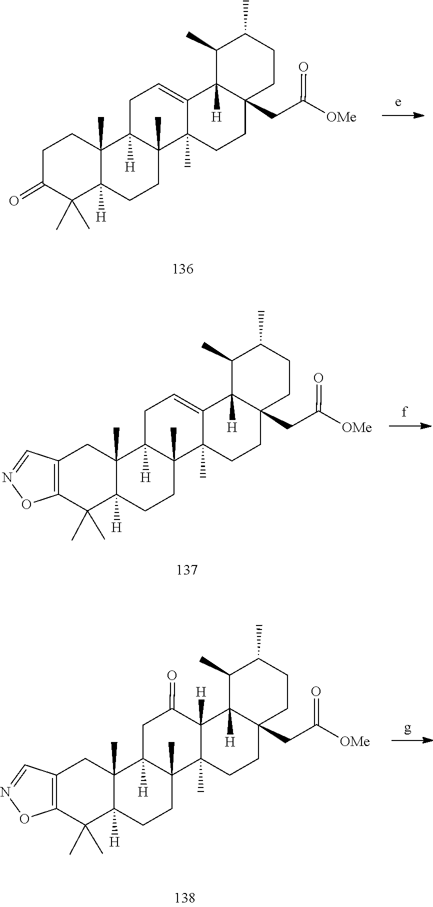

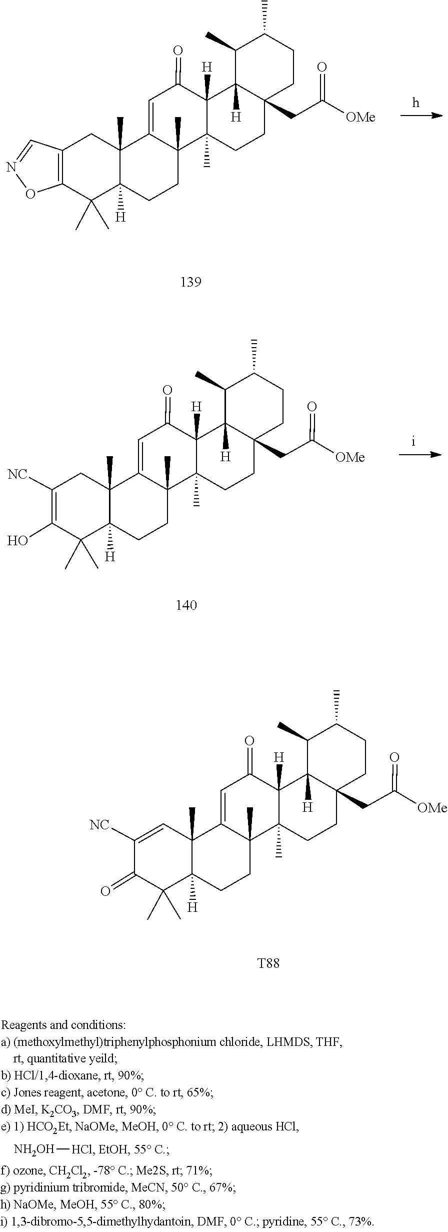

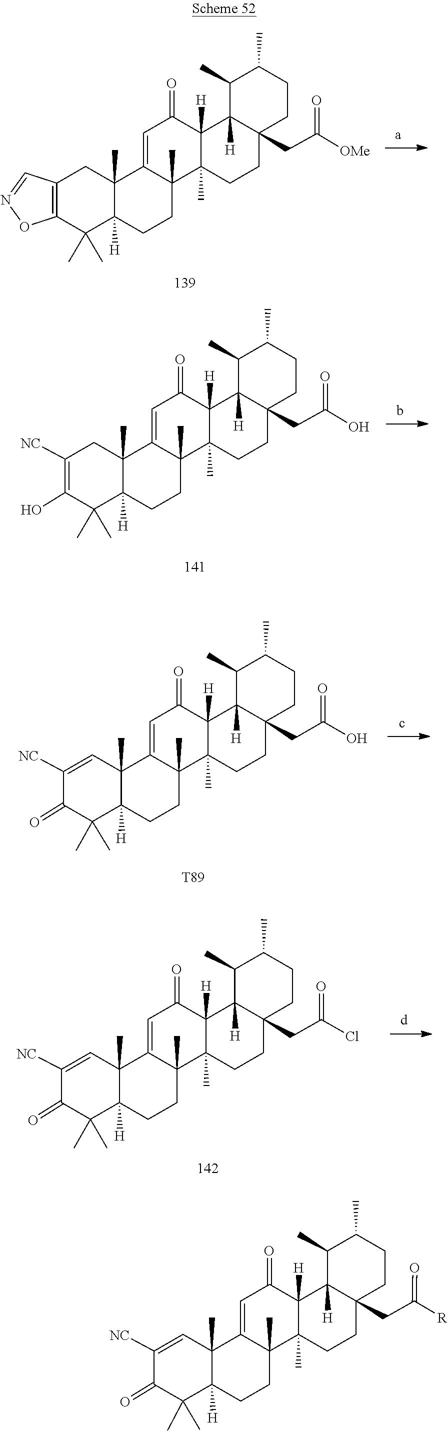

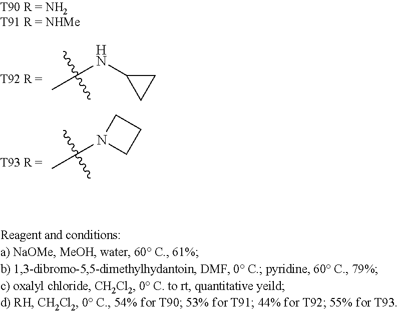

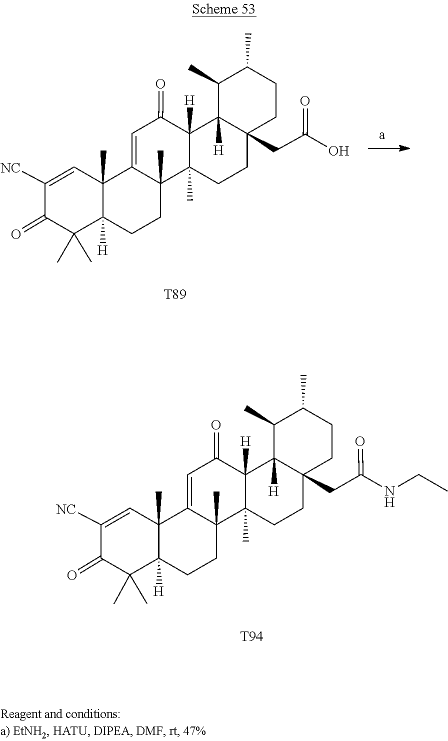

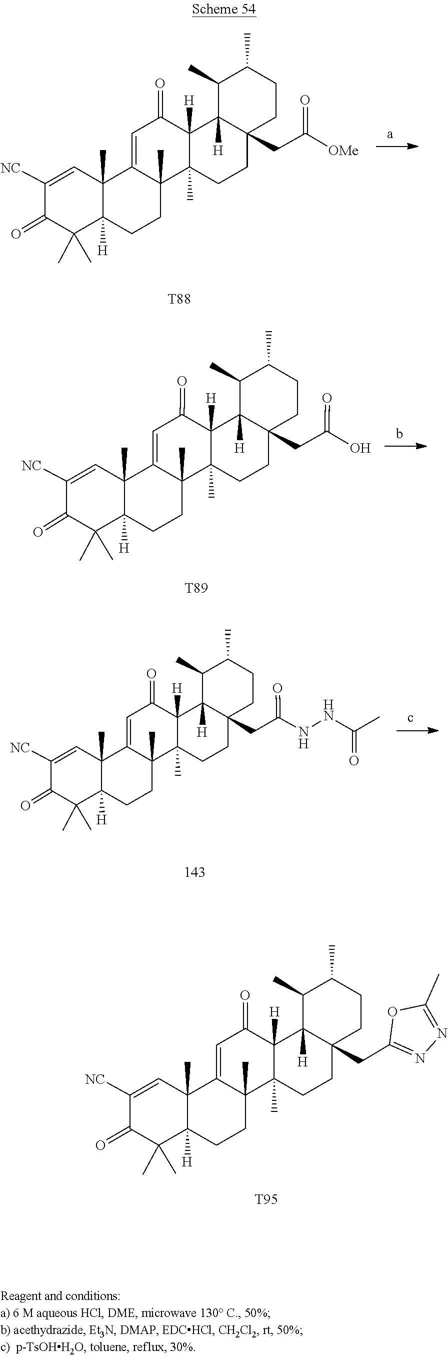

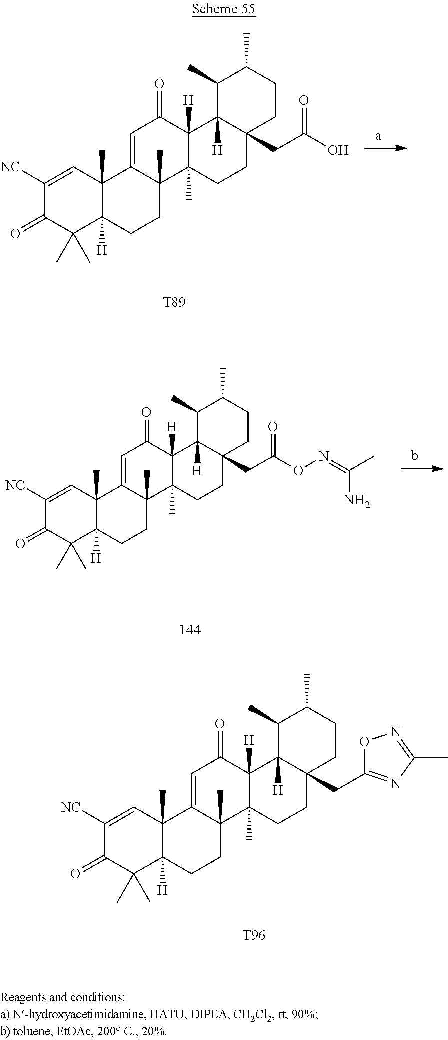

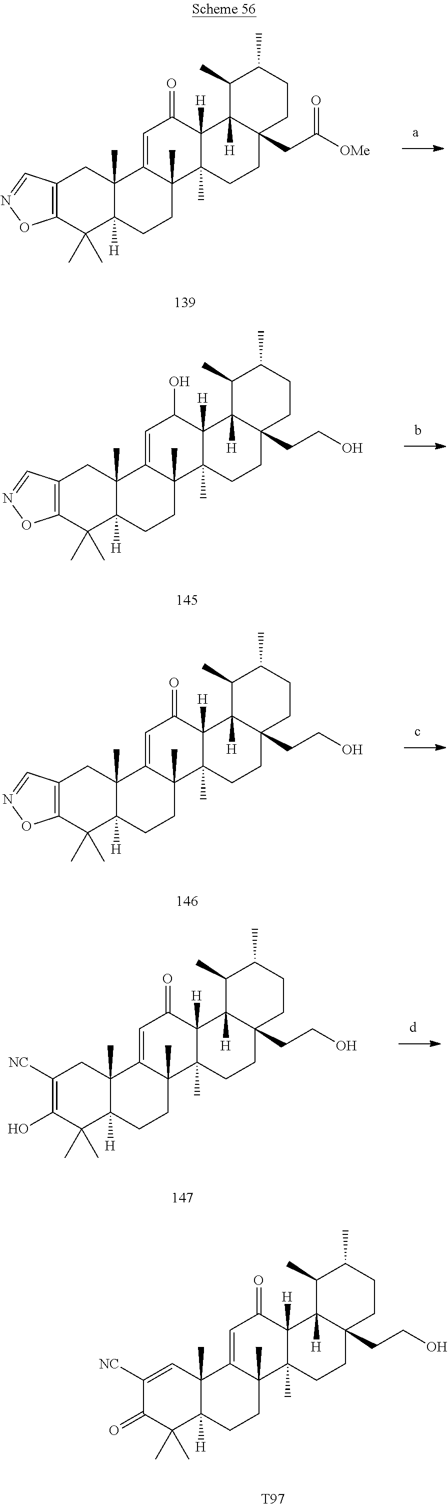

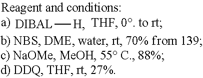

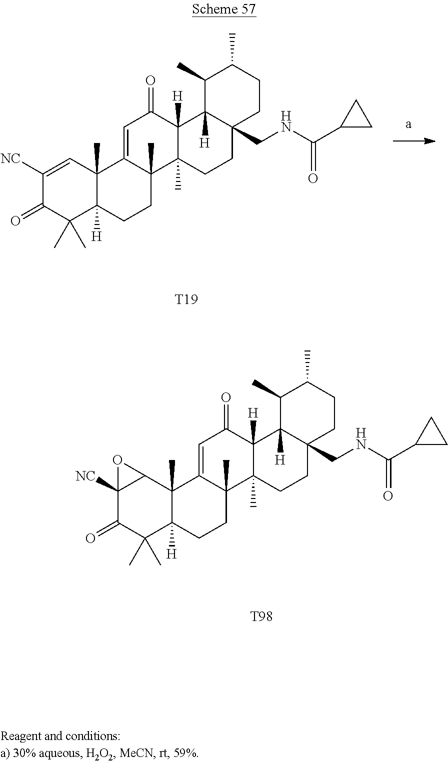

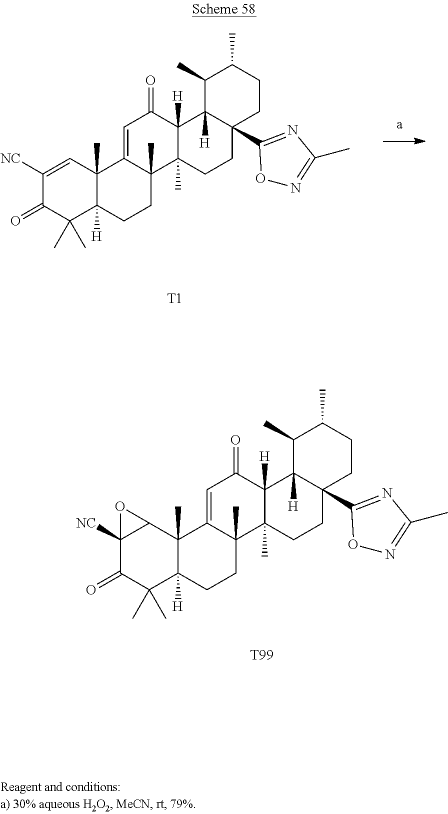

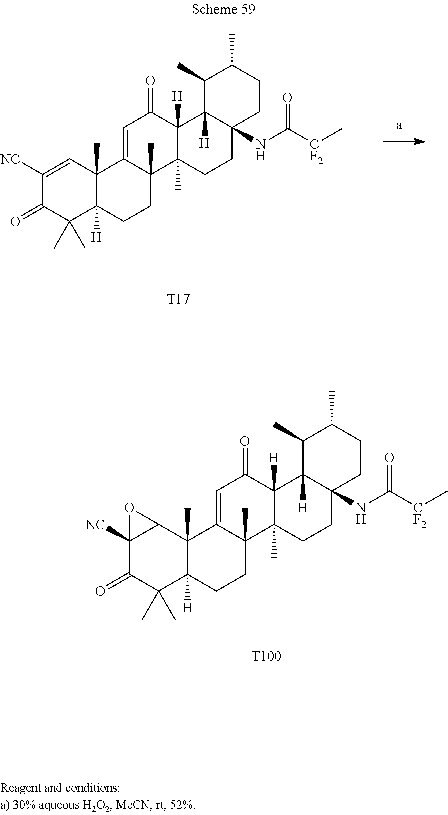

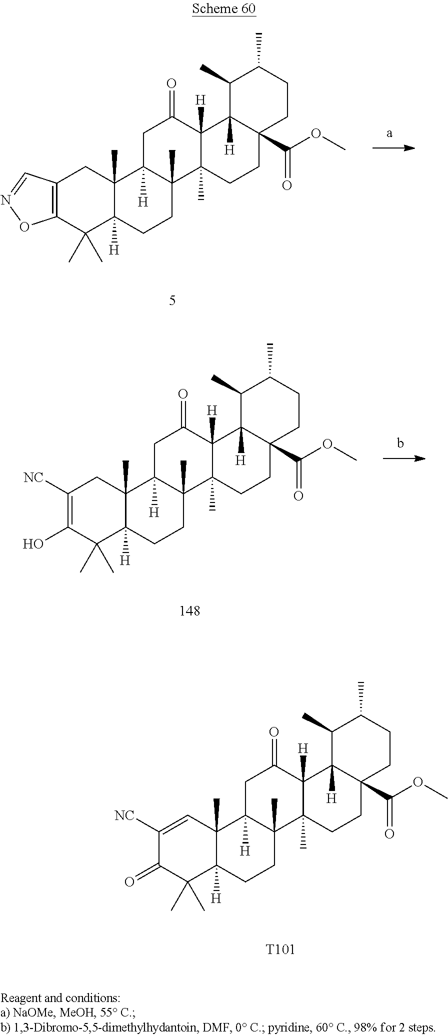

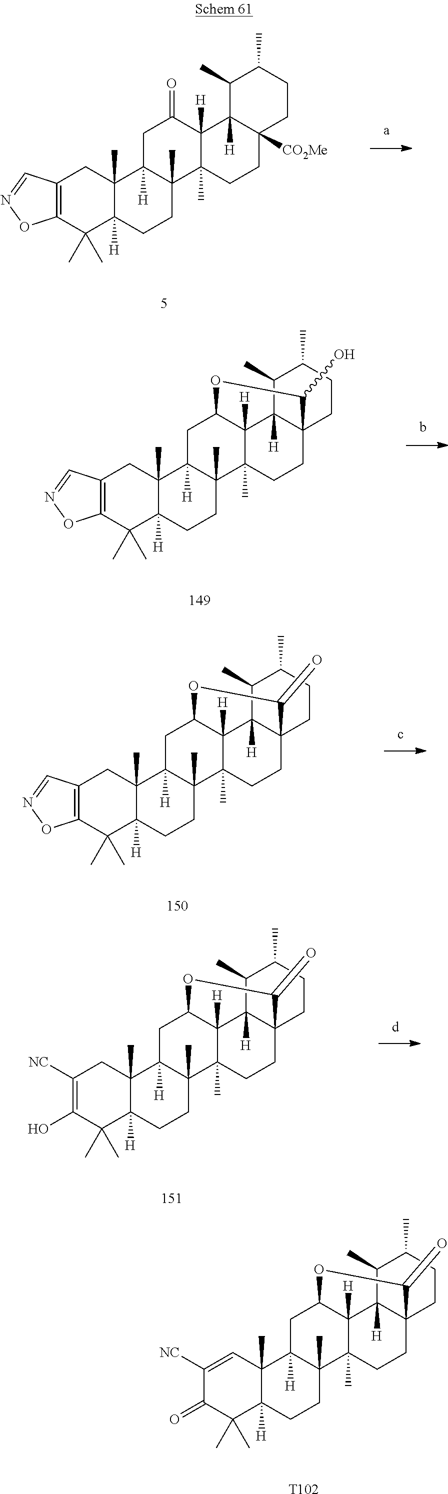

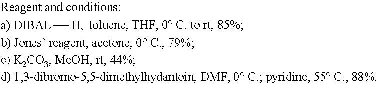

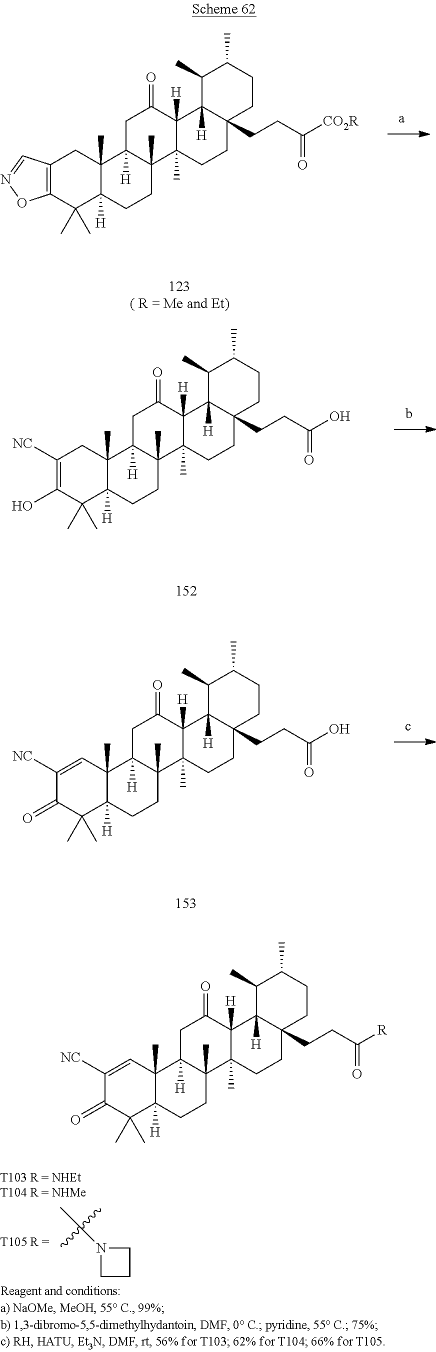

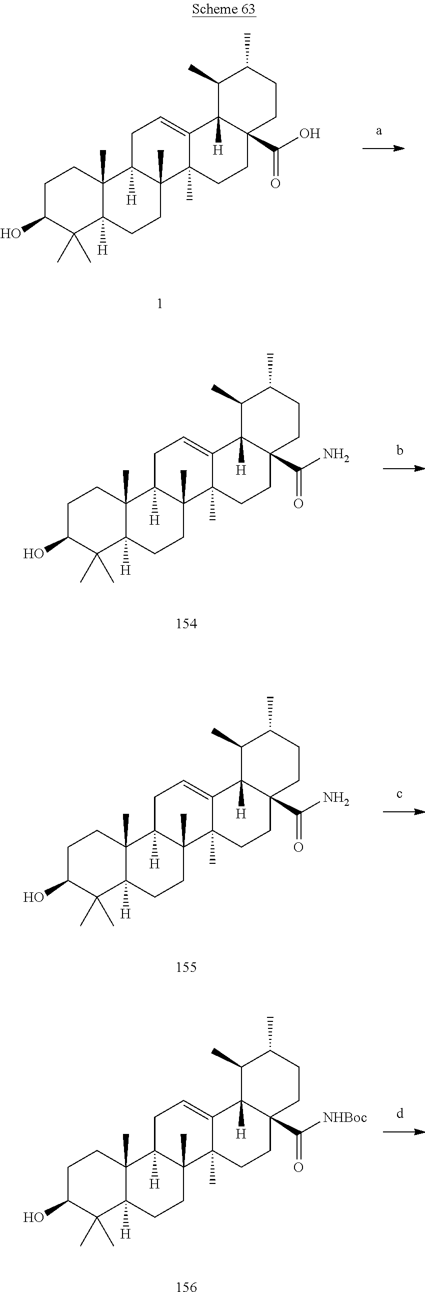

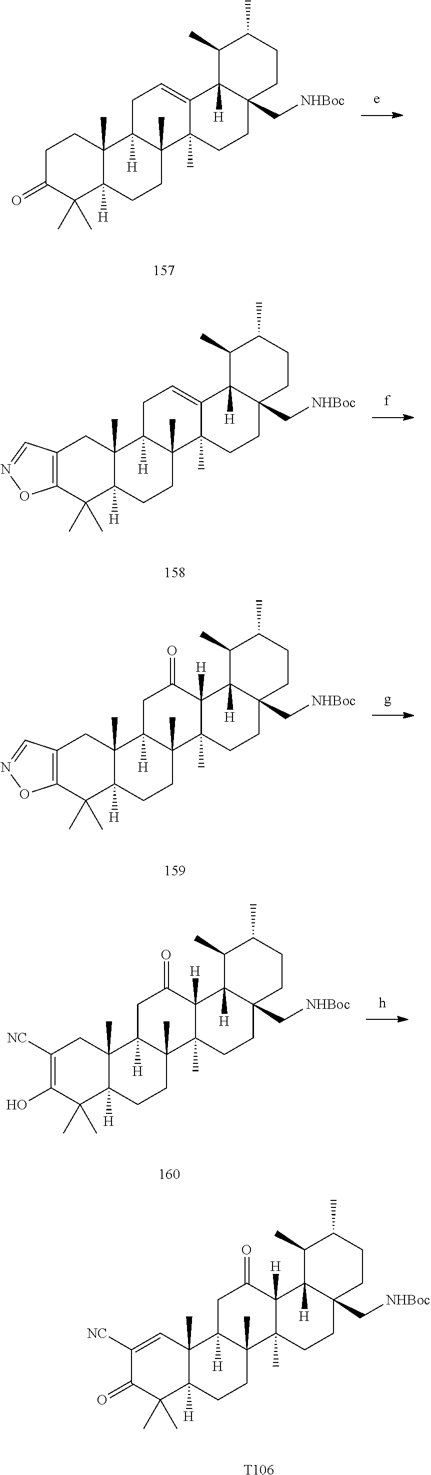

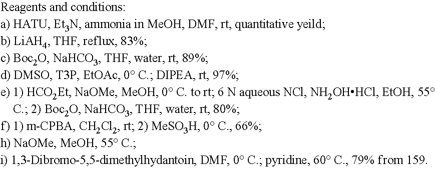

### C. Characterization Data

Compound 2: Ursolic acid 1 (500 g, 1.09 mol) was dissolved in CH₂Cl₂ (6 L). 4 Å MS (1000 g) and 4-methylmorpholine N-oxide (282 g, 2.41 mmol) were added. The mixture was stirred at room temperature for 10 min under N₂. Tetrapropylammonium perruthenate (TPAP, 38.6 g, 110 mmol) was added. The mixture was stirred at room temperature for 2 h and then quenched with 10% aqueous Na₂SO₃ (2 L). The mixture was extracted with CH₂Cl₂ (2×2 L). The organic extracts were combined; washed with water (2 L); dried with Na₂SO₄; filtered; and concentrated to give the crude product 2 (520 g, quantitative yield) as a white solid, which was used in the next step without further purification. m/z=455 (M+1).

Compound 3: To a mixture of compound 2 (520 g, <1.09 mol) in toluene (2.1 L) and MeOH (0.7 L) was added (trimethylsilyl)diazomethane (2.0 M solution in hexanes, 0.82 L, 1.64 mol) slowly at 0° C. The mixture was stirred at ambient temperature overnight; cooled to 0° C.; quenched with acetic acid (1 L); and stirred at ambient temperature for 10 min. The reaction mixture was concentrated. The residue was purified by column chromatography (silica gel, eluting with 0-100% EtOAc in hexanes) to give compound 3 (370 g, 72% yield) as a white solid.

Compound 4: To a mixture of compound 3 (120 g, 0.256 mol) and ethyl formate (586 mL, 7.25 mol) at 0° C. was added sodium methoxide (30 wt. % solution in MeOH, 768 mL, 3.84 mol). The mixture was stirred at ambient temperature for 2 h. Aqueous HCl (6 M, 640 mL, 3.84 mol), EtOH (2.4 L) and hydroxylamine hydrochloride (26.7 g, 0.384 mol) were added sequentially. The result mixture was heated at 55° C. for 4 h and then concentrated. The residue was diluted with water (1 L) and was extracted with EtOAc (3×2 L). The combined organic extracts were washed with brine; dried over anhydrous Na₂SO₄; filtered and concentrated. The residue was purified by column chromatography (silica gel, eluting with 33% EtOAc in hexanes) to give compound 4 (95 g, 75% yield) as a white solid. m/z=494 (M+1).

Compound 5: 30% aqueous hydrogen peroxide (205 mL, 2.01 mol) was added to formic acid (1 L, 26.5 mol) with stirring at room temperature. The resultant solution was added to a solution of compound 4 (90 g, 182 mmol) in formic acid (200 mL) with stirring at room temperature. The mixture was stirred at room temperature overnight; cooled to 10° C.; and quenched with 10% aqueous Na₂SO₃. The mixture was extracted with EtOAc (2 L). The organic extract was washed with brine; dried over Na₂SO₄; filtered and concentrated. The residue was purified by column chromatography (silica gel, eluting with 0-100% EtOAc in hexanes) to give compound 5 (70 g, 75% yield) as a white solid. m/z=510 (M+1).

Compound 6: To a solution of compound 5 (70 g, 137 mmol) in acetonitrile (1.3 L) was added pyridinium tribromide (57 g, 178 mmol) at room temperature. The reaction mixture was stirred at 50° C. for 3 h; cooled; and quenched with 10% aqueous Na₂SO₃ solution. The mixture was extracted with CH₂Cl₂ (3×1 L). The combined organic extracts were dried with MgSO₄; filtered and concentrated. The residue was purified by column chromatography (silica gel, eluting with 0-40% EtOAc in hexanes) to give compound 6 (61 g, 87% yield) as a white solid. m/z=508 (M+1).

Compound 7: To a stirring mixture of compound 6 (3.01 g, 5.93 mmol) in MeOH (30 mL) at room temperature was added sodium methoxide (25 wt. % in MeOH, 2.03 mL, 8.89 mmol). The mixture was heated at 55° C. for 2 h; cooled to 0° C.; and poured into 10% aqueous NaH₂PO₄ solution (150 mL) at 0° C. The mixture was stirred at 0° C. for 10 min; and then filtered. The wet cake was washed with water (50 mL); and then dissolved in EtOAc (100 mL). The mixture was washed with water (20 mL) and brine (20 mL) sequentially. The organic extract was dried with MgSO₄; filtered and concentrated to give compound 7 (3.14 g, quantitative yield) as a light pink solid. m/z=508 (M+1).

Compound 8: Compound 7 (3.14 g, 5 5.93 mmol) was dissolved in anhydrous DMF (15 mL) and cooled to 0° C. A solution of 1,3-dibromo-5,5-dimethylhydantoin (848 mg, 2.97 mmol) in anhydrous DMF (15 mL) was added under N₂. The mixture was stirred at 0° C. for 1 h, and then treated with pyridine (1.92 mL, 23.7 mmol). The mixture was stirred at 60° C. for 4 h; cooled to room temperature; and poured into 1 N aqueous HCl (100 mL). The mixture was stirred at room temperature for 10 min; and filtered. The solid was washed with water (2×20 mL); and was dissolved in EtOAc (100 mL). The solution was washed with 1 N aqueous HCl (50 mL) and water (50 mL) sequentially. The combined aqueous washes were extracted with EtOAc (50 mL), and was washed with water (20 mL). The combined organic extracts were dried with MgSO₄; filtered; and concentrated. The residue was purified by column chromatography (silica gel, eluting with 0-100% EtOAc in hexanes) to give compound 8 (2.58 g, 86% yield) as a light yellow solid. m/z=506 (M+1).

Compound 9: To a stirring mixture of compound 8 (1.00 g, 1.98 mmol) and NaOAc (406 mg, 4.94 mmol) in N,N-Dimethylacetamide (20 mL) at 0° C. was added LiBr (1.72 g, 19.8 mmol). The mixture was heated at 150° C. with N₂ bubbled through the reaction for 6 h; cooled to 0° C.; and treated with 1 N aqueous HCl (40 mL). The mixture was stirred at room temperature for 10 min, and then filtered. The wet cake was washed with water (3×20 mL), and was dissolved in EtOAc (80 mL). The solution was washed with water (3×20 mL) and brine (20 mL). The organic extract was dried with MgSO₄; filtered and concentrated. The residue was purified by column chromatography (silica gel, eluting with 0-70% EtOAc in hexanes) to give compound 9 (655 mg, 67% yield) as a brown solid. m/z=492 (M+1).

Compound 10: To a solution of compound 9 (339 mg, 0.689 mmol) in CH₂Cl₂ (6.9 mL) at 0° C. was added oxalyl chloride (0.175 mL, 2.00 mmol) and 1 drop of DMF sequentially. The mixture was stirred at room temperature for 2 h and then concentrated. The residue was dissolved in toluene (5 mL) and concentrated. The process was repeated four times to remove residual oxalyl chloride. The residue was dried under vacuum to give compound 10 as a dark brown solid, which was used in the next step without further purification. m/z=446 (M-COCl).

Compound 11 (T107): To a solution of compound 10 (all from the above, 0.689 mmol) in CH₂Cl₂ (6.9 mL) at 0° C. was added triethylamine (0.287 mL, 2.06 mmol) and N′-hydroxyacetimidamide (76.6 mg, 1.03 mmol). The mixture was stirred at room temperature for overnight; and then was washed with water. The aqueous wash was extracted with CH₂Cl₂. The combined organic extracts were dried with Na₂SO₄; filtered; and concentrated. The residue was purified by column chromatography (silica gel, eluting with 0-20% acetone in CH₂Cl₂) to give compound 11 (160 mg, 42% yield) as a tan solid. m/z=548 (M+1). ¹H NMR (400 MHz, CDCl₃) δ 8.03 (s, 1H), 6.12 (s, 1H), 4.81 (bs, 2H), 3.03 (dd, J=11.6, 3.2 Hz, 1H), 2.62 (d, J=3.6 Hz, 1H), 1.96 (s, 3H), 1.48 (s, 3H), 1.26 (s, 3H), 1.20 (s, 3H), 1.19 (s, 3H), 1.17-1.96 (m, 15H), 1.15 (s, 3H), 0.92 (d, J=6.1 Hz, 3H), 0.74 (d, J=6.5 Hz, 3H).

Compound T1: To a solution of compound 11 (130 mg, 0.237 mmol) in THF (7.1 mL) at room temperature was added tetrabutylammonium hydroxide solution (40 wt. % aqueous, 0.510 mL, 0.782 mmol). The mixture was stirred at room temperature for overnight; diluted with EtOAc; and washed with water. The aqueous was extracted with EtOAc. The combined organic extracts were dried with Na₂SO₄; filtered; and concentrated. The residue was purified by column chromatography (silica gel, eluting with 0-60% EtOAc in hexanes) to give compound T1 (51 mg, 41% yield) as a tan solid. m/z=530 (M+1); ¹H NMR (400 MHz, CDCl₃) δ 8.02 (s, 1H), 6.10 (s, 1H), 3.18 (m, 1H), 2.54 (d, J=3.8 Hz, 1H), 2.36 (s, 3H), 2.26 (td, J=13.8, 4.5 Hz, 1H), 2.07-1.99 (m, 1H), 1.90-1.20 (m, 13H), 1.44 (s, 3H), 1.25 (s, 3H), 1.17 (s, 3H), 1.16 (s, 3H), 1.00 (s, 3H), 0.95 (d, J=5.8 Hz, 3H), 0.80 (d, J=6.6 Hz, 3H).

Compound 13a: To a solution of compound 10 (125 mg, 0.245 mmol) in CH₂Cl₂ (2.4 mL) at 0° C. was added triethylamine (0.102 mL, 0.732 mmol) and compound 12a (38.2 mg, 0.382 mmol). The mixture was stirred at room temperature for 1 h; and then was partitioned between CH₂Cl₂ and water. The aqueous layer was separated and was extracted with CH₂Cl₂. The combined organic extracts were dried with Na₂SO₄; filtered; and concentrated. The residue was purified by column chromatography (silica gel, eluting with 0-20% acetone in CH₂Cl₂) to give compound 13a (85 mg, 60% yield) as a yellow solid. m/z=574 (M+1).

Compound T2: To a solution of compound 13a (55 mg, 0.096 mmol) in THF (1 mL) at room temperature was added tetrabutylammonium hydroxide solution (40 wt. % aqueous, 0.10 mL, 0.15 mmol). The mixture was stirred at room temperature for 2 h; diluted with EtOAc; and washed with water. The aqueous layer was extracted with EtOAc. The combined organic extracts were dried with Na₂SO₄; filtered; and concentrated. The residue was combined with the crude product obtained from compound 13a (30 mg, 0.052 mmol), and was purified by column chromatography (silica gel, eluting with 0-50% EtOAc in hexanes) to give compound T2 (46 mg, 56% yield) as a white solid. m/z=556 (M+1); ¹H NMR (400 MHz, CDCl₃) δ 8.02 (s, 1H), 6.09 (s, 1H), 3.12 (dd, J=11.2, 3.7 Hz, 1H), 2.60 (d, J=3.8 Hz, 1H), 2.22 (td, J=13.7, 4.5 Hz, 1H), 2.09-1.93 (m, 2H), 1.90-0.90 (m, 17H), 1.44 (s, 3H), 1.25 (s, 3H), 1.16 (s, 3H), 1.14 (s, 3H), 0.99 (s, 3H), 0.93 (d, J=5.6 Hz, 3H), 0.78 (d, J=6.6 Hz, 3H).

Compound 13b: To a solution of compound 10 (120 mg, 0.235 mmol) in CH₂Cl₂ (2.3 mL) at 0° C. was added triethylamine (0.098 mL, 0.70 mmol) and compound 12b (37 mg, 0.36 mmol). The mixture was stirred at room temperature for 2 h; and then was partitioned between CH₂Cl₂ and water. The aqueous layer was separated and was extracted with CH₂Cl₂. The combined organic extracts were dried with Na₂SO₄; filtered; and concentrated. The residue was purified by column chromatography (silica gel, eluting with 0-80% EtOAc in hexanes) to give compound 13b (98 mg, 72% yield) as a yellow solid. m/z=578 (M+1).

Compound T3: To a solution of compound 13b (98 mg, 0.17 mmol) in THF (5 mL) at room temperature was added tetrabutylammonium hydroxide solution (40 wt. % aqueous, 0.221 mL, 0.339 mmol). The mixture was stirred at room temperature for 8 h; diluted with EtOAc; and washed with water. The aqueous layer was extracted with EtOAc. The combined organic extracts were dried with Na₂SO₄; filtered; and concentrated. The residue was purified by column chromatography (silica gel, eluting with 0-80% EtOAc in CH₂Cl₂) to give compound T3 (29 mg, 30% yield) as a white solid. m/z=560 (M+1); ¹H NMR (400 MHz, CDCl₃) δ 8.02 (s, 1H), 6.10 (s, 1H), 4.54 (d, J=1.8 Hz, 2H), 3.42 (s, 3H), 3.21 (dd, J=11.2, 3.7 Hz, 1H), 2.47 (d, J=3.8 Hz, 1H), 2.28 (td, J=13.7, 4.5 Hz, 1H), 2.08 (m, 1H), 1.95-1.20 (m, 13H), 1.43 (s, 3H), 1.25 (s, 3H), 1.17 (s, 3H), 1.16 (s, 3H), 0.99 (s, 3H), 0.95 (d, J=5.7 Hz, 3H), 0.80 (d, J=6.6 Hz, 3H).

Compound 13c: To a solution of compound 10 (100 mg, 0.196 mmol) in CH₂Cl₂ (2 mL) at 0° C. was added triethylamine (0.082 mL, 0.59 mmol) and compound 12c (26 mg, 0.30 mmol). The mixture was stirred at room temperature for 2 h; and then was partitioned between CH₂Cl₂ and water. The aqueous layer was separated and was extracted with CH₂Cl₂. The combined organic extracts were dried with Na₂SO₄; filtered; and concentrated. The residue was purified by column chromatography (silica gel, eluting with 0-80% EtOAc in hexanes) to give compound 13c (100 mg, 91% yield) as a white solid. m/z=562 (M+1).

Compound T4: To a solution of compound 13c (100 mg, 0.178 mmol) in THF (5.3 mL) at room temperature was added tetrabutylammonium hydroxide solution (40 wt. % aqueous, 0.231 mL, 0.354 mmol). The mixture was stirred at room temperature for 2 h; diluted with EtOAc; and washed with water. The organic extract was dried with Na₂SO₄; filtered; and concentrated. The residue was purified by column chromatography (silica gel, eluting with 0-40% EtOAc in hexanes) to give compound T4 (35 mg, 36% yield) as a white solid. m/z=544 (M+1); ¹H NMR (400 MHz, CDCl₃) δ 8.02 (s, 1H), 6.10 (s, 1H), 3.18 (m, 1H), 2.72 (q, J=7.6 Hz, 2H), 2.60 (d, J=3.8 Hz, 1H), 2.26 (td, J=13.8, 4.5 Hz, 1H), 2.07-1.99 (m, 1H), 1.95-1.20 (m, 16H), 1.44 (s, 3H), 1.25 (s, 3H), 1.17 (s, 3H), 1.16 (s, 3H), 0.99 (s, 3H), 0.95 (d, J=5.7 Hz, 3H), 0.80 (d, J=6.6 Hz, 3H).

Compound 13d: To a solution of compound 10 [produced from compound 9 (365 mg), <0.742 mmol] in CH₂Cl₂ (7.4 mL) at 0° C. was added triethylamine (0.309 mL, 2.22 mmol) and compound 12d (116 mg, 1.11 mmol). The mixture was stirred at room temperature for 2 h; and then was partitioned between CH₂Cl₂ and water. The aqueous layer was separated and was extracted with CH₂Cl₂. The combined organic extracts were dried with Na₂SO₄; filtered; and concentrated. The residue was purified by column chromatography (silica gel, eluting with 0-80% EtOAc in CH₂C12) to give compound 13d (265 mg, 62% yield) as a white solid. m/z=578 (M+1).

Compound T5: To a solution of compound 13d (265 mg, 0.459 mmol) in THF (13 mL) at room temperature was added tetrabutylammonium hydroxide solution (40 wt. % aqueous, 0.893 mL, 1.37 mmol). The mixture was stirred at room temperature for 4 h; diluted with EtOAc; and washed with water. The organic extract was dried with Na₂SO₄; filtered; and concentrated. The residue was purified by column chromatography (silica gel, eluting with 0-70% EtOAc in CH₂Cl₂) to give partially purified product, which was purified again by column chromatography (silica gel, eluting with 0-90% EtOAc in hexanes) to give compound T5 (46 mg, 18% yield) as a yellow solid. m/z=560 (M+1); ¹H NMR (400 MHz, CDCl₃) S ¹H NMR (400 MHz, CDCl₃) δ 8.01 (s, 1H), 6.10 (s, 1H), 3.96 (q, J=5.9 Hz, 2H), 3.19 (m, 1H), 2.97 (t, J=5.8 Hz, 2H), 2.53 (d, J=3.8 Hz, 1H), 2.36 (t, J=6.3 Hz, 1H), 2.28 (td, J=13.7, 4.5 Hz, 1H), 2.03 (m, 1H), 1.95-1.10 (m, 13H), 1.44 (s, 3H), 1.25 (s, 3H), 1.17 (s, 3H), 1.16 (s, 3H), 0.99 (s, 3H), 0.95 (d, J=5.6 Hz, 3H), 0.80 (d, J=6.6 Hz, 3H).

Compound 13e: To a solution of compound 10 (120 mg, 0.235 mmol) in CH₂Cl₂ (2.3 mL) at 0° C. was added triethylamine (0.098 mL, 0.70 mmol) and compound 12e (41.6 mg, 0.352 mmol). The mixture was stirred at room temperature for 2 h; and then was partitioned between CH₂Cl₂ and water. The aqueous layer was separated and was extracted with CH₂Cl₂. The combined organic extracts were dried with Na₂SO₄; filtered; and concentrated. The residue was purified by column chromatography (silica gel, eluting with 0-70% EtOAc in hexanes) to give compound 13e (85 mg, 61% yield) as a yellow solid. m/z=592 (M+1).

Compound T6: To a solution of compound 13e (85 mg, 0.14 mmol) in THF (4.3 mL) at room temperature was added tetrabutylammonium hydroxide solution (40 wt. % aqueous, 0.186 mL, 0.285 mmol). The mixture was stirred at room temperature for 4 h; diluted with EtOAc; and washed with water. The organic extract was dried with Na₂SO₄; filtered; and concentrated. The residue was purified by column chromatography (silica gel, eluting with 0-70% EtOAc in CH₂Cl₂) to give partially purified product, which was purified again by column chromatography (silica gel, eluting with 0-100% EtOAc in hexanes) to give compound T6 (˜1:1 mixture of two diastereomers; 28 mg, 34% yield) as a yellow solid. m/z=574 (M+1); ¹H NMR (400 MHz, CDCl₃) δ 8.01 (s, 1H), 6.10 (s, 1H), 4.47-4.56 (m, 1H), 3.31 (s, 1.5H), 3.28 (s, 1.5H), 3.15-3.24 (m, 1H), 2.45-2.50 (m, 1H), 2.27 (td, J=13.7, 4.5 Hz, 1H), 2.15-2.04 (m, 1H), 1.98-1.20 (m, 16H), 1.43 (s, 3H), 1.25 (s, 3H), 1.16 (s, 6H), 0.99 (s, 3H), 0.95 (d, J=5.6 Hz, 3H), 0.80 (d, J=6.6 Hz, 3H).

Compound 15a: To a solution of compound 10 (125 mg, 0.245 mmol) in CH₂Cl₂ (2.45 mL) at 0° C. was added triethylamine (0.102 mL, 0.732 mmol) and compound 14a (59 mg, 0.38 mmol). The mixture was stirred at room temperature for 1 h; and then was partitioned between CH₂Cl₂ and water. The aqueous layer was separated and was extracted with CH₂Cl₂. The combined organic extracts were dried with Na₂SO₄; filtered; and concentrated. The residue was purified by column chromatography (silica gel, eluting with 0-50% EtOAc in CH₂Cl₂) to give compound 15a (80 mg, 55% yield) as a yellow solid. m/z=592 (M+1).

Compound T7: To a solution of compound 15a (80 mg, 0.14 mmol) in THF (4 mL) at room temperature was added tetrabutylammonium hydroxide solution (40 wt. % aqueous, 0.175 mL, 0.268 mmol). The mixture was stirred at room temperature for 2 h; diluted with EtOAc; and washed with water. The organic extracts were dried with Na₂SO₄; filtered; and concentrated. The residue was purified by column chromatography (silica gel, eluting with 0-50% EtOAc in hexanes) to give compound T7 (40 mg, 52% yield) as a white solid. m/z=574 (M+1); ¹H NMR (400 MHz, CDCl₃) δ 8.02 (s, 1H), 6.10 (s, 1H), S 3.72 (t, J=6.5 Hz, 2H), 3.30 (s, 3H), 3.18 (m, 1H), 2.97 (t, J=6.5 Hz, 2H), 2.60 (d, J=3.8 Hz, 1H), 2.26 (td, J=13.8, 4.5 Hz, 1H), 2.09-1.99 (m, 1H), 1.90-1.10 (m, 13H), 1.44 (s, 3H), 1.25 (s, 3H), 1.16 (s, 3H), 1.15 (s, 3H), 0.99 (s, 3H), 0.95 (d, J=5.8 Hz, 3H), 0.80 (d, J=6.6 Hz, 3H).

Compound 15b: To a solution of compound 10 (120 mg, 0.235 mmol) in CH₂Cl₂ (2.3 mL) at 0° C. was added triethylamine (0.098 mL, 0.70 mmol) and compound 14b (54 mg, 0.35 mmol). The mixture was stirred at room temperature for 2 h; and then was partitioned between CH₂Cl₂ and water. The aqueous layer was separated and was extracted with CH₂Cl₂. The combined organic extracts were dried with Na₂SO₄; filtered; and concentrated. The residue was purified by column chromatography (silica gel, eluting with 0-100% EtOAc in CH₂Cl₂) to give compound 15b (80 mg, 58% yield) as a yellow solid. m/z=591 (M+1).

Compound T8: To a solution of compound 15b (80 mg, 0.14 mmol) in THF (4 mL) at room temperature was added tetrabutylammonium hydroxide solution (40 wt. % aqueous, 0.176 mL, 0.270 mmol). The mixture was stirred at room temperature for 4 h; diluted with EtOAc; and washed with water. The organic extracts were dried with Na₂SO₄; filtered; and concentrated. The residue was purified by column chromatography (silica gel, eluting with 0-100% EtOAc in CH₂Cl₂) to give compound T8 (18 mg, 23% yield) as a white solid. m/z=573 (M+1); ¹H NMR (400 MHz, CDCl₃) δ 8.01 (s, 1H), 6.10 (s, 1H), 3.60 (d, J=1.8 Hz, 2H), 3.22 (m, 1H), 2.45 (d, J=3.7 Hz, 1H), 2.29 (s, 6H), 2.31-2.20 (m, 1H), 2.14-2.06 (m, 1H), 1.95-1.10 (m, 13H), 1.43 (s, 3H), 1.25 (s, 3H), 1.16 (s, 3H), 1.15 (s, 3H), 0.99 (s, 3H), 0.95 (d, J=5.7 Hz, 3H), 0.80 (d, J=6.6 Hz, 3H).

Compound 17: To a solution of compound 10 [produced from compound 9 (111 mg), <0.226 mmol] in CH₂Cl₂ (2.3 mL) at 0° C. was added triethylamine (0.075 mL, 0.564 mmol) and compound 16 (40 mg, 0.27 mmol). The mixture was stirred at room temperature for 3 h; diluted with EtOAc (30 mL); and washed with water (2×15 mL). The combined aqueous washes were extracted with EtOAc (20 mL). The combined organic extracts were dried with MgSO₄; filtered; and concentrated. The residue was purified by column chromatography (silica gel, eluting with 0-100% EtOAc in hexanes) to give compound 17 (101 mg, 72% yield) as a yellow solid. m/z=620 (M+1).

Compound T9: To a solution of compound 17 (101 mg, 0.163 mmol) in THF (1.6 mL) at room temperature was added tetrabutylammonium hydroxide (1.0 M solution in MeOH, 0.326 mL, 0.326 mmol). The mixture was stirred at room temperature for 72 h; diluted with EtOAc (30 mL); and washed with water (2×15 mL) and brine (10 mL). The aqueous washes were combined, and extracted with EtOAc (20 mL), which was washed with water (2×10 mL). The combined organic extracts were dried with MgSO₄; filtered; and concentrated. The residue was purified by column chromatography (silica gel, eluting with 0-50% EtOAc in hexanes) to give compound T9 (69 mg, 70% yield) as a white solid. m/z=602 (M+1); ¹H NMR (400 MHz, CDCl₃) δ 8.02 (s, 1H), 6.09 (s, 1H), 4.53 (s, 2H), 3.19 (dd, J=11.2, 3.7 Hz, 1H), 2.54 (d, J=3.8 Hz, 1H), 2.26 (td, J=13.7, 4.5 Hz, 1H), 2.10-2.02 (m, 1H), 1.95-1.20 (m, 13H), 1.44 (s, 3H), 1.25 (s, 9H), 1.25 (s, 3H), 1.17 (s, 3H), 1.15 (s, 3H), 1.00 (s, 3H), 0.95 (d, J=5.6 Hz, 3H), 0.80 (d, J=6.6 Hz, 3H).

Compound T10: To a solution of compound T9 (68 mg, 0.11 mmol) in CH₂Cl₂ (1.1 mL) at 0° C. was added trifluoroacetic acid (0.44 mL, 5.6 mmol). The mixture was stirred at room temperature for 4 h; and then concentrated. The residue was dissolved in EtOAc (2×10 mL) and concentrated. The crude product was purified by column chromatography (silica gel, eluting with 0-100% EtOAc in hexanes) to give compound T10 (48 mg, 78% yield) as a white solid. m/z=546 (M+1); ¹H NMR (400 MHz, CDCl₃) S ¹H NMR (400 MHz, CDCl₃) δ 8.02 (s, 1H), 6.11 (s, 1H), 4.75 (s, 2H), 3.19 (dd, J=11.8, 3.5 Hz, 1H), 2.55 (d, J=3.8 Hz, 1H), 2.33-2.24 (m, 2H), 2.10-2.00 (m, 1H), 1.95-1.10 (m, 13H), 1.44 (s, 3H), 1.25 (s, 3H), 1.17 (s, 3H), 1.16 (s, 3H), 0.99 (s, 3H), 0.95 (d, J=5.7 Hz, 3H), 0.80 (d, J=6.6 Hz, 3H).

Compound T11: Compound T10 (47 mg, 0.086 mmol) was dissolved in acetonitrile (0.4 mL) and was cooled to 0° C. Hunig's base (0.068 mL, 0.39 mmol), a solution of ethyldiisopropylamine trihydrofluoride (24 mg, 0.13 mmol) in acetonitrile (0.2 mL), and perfluoro-1-butanesulfonyl fluoride (52 mg, 0.17 mmol) were added sequentially. The mixture was stirred at 0° C. for 1 h; and then was diluted with EtOAc (20 mL). The mixture was washed with saturated aqueous NaHCO₃(10 mL), water (10 mL) and brine (10 mL). The organic extract was dried with MgSO₄, filtered and concentrated. The residue was purified by column chromatography (silica gel, eluting with 0-40% EtOAc in hexanes) to give partially purified compound product, which was purified again by column chromatography (silica gel, eluting with 0-35% acetone in hexanes) to give compound T11 (19 mg, 40% yield) as a white solid. m/z=548 (M+1); ¹H NMR (400 MHz, CDCl₃) δ 8.01 (s, 1H), 6.10 (s, 1H), 5.43 (d, J=46.4 Hz, 2H), 3.21 (m, 1H), 2.49 (d, J=3.8 Hz, 1H), 2.30 (td, J=13.8, 4.5 Hz, 1H), 2.12-2.04 (m, 1H), 1.98-1.20 (m, 13H), 1.44 (s, 3H), 1.25 (s, 3H), 1.17 (s, 6H), 0.99 (s, 3H), 0.96 (d, J=5.7 Hz, 3H), 0.81 (d, J=6.6 Hz, 3H).

Compound 18: To a solution of compound 6 (60 g, 118 mmol) in THF (600 mL) at 0° C. was added DIBAL-H (1.0 M solution in toluene, 946 mL, 946 mmol) slowly. The reaction mixture was stirred at room temperature for 5 h. After cooled to 0° C., the reaction was quenched with acetone (100 mL); and then treated carefully with aqueous Rochells salt (200 mL). The mixture was allowed to warm to room temperature; stirred for 30 min; and extracted with CH₂Cl₂ (3×1 L). The combined organic extracts were washed with brine (1 L); dried over Na₂SO₄; filtered; and concentrated to give the crude product (65 g) as a white solid. The crude product was dissolved in CH₂Cl₂ (1.2 L). 4A MS (130 g) and 4-methylmorpholine N-oxide (30.5 g, 260 mmol) were added. The resultant mixture was stirred at room temperature for 10 min under N₂.

Tetrapropylammonium perruthenate (TPAP, 4.1 g, 11.7 mmol) was added. The reaction mixture was stirred at room temperature for 2 h, and then quenched with 10% aqueous Na₂SO₃ (150 mL). The mixture was extracted with CH₂Cl₂(2×500 mL). The combined organic extracts were washed with water (1 L); dried with Na₂SO₄; filtered; and concentrated. The residue was purified by column chromatography (silica gel, eluting with 0-40% EtOAc in hexanes) to give compound 18 (27 g, 48% yield) as a white solid. m/z=478 (M+1).

Compound 19: To a mixture of compound 18 (200 mg, 0.419 mmol) in EtOH (4 mL) at 0° C. was added sodium borohydride (17 mg, 0.45 mmol). The mixture was stirred at 0° C. for 1 h; diluted with EtOAc (20 mL); treated with 1 N aq. HCl (10 mL); and stirred for 5 min. The organic extract was separated; washed with water (10 mL); dried with MgSO₄; filtered and concentrated. The residue was purified by column chromatography (silica gel, eluting with 0-80% EtOAc in hexanes) to give compound 19 (174 mg, 87% yield) as a white solid. m/z=480 (M+1).

Compound 20: To a stirring mixture of compound 19 (95 mg, 0.20 mmol) in MeOH (2 mL) at room temperature was added sodium methoxide (25 wt. % in MeOH, 0.091 mL, 0.40 mmol). The mixture was heated at 55° C. for 2 h; cooled to room temperature; and treated with 10% aqueous NaH₂PO₄ solution (20 mL). The mixture was extracted with EtOAc (2×20 mL). The combined organic extracts were dried with MgSO₄; filtered and concentrated. The residue was purified by column chromatography (silica gel, eluting with 0-70% EtOAc in hexanes) to give compound 20 (83 mg, 87% yield) as a white solid. m/z=480 (M+1).

Compound T12 and T13: Compound 20 (83 mg, 0.17 mmol) and 1,3-dibromo-5,5-dimethylhydantoin (25 mg, 0.087 mmol) were weighed in a flask and cooled to 0° C. Anhydrous DMF (0.85 mL) was added. The mixture was stirred at 0° C. for 1 h, and then treated with pyridine (0.056 mL, 0.69 mmol). The reaction was stirred at 60° C. for 4 h; cooled to room temperature; and diluted with EtOAc (20 mL). The mixture was washed with 1 N aqueous HCl (10 mL), water (2×10 mL), and brine (10 mL). The organic extract was dried with MgSO₄; filtered; and concentrated. The residue was purified by column chromatography (silica gel, eluting with 0-100% EtOAc in hexanes) to give compound T12 (19 mg, 23% yield) and partially purified compound T13, which was purified again by column chromatography (silica gel, eluting with 0-60% acetone in hexanes) to give compound T13(22 mg, 27% yield). Compound T12: light yellow solid; m/z=476 (M+1); ¹H NMR (400 MHz, CDCl₃) δ 8.04 (s, 1H), 6.19 (s, 1H), 3.79 (d, J=6.9 Hz, 1H), 3.45 (dd, J=6.9, 1.7 Hz, 1H), 2.18 (d, J=12.8 Hz, 1H), 2.06-1.00 (m, 15H), 1.50 (s, 3H), 1.48 (s, 3H), 1.26 (s, 3H), 1.19 (s, 3H), 1.03 (s, 3H), 0.94 (d, J=6.3 Hz, 3H), 0.80 (d, J=6.2 Hz, 3H). Compound T13: light yellow solid; m/z=478 (M+1); ¹H NMR (400 MHz, CDCl₃) δ 8.05 (s, 1H), 6.14 (s, 1H), 3.62 (d, J=11.1 Hz, 1H), 3.51 (d, J=11.1 Hz, 1H), 2.76 (d, J=3.9 Hz, 1H), 2.33 (dd, J=11.5, 3.8 Hz, 1H), 1.90 (td, J=13.7, 4.4 Hz, 1H), 1.85-1.05 (m, 15H), 1.50 (s, 3H), 1.35 (s, 3H), 1.26 (s, 3H), 1.19 (s, 3H), 1.14 (s, 3H), 0.90 (d, J=6.3 Hz, 3H), 0.71 (d, J=6.7 Hz, 3H).

Compound 21: tert-butyl 3-aminopropanoate hydrochloride (152 mg, 0.837 mmol) in THF (2 mL) was treated with Et₃N (0.12 mL, 0.84 mmol) and a solution of compound 18 (200 mg, 0.419 mmol) in THF (2 mL) sequentially. The reaction was stirred at room temperature for 2.5 h, and then sodium triacetoxyborohydride (27 mg, 0.13 mmol) was added. The reaction was stirred for another 5 h. NaBH₄ (32 mg, 0.84 mmol) and MeOH (4 mL) were added. The mixture was stirred at room temperature for 15 min, and then was cooled in an ice bath. Saturated aqueous NaHCO₃(20 mL) was added. The mixture was extracted with EtOAc (3×20 mL). The combined organic extracts were washed with brine (25 mL), dried with Na₂SO₄, filtered, and concentrated. The residue was purified by column chromatography (silica gel, eluting with 0-100% EtOAc in CH₂Cl₂) to give compound 21 (160 mg, 63% yield) as a white solid. m/z=607 (M+1).

Compound 22: Compound 21 (160 mg, 0.264 mmol) in CH₂Cl₂ (5 mL) was treated with trifluoroacetic acid (2.5 mL) at 0° C. The reaction was stirred at room temperature for 4 h. Upon completion, the reaction was concentrated. The residue was dissolved in toluene (3×20 mL) and concentrated. The residue was dried under vacuum to give crude compound 22 (145 mg), which was used in the next step without purification. m/z=551 (M+1 of free amine).

Compound 23: Compound 22 (145 mg, 0.263 mmol) was dissolved in CH₂Cl₂ (8 mL), and cooled to 0° C. Et₃N (110 μL, 0.790 mmol) and phosphorus(V) oxychloride (32 μL, 0.34 mmol) were added sequentially. The mixture was stirred at 0° C. for 20 min; quenched with saturated aqueous NaHCO₃(20 mL); and stirred at ambient temperature for 5 min. The mixture was extracted with CH₂Cl₂ (2×20 mL). The combined organic extracts were washed with brine (20 mL), dried with Na₂SO₄, filtered and concentrated. The residue was purified by column chromatography (silica gel, eluting with 0-100% EtOAc in hexanes) to give compound 23 (53 mg, 38% yield from compound 21) as a white solid. m/z=533 (M+1).

Compound 24: Compound 23 (53 mg, 0.099 mmol) was mixed with MeOH (2 mL) at room temperature. Sodium methoxide (25 wt. % solution in MeOH, 46 μL, 0.20 mmol) was added at room temperature. The mixture was stirred at 55° C. for 2 h. After cooled to 0° C., 10% aqeuous NaH₂PO₄(20 mL) was added. The mixture was extracted with EtOAc (2×20 mL). The combined organic extracts were washed with brine (20 mL), dried with Na₂SO₄, filtered and concentrated to give compound 24 (50 mg, 94% yield), which was used in the next step without further purification. m/z=533 (M+1).

Compound T14: Compound 24 (50 mg, 0.094 mmol) was dissolved in DMF (2 mL) and cooled to 0° C. under N₂. A solution of 1,3-dibromo-5,5-dimethylhydantoin (13 mg, 0.047 mmol) in DMF (0.5 mL) was added. The mixture was stirred at 0° C. for 1 h. Pyridine (30 μL, 0.38 mmol) was added. The mixture was heated at 60° C. for 4 h. After cooled to room temperature, the mixture was diluted with EtOAc (20 mL) and washed with 1 N aqueous HCl (10 mL), water (2×10 mL) and brine (20 mL). The organic extract was dried with Na₂SO₄, filtered, and concentrated. The residue was purified by column chromatography (silica gel, eluting with 0-50% acetone in hexanes) to give compound T14 (28 mg, 56% yield) as a white solid. m/z=531 (M+1); H NMR (400 MHz, CDCl₃) δ 8.05 (s, 1H), 6.14 (s, 1H), 3.47-3.39 (m, 1H), 3.38-3.30 (m, 1H), 3.25 (d, J=14.5 Hz, 1H), 3.17 (d, J=14.5 Hz, 1H), 3.00 (t, J=4.1 Hz, 2H), 2.91 (d, J=3.7 Hz, 1H), 2.24 (dd, J=10.9, 2.8 Hz, 1H), 2.04-1.89 (m, 2H), 1.90-1.00 (m, 13H), 1.50 (s, 3H), 1.44 (s, 3H), 1.26 (s, 3H), 1.19 (s, 3H), 1.13 (s, 3H), 0.89 (d, J=6.3 Hz, 3H), 0.70 (d, J=6.6 Hz, 3H).

Compound 25: To a suspension of methyl 4-aminobutanoate hydrochloride (129 mg, 0.837 mmol) in THF (2 mL) was added Et₃N (0.12 mL, 0.84 mmol). After the mixture was stirred at room temperature for 10 min, a solution of compound 18 (200 mg, 0.42 mmol) in THF (2 mL) was added at room temperature. The mixture was stirred at room temperature for 2.5 h; treated with sodium triacetoxyborohydride (355 mg, 1.67 mmol); and stirred at room temperature for another 5 h. MeOH (4 mL) and sodium borohydride (38 mg, 0.96 mmol) were added sequentially, and the mixture was stirred at room temperature for 15 min. Saturated aqueous NaHCO₃(20 mL) was added. The mixture was extracted with EtOAc (3×30 mL). The combined organic extracts were washed with brine (30 mL), dried with Na₂SO₄, filtered and concentrated to give crude compound 25 (259 mg) which was used in the next step without further purification. m/z=579 (M+1).

Compound 26: Compound 25 (259 mg, <0.42 mmol) in toluene (6 mL) was heated at 140° C. for 6 h, and then concentrated. The residue was purified by column chromatography (silica gel, eluting with 0-100% EtOAc in hexanes) to give compound 26 (140 mg, 57% yield from compound 18) as a white solid. m/z=547 (M+1).

Compound 27: A solution of compound 26 (477 mg, 0.872 mmol) in MeOH (5 mL) was treated with sodium methoxide (25 wt. % solution in MeOH, 400 μL, 1.74 mmol) at room temperature. The mixture was heated at 55° C. for 2 h; cooled to room temperature; treated with 10% aqueous NaH₂PO₄ (20 mL). The mixture was extracted with EtOAc (2×20 mL). The combined organic extracts were washed with brine (20 mL), dried with Na₂SO₄, filtered and concentrated. The residue was purified by column chromatography (silica gel, eluting with 0-60% acetone in hexanes) to give compound 27 (380 mg, 80% yield) as a white solid. m/z=547 (M+1).

Compound T15: Compound 27 (50 mg, 0.091 mmol) in DMF (3 mL) was cooled to 0° C. A solution of 1,3-dibromo-5,5-dimethylhydantoin (13 mg, 0.046 mmol) in DMF (0.5 mL) was added. The mixture was stirred at 0° C. for 1 h. Pyridine (30 μL, 0.37 mmol) was added. The reaction was heated at 60° C. for 4 h, and then was cooled to room temperature. The mixture was diluted with EtOAc (20 mL); and washed with 1 N aqueous HCl (10 mL), water (2×10 mL) and brine (20 mL) sequentially. The organic extract was dried with Na₂SO₄, filtered and concentrated. The residue was purified by column chromatography (silica gel, eluting with 0-50% acetone in hexanes) to give compound T15 (27 mg, 54% yield) as a white solid. m/z=545 (M+1). ¹H NMR (400 MHz, CDCl₃) δ 8.05 (s, 1H), 6.13 (s, 1H), 3.58 (dt, J=9.5, 6.6 Hz, 1H), 3.44 (dt, J =9.3, 7.3 Hz, 1H), 3.37 (d, J=14.0 Hz, 1H), 3.23 (d, J=14.0 Hz, 1H), 3.13 (d, J=3.7 Hz, 1H), 2.38 (t, J=8.4 Hz, 2H), 2.25-2.20 (m, 1H), 2.10-1.00 (m, 17H), 1.50 (s, 3H), 1.46 (s, 3H), 1.26 (s, 3H), 1.19 (s, 3H), 1.13 (s, 3H), 0.88 (d, J=6.4 Hz, 3H), 0.70 (d, J=6.6 Hz, 3H).

Compound 28: To a suspension of compound 9 (290 mg, 0.590 mmol) in toluene (6 mL) at 0° C. was added triethylamine (1.64 mL, 11.8 mmol). Diphenylphosphoryl azide (1.27 mL, 5.90 mmol) was then added slowly at 0° C. The mixture was stirred at 0° C. for 2 h; warmed up to room temperature; and stirred for another 18 h. The reaction mixture was directly loaded onto a silica gel column and purified by flash chromatography (eluting with 0-30% EtOAc in CH₂Cl₂) to give compound 28 (266 mg, 87% yield) as a colorless semisolid. m/z=517 (M+1).

Compound 29: A mixture of compound 28 (266 mg, 0.515 mmol) in toluene (4 mL) was stirred at 80° C. for 2.5 h, and then concentrated to give compound 29 (252 mg) as a white solid, which was used in next step without further purification. m/z=489 (M+1).

Compound T16: To a solution of compound 29 (252 mg, 0.52 mmol) in MeCN (20 mL) at 0° C. was added aqueous HCl (12 M, 1.29 mL, 15.5 mmol). The mixture was stirred at 0° C. for 2 h; warmed up to room temperature; and stirred for another 16 h. The reaction mixture was diluted with water (10 mL) and cooled to 0° C. NaOH (619 mg, 15.5 mmol) was added and the resultant mixture was stirred for 5 min. The mixture was concentrated. The residue was extracted with EtOAc (3×50 mL). The combined organic extracts were washed with brine (100 mL), dried with Na₂SO₄, filtered, and concentrated. The residue was purified by column chromatography (silica gel, eluting with 0-100% EtOAc) to give compound T16 (146 mg, 61% yield) as a white solid. m/z=463 (M+1); ¹H NMR (400 MHz, CDCl₃) δ 8.04 (s, 1H), 6.10 (s, 1H), 3.88 (d, J=3.8 Hz, 1H), 1.48 (s, 3H), 1.37 (s, 3H), 1.25 (s, 3H), 1.16 (s, 3H), 1.06 (s, 3H), 2.21-0.96 (m, 18H), 0.86 (d, J=5.9 Hz, 3H), 0.69 (d, J=6.7 Hz, 3H).

Compound T17: To a solution of compound T16 (68 mg, 0.15 mmol) in CH₂Cl₂ (2 mL) at room temperature was added 2,2-difluoropropionic acid (24 mg, 0.22 mmol). Propylphosphonic anhydride (T3P, 50 wt. % solution in EtOAc, 310 μL, 0.51 mmol) and triethylamine (100 μL, 0.73 mmol) were then added sequentially at room temperature. The mixture was stirred at room temperature for 3 h; and then was quenched with saturated aqueous NaHCO₃(10 mL). The mixture was stirred for 10 min, and then extracted with EtOAc (3×30 mL). The combined organic extracts were washed with brine (10 mL), dried over Na₂SO₄, filtered and concentrated. The residue was purified by column chromatography (silica gel, eluting with 0-40% acetone in hexane) to give compound T17 (38 mg, 47% yield) as a white solid. m/z=555 (M+1); ¹H NMR (400 MHz, CDCl₃) δ 8.03 (s, 1H), 6.13 (s, 1H), 5.96 (br s, 1H), 2.91 (d, J=3.8 Hz, 1H), 2.54 (m, 1H), 2.34 (m, 1H), 2.20 (m, 1H), 1.96 (m, 1H), 1.73 (t, J=19.3 Hz, 3H), 1.83-0.80 (m, 13H), 1.47 (s, 3H), 1.28 (s, 3H), 1.23 (s, 3H), 1.16 (s, 3H), 1.11 (s, 3H), 0.88 (d, J=5.7 Hz, 3H), 0.72 (d, J=6.6 Hz, 3H).

Compound T18: To a solution of compound T16 (58 mg, 0.13 mmol) in CH₂Cl₂ (2 mL) at 0° C., was added triethylamine (87 μL, 0.63 mmol) and acetyl chloride (22 μL, 0.31 mmol) sequentially. The mixture was stirred at 0° C. for 2 h.; and then was quenched with saturated aqueous NaHCO₃(10 mL). The resultant mixture was extracted with EtOAc (3×10 mL). The combined organic extracts were washed with brine (10 mL), dried with Na₂SO₄, filtered and concentrated. The residue was purified by column chromatography (silica gel, eluting with 0 to 40% acetone in hexane) to give compound T18 (15.5 mg, 24% yield) as a white solid. m/z=505 (M+1); ¹H NMR (400 MHz, CDCl₃) δ 8.05 (s, 1H), 6.08 (s, 1H), 5.57 (s, 1H), 3.06 (d, J=3.8 Hz, 1H), 2.56-2.47 (m, 1H), 2.39-2.27 (m, 2H), 1.92 (s, 3H), 1.90-1.06 (m, 13H), 1.45 (s, 3H), 1.29 (s, 3H), 1.23 (s, 3H), 1.16 (s, 3H), 1.09 (s, 3H), 0.87 (d, J=5.8 Hz, 3H), 0.69 (d, J=6.6 Hz, 3H).

Compound 30: A mixture of compound 18 (15.0 g, 31.4 mmol), (S)-2-methylpropane-2-sulfinamide (11.4 g, 94.2 mmol), MgSO₄ (3.77 g, 30.6 mmol) and Titanium(IV) ethoxide (21.5 g, 94.2 mmol) in THF (300 mL) was heated at reflux for 2 h. After cooling down, the mixture was quenched with brine (100 mL) and was extracted with ethyl acetate (3×100 mL). The combined organic extracts were dried with anhydrous Na₂SO₄; filtered and concentrated. The residue was purified by column chromatography (silica gel, eluting with 16% EtOAc in petroleum ether) to give the product 30 (16.7 g, 92% yield) as a white solid. m/z=581 (M+1).

Compound 31: To a stirring solution of compound 30 (2.0 g, 3.4 mmol) in THF (20 mL) was added NaBH₄ (525 mg, 13.8 mmol) in MeOH (20 mL) at 0° C. The mixture was stirred at 0° C. for 2 h, and then quenched with acetone. The solvent was removed. The residue was purified by column chromatography (silica gel, eluting with 50% EtOAc in petroleum ether) to give compound 31 (1.64 g, 82% yield) as a white solid. m/z=583 (M+1).

Compound 32: A solution of compound 31(6.7 g, 11.5 mmol)in HCl (6 M in 1,4-dioxane, 60 mL) was stirred at 0° C. for 2 h. The solvent was removed. The residue was purified by preparative reversed phase HPLC (C18, eluting with 5-80% MeCN in water containing 0.1% HCl) to give the compound 32 HCl salt (4.4 g, 75% yield) as a white solid. m/z=479 (M+1).

Compound 33: To a solution of compound 32 (402 mg, 0.840 mmol) in THF (9 mL) and water (3 mL) were added NaHCO₃(212 mg, 2.52 mmol) and di-tert-butyl dicarbonate (220 mg, 1.01 mmol) sequentially at room temperature. The reaction was stirred at room temperature for 2 h. The mixture was diluted with EtOAc (50 mL) and was washed with saturated aqueous NaHCO₃(2×20 mL) and water (20 mL). The organic extract was dried with MgSO₄, filtered and concentrated. The residue was purified by column chromatography (silica gel, eluting with 40% EtOAc in hexanes) to give compound 33 (207 mg, 43% yield) as a white solid. m/z=579 (M+1).

Compound 34: Compound 33 (423 mg, 0.73 mmol) in MeOH (6 mL) was treated with sodium methoxide solution (25 wt. % in MeOH, 334 μL, 1.46 mmol) at room temperature under nitrogen. The mixture was heated at 55° C. for 1.5 h, and then cooled to 0° C. The mixture was treated with 10% aqueousNaH2PO₄ (10 mL) and extracted with EtOAc (2×20 mL). The combined organic extracts were washed with brine (20 mL), dried with Na₂SO₄, filtered and concentrated. The residue was purified by column chromatography (silica gel, eluting with 0-80% EtOAc in hexanes) to give compound 34 (347 mg, 82% yield). m/z=523 (M-C4H7).

T20: Compound 34 (347 mg, 0.599 mmol) was dissolved in DMF (6 mL) and cooled to 0° C. under nitrogen. 1,3-Dibromo-5,5-dimethylhydantoin (86 mg, 0.30 mmol) was dissolved in DMF (1 mL) in a vial. The solution was added to the reaction dropwise. DMF (1 mL) was used to rinse the vial and added to the reaction mixture. The mixture was stirred at 0° C. for 1 h. Pyridine (145 μL, 1.80 mmol) was added. The mixture was heated at 60° C. for 8 h. After cooled to 0° C., the mixture was diluted with EtOAc (20 mL) and washed with water (2×10 mL) and brine (20 mL). The organic extract was dried with Na₂SO₄, filtered, and concentrated. The residue was purified by column chromatography (silica gel, eluting with 0-50% EtOAc in hexanes) to give compound T20 (336 mg, 97% yield). m/z=521 (M-C₄H₇); ¹H NMR (400 MHz, CDCl₃) δ 8.05 (s, 1H), 6.12 (s, 1H), 4.63 (t, J=6.3 Hz, 1H), 3.19 (d, J=6.6 Hz, 2H), 2.93 (d, J=3.7 Hz, 1H), 2.20 (m, 1H), 1.51 (s, 3H), 1.45 (s, 3H), 1.43 (s, 9H), 1.27 (s, 3H), 1.20 (s, 3H), 1.12 (s, 3H), 1.02-2.02 (m, 1OH), 0.89 (d, J=6.3 Hz, 3H), 0.71 (d, J=6.6 Hz, 3H).

T21: Compound T20 (336 mg, 0.58 mmol) in CH₂Cl₂ (6 mL) was treated with trifluoroacetic acid (1.2 mL, 16 mmol) at 0° C. The reaction was stirred at 0° C. for 3 h. The reaction was diluted with EtOAc (30 mL) and saturated aqueous NaHCO₃(30 mL). Layers were separated and the organic layer was washed with saturated aqueous NaHCO₃(2×20 mL) and water (30 mL). The aqueous phase was extracted with EtOAc (20 mL). The combined organic extracts were washed with brine (30 mL), dried with Na₂SO₄, filtered, and concentrated to give crude compound T21 (234 mg, 84% yield) as a white solid. Analytical sample was obtained by purifying the crude product (20 mg) by column chromatography [silica gel, eluting with 0-15% (1% Et₃N in MeOH) in CH₂Cl₂] to give compound T21 (15 mg, 75% yield) as an off-white solid. m/z=477 (M+1); ¹H NMR (400 MHz, CDCl₃) 8.07 (s, 1H), 6.13 (s, 1H), 3.06 (d, J=13.2 Hz, 1H), 2.92 (d, J=13.4 Hz, 1H), 2.76 (d, J=3.7 Hz, 1H), 2.28 (d, J=11.1 Hz, 1H), 2.06 (m, 1H), 1.52 (s, 3H), 1.38 (s, 3H), 1.27 (s, 3H), 1.19 (s, 3H), 1.14 (s, 3H), 1.09-1.88 (m, 14H), 0.90 (d, J=6.3 Hz, 3H), 0.69 (d, J=6.5 Hz, 3H).

T22: To a solution of crude compound T21 (44 mg, 0.092 mmol) in CH₂Cl₂ (1 mL) at 0° C. was added Et₃N (26 μL, 0.19 mmol) and acetic anhydride (13 μL, 0.14 mmol) sequentially. The reaction was stirred at 0° C. for 30 min. The reaction was diluted with EtOAc (10 mL) and water (10 mL). Layers were separated and the organic extract was washed with water (2×10 mL). The aqueous washes were combined and extracted with EtOAc (20 mL). The combined organic extracts were washed with brine (30 mL), dried with Na₂SO₄, filtered, and concentrated. The residue was purified by column chromatography (silica gel, eluting with 0-100% EtOAc in hexanes) to give compound T22 (28 mg, 58% yield) as an off-white solid. m/z=519 (M+1); ¹H NMR (400 MHz, CDCl₃) δ 8.10 (s, 1H), 6.15 (s, 1H), 5.58 (t, J=6.4 Hz, 1H), 3.34 (d, J=6.4 Hz, 2H), 3.00 (d, J=3.7 Hz, 1H), 2.20 (m, 1H), 2.02 (s, 3H), 1.93 (td, J=13.4, 3.9 Hz, 1H), 1.50 (s, 3H), 1.47 (s, 3H), 1.26 (s, 3H), 1.19 (s, 3H), 1.12 (s, 3H), 1.01-2.07 (m, 14H), 0.89 (d, J=6.3 Hz, 3H), 0.70 (d, J=6.6 Hz, 3H).

T23: To a solution of crude compound T21 (30 mg, <0.064 mmol) in CH₂Cl₂ (1 mL) at 0° C. was added Et₃N (26 μL, 0.18 mmol) and propionyl chloride (6 μL, 0.069 mmol) sequentially. The reaction was stirred at 0° C. for 30 min. The reaction was partitioned between EtOAc (10 mL) and water (10 mL). The organic extract was washed with water (2×10 mL). The combined aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give compound T23 (16 mg, 48% yield) as an off-white solid. m/z=533 (M+1); ¹H NMR (400 MHz, CDCl₃) δ 8.06 (s, 1H), 6.12 (s, 1H), 5.53 (t, J=6.0 Hz, 1H), 3.43 (dd, J=13.9, 6.9 Hz, 1H), 3.26 (dd, J=13.9, 6.1 Hz, 1H), 2.98 (d, J=3.7 Hz, 1H), 2.25 (q, J=7.6 Hz, 2H), 2.19 (m, 1H), 2.06 (m, 1H), 1.91 (td, J=13.5, 4.3 Hz, 1H), 1.50 (s, 3H), 1.48 (s, 3H), 1.26 (s, 3H), 1.19 (s, 3H), 1.18 (t, J=7.6 Hz, 3H), 1.12 (s, 3H), 1.00-1.84 (m, 13H), 0.90 (d, J=6.4 Hz, 3H), 0.71 (d, J=6.6 Hz, 3H).

T19: To a solution of crude compound T21 (88% pure, 31 mg, 0.057 mmol) in CH₂Cl₂ (1 mL) at 0° C. was added Et₃N (24 μL, 0.17 mmol) and cyclopropanecarbonyl chloride (6 μL, 0.068 mmol) sequentially. The reaction was stirred at room temperature for 1 h, and then was quenched with saturated aqueous NaHCO₃(20 mL). The mixture was extracted with CH₂Cl₂ (3×10 mL). The combined organic extracts were washed with brine (20 mL), dried with Na₂SO₄, filtered, and concentrated. The residue was purified by column chromatography (silica gel, eluting with 0-100% EtOAc in hexanes) to give a partially purified product which was further purified again by column chromatography (silica gel, eluting with 0-60% acetone in hexanes) to give compound T19 (14 mg, 44% yield) as an off-white solid. m/z=545 (M+1); ¹H NMR (400 MHz, CDCl₃) δ 8.08 (s, 1H), 6.13 (s, 1H), 5.71 (t, J=6.4 Hz, 1H), 3.48 (dd, J=13.9, 7.1 Hz, 1H), 3.24 (dd, J=13.9, 5.9 Hz, 1H), 2.96 (d, J=3.7 Hz, 1H), 2.21 (dd, J=11.5, 3.3 Hz, 1H), 2.04 (td, J=13.5, 4.8 Hz, 1H), 1.90 (td, J=13.6, 4.5 Hz, 1H), 1.70-1.84 (m, 4H), 1.50 (s, 3H), 1.45 (s, 3H), 1.26 (s, 3H), 1.19 (s, 3H), 1.12 (s, 3H), 1.02-1.61 (m, 1OH), 0.99-0.93 (m, 2H), 0.90 (d, J=6.3 Hz, 3H), 0.72-0.78 (m, 2H), 0.71 (d, J=6.6 Hz, 3H).

T24: To a solution of crude compound T21 (40 mg, <0.084 mmol) in CH₂Cl₂ (1 mL) at 0° C. was added Et₃N (35 μL, 0.25 mmol) and d3-acetyl chloride (7 μL, 0.092 mmol) sequentially. The reaction was stirred at 0° C. for 30 min. The reaction was diluted with EtOAc (10 mL) and water (10 mL). Layers were separated and the organic extract was washed with water (2×10 mL). The combined aqueous washes were extracted with EtOAc (20 mL). The combined organic extracts were washed with brine (20 mL), dried with Na₂SO₄, filtered, and concentrated.

The residue was purified by column chromatography (silica gel, eluting with 0-80% acetone in hexanes) to give compound T24 (23 mg, 53% yield) as an off-white solid. m/z=522 (M+1); ¹H NMR (400 MHz, CDCl₃) δ 8.09 (s, 1H), 6.14 (s, 1H), 5.57 (t, J=6.4 Hz, 1H), 3.33 (m, 2H), 3.00 (d, J=3.7 Hz, 1H), 2.20 (m, 1H), 2.03 (m, 1H), 1.93 (td, J=13.3, 3.9 Hz, 1H), 1.50 (s, 3H), 1.47 (s, 3H), 1.26 (s, 3H), 1.19 (s, 3H), 1.12 (s, 3H), 1.00-1.84 (m, 13H), 0.89 (d, J=6.4 Hz, 3H), 0.71 (d, J=6.6 Hz, 3H).

T25: To a solution of crude compound T21 (88% pure, 40 mg, 0.074 mmol) in CH₂Cl₂ (3 mL) at 0° C. was added Et₃N (31 μL, 0.22 mmol) and cyclobutanecarbonyl chloride (10 μL, 0.089 mmol) sequentially. The reaction was stirred at room temperature for 2 h, and then was quenched with saturated aqueous NaHCO₃(20 mL). The mixture was extracted with EtOAc (3×20 mL). The combined organic extracts were washed with brine (20 mL), dried with Na₂SO₄, filtered, and concentrated. The residue was purified by column chromatography (silica gel, eluting with 0-60% acetone in hexanes) to give compound T25 (23 mg, 56% yield) as an off-white solid. m/z=559 (M+1); ¹H NMR (400 MHz, CDCl₃) 8.06 (d, J=0.6 Hz, 1H), 6.12 (s, 1H), 5.43 (t, J=6.5 Hz, 1H), 3.45 (dd, J=13.8, 7.0 Hz, 1H), 3.22 (dd, J=13.8, 6.1 Hz, 1H), 2.96-3.10 (m, 2H), 1.51 (s, 3H), 1.49 (s, 3H), 1.26 (s, 3H), 1.20 (s, 3H), 1.12 (s, 3H), 1.00-2.34 (m, 22H), 0.89 (d, J=6.4 Hz, 3H), 0.70 (d, J=6.7 Hz, 3H).

T26: To a solution of crude compound T21 (40 mg, <0.084 mmol) in CH₂Cl₂ (1 mL) at 0° C. was added Et₃N (35 μL, 0.25 mmol) and methylaminoformyl chloride (10 mg, 0.11 mmol) sequentially. The reaction was stirred at 0° C. for 30 min. The reaction was diluted with EtOAc (10 mL) and water (10 mL). Layers were separated and the organic extract was washed with water (2×10 mL). The combined aqueous washes were extracted with EtOAc (20 mL). The combined organic extracts were washed with brine (20 mL), dried with Na₂SO₄, filtered, and concentrated.

The residue was purified by column chromatography (silica gel, eluting with 0-80% acetone in hexanes) to give partially purified product which was purified again by column chromatography (silica gel, eluting with 0-80% acetone in hexanes) to give compound T26 (11 mg, 25% yield) as an off-white solid. m/z=534 (M+1); ¹H NMR (400 MHz, CDCl₃) δ 8.21 (s, 1H), 6.20 (s, 1H), 4.41 (m, 1H), 4.31 (m, 1H), 3.33 (dd, J=14.0, 6.8 Hz, 1H), 3.25 (dd, J=13.9, 5.6 Hz, 1H), 2.98 (d, J=3.7 Hz, 1H), 2.77 (d, J=4.8 Hz, 3H), 2.22 (m, 1H), 2.03 (m, 1H), 1.91 (td, J=13.6, 4.5 Hz, 1H), 1.51 (s, 3H), 1.46 (s, 3H), 1.27 (s, 3H), 1.20 (s, 3H), 1.11 (s, 3H), 1.01-1.84 (m, 13H), 0.88 (d, J=6.3 Hz, 3H), 0.68 (d, J=6.6 Hz, 3H).

T27: To a solution of crude compound T21 (88% pure, 40 mg, 0.074 mmol) in CH₂Cl₂ (3 mL) at 0° C. was added Et₃N (31 μL, 0.22 mmol) and azetidine-1-carbonyl chloride (11 mg, 0.089 mmol) sequentially. The reaction was stirred at room temperature for 3 h. After which, additional amount of azetidine-1-carbonyl chloride (15 mg, 0.13 mmol) was added. The reaction was stirred at room temperature for overnight. The reaction was quenched with saturated aqueous NaHCO₃(20 mL). The mixture was extracted with EtOAc (3×20 mL). The combined organic extracts were washed with brine (20 mL), dried with Na₂SO₄, filtered, and concentrated. The residue was purified by column chromatography (silica gel, eluting with 0-100% acetone in hexanes) to give compound T27 (20 mg, 48% yield) as an off-white solid. m/z=560 (M+1); ¹H NMR (400 MHz, CDCl₃) 8.06 (s, 1H), 6.11 (s, 1H), 4.10 (t, J=6.4 Hz, 1H), 3.96 (t, J=7.5 Hz, 4H), 3.40 (dd, J=14.0, 7.1 Hz, 1H), 3.15 (dd, J=13.9, 6.0 Hz, 1H), 3.04 (d, J=3.8 Hz, 1H), 2.27 (m, 2H), 2.18 (m, 1H), 2.10 (m, 1H), 1.50 (s, 3H), 1.48 (s, 3H), 1.26 (s, 3H), 1.19 (s, 3H), 1.12 (s, 3H), 1.01-1.92 (m, 14H), 0.89 (d, J=6.4 Hz, 3H), 0.71 (d, J=6.6 Hz, 3H).

T28: To a solution of crude compound T21 (37 mg, <0.078 mmol) in CH₂Cl₂ (1 mL) at 0° C. was added 2,2-difluoroacetic acid (11 mg, 0.12 mmol), Et₃N (27 μL, 0.19 mmol) and propylphosphonic anhydride (T3P, 50% in EtOAc solution, 55 μL, 0.093 mmol) sequentially. The reaction was stirred at 0° C. for 30 min, and then was diluted with EtOAc (10 mL) and water (10 mL). The organic extract was washed with water (2×10 mL). The combined aqueous washes were extracted with EtOAc (20 mL). The combined organic extracts were washed with brine (20 mL), dried with Na₂SO₄, filtered, and concentrated. The residue was purified by column chromatography (silica gel, eluting with 0-40% acetone in hexanes) to give compound T28 (14 mg, 33% yield) as an off-white solid. m/z=555 (M+1); ¹H NMR (400 MHz, CDCl₃) δ 8.05 (s, 1H), 6.40 (bs, 1H), 6.13 (s, 1H), 5.94 (t, J=54.4 Hz, 1H), 3.55 (dd, J=13.8, 7.2 Hz, 1H), 3.31 (dd, J=13.8, 6.1 Hz, 1H), 2.94 (d, J=3.7 Hz, 1H), 2.19 (m, 1H), 2.07-1.89 (m, 2H), 1.51 (s, 3H), 1.47 (s, 3H), 1.27 (s, 3H), 1.20 (s, 3H), 1.13 (s, 3H), 0.90 (d, J=6.4 Hz, 3H), 1.02-1.85 (m, 13H), 0.71 (d, J=6.6 Hz, 3H).

T29: To a solution of crude compound T21 (37 mg, <0.078 mmol) in CH₂Cl₂ (1 mL) at 0° C. was added 2,2-difluoropropanoic acid (13 mg, 0.12 mmol), Et₃N (27 μL, 0.19 mmol) and propylphosphonic anhydride (T3P, >50 wt. % in EtOAc solution, 55 μL, 2 0.093 mmol) sequentially. The reaction was stirred at 0° C. for 30 min, and then was diluted with EtOAc (10 mL) and water (10 mL). The organic extract was washed with water (2×10 mL). The combined aqueous washes were extracted with EtOAc (20 mL). The combined organic extracts were washed with brine (20 mL), dried with Na₂SO₄, filtered, and concentrated. The residue was purified by column chromatography (silica gel, eluting with 0-100% acetone in hexanes) to give partially purified product which was purified again by column chromatography (silica gel, eluting with 0-40% acetone in hexanes) to give compound T29 (21 mg, 48% yield) as an off-white solid. m/z=569 (M+1); ¹H NMR (400 MHz, CDCl₃) δ 8.05 (s, 1H), 6.45 (bs, 1H), 6.12 (s, 1H), 3.48 (dd, J=13.8, 6.9 Hz, 1H), 3.33 (dd, J=13.8, 6.4 Hz, 1H), 2.93 (d, J=3.8 Hz, 1H), 2.18 (m, 1H), 1.83 (t, J=19.6 Hz, 3H), 1.51 (s, 3H), 1.47 (s, 3H), 1.26 (s, 3H), 1.20 (s, 3H), 1.13 (s, 3H), 1.01-2.08 (m, 15H), 0.90 (d, J=6.3 Hz, 3H), 0.71 (d, J=6.5 Hz, 3H).

Compound 35: In a 20 mL microwave vial, compound 18 (200 mg, 0.42 mmol) and methyl carbamate (126 mg, 1.67 mmol) were combined and dissolved in acetonitrile (9 mL). Trifluoroacetic acid (0.26 mL, 3.35 mmol) and tert-butyldimethylsilane (0.56 mL, 3.35 mmol) were added sequentially at 0° C. The vial was sealed and heated at 100° C. for 2 days. The reaction was cooled to room temperature; diluted with EtOAc (20 mL); and quenched with saturated aqueous NaHCO₃(20 mL). The organic extract was washed with water (2×20 mL). The combined aqueous washes were extracted with EtOAc (30 mL). The combined organic extracts were washed with brine (30 mL), dried with Na₂SO₄, filtered, and concentrated. The residue was purified by column chromatography (silica gel, eluting with 0-80% EtOAc in hexanes) to give compound 35 (160 mg, 71% yield). m/z=537 (M+1).

Compound 32: In a microwave vial, compound 35 (100 mg, 0.186 mmol) in acetic acid (1 mL) was added aqueous HCl (12 M, 0.5 mL, 6 mmol) at room temperature. The vial was sealed and heated at 120° C. in Biotage microwave synthesizer for 30 min. After cooled to room temperature, the mixture was concentrated. The residue was diluted with EtOAc (20 mL). The mixture was washed with saturated aqueous NaHCO₃(20 mL). The organic extract was washed with water (2×10 mL). The combined aqueous washes were extracted with EtOAc (20 mL). The combined organic extracts were washed with brine (20 mL), dried with Na₂SO₄, filtered, and concentrated. The residue was purified by column chromatography [(silica gel, eluting with 0-20% MeOH in (1% Et₃N in CH₂Cl₂)] to give compound 32 (91% pure, 90 mg, 92% yield). m/z=479 (M+1).

Compound 36: Compound 35 (97 mg, 0.18 mmol) in MeOH (4 mL) was treated with sodium methoxide solution (25 wt. % in MeOH, 83 μL, 0.36 mmol) at room temperature under nitrogen. The mixture was heated at 55° C. for 1.5 h, and then cooled to 0° C. The mixture was treated with 10% aqueous NaH₂PO₄ (10 mL). The mixture was extracted with EtOAc (2×20 mL). The combined organic extracts were washed with brine (20 mL), dried with Na₂SO₄, filtered and concentrated. The crude was purified by column chromatography (silica gel, eluting with 0-100% EtOAc in hexanes) to give compound 36 (81 mg, 84% yield). m/z=537 (M+1).

T30: Compound 36 (81 mg, 0.15 mmol) was dissolved in DMF (3 mL) and cooled to 0° C. under nitrogen. 1,3-Dibromo-5,5-dimethylhydantoin (22 mg, 0.075 mmol) was dissolved in DMF (0.5 mL) in a vial. The solution was added dropwise. DMF (0.5 mL) was used to rinse the vial and added to the reaction mixture. The mixture was stirred at 0° C. for 1 h. Pyridine (36 μL, 0.45 mmol) was added. The mixture was heated at 60° C. for 8 h. After cooled to 0° C., the mixture was diluted with EtOAc (20 mL) and washed with water (2×10 mL) and brine (20 mL). The organic extract was dried with Na₂SO₄, filtered, and concentrated. The residue was purified by column chromatography (silica gel, eluting with EtOAc in hexanes) to give compound T30 (58 mg, 72% yield) as an off-white solid. m/z=535 (M+1); ¹H NMR (400 MHz, CDCl₃) δ 8.06 (s, 1H), 6.13 (s, 1H), 4.80 (t, J=6.5 Hz, 1H), 3.66 (s, 3H), 3.30 (dd, J=14.1, 7.1 Hz, 1H), 3.21 (dd, J=13.9, 6.3 Hz, 1H), 2.97 (d, J=3.8 Hz, 1H), 2.20 (dd, J=11.2, 3.8 Hz, 1H), 2.01 (m, 1H), 1.91 (td, J=13.5, 4.3 Hz, 1H), 1.51 (s, 3H), 1.46 (s, 3H), 1.26 (s, 3H), 1.19 (s, 3H), 1.13 (s, 3H), 1.02-1.85 (m, 13H), 0.89 (d, J=6.3 Hz, 3H), 0.71 (d, J=6.6 Hz, 3H).

T31: To a solution of crude compound T21 (40 mg, <0.084 mmol) in CH₂Cl₂ (3 mL) at 0° C. was added Et₃N (35 μL, 0.25 mmol) and methanesulfonyl chloride (12 mg, 0.10 mmol) sequentially. The reaction was stirred at room temperature for 1 h, and then was diluted with EtOAc (20 mL). The mixture was washed with saturated aqueous NaHCO₃(20 mL) and water (2×10 mL). The combined aqueous washes were extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give compound T31 (24 mg, 52% yield) as an off-white solid. m/z=555 (M+1); ¹H NMR (400 MHz, CDCl₃) 8.06 (s, 1H), 6.15 (s, 1H), 4.38 (t, J=6.8 Hz, 1H), 3.10-3.23 (m, 2H), 2.99 (s, 3H), 2.77 (d, J=3.8 Hz, 1H), 2.24 (dd, J=11.1, 3.7 Hz, 1H), 1.50 (s, 3H), 1.40 (s, 3H), 1.27 (s, 3H), 1.20 (s, 3H), 1.14 (s, 3H), 1.04-1.98 (m, 15H), 0.90 (d, J=6.4 Hz, 3H), 0.71 (d, J=6.7 Hz, 3H).

T32: To a solution of crude compound T21 (40 mg, <0.084 mmol) in CH₂Cl₂ (3 mL) at 0° C. was added Et₃N (35 μL, 0.25 mmol) and ethanesulfonyl chloride (13 mg, 0.10 mmol) sequentially. The reaction was stirred at room temperature for 1 h, and then was diluted with EtOAc (20 mL). The mixture was washed with saturated aqueous NaHCO₃(20 mL) and water (2×10 mL). The combined aqueous washes were extracted with EtOAc (20 mL). The combined organic extracts were washed with brine (20 mL), dried with Na₂SO₄, filtered, and concentrated.

The residue was purified by column chromatography (silica gel, eluting with 0-60% acetone in hexanes) to give compound T32 (22 mg, 46% yield) as an off-white solid. m/z=569 (M+1); ¹H NMR (400 MHz, CDCl₃) 8.05 (s, 1H), 6.14 (s, 1H), 4.23 (dd, J=5.6, 7.6 Hz, 1H), 3.22 (dd, J=13.0, 8.2 Hz, 1H), 3.01-3.11 (m, 3H), 2.76 (d, J=3.8 Hz, 1H), 2.21 (dd, J=11.1, 3.6 Hz, 1H), 1.98-1.69 (m, 6H), 1.50 (s, 3H), 1.39 (s, 3H), 1.38 (t, J=7.2 Hz, 3H), 1.27 (s, 3H), 1.20 (s, 3H), 1.14 (s, 3H), 1.02-1.60 (m, 9H), 0.90 (d, J=6.4 Hz, 3H), 0.71 (d, J=6.6 Hz, 3H).

T33: To a solution of crude compound T21 (40 mg, <0.084 mmol) in CH₂Cl₂ (3 mL) at 0° C. was added Et₃N (35 μL, 0.25 mmol) and cyclopropanesulfonyl chloride (14 mg, 0.10 mmol) sequentially. The reaction was stirred at room temperature for 1 h. Additional amount of cyclopropanesulfonyl chloride (14 mg, 0.10 mmol) was added. After stirred for overnight, the reaction was not complete. Additional amount of Et₃N (35 μL, 0.25 mmol) and cyclopropanesulfonyl chloride (14 mg, 0.10 mmol) were added. The reaction was stirred at room temperature for another 24 h. The reaction was diluted with EtOAc (20 mL). The mixture was washed with saturated aqueous NaHCO₃(20 mL) and water (2×10 mL). The combined aqueous washes were extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give compound T33 (9 mg, 20% yield) as an off-white solid. m/z=581 (M+1); ¹H NMR (400 MHz, CDCl₃) 8.06 (s, 1H), 6.15 (s, 1H), 4.39 (dd, J=8.2, 5.6 Hz, 1H), 3.26 (dd, J=12.9, 8.3 Hz, 1H), 3.10 (dd, J=12.9, 5.4 Hz, 1H), 2.79 (d, J=3.8 Hz, 1H), 2.45 (tt, J=8.0, 4.8 Hz, 1H), 2.25 (dd, J=11.4, 3.0 Hz, 1H), 1.50 (s, 3H), 1.40 (s, 3H), 1.27 (s, 3H), 1.20 (s, 3H), 1.14 (s, 3H), 0.92-1.96 (m, 19H), 0.90 (d, J=6.3 Hz, 3H), 0.71 (d, J=6.6 Hz, 3H).

Compound 37: To a solution of compound 18 (250 mg, 0.52 mmol) in THF (5 mL) was added methylamine (2 M in THF, 3.9 mL, 7.8 mmol) at 0° C. The reaction was stirred at room temperature for 2 h, and then was cooled to 0° C. Acetic acid (0.45 mL, 7.85 mmol) was added. The reaction was stirred for another 5 min, and then a solution of sodium cyanoborohydride (493 mg, 7.85 mmol) in MeOH (5 mL) was added. The reaction was stirred at room temperature for another 2 h. EtOAc (20 mL) and saturated aqueous NaHCO₃(20 mL) were added. The organic extract was separated and washed with water (2×20 mL). The combined aqueous phase was extracted with EtOAc (20 mL). The combined organic extracts were washed with brine (20 mL), dried with Na₂SO₄, filtered, and concentrated to give the crude compound 37 (338 mg), which was used in the next step without purification. m/z=493 (M+1).

Compound 38: To a mixture of crude compound 37 (338 mg, ≤0.52 mmol) and NaHCO₃(69 mg, 0.82 mmol) in THF (3 mL) and H₂O (1 mL) was added di-tert-butyl dicarbonate (225 mg, 1.03 mmol) at room temperature. The reaction was stirred at room temperature for 30 min. EtOAc (20 mL) and water (20 mL) were added. The organic extract was separated and washed with water (2×20 mL) and brine (20 mL); dried with Na₂SO₄; filtered and concentrated. The residue was purified by column chromatography (silica gel, eluting with 0-80% EtOAc in hexanes) to give compound 38 (196 mg, 64% yield from 18). m/z=537 (M-C₄H₇).

Compound 39: Compound 38 (874 mg, 1.47 mmol) in MeOH (9 mL) was treated with sodium methoxide solution (25 wt. % in MeOH, 674 μL, 2.95 mmol) at room temperature under nitrogen. The mixture was heated at 55° C. for 1.5 h, and then cooled to 0° C. The mixture was treated with 10% aqueous NaH₂PO₄ (10 mL) and extracted with EtOAc (2×20 mL). The combined organic extracts were washed with brine (20 mL), dried with Na₂SO₄, filtered and concentrated. The crude was purified by column chromatography (silica gel, eluting with EtOAc in hexanes) to give compound 39 (584 mg, 67% yield). m/z=537 (M-C₄H₇).

Compound 40: Compound 39 (584 mg, 0.98 mmol) was dissolved in DMF (8 mL) and cooled to 0° C. under nitrogen. 1,3-Dibromo-5,5-dimethylhydantoin (141 mg, 0.49 mmol) was dissolved in DMF (1 mL) in a vial. The solution was added dropwise. DMF (1 mL) was used to rinse the vial and added to the reaction mixture. The mixture was stirred at 0° C. for 1 h. Pyridine (238 μL, 2.96 mmol) was added. The mixture was heated at 60° C. for 8 h. After cooled to 0° C., the mixture was diluted with EtOAc (20 mL) and washed with water (2×10 mL) and brine (20 mL). The organic extract was dried with Na₂SO₄, filtered, and concentrated. The residue was purified by column chromatography (silica gel, eluting with EtOAc in hexanes) to give compound (327 mg, 56% yield). m/z=535 (M-C₄H₇).

T34: Compound 40 (327 mg, 0.55 mmol)in CH₂Cl₂ (6 mL) was treated with trifluoroacetic acid (1.2 mL, 16 mmol) at 0° C. The reaction was stirred at 0° C. for 3 h. and then was diluted with EtOAc (30 mL) and saturated aqueous NaHCO₃(30 mL). The organic extract was separated and washed with saturated aqueous NaHCO₃(2×20 mL) and H₂O (30 mL). The aqueous phase was extracted with EtOAc (20 mL). The combined organic extracts were washed with brine (30 mL), dried with Na₂SO₄, filtered, and concentrated to give crude compound T34 (234 mg, 86% yield) as a yellow solid. Analytical sample was obtained by purifying the crude product (30 mg) by column chromatography [silica gel, eluting with 0-15% (1% Et₃N in MeOH) in CH₂Cl₂] three times to give compound T34 (20 mg, 67% yield) as a pale-yellow solid. m/z=491 (M+1); ¹H NMR (400 MHz, CDCl₃) δ 8.05 (s, 1H), 6.14 (s, 1H), 2.82 (d, J=3.7 Hz, 1H), 2.57 (d, J=11.9 Hz, 1H), 2.47 (d, J=11.8 Hz, 1H), 2.43 (s, 3H), 2.27 (dd, J=3.2, 11.2 Hz, 1H), 1.50 (s, 3H), 1.37 (s, 3H), 1.27 (s, 3H), 1.20 (s, 3H), 1.13 (s, 3H), 1.03-1.96 (m, 15H), 0.88 (d, J=6.3 Hz, 4H), 0.70 (d, J=6.6 Hz, 4H).

T35: To a solution of crude compound T34 (40 mg, 0.082 mmol) in CH₂Cl₂ (2 mL) at 0° C. was added Et₃N (26 μL, 0.18 mmol) and acetic anhydride (13 μL, 0.14 mmol) sequentially. The reaction was stirred at 0° C. for 30 min, and then was diluted with EtOAc (10 mL) and H₂O (10 mL). The organic extract was separated and washed with water (2×10 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give a partially purified product which was purified again by column chromatography (silica gel, eluting with 0-60% acetone in hexanes) to give compound T35 (18 mg, 41% yield) as an off-white solid. m/z=533 (M+1); ¹H NMR (400 MHz, CDCl₃) δ 8.06 (s, 1H), 6.13 (s, 1H), 3.80 (d, J=13.9 Hz, 1H), 3.18 (d, J=3.7 Hz, 1H), 3.12 (d, J=13.9 Hz, 1H), 3.08 (s, 3H), 2.18-2.28 (m, 2H), 2.12 (s, 3H), 1.90 (td, J=13.9, 4.9 Hz, 1H), 1.72-1.84 (m, 4H), 1.50 (s, 3H), 1.45 (s, 3H), 1.26 (s, 3H), 1.19 (s, 3H), 1.12 (s, 3H), 1.04-1.65 (m, 9H), 0.88 (d, J=6.3 Hz, 3H), 0.70 (d, J=6.6 Hz, 3H).

T36: To a solution of crude compound T34 (40 mg, <0.082 mmol) in CH₂Cl₂ (2 mL) at 0° C. was added Et₃N (34 μL, 0.24 mmol) and methylaminoformyl chloride (10 mg, 0.11 mmol) sequentially. The reaction was stirred at 0° C. for 30 min, and then was diluted with EtOAc (10 mL) and water (10 mL). The organic extract was separated and washed with water (2×10 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give partially purified product which was purified again by column chromatography (silica gel, eluting with 0-60% acetone in hexanes) to give compound T36 (17 mg, 38% yield) as an off-white solid. m/z=548 (M+1); 1H NMR (400 MHz, CDCl₃) δ 8.06 (s, 1H), 6.13 (s, 1H), 4.37 (q, J=4.9 Hz, 1H), 3.90 (d, J=14.3 Hz, 1H), 3.06 (d, J=3.7 Hz, 1H), 2.90-3.01 (m, 4H), 2.81 (d, J=4.6 Hz, 3H), 2.14-2.31 (m, 2H), 1.50 (s, 3H), 1.45 (s, 3H), 1.26 (s, 3H), 1.19 (s, 3H), 1.12 (s, 3H), 1.01-1.91 (m, 14H), 0.89 (d, J=6.3 Hz, 3H), 0.69 (d, J=6.6 Hz, 3H).

T37: To a solution of crude compound T34 (62 mg, <0.13 mmol) in CH₂Cl₂ (1 mL) at 0° C. was added Et₃N (53 μL, 0.38 mmol) and d3-acetyl chloride (11 μL, 0.16 mmol) sequentially. The reaction was stirred at 0° C. for 30 min, and then was diluted with EtOAc (10 mL) and water (10 mL). The organic extract was separated and washed with water (2×10 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-40% acetone in hexanes) to give compound T37 (26 mg, 38% yield) as an off-white solid. m/z=536 (M+1); ¹H NMR (400 MHz, CDCl₃) δ 8.06 (s, 1H), 6.13 (s, 1H), 3.78 (d, J=13.8 Hz, 1H), 3.18 (d, J=3.7 Hz, 1H), 3.13 (d, J=13.8 Hz, 1H), 3.07 (s, 3H), 2.16-2.27 (m, 2H), 1.89 (td, J=13.9, 4.9 Hz, 1H), 1.72-1.83 (m, 4H), 1.49 (s, 3H), 1.44 (s, 3H), 1.25 (s, 3H), 1.18 (s, 3H), 1.12 (s, 3H), 1.03-1.67 (m, 9H), 0.88 (d, J=6.3 Hz, 3H), 0.69 (d, J=6.6 Hz, 3H).

T38: To a solution of crude compound T34 (40 mg, <0.082 mmol) and 2,2-difluoropropanoic acid (13 mg, 0.12 mmol) in CH₂Cl₂ (2 mL) at 0° C. was added Et₃N (28 μL, 0.20 mmol) and propylphosphonic anhydride (T3P, 50% in EtOAc solution, 58 μL, 0.098 mmol) sequentially. The reaction was stirred at 0° C. for 30 min and then at room temperature for overnight. Additional amount of 2,2-difluoropropanoic acid (13 mg, 0.12 mmol), Et₃N (28 μL, 0.2 mmol) and propylphosphonic anhydride (T3P, 50% in EtOAc solution, 58 μL, 0.098 mmol) were added at 0° C. The reaction was stirred at room temperature for another 2 h. The reaction was then heated in a Biotage microwave synthesizer at 100° C. for 30 min to drive the reaction to completion. After cooled to room temperature, the reaction was diluted with EtOAc (10 mL) and quenched with saturated aqueousNaHCO3 (10 mL). The organic extract was separated and washed with water (2×10 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-80% EtOAc in hexanes) to give partially purified product which was purified again by column chromatography (silica gel, eluting with 0-40% acetone in hexanes) to give compound T38 (12 mg, 25% yield) as an off-white solid. m/z=583 (M+1); ¹H NMR (400 MHz, CDCl₃) δ 8.05 (s, 1H), 6.14 (s, 1H), 3.93 (d, J=13.6 Hz, 1H), 3.26 (s, 3H), 3.11 (d, J=13.7 Hz, 1H), 2.97 (d, J=3.8 Hz, 1H), 2.11-2.27 (m, 2H), 1.84 (t, J=19.6 Hz, 3H), 1.51 (s, 3H), 1.43 (s, 3H), 1.27 (s, 3H), 1.20 (s, 3H), 1.14 (s, 3H), 1.04-1.98 (m, 14H), 0.89 (d, J=6.2 Hz, 3H), 0.70 (d, J=6.5 Hz, 3H).

T39: To a solution of crude compound T34 (40 mg, <0.082 mmol) and 2,2-difluoroacetic acid (12 mg, 0.12 mmol) in CH₂Cl₂ (1 mL) at 0° C. was added Et₃N (28 μL, 0.20 mmol) and propylphosphonic anhydride (T3P, 50% in EtOAc solution, 58 μL, 0.098 mmol) sequentially. The reaction was stirred at 0° C. for 30 min and then at room temperature for overnight. Additional amount of 2,2-difluoroacetic acid (12 mg, 0.12 mmol), Et₃N (28 μL, 0.20 mmol) and propylphosphonic anhydride (T3P, 50% in EtOAc solution, 58 μL, 0.098 mmol) were added. The reaction was stirred at room temperature for another 2 h, and then was heated in a Biotage microwave synthesizer at 100° C. for 30 min to drive the reaction to completion. After cooled to room temperature, the reaction was diluted with EtOAc (10 mL) and quenched with saturated aqueous NaHCO₃(10 mL). The organic extract was separated and washed with water (2×10 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-100% EtOAc in hexanes) to give partially purified product which was purified again by column chromatography (silica gel, eluting with 0-40% acetone in hexanes) to give compound T39 (7 mg, 20% yield) as an off-white solid. m/z=569 (M+1); ¹H NMR (400 MHz, CDCl₃) δ 8.05 (s, 1H), 6.14 (s, 1H), 6.14 (t, J=53.2 Hz, 1H), 3.94 (d, J=13.7 Hz, 1H), 3.21 (s, 3H), 3.09 (d, J=13.7 Hz, 1H), 3.00 (d, J=3.8 Hz, 1H), 2.12-2.27 (m, 2H), 1.93 (td, J=14.1, 4.8 Hz, 1H), 1.73-1.86 (m, 4H), 1.50 (s, 3H), 1.43 (s, 3H), 1.26 (s, 3H), 1.19 (s, 3H), 1.13 (s, 3H), 1.03-1.65 (m, 9H), 0.89 (d, J=6.4 Hz, 3H), 0.69 (d, J=6.5 Hz, 3H).

Compound 41: To a solution of compound 18 (400 mg, 0.84 mmol) in THF (10 mL) at room temperature was added ethanolamine (253 μL, 4.19 mmol) under argon atmosphere. The mixture was stirred at room temperature for 2 h, and then acetic acid (240 μL, 4.19 mmol) was added at room temperature. The resultant mixture was stirred at room temperature for another 2 h. A solution of sodium cyanoborohydride (263 mg, 4.19 mmol)in MeOH (10 mL) was added slowly at room temperature. The reaction mixture was stirred at room temperature for additional 16 h, and then quenched with saturated aqueous NaHCO₃(10 mL). The mixture was concentrated under reduced pressure. The residue was extracted with EtOAc (3×10 mL). The combined organic extracts were dried over Na₂SO₄, filtered, and concentrated under reduced pressure. The residue was purified by column chromatography (silica gel, eluting with 0-20% MeOH in EtOAc) to give compound 41 (400 mg, 91% yield) as a white solid. m/z=523 (M+1).

Compound 42: To a solution of compound 41(392 mg, 0.75 mmol) in CH₂Cl₂ (10 mL) at room temperature was added di-tert-butyl dicarbonate (180 mg, 0.82 mmol) and Et₃N (209 μL, 1.50 mmol) sequentially. The mixture was stirred at room temperature for 16 h and then concentrated under reduced pressure. The residue was purified by column chromatography (silica gel, eluting with 0-40% acetone in hexanes) to give compound 42 (378 mg, 81% yield) as a colorless solid. m/z=623 (M+1).

Compound 43a and 43b: To a solution of compound 42 (378 mg, 0.61 mmol) in MeOH (10 mL) at room temperature was added potassium carbonate (168 mg, 1.21 mmol). The mixture was stirred at room temperature for 19 h and then concentrated under reduced pressure. The residue was dissolved in a mixture of EtOAc (10 mL) and aqueous HCl (1 N, 10 mL), which was stirred for 20 min at room temperature. The aqueous phase was separated and extracted with EtOAc (2×10 mL). The organic extracts were combined, dried over Na₂SO₄, filtered, and concentrated under reduced pressure. The residue was purified by column chromatography (silica gel, eluting with 0-50% acetone in hexanes) to give compound 43a (230 mg, 61% yield) as a white solid and compound 43b (120 mg, 30% yield) as a white solid. 43a: m/z=623 (M+1); 43b: m/z=665 (M+1).

Compound 44: To a solution of compound 43a (230 mg, 0.37 mmol) in DMF (2 mL) at 0° C. was added 1,3-dibromo-5,5-dimethylhydantoin (51 mg, 0.18 mmol) under argon atmosphere. The mixture was stirred at 0° C. for 1 h. Pyridine (209 μL, 2.58 mmol) was then added at 0° C. The reaction mixture was stirred at 55° C. for another 4 h, and then cooled to room temperature. The mixture was poured into aqueous HCl (1 N, 10 mL). The resultant mixture was extracted with EtOAc (3×10 mL). The combined organic extracts were washed with brine (2×10 mL), dried over Na₂SO₄, filtered, and concentrated under reduced pressure. The residue was purified by column chromatography (silica gel, eluting with 0-40% acetone in hexanes) to give compound 44 (124 mg, 54% yield) as a white solid. m/z=521 (M-C₅H₇O₂).

T40: To a solution of compound 44 (124 mg, 0.20 mmol) in CH₂Cl₂ (3 mL) at room temperature was added trifluoracetic acid (0.77 mL, 9.99 mmol). The mixture was stirred at room temperature for 2 h, and then quenched by the slow addition of saturated aqueous NaHCO₃(10 mL) at room temperature. The mixture was extracted with CH₂Cl₂ (2×10 mL). The combined organic extracts were dried over Na₂SO₄, filtered, and concentrated under reduced pressure. The residue was purified by column chromatography (silica gel, eluting with 0-90% MeOH in CH₂Cl₂ with 0.5% Et₃N added) to give compound T40 (80 mg, 77% yield) as a yellow solid. m/z=521 (M+1); ¹H NMR (400 MHz, CDCl₃) δ 8.08 (s, 1H), 6.16 (s, 1H), 3.62 (t, J=5.1 Hz, 2H), 2.84 (m, 2H), 2.76 (d, J=3.7 Hz, 1H), 2.72 (d, J=12.1 Hz, 1H), 2.54 (d, J=12.0 Hz, 1H), 2.37 (m, 1H), 1.94 (m, 1H), 1.64 (td, J=13.2, 4.4 Hz, 1H), 1.50 (s, 3H), 1.37 (s, 3H), 1.27 (s, 3H), 1.20 (s, 3H), 1.14 (s, 3H), 0.91-3.24 (m, 15H), 0.89 (d, J=6.4 Hz, 3H), 0.70 (d, J=6.6 Hz, 3H).

T41: To a solution of compound T40 (65 mg, 0.12 mmol) in anhydrous CH₂Cl₂ (2 mL) at room temperature was added 1,1′-carbonyldiimidazole (20 mg, 0.12 mmol). The mixture was stirred at room temperature for 3 h, and then concentrated under reduced pressure. The residue was purified by column chromatography (silica gel, eluting with 0-40% acetone in hexanes) to give compound T41 (23 mg, 34% yield) as a white solid. m/z=547 (M+1); ¹H NMR (400 MHz, CDCl₃) δ 8.08 (s, 1H), 6.16 (s, 1H), 4.34 (dd, J=9.3, 6.8 Hz, 2H), 3.78 (q, J=7.7 Hz, 1H), 3.57 (q, J=8.6 Hz, 1H), 3.46 (d, J=14.4 Hz, 1H), 3.09 (d, J=14.4 Hz, 1H), 2.97 (d, J=3.7 Hz, 1H), 2.26 (m, 1H), 2.09 (m, 1H), 1.97 (td, J=13.8, 4.6 Hz, 1H), 1.51 (s, 3H), 1.44 (s, 3H), 1.27 (s, 3H), 1.20 (s, 3H), 1.14 (s, 3H), 1.02-1.85 (m, 13H), 0.89 (d, J=6.3 Hz, 3H), 0.71 (d, J=6.6 Hz, 3H).

T42: To a solution of compound T40 (25 mg, 0.048 mmol) in THF (2 mL) at room temperature was added paraformaldehyde (2.2 mg, 0.072 mmol) under argon atmosphere. The reaction flask was sealed, and the mixture was heated to 75° C. and stirred at 75° C. for 48 h. The reaction mixture was cooled to room temperature and concentrated under reduced pressure. The residue was first purified by column chromatography (silica gel, eluting with 0-40% acetone in hexane) to give partially purified product which was purified again by column chromatography (silica gel, eluting with 0-60% EtOAc in hexanes) to give compound T42 (1.98 mg, 8% yield) as a white solid. m/z=533 (M+1); ¹H NMR (400 MHz, CDCl₃) δ 8.07 (s, 1H), 6.04 (s, 1H), 3.74 (ddd, J=4.0, 10.0, 11.6 Hz, 1H), 3.56 (dt, J=11.3, 4.4 Hz, 1H), 3.15 (d, J=12.7 Hz, 1H), 2.78-2.90 (m, 2H), 2.49 (ddd, J=12.5, 9.7, 4.9 Hz, 1H), 1.65 (s, 3H), 1.52 (s, 3H), 1.28 (s, 3H), 1.20 (s, 3H), 1.17 (s, 3H), 1.00-2.14 (m, 19H), 0.89 (d, J=6.4 Hz, 3H), 0.79 (d, J=6.5 Hz, 3H).

Compound 45: To a solution of compound 43b (124 mg, 0.19 mmol) in DMF (1 mL) at 0° C. was added 1,3-dibromo-5,5-dimethylhydantoin (25.6 mg, 0.090) under argon atmosphere. The mixture was stirred at 0° C. for 1 h, then pyridine (106 μL, 1.31 mmol) was added at 0° C. The resultant mixture was stirred at 55° C. for 4.5 h, then cooled to room temperature. The mixture was diluted with water (5 mL), and was extracted with EtOAc (3×5 mL). The combined organic extracts were washed with brine (2×5 mL), dried with Na₂SO₄, filtered, and concentrated under reduced pressure. The residue was purified by column chromatography (silica gel, eluting with 0-40% acetone in hexanes) to give compound 45 (87.5 mg, 71% yield) as a white solid. m/z=607 (M-C₄H₇) T43: To a solution of compound 45 (87.5 mg, 0.13 mmol) in CH₂Cl₂ (2 mL) at room temperature was added trifluoracetic acid (0.51 mL, 6.60 mmol). The mixture was stirred at room temperature for 4 h, then quenched by slow addition of saturated aqueous NaHCO₃(10 mL). The mixture was extracted with CH₂Cl₂ (2×10 mL). The combined organic extracts were dried over Na₂SO₄, filtered, and concentrated under reduced pressure. The residue was purified by column chromatography (silica gel, eluting with 0-90% MeOH in CH₂Cl₂ with 0.5% Et₃N added) to give compound T43 (67 mg, 90% yield) as a yellow solid. m/z=563 (M+1); ¹H NMR (400 MHz, CDCl₃) δ 8.07 (s, 1H), 6.15 (s, 1H), 4.14 (m, 2H), 2.85 (dd, J=6.2, 5.0 Hz, 2H), 2.78 (d, J=3.8 Hz, 1H), 2.67 (d, J=11.8 Hz, 1H), 2.47 (d, J=11.9 Hz, 1H), 2.31 (dd, J=11.2, 2.8 Hz, 1H), 2.04 (s, 3H), 1.91 (td, J=13.5, 4.3 Hz, 1H), 1.65 (td, J=13.1, 4.4 Hz, 1H), 1.50 (s, 3H), 1.36 (s, 3H), 1.27 (s, 3H), 1.20 (s, 3H), 1.13 (s, 3H), 1.05-1.85 (m, 13H), 0.88 (d, J=6.3 Hz, 3H), 0.70 (d, J=6.6 Hz, 3H).

Compound 46a: To a solution of compound 18 (250 mg, 0.52 mmol) in THF (7.5 mL) at room temperature was added N-Boc-N-methylethylenediamine (468 μL, 2.62 mmol) under argon atmosphere. The mixture was stirred at room temperature for 3 h, and then acetic acid (150 μL, 2.62 mmol) was added. The resultant mixture was stirred at room temperature for another 5 min, then sodium cyanoborohydride (164 mg, 2.62 mmol) was added as a solution in MeOH (10 mL) dropwise at room temperature. The reaction mixture was stirred at room temperature for additional 16 h, then quenched with saturated aqueous NaHCO₃(10 mL). The mixture was concentrated under reduced pressure. The residue was extracted with CH₂Cl₂ (2×10 mL) and EtOAc (2×10 mL). The combined organic extracts were dried over Na₂SO₄, filtered, and concentrated under reduced pressure. The residue was purified by column chromatography (silica gel, eluting with 0-30% acetone in hexane) to give compound 46a (364 mg) as a white solid. m/z=636 (M+1).

Compound 47a: To a solution of compound 46a (364 mg, <0.52 mmol) in CH₂Cl₂ (5 mL) at room temperature was added trifluoracetic acid (2.0 mL, 26.2 mmol). The mixture was stirred at room temperature for 4 h, then quenched with saturated aqueous NaHCO₃(15 mL) carefully. The two phases were separated, and the aqueous phase was extracted with CH₂Cl₂ (2×15 mL). The combined organic extracts were dried over Na₂SO₄, filtered, and concentrated under reduced pressure to give compound 47a (282 mg) as a white solid. m/z=536 (M+1).

Compound 48a: To a solution of compound 47a (282 mg, 0.52 mmol) in anhydrous CH₂Cl₂ (5 mL) at room temperature was added phosgene solution (20% in toluene, 418 μL, 0.79 mmol) dropwise. The mixture was stirred at room temperature for 20 h, then N,N-diisopropylethylamine (92 μL, 0.53 mmol) was added at room temperature. The reaction mixture was stirred at room temperature for additional 3 h and then concentrated under reduced pressure. The residue was purified by column chromatography (silica gel, eluting with 0-60% acetone in hexanes) to give compound 48a (123 mg, 42% yield from compound 18) as a white solid. m/z=562 (M+1).

Compound 49a: To a solution of compound 48a (123 mg, 0.22 mmol) in MeOH (2 mL) at room temperature was added sodium methoxide (25 wt. % in MeOH, 0.125 mL, 0.55 mmol). The mixture was stirred at 55° C. for 3.5 h, then cooled to room temperature and quenched with acetic acid (5 drops). The resultant mixture was concentrated, and the residue was partitioned between water (10 mL) and CH₂Cl₂ (10 mL). The aqueous phase was extracted with CH₂Cl₂ (10 mL). The combined organic extracts were washed with water (10 mL), dried over Na₂SO₄, filtered, and concentrated under reduced pressure to give compound 49a (114 mg, 93% yield) as a white solid, which was used in next step without further purification. m/z=562 (M+1).

T44: To a solution of compound 49a (114 mg, 0.20 mmol) in DMF (2 mL) at 0° C. was added 1,3-dibromo-5,5-dimethylhydantoin (27.8 mg, 0.097 mmol) under argon atmosphere. The mixture was stirred at 0° C. for 1.5 h, and then pyridine (66 μL, 0.81 mmol) was added. The resultant mixture was heated to 55° C. and stirred at 55° C. for another 3 h. The reaction mixture was cooled to room temperature and partitioned between EtOAc (10 mL) and brine (10 mL). The aqueous phase was extracted with EtOAc (2×10 mL). The combined organic extracts were washed with brine (2×10 mL), dried over Na₂SO₄, filtered, and concentrated under reduced pressure. The residue was purified by column chromatography (silica gel, eluting with 0-60% acetone in hexanes) to give compound T44 (28 mg, 25% yield) as an off-white solid. m/z=560 (M+1); ¹H NMR (400 MHz, CDCl₃) δ 8.07 (s, 1H), 6.14 (s, 1H), 3.53 (m, 1H), 3.44 (d, J=14.3 Hz, 1H), 3.21-3.35 (m, 3H), 3.09 (d, J=3.7 Hz, 1H), 2.88 (d, J=14.2 Hz, 1H), 2.80 (s, 3H), 2.25 (m, 1H), 2.17 (m, 1H), 1.91 (td, J=13.6, 4.6 Hz, 1H), 1.72-1.85 (m, 4H), 1.50 (s, 3H), 1.46 (s, 4H), 1.26 (s, 4H), 1.19 (s, 3H), 1.13 (s, 3H), 1.01-1.67 (m, 9H), 0.88 (d, J=6.3 Hz, 3H), 0.70 (d, J=6.6 Hz, 3H).

Compound 46b: To a solution of compound 18 (300 mg, 0.63 mmol) in THF (10 mL) at room temperature was added N-Boc-ethylenediamine (497 μL, 3.14 mmol) under argon atmosphere. The mixture was stirred at room temperature for 3 h, and then acetic acid (180 μL, 3.14 mmol) was added. The resultant mixture was stirred at room temperature for another 5 min, then sodium cyanoborohydride (197 mg, 3.13 mmol) was added as a solution in MeOH (10 mL) dropwise at room temperature. The reaction mixture was stirred at room temperature for additional 16 h, and then quenched with saturated aqueous NaHCO₃(10 mL). The mixture was concentrated under reduced pressure. The residue was extracted with EtOAc (3×10 mL). The combined organic extracts were dried over Na₂SO₄, filtered, and concentrated under reduced pressure. The residue was purified by column chromatography (silica gel, eluting with 0-30% acetone in hexanes) to give compound 46b (440 mg) as a white solid. m/z=622 (M+1).

Compound 47b: To a solution of compound 46b (440 mg, <0.63 mmol) in CH₂Cl₂ (5 mL) at room temperature was added trifluoracetic acid (2.43 mL, 31.4 mmol). The mixture was stirred at room temperature for 4 h, then quenched with slow addition of saturated aqueous NaHCO₃(10 mL). The two phases were separated, and the aqueous phase was extracted with CH₂Cl₂ (2×10 mL). The combined organic extracts were dried over Na₂SO₄, filtered, and concentrated under reduced pressure to give compound 47b (320 mg) as a white solid, which was used in next step without further purification. m/z=522 (M+1).

Compound 48b: To a solution of compound 47b (320 mg, 0.61 mmol) in anhydrous CH₂Cl₂ (8 mL) at room temperature was added phosgene solution (20% in toluene, 487 μL, 0.92 mmol) dropwise. The mixture was stirred at room temperature for 20 h and N,N-diisopropylethylamine (107 μL, 0.61 mmol) was added. The reaction mixture was stirred for another 3 h and then concentrated under reduced pressure. The residue was purified by column chromatography (silica gel, eluting with 0-100% acetone in hexanes) to give compound 48b (48 mg, 14% yield from compound 18) as a white solid. m/z=548 (M+1).

Compound 49b: To a suspension of compound 48b (48 mg, 0.088 mmol) in MeOH (2 mL) at room temperature was added sodium methoxide (25 wt. % in MeOH, 0.05 mL, 0.22 mmol). The mixture was stirred at 55° C. for 2.5 h, then cooled to room temperature and quenched with acetic acid (2 drops). The resultant mixture was concentrated, and the residue was partitioned between water (10 mL) and CH₂Cl₂ (10 mL). The aqueous phase was extracted with CH₂Cl₂ (2×5 mL). The combined organic extracts were washed with water (10 mL), dried over Na₂SO₄, filtered, and concentrated under reduced pressure to give compound 49b (40 mg, 83% yield) as a white solid. m/z=548 (M+1).

T45: To a solution of compound 49b (40 mg, 0.073 mmol) in DMF (1 mL) at 0° C. was added 1,3-dibromo-5,5-dimethylhydantoin (10 mg, 0.035 mmol) under argon atmosphere. The mixture was stirred at 0° C. for 1.5 h, then pyridine (24 μL, 0.29 mmol) was added. The resultant mixture was then heated to 55° C. and stirred at 55° C. for another 3.5 h. The reaction mixture was cooled to room temperature and partitioned between EtOAc (10 mL) and brine (10 mL). The aqueous phase was extracted with EtOAc (2×10 mL). The combined organic extracts were washed with brine (2×10 mL), dried over Na₂SO₄, filtered, and concentrated under reduced pressure. The residue was purified by column chromatography (silica gel, eluting with 0-60% acetone in hexanes) to give compound T45 (4 mg, 10% yield) as an off-white solid. m/z=546 (M+1); ¹H NMR (400 MHz, CDCl₃) δ 8.06 (s, 1H), 6.14 (s, 1H), 4.40 (s, 1H), 3.63 (m, 1H), 3.38-3.49 (m, 3H), 3.24 (d, J=14.4 Hz, 1H), 3.16 (d, J=3.7 Hz, 1H), 3.09 (d, J=14.4 Hz, 1H), 2.29 (dd, J=11.2, 3.7 Hz, 1H), 2.09 (m, 1H), 1.95 (td, J=13.8, 4.8 Hz, 1H), 1.71-1.86 (m, 4H), 1.50 (s, 3H), 1.45 (s, 3H), 1.26 (s, 3H), 1.19 (s, 3H), 1.14 (s, 3H), 1.02-1.71 (m, 9H), 0.89 (d, J=6.3 Hz, 3H), 0.71 (d, J=6.6 Hz, 3H).

Compound 50: To a solution of compound 18 (600 mg, 1.26 mmol) in THF (10 mL) at room temperature was added tert-butyl carbazate (498 mg, 3.76 mmol)under nitrogen atmosphere. The mixture was heated to 70° C. and stirred at 70° C. for 18 h, then cooled to room temperature. Acetic acid (290 μL, 5.07 mmol) was added, following by the slow addition of sodium cyanoborohydride (319 mg, 5.07 mmol) as a solution in MeOH (10 mL). The resultant mixture was stirred at room temperature for 6 h, then at 60° C. for 26 h. The reaction mixture was cooled to room temperature, then concentrated under reduced pressure. The residue was partitioned between EtOAc (20 mL) and saturated aqueous NaHCO₃(20 mL). The organic phase was washed with brine (2×20 mL). The combined aqueous phase was extracted with EtOAc (2×10 mL). The combined organic extracts were dried over Na₂SO₄, filtered, and concentrated under reduced pressure. The residue was purified by column chromatography (silica gel, eluting with 0-40% acetone in hexanes) to give compound 50 (780 mg, quantitative yield) as a white solid. m/z=538 (M-C₄H₇).

Compound 51: To a solution of compound 50 (530 mg, <0.85 mmol) in THF (5 mL) at room temperature was added HCl (4 M in 1,4-dioxane, 2.58 mL, 10.3 mmol) dropwise. The resultant mixture was stirred at room temperature for 2 h, then at 60° C. for 16 h. The reaction mixture was concentrated under vacuum to give compound 51 HCl salt (680 mg) as an off-white semisolid. m/z=494 (M+1).

Compound 52: To a solution of compound 51 HCl salt (350 mg, <0.44 mmol) at room temperature in EtOH (5 mL) was added 1.1.3.3-tetramethoxypropane (72 μL, 0.43 mmol) and aqueous HCl (12 M, 99 μL, 1.18 mmol) sequentially. The resultant mixture was heated up to 70° C. and stirred at 70° C. for 17 h, then concentrated under reduced pressure. The residue was partitioned between EtOAc (20 mL) and saturated aqueous NaHCO₃(10 mL). The aqueous phase was extracted with EtOAc (10 mL). The combined organic extracts were washed with brine (10 mL), dried over Na₂SO₄, filtered, and concentrated under reduced pressure. The residue was purified by column chromatography (silica gel, eluting with 0-40% acetone in hexanes) to give compound 52 (131 mg, 56% yield from 50) as a yellow solid. m/z=530 (M+1).

Compound 53: To a suspension of compound 52 (131 mg, 0.25 mmol) in MeOH (2 mL) at room temperature was added sodium methoxide (25 wt. % in MeOH, 141 μL, 0.62 mmol). The mixture was stirred at 55° C. for 4 h, and then concentrated under reduced pressure. The residue was partitioned between water (10 mL) and CH₂Cl₂ (10 mL). The aqueous phase was extracted with CH₂Cl₂ (2×5 mL). The combined organic extracts were washed with water (10 mL), dried over Na₂SO₄, filtered, and concentrated under reduced pressure to give compound 53 (55 mg, 42% yield) as a white solid. m/z=530 (M+1).

T46: To a solution of compound 53 (55 mg, 0.10 mmol) in DMF (1 mL) at 0° C. was added 1,3-dibromo-5,5-dimethylhydantoin (14 mg, 0.050 mmol) under argon atmosphere. The mixture was stirred at 0° C. for 1.5 h, then pyridine (34 μL, 0.42 mmol) was added at 0° C. The resultant mixture was stirred at 55° C. for 20 h. The reaction mixture was cooled to room temperature; and partitioned between EtOAc (10 mL) and brine (10 mL). The aqueous phase was extracted with EtOAc (10 mL). The combined organic extracts were washed with brine (2×10 mL), dried over Na₂SO₄, filtered, and concentrated under reduced pressure. The residue was purified by column chromatography (silica gel, eluting with 0-60% acetone in hexanes) to give compound T46 (21.6 mg, 39% yield) as an off-white solid. m/z=528 (M+1); ¹H NMR (400 MHz, CDCl₃) δ 8.07 (s, 1H), 7.51 (d, J=1.6 Hz, 1H), 7.35 (d, J=2.0 Hz, 1H), 6.25 (t, J=2.1 Hz, 1H), 6.18 (s, 1H), 4.38 (d, J=14.1 Hz, 1H), 3.97 (d, J=14.0 Hz, 1H), 3.07 (d, J=3.8 Hz, 1H), 2.10-2.25 (m, 2H), 1.93 (td, J=13.8, 4.5 Hz, 1H), 1.77-1.87 (m, 4H), 1.52 (s, 3H), 1.47 (s, 3H), 1.28 (s, 3H), 1.21 (s, 3H), 1.18 (s, 3H), 1.00-1.69 (m, 9H), 0.87 (d, J=6.3 Hz, 3H), 0.71 (d, J=6.6 Hz, 3H).

Compound 54: To a solution of compound 51(174 mg, 0.35 mmol) in formic acid (5 mL) at room temperature was added 1,3,5-triazine (160 mg, 1.97 mmol). The mixture was stirred for 16 h at room temperature, and then concentrated under reduced pressure. The residue was diluted with EtOAc (20 mL), then washed with saturated aqueous NaHCO₃(2×10 mL). The aqueous phase was extracted with EtOAc (10 mL). The combined organic extracts were washed with brine (10 mL), dried over Na₂SO₄, filtered, and concentrated under reduced pressure. The residue was purified by column chromatography (silica gel, eluting with 0-40% acetone in hexanes) to give compound 54 (83 mg, 45% yield) as a white solid. m/z=531 (M+1).

Compound 55: To a solution of compound 54 (83 mg, 0.16 mmol) in MeOH (3 mL) was added potassium carbonate (65 mg, 0.47 mmol). The mixture was stirred at room temperature for 20 h, then at 45° C. for 4 h. The solvent was removed under reduced pressure and the residue was partitioned between EtOAc (10 mL) and aqueous HCl (0.5 N, 10 mL). The aqueous phase was extracted with EtOAc (2×10 mL). The combined organic extracts were washed with brine (10 mL), dried over Na₂SO₄, filtered, and concentrated under reduced pressure. The residue was purified by column chromatography (silica gel, eluting with 0-70% acetone in hexanes) to give compound 55 (44 mg, 53% yield) as a white solid. m/z=531 (M+1).

T47: To a solution of compound 55 (44 mg, 0.083 mmol) in DMF (1 mL) at 0° C. was added 1,3-dibromo-5,5-dimethylhydantoin (11 mg, 0.040 mmol) under argon atmosphere. The mixture was stirred at 0° C. for 1 h. Additional amount of 1,3-dibromo-5,5-dimethylhydantoin (2 mg, 0.007 mmol) was added, and the mixture was allowed to stir for additional 1.5 h. Then pyridine (27 μL, 0.33 mmol) was added at 0° C. The resultant mixture was stirred at 55° C. for 3.5 h; cooled to room temperature; and partitioned between EtOAc (10 mL) and brine (10 mL). The aqueous phase was extracted with EtOAc (10 mL). The combined organic extracts were washed with brine (2×10 mL), dried over Na₂SO₄, filtered, and concentrated under reduced pressure. The residue was purified by column chromatography (silica gel, eluting with 0-40% acetone in hexanes) to give compound T47 (27 mg, 62% yield) as a white solid. m/z=529 (M+1); ¹H NMR (400 MHz, CDCl₃) 8.07 (s, 1H), 8.03 (s, 1H), 7.94 (s, 1H), 6.19 (s, 1H), 4.37 (d, J=14.1 Hz, 1H), 4.05 (d, J=14.0 Hz, 1H), 3.03 (d, J=3.8 Hz, 1H), 2.22 (m, 1H), 2.12 (m, 1H), 1.97 (td, J=14.0, 4.6 Hz, 1H), 1.78-1.88 (m, 4H), 1.53 (s, 3H), 1.47 (s, 3H), 1.28 (s, 3H), 1.21 (s, 3H), 1.18 (s, 3H), 0.99-1.71 (m, 9H), 0.88 (d, J=6.3 Hz, 3H), 0.71 (d, J=6.6 Hz, 3H).

Compound 56: To a suspension of compound 54 (52 mg, 0.098 mmol) in MeOH (1 mL) at room temperature was added sodium methoxide (25 wt. % in MeOH, 56 μL, 0.24 mmol). The mixture was stirred at 55° C. for 4 h, and then concentrated under reduced pressure. The residue was partitioned between water (10 mL) and CH₂Cl₂ (10 mL). The aqueous phase was extracted with CH₂Cl₂ (2×5 mL). The combined organic extracts were washed with water (10 mL), dried over Na₂SO₄, filtered, and concentrated under reduced pressure to give compound 56 (8 mg, 10% yield) as a white solid. m/z=547 (M+1).

T48: To a solution of compound 56 (8.5 mg, 0.016 mmol) in DMF (1 mL) at 0° C. was added 1,3-dibromo-5,5-dimethylhydantoin (2.1 mg, 0.0075 mmol) under argon atmosphere. The mixture was stirred at 0° C. for 1.5 h, then pyridine (5 μL, 0.062 mmol) was added at 0° C. The resultant mixture was stirred at 55° C. for 5 h; cooled to room temperature; and partitioned between EtOAc (10 mL) and brine (10 mL). The aqueous phase was extracted with EtOAc (10 mL). The combined organic extracts were washed with brine (2×10 mL), dried over Na₂SO₄, filtered, and concentrated under reduced pressure. The residue was purified by column chromatography (silica gel, eluting with 0-60% acetone in hexanes) to give compound T48 (2.5 mg, 30% yield) as a white solid. m/z=545 (M+1); ¹H NMR (400 MHz, CDCl₃) δ 8.15 (s, 1H), 8.05 (s, 1H), 8.01 (s, 1H), 6.20 (s, 1H), 5.41 (d, J=13.8 Hz, 1H), 4.88 (s, 1H), 3.85 (d, J=13.8 Hz, 1H), 2.43 (d, J=10.5 Hz, 1H), 2.29 (m, 1H), 1.73-2.03 (m, 5H), 1.58 (s, 3H), 1.55 (s, 3H), 1.27 (s, 3H), 1.19 (s, 3H), 1.19 (s, 3H), 0.94-1.64 (m, 9H), 0.82 (d, J=5.7 Hz, 3H), 0.70 (d, J=6.6 Hz, 3H).

Compound 58: To a solution of compound 32(91% pure, 39 mg, 0.074 mmol, 91% purity) in ethanol (3 mL) at 0° C. was added N,N-Diisopropylethylamine (77 μL, 0.44 mmol). The reaction was stirred for 10 min. A solution of compound 57 (33 mg, 0.11 mmol) in acetonitrile (0.5 mL) was added. The reaction was stirred at room temperature for 3 h, and then was concentrated on rotovap. The residue was diluted with EtOAc (30 mL). The mixture was washed with saturated aqueous NaHCO₃(2×20 mL) and brine (20 mL). The organic extract was dried with Na₂SO₄, filtered and concentrated. The crude was purified by column chromatography (silica gel, eluting with 0-100% EtOAc in CH₂Cl₂) to give compound 58 (21 mg, 52% yield). m/z=545 (M+1).

Compound 59: To a solution of compound 58 (34 mg, 0.062 mmol) in MeOH (1 mL) was added K₂CO₃ (26 mg, 0.19 mmol) at room temperature. The reaction was stirred at room temperature for overnight, and then was diluted with EtOAc (10 mL) and 10% aqueous NaH₂PO₄ (10 mL). The organic extract was separated and washed with water (2×20 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give compound 59 (22 mg, 65% yield). m/z=545 (M+1).

T49: Compound 59 (33 mg, 0.061 mmol) was dissolved in DMF (3 mL) and cooled to 0° C. under nitrogen. 1,3-Dibromo-5,5-dimethylhydantoin (8.7 mg, 0.030 mmol) was dissolved in DMF (0.5 mL) in a vial. The solution was added to the reaction mixture. DMF (0.5 mL) was used to rinse the vial and was added to the reaction mixture. The mixture was stirred at 0° C. for 1 h. Pyridine (15 μL, 0.18 mmol) was added. The mixture was heated at 60° C. for 8 h. After cooled to 0° C., the mixture was diluted with EtOAc (20 mL) and washed with water (2×10 mL) and brine (20 mL). The organic extract was dried with Na₂SO₄, filtered, and concentrated. The residue was purified by column chromatography (silica gel, eluting with 0-100% acetone in hexanes) to give compound T49 (19 mg, 58% yield) as an off-white solid. m/z=543 (M+1); ¹H NMR (400 MHz, CDCl₃) 8.06 (s, 1H), 7.24 (d, J=0.9 Hz, 1H), 6.18 (s, 1H), 4.65 (d, J=14.0 Hz, 1H), 4.03 (d, J=14.0 Hz, 1H), 2.98 (d, J=3.8 Hz, 1H), 2.36 (s, 3H), 2.24 (dd, J=11.4, 3.5 Hz, 1H), 2.17 (m, 1H), 1.94 (td, J=13.9, 4.5 Hz, 1H), 1.78-1.87 (m, 4H), 1.53 (s, 3H), 1.47 (s, 3H), 1.28 (s, 3H), 1.21 (s, 3H), 1.18 (s, 3H), 1.00-1.69 (m, 9H), 0.88 (d, J=6.3 Hz, 3H), 0.72 (d, J=6.6 Hz, 3H).

Compound 61: To a solution of compound 32 (86% pure, 60 mg, 0.11 mmol) in ethanol (3 mL) at 0° C. was added N,N-Diisopropylethylamine (0.11 mL, 0.65 mmol). The reaction was stirred for 10 min. A solution of compound 60 (45 mg, 0.16 mmol) in acetonitrile (0.5 mL) was added. The reaction was stirred at room temperature for 3 h, and then was concentrated on rotovap. The residue was diluted with EtOAc (30 mL). The mixture was washed with saturated aqueous NaHCO₃(2×20 mL) and brine (20 mL). The organic extract was dried with Na₂SO₄, filtered and concentrated. The crude was purified by column chromatography (silica gel, eluting with 0-100% EtOAc in CH₂Cl₂) to give compound 61 (30 mg, 52% yield). m/z=531 (M+1).

Compound 62: To a solution of compound 61 (60 mg, 0.11 mmol) in MeOH (1 mL) was added K₂CO₃ (47 mg, 0.34 mmol) at room temperature. The reaction was stirred at room temperature for overnight, and then was diluted with EtOAc (10 mL) and 10% aqueous NaH₂PO₄ (10 mL). The organic extract was separated and washed with water (2×20 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give compound 62 (41 mg, 68% yield). m/z=531 (M+1).

T50: Compound 62 (41 mg, 0.077 mmol) was dissolved in DMF (3 mL) and cooled to 0° C. under nitrogen. 1,3-Dibromo-5,5-dimethylhydantoin (11 mg, 0.039 mmol) was dissolved in DMF (0.5 mL) in a vial. The solution was added to the reaction mixture. DMF (0.5 mL) was used to rinse the vial and added to the reaction mixture. The mixture was stirred at 0° C. for 1 h. Pyridine (19 μL, 0.23 mmol) was added. The mixture was heated at 60° C. for 7 h. After cooled to 0° C., the mixture was diluted with EtOAc (20 mL) and washed with water (2×10 mL) and brine (20 mL). The organic extract was dried with Na₂SO₄, filtered, and concentrated. The residue was purified by column chromatography (silica gel, eluting with 0-80% acetone in hexanes) to give compound T50 (32.8 mg, 80% yield) as an off-white solid. m/z=529 (M+1); ¹H NMR (400 MHz, CDCl₃) 8.06 (s, 1H), 7.71 (d, J=1.0 Hz, 1H), 7.53 (d, J=1.1 Hz, 1H), 6.19 (s, 1H), 4.76 (d, J=13.9 Hz, 1H), 4.10 (d, J=13.9 Hz, 1H), 2.99 (d, J=3.8 Hz, 1H), 2.14-2.28 (m, 2H), 1.95 (td, J=14.1, 4.7 Hz, 1H), 1.80-1.87 (m, 4H), 1.53 (s, 3H), 1.48 (s, 3H), 1.28 (s, 3H), 1.21 (s, 3H), 1.19 (s, 3H), 1.01-1.69 (m, 9H), 0.89 (d, J=6.3 Hz, 3H), 0.72 (d, J=6.6 Hz, 3H).

Compound 63: To a mixture of paraformaldehyde (41 mg, 1.38 mmol), ammonium carbonate (64 mg, 0.66 mmol) and trimeric glyoxal dihydrate (116 mg, 0.55 mmol) in MeOH (6 mL) was added compound 32 (88% pure, 100 mg, 0.18 mmol). The mixture was stirred at room temperature for overnight, and then was concentrated on rotovap. The residue was partitioned between EtOAc (20 mL) and water (20 mL). The organic extract was separated and washed with water (2×20 mL). The aqueous phase was extracted with EtOAc (20 mL). The combined organic extracts were washed with brine (20 mL), dried with Na₂SO₄, filtered, and concentrated. The residue was purified by column chromatography [silica gel, eluting with 0-10% (1% Et₃N in MeOH) in CH₂Cl₂] twice to give compound 63 (83 mg, 85% yield). m/z=530 (M+1).

Compound 64: To a solution of compound 63 (83 mg, 0.16 mmol) in MeOH (4 mL) was added K₂CO₃ (65 mg, 0.47 mmol) at room temperature. The reaction was stirred at room temperature for overnight. The mixture was partitioned between EtOAc (10 mL) and 10% aqueous NaH₂PO₄ (10 mL). The organic extract was separated and washed with water (2×20 ML). The aqueous phase was extracted with EtOAc (20 mL). The combined organic extracts were washed with brine (20 mL), dried with Na₂SO₄, filtered, and concentrated to give compound 64 (83 mg, quantitative yield), which was used in the next step without further purification. m/z=530 (M+1).

T51: Compound 64 (83 mg, 0.16 mmol) was dissolved in DMF (3 mL) and cooled to 0° C. under nitrogen. 1,3-Dibromo-5,5-dimethylhydantoin (22 mg, 0.078 mmol) was dissolved in DMF (0.5 mL) in a vial. The solution was added to the reaction mixture. DMF (0.5 mL) was used to rinse the vial and added to the reaction mixture. The mixture was stirred at 0° C. for 1 h. Pyridine (38 μL, 0.47 mmol) was added. The mixture was heated at 60° C. for 8 h. After cooled to 0° C., the mixture was diluted with EtOAc (20 mL) and washed with water (2×10 mL) and brine (20 mL). The organic extract was dried with Na₂SO₄, filtered, and concentrated. The residue was purified by column chromatography [silica gel, eluting with 0-10% (1% Et₃N in MeOH) in CH₂Cl₂] to give a partially purified product which was purified again by column chromatography (silica gel, eluting with 0-100% acetone in hexanes) to give compound T51 (17 mg, 21% yield) as an off-white solid. m/z=528 (M+1); ¹H NMR (400 MHz, CDCl₃) 8.06 (s, 1H), 7.42 (s, 1H), 7.07 (s, 1H), 6.87 (s, 1H), 6.20 (s, 1H), 4.04 (d, J=14.4 Hz, 1H), 3.89 (d, J=14.3 Hz, 1H), 2.90 (d, J=3.8 Hz, 1H), 2.31 (dd, J=11.3, 2.3 Hz, 1H), 2.02 (td, J=14.1, 4.5 Hz, 1H), 1.91 (td, J=13.6, 4.4 Hz, 1H), 1.80-1.88 (m, 4H), 1.53 (s, 3H), 1.43 (s, 3H), 1.28 (s, 3H), 1.21 (s, 3H), 1.19 (s, 3H), 0.99-1.74 (m, 9H), 0.87 (d, J=6.5 Hz, 3H), 0.71 (d, J=6.6 Hz, 3H).

Compound 65: To a solution of compound 32 (88% pure, 54 mg, 0.099 mmol) in glacial acetic acid (2 mL) at room temperature was added trimethyl orthoformate (0.12 mL, 1.1 mmol) and the reaction was stirred for 20 min. Sodium azide (97 mg, 1.5 mmol) was added and the reaction was heated at 80° C. for 2h. After cooled to room temperature, EtOAc (20 mL) was added and the reaction mixture was washed with water (20 mL), saturated aqueous NaHCO₃(2×20 mL), and brine (20 mL). The organic extract was dried with Na₂SO₄, filtered and concentrated. The residue was purified by column chromatography (silica gel, eluting with 0-100% EtOAc in hexanes) to give compound 65 (37 mg, 70% yield). m/z=532 (M+1).

Compound 66: Compound 65 (37 mg, 0.070 mmol) in MeOH (1 mL) was treated with sodium methoxide solution (25 wt. % in MeOH, 32 μL, 0.14 mmol) at room temperature under nitrogen. The mixture was heated at 55° C. for 1.5 h, and then cooled to 0° C. The mixture was treated with 10% aqueous NaH₂PO₄ (10 mL) and extracted with EtOAc (2×20 mL). The combined organic extracts were washed with brine (20 mL), dried with Na₂SO₄, filtered and concentrated. The crude was purified by column chromatography (silica gel, eluting with 0-60% acetone in hexanes) to give compound 66 (21 mg, 57% yield). m/z=532 (M+1).

T52: Compound 66 (21 mg, 0.039 mmol) was dissolved in DMF (2 mL) and cooled to 0° C. under nitrogen. 1,3-Dibromo-5,5-dimethylhydantoin (5.6 mg, 0.020 mmol) was dissolved in DMF (0.5 mL) in a vial. The solution was added to the reaction mixture. DMF (0.5 mL) was used to rinse the vial and added to the reaction mixture. The mixture was stirred at 0° C. for 1 h. Pyridine (10 μL, 0.12 mmol) was added. The mixture was heated at 60° C. for 4 h. After cooled to 0° C., the mixture was diluted with EtOAc (20 mL) and washed with water (2×10 mL) and brine (20 mL). The organic extract was dried with Na₂SO₄, filtered, and concentrated. The residue was purified by column chromatography (silica gel, eluting with 0-80% acetone in hexanes) to give compound T52 (16.6 mg, 79% yield) as an off-white solid. m/z=530 (M+1); ¹H NMR (600 MHz, CDCl₃) 8.57 (s, 1H), 8.07 (s, 1H), 6.20 (s, 1H), 4.78 (d, J=14.1 Hz, 1H), 4.17 (d, J=14.1 Hz, 1H), 2.92 (d, J=3.9 Hz, 1H), 2.26 (ddd, J=11.3, 3.9, 1.7 Hz, 1H), 2.15 (td, J=13.8, 4.6 Hz, 1H), 1.98 (td, J=14.1, 4.6 Hz, 1H), 1.82-1.88 (m, 4H), 1.54 (s, 3H), 1.48 (s, 3H), 1.29 (s, 3H), 1.22 (s, 3H), 1.20 (s, 3H), 1.04-1.69 (m, 9H), 0.90 (d, J=6.5 Hz, 3H), 0.73 (d, J=6.6 Hz, 3H).

Compound 67: To a solution of compound 50 (247 mg, 0.42 mmol) in CH₂Cl₂ (3 mL) at 0° C. was added 3-chloropropionyl chloride (79.4 μL, 0.83 mmol). The mixture was stirred at 0° C. for 30 min then at room temperature for 1.5 h. The reaction mixture was concentrated, and the residue was partitioned between EtOAc (20 mL) and saturated aqueous NaHCO₃(20 mL). The aqueous layer was extracted with EtOAc (2×20 mL). The combined organic extracts were washed with brine (20 mL), dried over Na₂SO₄, filtered, and concentrated under reduced pressure. The residue was purified by column chromatography (silica gel, eluting with 0-60% EtOAc in hexanes) to give compound 67 (268 mg, 94% yield) as a white solid. m/z=684 (M+1).

Compound 68: To a solution of compound 67 (268 mg, 0.39 mmol) in DMF (5 mL) at room temperature was added potassium carbonate (271 mg, 1.96 mmol). The mixture was stirred at room temperature for 20 h, and then partitioned between EtOAc (20 mL) and water (20 mL). The organic extract was washed with water (2×20 mL) and brine (20 mL), dried with Na₂SO₄, filtered and concentrated under reduced pressure. The residue was purified by column chromatography (silica gel, eluting with 0-100% EtOAc in hexanes) to give compound 68 (172 mg, 68% yield) as an off-white solid. m/z=648 (M+1).

Compound 69a and 69b: To a suspension of compound 68(172 mg, 0.26 mmol)in MeOH (2 mL) at room temperature was added sodium methoxide (25 wt. % in MeOH, 152 μL, 0.66 mmol). The mixture was stirred at 55° C. for 4 h, and then concentrated under reduced pressure. The residue was partitioned between brine (10 mL) and EtOAc (10 mL). The aqueous phase was extracted with EtOAc (10 mL). The combined organic extracts were washed with brine (10 mL), dried over Na₂SO₄, filtered, and concentrated under reduced pressure to give a mixture of compound 69a and 69b (total 183 mg in approximately 6:1 ratio) as a pink solid, which was used in next step without further purification. m/z=648 (M+1) for 69a and m/z=606 (M+1) for 69b.

Compound 70 and T53: To a solution of compound 69a and 69b (183 mg, <0.26 mmol) in DMF (2 mL) at 0° C. was added 1,3-dibromo-5,5-dimethylhydantoin (36.4 mg,0.127 mmol) under argon atmosphere. The mixture was stirred at 0° C. for 1.5 h, then pyridine (86 μL, 1.06 mmol) was added at 0° C. The resultant mixture was stirred at 55° C. for 4 h; cooled to room temperature; and partitioned between EtOAc (10 mL) and brine (10 mL). The aqueous phase was extracted with EtOAc (10 mL). The combined organic extracts were washed with brine (2×10 mL), dried over Na₂SO₄, filtered, and concentrated under reduced pressure. The residue was first purified by column chromatography (silica gel, eluting with 0-60% acetone in hexanes) to give compound 70 (86 mg, 50% yield from compound 68) as an off-white solid and partially purified compound T53 (16 mg). Compound T53 was purified again by column chromatography (silica gel, eluting with 0-35% acetone in hexanes) to give compound T53 (9.3 mg, 6% yield from compound 68) as a white solid. Compound 70: m/z=646 (M+1). Compound T53: m/z=604 (M+1); ¹H NMR (400 MHz, CDCl₃) δ 8.06 (s, 1H), 6.11 (s, 1H), 3.98-4.16 (m, 3H), 3.78 (s, 3H), 3.73 (d, J=15.1 Hz, 1H), 2.99 (d, J=3.6 Hz, 1H), 2.48-2.66 (m, 2H), 2.33 (m, 1H), 1.90-2.02 (m, 2H), 1.51 (s, 3H), 1.44 (s, 3H), 1.27 (s, 3H), 1.20 (s, 3H), 1.12 (s, 3H), 0.88 (d, J=6.4 Hz, 3H), 0.98-1.85 (m, 13H), 0.68 (d, J=6.6 Hz, 3H).

T54: To a solution of compound 70 (86 mg, 0.13 mmol) in CH₂Cl₂ (1 mL) at 0° C. was added trifluoroacetic acid (0.31 mL, 4.0 mmol). The mixture was stirred at room temperature for 3 h, and then concentrated under reduced pressure. The residue was purified by column chromatography (silica gel, eluting with 0-70% acetone in hexanes) to give partially purified product. The obtained product was purified again by column chromatography (silica gel, eluting with 0-80% acetone in hexanes). The obtained product was purified for a third time by column chromatography (silica gel, eluting with 0-60% acetone in hexanes) to give compound T54 (9.7 mg, 11% yield) as a white solid. m/z=546 (M+1); 1H NMR (400 MHz, CDCl₃) δ 8.21 (s, 1H), 6.22 (s, 1H), 3.58 (d, J=14.5 Hz, 1H), 3.37 (t, J=7.7 Hz, 3H), 3.26 (d, J=3.6 Hz, 1H), 2.40-2.66 (m, 2H), 2.17 (m, 1H), 1.95-2.01 (m, 2H), 1.72-1.88 (m, 5H), 1.53 (s, 3H), 1.45 (s, 3H), 1.27 (s, 3H), 1.20 (s, 3H), 1.12 (s, 3H), 0.99-1.59 (m, 8H), 0.87 (d, J=6.3 Hz, 3H), 0.67 (d, J=6.6 Hz, 3H).

Compound 71: To a solution of compound 18 (300 mg, 0.63 mmol) in THF (8 mL) at room temperature was added azetidine (212 μL, 3.14 mmol) under argon atmosphere. The mixture was stirred at room temperature for 2 h, then acetic acid (180 μL, 3.14 mmol) was added at room temperature. The resultant mixture was stirred at room temperature for another 1 h and then sodium cyanoborohydride (197 mg, 3.14 mmol) was added as a solution in MeOH (8 mL) dropwise. The reaction mixture was stirred at room temperature for additional 16 h, then quenched with saturated aqueous NaHCO₃(20 mL). The mixture was concentrated under reduced pressure. The residue was extracted with EtOAc (3×10 mL). The combined organic extracts were dried over Na₂SO₄, filtered, and concentrated under reduced pressure. The residue was purified by column chromatography (silica gel, eluting with 0-20% MeOH in EtOAc) to give compound 71 (145 mg, 44% yield) as a white solid. m/z=519 (M+1).

Compound 72: To a suspension of compound 71 (145 mg, 0.28 mmol) in MeOH (2 mL) at room temperature was added sodium methoxide (25 wt. % in MeOH, 0.16 mL, 0.70 mmol). The mixture was stirred at 55° C. for 2.5 h, then cooled to room temperature and quenched with acetic acid (5 drops). The resultant mixture was concentrated, and the residue was partitioned between water (10 mL) and CH₂Cl₂ (10 mL). The aqueous phase was extracted with CH₂Cl₂ (2×10 mL). The combined organic extracts were dried over Na₂SO₄, filtered, and concentrated under reduced pressure. The residue was purified by column chromatography (silica gel, eluting with 0-100% acetone in CH₂Cl₂) to give compound 72 (35 mg, 24% yield) as a white solid. m/z=519 (M+1).

T55 and T56: To a solution of compound 72 (35 mg, 0.067 mmol) in toluene (4 mL) at room temperature was added DDQ (15.1 mg, 0.067 mmol). The mixture was stirred at 55° C. for 1 h, then cooled to room temperature and partitioned between EtOAc (10 mL) and saturated aqueous NaHCO₃(10 mL). The aqueous phase was extracted EtOAc (10 mL). The combined organic extracts were washed with saturated aqueous NaHCO₃(2×10 mL), dried over Na₂SO₄, filtered, and concentrated under reduced pressure. The residue was first purified by column chromatography (silica gel, eluting with 0-100% acetone in hexanes containing 0.5% Et₃N), then purified again by column chromatography (silica gel, eluting with 0-70% acetone in CH₂Cl₂ containing 0.5% Et₃N) and finally purified by column chromatography (Redi Sep amine column, eluting with 0-100% acetone in hexanes, then 0-10% MeOH in CH₂Cl₂) to give compound T55 (4.3 mg, 12% yield) as a white solid and T56 (2.5 mg, 7% yield) as a white solid. T55: m/z=517 (M+1); ¹H NMR (400 MHz, CDCl₃) δ 8.05 (s, 1H), 6.13 (s, 1H), 3.14-3.34 (m, 4H), 2.80 (d, J=3.6 Hz, 1H), 2.45 (d, J=13.4 Hz, 1H), 2.23-2.35 (m, 2H), 2.04 (m, 2H), 1.50 (s, 3H), 1.37 (s, 3H), 1.26 (s, 3H), 1.19 (s, 3H), 1.11 (s, 3H), 1.04-1.90 (m, 15H), 0.87 (d, J=6.0 Hz, 3H), 0.68 (d, J=6.6 Hz, 3H). T56: m/z=533 (M+1); ¹H NMR (400 MHz, CDCl₃) δ 6.20 (s, 1H), 4.36 (s, 1H), 3.12-3.40 (m, 4H), 2.80 (s, 1H), 2.46 (d, J=13.3 Hz, 1H), 2.22-2.38 (m, 2H), 1.98-2.10 (m, 3H), 1.31 (s, 3H), 1.28 (s, 3H), 1.21 (s, 3H), 1.17 (s, 3H), 1.14 (s, 3H), 1.12-1.92 (m, 14H), 0.87 (d, J=6.1, 3H), 0.69 (d, J=6.5 Hz, 3H).

Compound 73: To a solution of methoxyamine hydrochloride (44 mg, 0.53 mmol) in MeOH (2 mL) at room temperature was added Et₃N (73 μL, 0.52 mmol). Compound 18 (50 mg, 0.10 mmol) was dissolved in THF (1 mL)in a vial. The solution was added to the reaction mixture. THF (1 mL) was used to rinse the vial and added to the reaction mixture. The reaction was stirred at room temperature for 4 h, and then was diluted with EtOAc (20 mL) and H₂O (10 mL). The organic extract was separated and washed with water (2×20 mL). The aqueous phase was extracted with EtOAc (20 mL). The combined organic extracts were washed with brine (20 mL), dried with Na₂SO₄, filtered, and concentrated to give compound 73 (62 mg, quantitative yield), which was used in the next step without further purification. m/z=507 (M+1).

Compound 74: To a solution of compound 73 (100 mg, 0.197 mmol) in MeOH (3 mL) was added K₂CO₃ (82 mg, 0.59 mmol) at room temperature. The reaction was stirred at room temperature for overnight, and then was diluted with EtOAc (10 mL) and 10% aqueous NaH₂PO₄ (10 mL). The organic extract was separated and washed with water (2×20 mL). The aqueous phase was extracted with EtOAc (20 mL). The combined organic extracts were washed with brine (20 mL), dried with Na₂SO₄, filtered, and concentrated to give crude compound 74 (109 mg, quantitative yield), which was used in the next step without further purification. m/z=507 (M+1).

T57: Compound 74 (109 mg, 0.215 mmol) was dissolved in DMF (5 mL) and cooled to 0° C. under nitrogen. 1,3-Dibromo-5,5-dimethylhydantoin (31 mg, 0.11 mmol) was dissolved in DMF (1 mL) in a vial. The solution was added to the reaction mixture. DMF (1 mL) was used to rinse the vial and added to the reaction mixture. The mixture was stirred at 0° C. for 1 h. Pyridine (52 μL, 0.65 mmol) was added. The mixture was heated at 60° C. for 4 h. After cooled to 0° C., the mixture was diluted with EtOAc (20 mL) and washed with water (2×10 mL) and brine (20 mL). The organic extract was dried with Na₂SO₄, filtered, and concentrated. The residue was purified by column chromatography (silica gel, eluting with 0-60% acetone in hexanes) to give compound T57 (66 mg, 61% yield) as an off-white solid. m/z=505 (M+1); ¹H NMR (600 MHz, CDCl₃) 8.04 (s, 1H), 7.13 (d, J=1.5 Hz, 1H), 6.12 (s, 1H), 3.79 (s, 3H), 2.74 (d, J=3.8 Hz, 1H), 2.40 (ddd, J=11.5, 3.8, 1.6 Hz, 1H), 2.14 (td, J=13.2, 4.9 Hz, 1H), 2.00 (m, 1H), 1.72-1.84 (m, 4H), 1.64 (m, 1H), 1.49 (s, 3H), 1.30 (s, 3H), 1.27 (s, 3H), 1.19 (s, 3H), 1.12 (s, 3H), 1.08-1.61 (m, 8H), 0.91 (d, J=6.5 Hz, 3H), 0.73 (d, J=6.6 Hz, 3H).

Compound 75: To a flame-dried round bottom equipped with a stir bar under nitrogen was added Ursolic acid 1 (250.0 g, 0.548 mol), K₂CO₃ (151.0 g, 1.096 mol) and DMF (1.2 L). The reaction mixture was stirred for 30 min at room temperature. Iodomethane (51.3 mL, 0.822 mol) was then added. The reaction mixture was stirred for 16 h; poured into water (2 L); and stirred for 30 min at room temperature. The precipitated solid was collected by filtration; and was washed with water (200 mL) and diethyl ether (100 mL). The wet cake was dried under vacuum to give crude compound 75 (270.0 g), which was used in the next step without further purification.

Compound 76: A solution of crude compound 75 (270.0 g, <0.548 mol) in Ac20 (1.5 L, 15.9 mol) was stirred at 100° C. for 5 h. AcOH (600 mL) and water (900 mL) were added to the hot reaction mixture, and then the reaction mixture was cooled to room temperature. The precipitated solid was collected by filtration; and was washed with water (500 mL) and diethyl ether (100 mL). The wet cake was dried under vacuum to give crude compound 76 (260.0 g), which was used in the next step without further purification.

Compound 77: Aqueous hydrogen peroxide (620 mL, 30 wt. %, 6.03 mol) was added to formic acid (3 L) with stirring at room temperature. The resultant solution was added to a solution of compound 76 (260.0 g) in CH₂Cl₂ (3 L) with stirring at room temperature. The mixture was stirred at room temperature for 48 h, and then cooled to 10° C. The reaction was quenched with 10% aqueous Na₂SO₃ (3 L). The aqueous phase was separated; and extracted with CH₂Cl₂ (2 L). The combined organic extracts were washed with brine (3×1 L); dried over anhydrous Na₂SO₄; filtered and concentrated. The residue was purified by column chromatography (silica gel, eluting with 0-20% EtOAc in hexanes) to give compound 75 (170.0 g, yield: 59% from compound 1) as a white solid. m/z=529 (M+1).

Compound 78: To a solution of compound 77 (30.0 g, 56.7 mmol) in acetonitrile (600 mL) was added pyridinium tribromide (24.4 g, 76.6 mmol) at room temperature. The reaction mixture was stirred at 50° C. for 3 h; cooled to room temperature; and quenched with 10% aqueous Na₂SO₃ solution (200 mL). The mixture was extracted with CH₂Cl₂(3×100 mL). The combined organic extracts were dried over MgSO₄, filtered and concentrated. The residue was purified by column chromatography (silica gel, eluting with 0-20% EtOAc in hexanes) to give compound 78 (30 g, quantitative yield) as a white solid, which was used in the next step without further purification. m/z=527 (M+1).

Compound 79: To a solution of crude compound 78 (30 g, ≤5 56.7 mmol) in MeOH (400 L) was added K₂CO₃ (40.6 g, 294.5 mmol). The mixture was stirred at room temperature overnight. The solvent was removed, and the residue was partitioned between EtOAc (200 mL) and water (300 mL). The aqueous phase was extracted with EtOAc (2×200 mL). The combined organic extracts were washed with water (300 mL) and brine (300 mL); dried over Na₂SO₄; filtered and concentrated to give crude compound 79 (26.0 g) as a white solid, which was used in the next step without further purification. m/z=485 (M+1).

Compound 80: To a flame-dried round bottom flask equipped with a stir bar under nitrogen was added compound 79 (26.0 g, 53.6 mmol), DMSO (150 mL) and EtOAc (150 mL). The reaction mixture was stirred for 30 min at room temperature. Propylphosphonic anhydride (T3P, 50 wt. % in EtOAc, 68.2 g, 0.107 mol) and triethylamine (14.6 mL, 0.107 mol) were added.

The reaction mixture was stirred at room temperature for 2 h. The reaction mixture was poured into water (500 mL) and extracted with EtOAc (3×500 mL). The combined organic extracts were washed with water (3×500 mL) and brine (500 mL), dried over Na₂SO₄; filtered and concentrated. The residue was purified by column chromatography (silica gel, eluting with 0-30% EtOAc in hexanes) to give compound 80 (21 g, 76% yield from compound 78) as a white solid. m/z=483 (M+1).

Compound 81: Compound 80 (50 g, 103.6 mmol) and NaOAc (21.2 g, 258 mmol) were weighted in a 3 neck round bottom flask. The mixture was mixed with dimethylacetamide (300 mL). LiBr (81.0 g, 932 mmol) was added. The mixture was heated in an oil bath (preheated to 150° C.) with nitrogen bubbled through the reaction mixture to remove MeBr formed for 16 h. The mixture was cooled in a water bath at room temperature. Aqueous HCl (1 M, 1.5 L) was added. The mixture was stirred at ambient temperature for 1 h. The precipitated white solid was collected by filtration. The solid was washed with water (150 mL). The wet cake was dissolved in CH₂Cl₂ (500 mL). The mixture was washed with water (2×500 mL). The organic extract was dried with MgSO₄, filtered and concentrated to give compound 81 (36 g, 74% yield) as a white solid. m/z=469 (M+1).

Compound 82: Compound 81 (20 g, 43 mmol) was mixed with ethyl formate (100 mL, 1.24 mol). The mixture was cooled to 0° C. Sodium methoxide (5 M solution in MeOH, 128 mL, 0.64 mol) was added. The reaction mixture was stirred at room temperature for 3 h, and then was cooled to 0° C. HCl (6 M aqueous solution, −79 mL, 0.47 mol) was added slowly to adjust pH to 1-2. EtOH (400 mL) and hydroxylamine hydrochloride (4.4 g, 63 mmol) were added. The reaction mixture was stirred at 55° C. for 3 h. EtOH was removed. The residue was partitioned between CH₂Cl₂ (1 L) and water (1 L). The aqueous phase was separated and extracted with CH₂Cl₂ (2×500 mL). The combined organic extracts were washed with water (500 mL) and brine (500 mL), dried over Na₂SO₄, filtered and concentrated. The residue was purified by column chromatography (silica gel, eluting with 0-70% EtOAc in hexanes) to give compound 82 (19 g, 90% yield) as a white solid. m/z=494 (M+1).

Compound 83: To the solution of compound 82 (11 g, 22.3 mmol) and Et₃N (6.8 g, 66.9 mmol) in toluene (100 mL) at 0° C. under nitrogen was added diphenyl phosphoryl azide (9.2 g, 33.5 mmol). The mixture was stirred at room temperature for 3 h. The mixture was diluted with EtOAc (300 mL) and washed with water (300 mL) and brine (300 mL). The organic extract was dried over Na₂SO₄, filtered and concentrated to give crude compound 83.

Compound 83 was dissolved in toluene (100 mL) and heated at 80° C. for 3 h under nitrogen. The reaction mixture was concentrated to give crude compound 84 as a white solid.

Compound 84 was dissolved in acetonitrile (100 mL) and cooled to 0° C. Concentrated HCl (12 N aqueous, 41 mL, 0.49 mmol) was added. The mixture was stirred at ambient temperature for 3 h. The precipitated solid was collected by filtration; washed with acetonitrile (20 mL); and dried under vacuum to give compound 85 HCl salt (8 g, 71% yield) as a white solid. m/z=465 (M+1).

Compound 86: To a solution of compound 85 HCl salt (210 mg, 0.42 mmol) in CH₂Cl₂ (9 mL) was added Et₃N (0.63 mL, 4.52 mmol) at room temperature. The reaction was cooled down to 0° C. and 4-chlorobutyryl chloride (0.15 mL, 1.36 mmol) was added. The reaction was run at room temperature for 2 h, and then was diluted with EtOAc (20 mL) and aqueous HCl (1 M, 20 mL). The organic extract was separated and washed with water (2×20 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% EtOAc in hexanes) to give compound 86 (210 mg, 82% yield). m/z=569 (M+1).

Compound 87: To a solution of compound 86 (65 mg, 0.11 mmol) in DMF (3 mL) was added NaH (60% dispersion in mineral oil (14 mg, 0.34 mmol) at 0° C. The reaction was stirred at room temperature for 2 h, and then was cooled down to 0° C. The reaction was quenched with aqueous HCl (1 M, 10 mL). EtOAc (20 mL) and water (20 mL) were then added. The organic extract was separated and washed with water (2×20 mL). The aqueous phase was extracted with EtOAc (20 mL). The combined organic extracts were washed with brine (20 mL), dried with Na₂SO₄, filtered, and concentrated. The residue was combined with another batch synthesized from compound 86 (156 mg, 0.27 mmol) using the same procedure, and was purified by column chromatography (silica gel, eluting with 0-70% EtOAc in hexanes) to give compound 87 (112 mg, 54% yield). m/z=533 (M+1).

T58: Compound 87 (112 mg, 0.21 mmol) was dissolved in DMF (7 mL) and cooled to 0° C. under nitrogen. 1,3-Dibromo-5,5-dimethylhydantoin (30 mg, 0.105 mmol) was added. The mixture was stirred at 0° C. for 1 h. Pyridine (51 μL, 0.63 mmol) was added. The mixture was heated at 60° C. for 4 h. After cooled to 0° C., the reaction was diluted with EtOAc (20 mL) and 10% aqueous NaH₂PO₄ (10 mL) was added. The organic extract was separated and washed with water (2×20 mL). The aqueous phase was extracted with EtOAc (20 mL). The combined organic extracts were washed with brine (20 mL), dried with Na₂SO₄, filtered, and concentrated. The residue was purified twice by column chromatography (silica gel, eluting with 0-50% acetone in hexanes) to give partially purified product which was repurified again by column chromatography (silica gel, eluting with 0-100% EtOAc in hexanes) to give compound T58 (13 mg, 9% yield) as an off-white solid. m/z=531 (M+1); ¹H NMR (400 MHz, CDCl₃) 8.05 (s, 1H), 6.14 (s, 1H), 3.27-3.42 (m, 2H), 3.10 (m, 1H), 2.65 (bs, 1H), 2.47 (m, 1H), 2.26-2.38 (m, 2H), 1.49 (s, 3H), 1.30 (s, 3H), 1.27 (s, 3H), 1.19 (s, 3H), 1.15 (s, 3H), 1.05-2.02 (m, 16H), 0.90 (d, J=6.2 Hz, 3H), 0.74 (d, J=6.6 Hz, 3H).

Compound 88: To a solution of compound 85 HCl salt (210 mg, 0.42 mmol) in CH₂Cl₂ (9 mL) was added Et₃N (0.25 mL, 1.81 mmol) at room temperature. The reaction was cooled down to 0° C. and 2-Chloroethyl chloroformate (0.14 mL, 1.36 mmol) was added. The reaction was stirred at room temperature for 4 h. The mixture was diluted with EtOAc (20 mL) and saturated aqueous NH₄C1 (20 mL). The organic extract was separated and washed with water (2×20 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% EtOAc in hexanes) to give compound 88 (120 mg, 46% yield). m/z=571 (M+1).

Compound 89: To a solution of compound 88 (120 mg, 0.21 mmol) in THF (2 mL) at 0° C. was added potassium tert-butoxide (28 mg, 0.25 mmol) in THF (2 mL). The reaction was stirred at 0° C. for 30 min. The reaction was diluted with EtOAc (20 mL) and quenched with saturated aqueous NH₄C1 (20 mL). The organic extract was separated and washed with water (2×10 mL), brine (20 mL), dried with Na₂SO₄, filtered, and concentrated to give compound 89 (112 mg), which was used in the next step without further purification. m/z=535 (M+1).

Compound 90: To a solution of compound 89 (112 mg, 0.21 mmol) in MeOH (3 mL) was added K₂CO₃ (116 mg, 0.84 mmol) at room temperature. The reaction was stirred at room temperature for overnight, and then was diluted with EtOAc (20 mL) and 10% aqueous NaH₂PO₄ (10 mL). The organic extract was separated and washed with water (2×20 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-80% EtOAc in hexanes) to give compound 90 (54 mg, 48% yield from compound 88). m/z=535 (M+1).

T59: Compound 90 (54 mg, 0.10 mmol) was dissolved in DMF (4 mL) and cooled to 0° C. under nitrogen. 1,3-Dibromo-5,5-dimethylhydantoin (14 mg, 0.050 mmol) was added. The mixture was stirred at 0° C. for 1 h. Pyridine (24 μL, 0.30 mmol) was added. The mixture was heated at 60° C. for 4 h. After cooled to 0° C., the reaction was diluted with EtOAc (20 mL) and 10% aqueous NaH₂PO₄ (10 mL) was added. The organic extract was separated and washed with water (2×10 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-50% acetone in hexanes) to give compound T59 (39 mg, 72% yield) as an off-white solid. m/z=533 (M+1); ¹H NMR (400 MHz, CDCl₃) 8.04 (s, 1H), 6.14 (s, 1H), 4.27 (m, 1H), 4.06 (q, J=9.0 Hz, 1H), 3.61 (q, J=9.5 Hz, 1H), 3.36 (t, J=8.3 Hz, 1H), 2.76 (s, 1H), 2.37 (bs, 1H), 1.48 (s, 3H), 1.32 (s, 3H), 1.25 (s, 3H), 1.18 (s, 3H), 1.14 (s, 3H), 1.02-2.01 (m, 15H), 0.89 (d, J=6.3 Hz, 3H), 0.72 (d, J=6.6 Hz, 3H).

Compound 91: To a solution of compound 85 HCl salt (220 mg, 0.44 mmol) in EtOH (3 mL) at 0° C. was added N,N-Diisopropylethylamine (242 μL, 1.39 mmol). The mixture was stirred at 0° C. for 10 min, then compound 60 (260 mg, 0.92 mmol) was added as a solution in MeCN (3 mL) and EtOH (1 mL) dropwise at 0° C. The resultant mixture was stirred at 0° C. for 1 h, then at room temperature for 5 h. The reaction mixture was concentrated, and the residue was purified by column chromatography (silica gel, eluting with 0-40% acetone in hexanes) to give compound 91 (163 mg, 68% yield) as a pale-yellow solid. m/z=517 (M+1).

Compound 92: To a suspension of compound 91(163 mg, 0.32 mmol) in MeOH (10 mL) at room temperature was added potassium carbonate (131 mg, 0.95 mmol). The mixture was stirred at room temperature for 20 h, then concentrated under reduced pressure. The residue was partitioned between EtOAc (10 mL) and aqueous HCl (0.2 N, 10 mL). The aqueous phase was extracted with EtOAc (2×10 mL). The combined organic extracts were dried over Na₂SO₄, filtered, and concentrated under reduced pressure to give compound 92 (158 mg, 97% yield) as a white solid, which was used in next step without further purification. m/z=517 (M+1).

T60: To a solution of compound 92(158 mg, 0.31 mmol) in toluene (3 mL) and chloroform (2 mL) at room temperature was added DDQ (69.4 mg, 0.31 mmol). The mixture was stirred at 50° C. for 45 min, then cooled to room temperature and quenched with saturated aqueous NaHCO₃(10 mL). The aqueous phase was separated and extracted with EtOAc (2×20 mL). The combined organic extracts were washed with saturated aqueous NaHCO₃(2×10 mL), dried over Na₂SO₄, filtered, and concentrated under reduced pressure. The residue was purified by column chromatography (silica gel, eluting with 0-60% acetone in CH₂Cl₂) to give compound T60 (105 mg, 67% yield) as a white solid. m/z=515 (M+1); ¹H NMR (400 MHz, CDCl₃) 8.40 (s, 1H), 7.83 (s, 1H), 7.65 (s, 1H), 6.29 (s, 1H), 3.16 (d, J=11.4 Hz, 1H), 2.54 (m, 1H), 2.36 (td, J=13.9, 4.8 Hz, 1H), 2.28 (d, J=3.6 Hz, 1H), 2.00 (m, 1H), 1.91 (m, 1H), 1.53-1.79 (m, 7H), 1.38 (s, 3H), 1.20 (s, 3H), 1.13 (s, 3H), 1.11 (s, 3H), 1.02-1.45 (m, 4H), 0.90 (d, J=5.7 Hz, 3H), 0.79 (s, 3H), 0.72 (d, J=6.6 Hz, 3H).

Compound 93: To a solution of compound 85 (170 mg, 0.37 mmol) in AcOH (4 mL) at room temperature was added trimethyl orthoformate (370 μL, 3.4 mmol) and sodium azide (298 mg, 4.58 mmol) sequentially. The resultant mixture was stirred at 80° C. for 1 h, then at room temperature for 16 h. The reaction mixture was partitioned between EtOAc (20 mL) and H₂O (10 mL). The aqueous phase was extracted with EtOAc (10 mL). The combined organic extracts were washed with saturated aqueous NaHCO₃(10 mL) and brine (10 mL), dried over Na₂SO₄, filtered, and concentrated under reduced pressure. The residue was purified by column chromatography (silica gel, eluting with 0-60% acetone in hexanes) to give compound 93 (73 mg, 38% yield) as a white solid. m/z=518 (M+1).

Compound 94: To a solution of compound 93 (73 mg, 0.14 mmol) in MeOH (3 mL) at room temperature was added potassium carbonate (58 mg, 0.42 mmol). The mixture was stirred at room temperature for 18 h, then at 55° C. for 2 h. The reaction mixture was concentrated under reduced pressure. The residue was partitioned between EtOAc (10 mL) and water (10 mL). The aqueous phase was extracted with EtOAc (2×10 mL). The combined organic extracts were washed with brine (10 mL), dried over Na₂SO₄, filtered, and concentrated under reduced pressure to give compound 94 (65 mg, 89% yield) as a white solid, which was used in next step without further purification. m/z=448 (M-CHN₄).

T61: To a solution of compound 94 (65 mg, 0.12 mmol) in chloroform (3 mL) at room temperature was added DDQ (28 mg, 0.12 mmol). The mixture was stirred at 50° C. for 1 h, then cooled to room temperature. Saturated aqueous NaHCO₃(10 mL) was added. The aqueous phase was separated and extracted with EtOAc (2×20 mL). The combined organic extracts were washed with saturated aqueousNaHCO3 (2×10 mL), dried over Na₂SO₄, filtered, and concentrated under reduced pressure. The residue was purified by column chromatography (silica gel, eluting with 0-50% acetone in hexanes) to give compound T61 (40 mg, 62% yield) as a white solid. m/z=446 (M-CHN₄); ¹H NMR (400 MHz, CDCl₃) 8.37 (s, 1H), 6.34 (s, 1H), 3.30 (dd, J=11.2, 3.6 Hz, 1H), 2.46-2.54 (m, 2H), 2.22 (d, J=3.7 Hz, 1H), 2.02 (dt, J=13.1, 3.1 Hz, 1H), 1.90 (m, 1H), 1.67-1.85 (m, 7H), 1.46 (s, 3H), 1.24-1.52 (m, 4H), 1.27 (s, 3H), 1.20 (s, 3H), 1.17 (s, 3H), 0.98 (d, J=5.9 Hz, 3H), 0.87 (s, 3H), 0.80 (d, J=6.5 Hz, 3H).

Compound 95: To a solution of paraformaldehyde (183 mg, 6.09 mmol), ammonium carbonate (292 mg, 3.04 mmol) and glyoxal trimer dihydrate (548 mg, 2.61 mmol) in MeOH (7 mL) at room temperature was added compound 85 (202 mg, 0.44 mmol). The resultant mixture was stirred at room temperature for 3 days, then concentrated under reduced pressure. The residue was partitioned between EtOAc (20 mL) and H₂O (20 mL). The aqueous phase was extracted with EtOAc (2×10 mL). The combined organic extracts were washed with brine (20 mL), dried over Na₂SO₄, filtered, and concentrated under reduced pressure. The residue was purified by column chromatography (silica gel, eluting with 0-60% acetone in hexanes) to give compound 95 (94 mg, 42% yield) as yellow semi-solid. m/z=516 (M+1).

Compound 96: To a solution of compound 95 (94 mg, 0.18 mmol) in MeOH (5 mL) at room temperature was added potassium carbonate (76 mg, 0.55 mmol). The mixture was stirred at room temperature for 16 h, then concentrated under reduced pressure. The residue was partitioned between EtOAc (10 mL) and water (10 mL). The aqueous phase was extracted with EtOAc (2×10 mL). The combined organic extracts were washed with brine (10 mL), dried over Na₂SO₄, filtered and concentrated under reduced pressure. The residue was purified by column chromatography (silica gel, eluting with 0-70% acetone in hexanes) to give compound % (32 mg, 34% yield) as a white solid. m/z=516 (M+1).

T62: To a solution of compound 96 (32 mg, 0.062 mmol) in chloroform (3 mL) at room temperature was added DDQ (18 mg, 0.062 mmol). The mixture was stirred at 50° C. for 45 min, then cooled to room temperature. Saturated aqueous NaHCO₃(10 mL) was added. The aqueous phase was separated and extracted with EtOAc (2×20 mL). The combined organic extracts were washed with saturated aqueous NaHCO₃(2×10 mL), dried over Na₂SO₄, filtered, and concentrated under reduced pressure. The residue was first purified by column chromatography (Redi Sep amine column, eluting with 0-100% acetone in CH₂Cl₂) to give partially purified product, which was purified again by column chromatography (silica gel, 0-80% acetone in CH₂Cl₂ containing 0.5% Et₃N) to give compound T62 (14 mg, 42% yield) as a white solid. m/z=514 (M+1); ¹H NMR (400 MHz, CDCl₃) 8.05 (s, 1H), 7.70 (s, 1H), 7.10 (s, 1H), 7.08 (s, 1H), 6.13 (s, 1H), 3.05 (d, J=11.3 Hz, 1H), 2.73 (d, J=3.6 Hz, 1H), 2.48 (m, 1H), 2.00 (m, 1H), 1.57-1.91 (m, 9H), 1.43 (s, 3H), 1.25 (s, 3H), 1.20 (s, 3H), 1.17 (s, 3H), 1.14-1.49 (m, 4H), 0.96 (d, J=5.6 Hz, 3H), 0.90 (s, 3H), 0.83 (d, J=6.7 Hz, 4H).

Compound 97: To a solution of compound 85 HCl salt (400 mg, 0.80 mmol) in CH₂Cl₂ (5 mL) at −78° C. was added tert-butyl 3-(trichloromethyl)-1,2-oxaziridine-2-carboxylate (230 mg, 0.88 mmol). The mixture was stirred at −78° C. for 5 h, then slowly warmed up to room temperature and stirred at room temperature for 15 h. The reaction mixture was concentrated, and the residue was purified by column chromatography (silica gel, 0-50% eluting with acetone in hexanes) to give compound 97 (92 mg, 20% yield) as a white solid. m/z=580 (M+1).

Compound 98: To a solution of compound 97(92 mg, 0.16 mmol) in THF (2 mL) at room temperature was added HCl (4 M in 1,4-dioxane, 0.60 mL, 2.4 mmol) dropwise. The mixture was stirred at room temperature for 3 h, then at 55° C. for 40 h. The reaction mixture was concentrated under reduced pressure to give compound 98 HCl salt (100 mg) as a dark green solid, which was used in next step without further purification.

Compound 99: To a solution of compound 98 HCl salt (100 mg, S 0.16 mmol) in EtOH (1 mL) at room temperature was added 1.1.3.3-tetramethoxypropane (29 μL, 0.18 mmol) and aqueous HCl (12 N, 40 μL, 0.48 mmol) sequentially. The resultant mixture was stirred at 70° C. for 4 h, then concentrated under reduced pressure. The residue was diluted with EtOAc (20 mL) and washed with saturated aqueous NaHCO₃(10 mL). The aqueous phase was separated and extracted with EtOAc (10 mL). The combined organic extracts were washed with brine (10 mL), dried over Na₂SO₄, filtered, and concentrated under reduced pressure. The residue was purified by column chromatography (silica gel, eluting with 0-40% acetone in hexanes) to give compound 99 (41 mg, 49% yield from 97) as brown-yellow semi-solid. m/z=516 (M+1).

Compound 100: To a solution of compound 99 (41 mg, 0.079 mmol) in MeOH (3 mL) at room temperature was added potassium carbonate (33 mg, 0.24 mmol). The mixture was stirred at room temperature for 16 h, then concentrated under reduced pressure. The residue was purified by column chromatography (silica gel, eluting with 0-70% acetone in hexanes) to give compound 100 (15 mg, 37% yield) as a white solid. m/z=516 (M+1).

T63 and T64: To a solution of compound 100 (14 mg, 0.027 mmol) in DMF (0.3 mL) at 0° C. was added 1,3-dibromo-5,5-dimethylhydantoin (3.7 mg, 0.013 mmol) under argon atmosphere. The mixture was stirred at 0° C. for 1 h, then pyridine (9 μL, 0.11 mmol) was added at 0° C. The resultant mixture was stirred at 55° C. for 4 h, then at 40° C. for 16 h under argon atmosphere. The reaction mixture was cooled to room temperature and partitioned between EtOAc (10 mL) and brine (10 mL). The aqueous phase was separated and extracted with EtOAc (10 mL).

The combined organic extracts were washed with brine (2×10 mL), dried over Na₂SO₄, filtered, and concentrated under reduced pressure. The residue was purified by column chromatography (silica gel, eluting with 0-60% acetone in hexanes) to give compound T63 (2.6 mg, 16% yield) as a white solid and compound T64 (2.8 mg, 20% yield) as a white solid. T63: m/z=592/594 (M+1); H NMR (400 MHz, CDCl₃) 8.01 (s, 1H), 7.63 (s, 1H), 7.47 (s, 1H), 6.10 (s, 1H), 3.22 (d, J=11.3 Hz, 1H), 2.66 (d, J=3.4 Hz, 1H), 2.21-2.42 (m, 2H), 1.43 (s, 3H), 1.25 (s, 3H), 1.19 (s, 3H), 1.16 (s, 3H), 1.14-1.94 (m, 13H), 0.95 (d, J=5.7 Hz, 3H), 0.90 (s, 3H), 0.84 (d, J=6.6 Hz, 3H). T64: m/z=514 (M+1); ¹H NMR (400 MHz, CDCl₃) 8.02 (s, 1H), 7.62 (dd, J=2.4, 0.7 Hz, 1H), 7.52 (dd, J=1.7, 0.6 Hz, 1H), 6.28 (dd, J=2.4, 1.7 Hz, 1H), 6.09 (s, 1H), 3.26 (dd, J=11.2, 3.7 Hz, 1H), 2.71 (d, J=3.7 Hz, 1H), 2.31-2.38 (m, 2H), 1.41 (s, 3H), 1.25 (s, 3H), 1.19 (s, 3H), 1.16 (s, 3H), 1.10-1.98 (m, 13H), 0.96 (d, J=5.9 Hz, 3H), 0.85 (s, 3H), 0.84 (d, J=5.4 Hz, 3H).

Compound 101: To a solution of compound 85 HCl salt (150 mg, 0.30 mmol) in toluene (5 mL) at room temperature was added TEA (92 μL, 0.66 mmol) and ethyl isocyanate (35 μL, 0.45 mmol) sequentially. The mixture was stirred at room temperature for 18 h, then concentrated under reduced pressure. The residue was purified by column chromatography (silica gel, eluting with 0-40% acetone in hexanes) to give compound 101 (140 mg, 87% yield) as a white solid. m/z=536 (M+1).

Compound 102: To a solution of compound 101 (140 mg, 0.26 mmol) in MeOH (5 mL) at room temperature was added potassium carbonate (108 mg, 0.78 mmol). The mixture was stirred at room temperature for 20 h, then concentrated under reduced pressure. The residue was partitioned between EtOAc (10 mL) and aqueousHCl (0.2 N, 10 mL). The aqueous phase was extracted with EtOAc (2×10 mL). The combined organic extracts were dried over Na₂SO₄, filtered, and concentrated under reduced pressure. The residue was purified by column chromatography (silica gel, eluting with 0-65% acetone in hexanes) to give compound 102 (53 mg, 38% yield) as a white solid. m/z=536 (M+1).

T65: To a solution of compound 102 (53 mg, 0.099 mmol) in toluene (2 mL) and chloroform (2 mL) at room temperature was added DDQ (57 mg, 0.099 mmol). The mixture was stirred at 50° C. for 45 min, then cooled to room temperature and quenched with saturated aqueousNaHCO3 (10 mL). The aqueous phase was extracted with EtOAc (2×20 mL). The combined organic extracts were washed with saturated aqueousNaHCO3 (2×10 mL), dried over Na₂SO₄, filtered, and concentrated under reduced pressure. The residue was first purified by column chromatography (silica gel, eluting with 0-60% acetone in CH₂Cl₂), then purified again by column chromatography (silica gel, eluting with 0-100% EtOAc in hexanes) to give compound T65 (9.9 mg, 19% yield) as a white solid. m/z=534 (M+1); 8.08 (s, 1H), 6.11 (s, 1H), 4.71 (bs, 1H), 4.50 (bs, 1H), 3.28 (d, J=3.7 Hz, 1H), 3.17 (m, 2H), 2.51 (m, 1H), 2.39 (m, 1H), 2.22 (m, 1H), 1.49 (s, 3H), 1.33 (s, 3H), 1.27 (s, 3H), 1.19 (s, 3H), 1.12 (s, 3H), 1.10 (t, J=7.2 Hz, 3H), 1.06-1.94 (m, 13H), 0.90 (d, J=5.7 Hz, 3H), 0.72 (d, J=6.5 Hz, 3H).

Compound 103: To a solution of compound 84 (167 mg, 0.34 mmol) in MeOH (2 mL) at 0° C. was added sodium methoxide (25 wt. % in MeOH, 389 μL, 1.70 mmol). The mixture was stirred at 0° C. for 2 h, then at room temperature for 1 h. The reaction mixture was concentrated, and the residue was partitioned between EtOAc (10 mL) and water (10 mL). The aqueous phase was extracted with EtOAc (2×5 mL). The combined organic extracts were washed with brine (10 mL), dried over Na₂SO₄, filtered, and concentrated under reduced pressure. The residue was purified by column chromatography (silica gel, eluting with 0-50% acetone in hexanes) to give compound 103 (318 mg) as a colorless semisolid, which was used in next step without further purification. m/z=523 (M+1).

Compound 104: To a solution of compound 103 (318 mg, <0.34 mmol) in MeOH (3 mL) at room temperature was added potassium carbonate (141 mg, 1.02 mmol). The mixture was stirred at room temperature for 20 h. Additional amount of potassium carbonate (70 mg, 0.50 mmol) was added. The reaction mixture was stirred for additional 4 h, then concentrated under reduced pressure. The residue was partitioned between EtOAc (10 mL) and water (10 mL). The aqueous phase was separated and extracted with EtOAc (2×10 mL). The combined organic extracts were washed with water (2×10 mL), dried over Na₂SO₄, filtered, and concentrated under reduced pressure. The residue was purified by column chromatography (silica gel, eluting with 0-60% acetone in hexane) to give compound 104 (115 mg, 65% yield from compound 29) as a white solid. m/z=523 (M+1).

T66: To a solution of compound 104 (115 mg, 0.22 mmol) in DMF (1 mL) at 0° C. was added 1,3-dibromo-5,5-dimethylhydantoin (30 mg, 0.11 mmol) under argon atmosphere. The mixture was stirred at 0° C. for 1 h, then pyridine (71 μL, 0.88 mmol) was added at 0° C. The resultant mixture was stirred at 55° C. for 3.5 h, then cooled to room temperature. The mixture was partitioned between EtOAc (10 mL) and brine (10 mL). The aqueous phase was separated and extracted with EtOAc (10 mL). The combined organic extracts were washed with brine (2×10 mL), dried over Na₂SO₄, filtered, and concentrated under reduced pressure. The residue was purified by column chromatography (silica gel, eluting with 0-60% acetone in hexanes) to give compound T66 (100 mg, 88% yield) as a white solid. m/z=446 (M-CH₃₀CONH); ¹H NMR (400 MHz, CDCl₃) 8.05 (s, 1H), 6.13 (s, 1H), 4.39 (s, 1H), 3.62 (s, 3H), 3.11 (d, J=3.8 Hz, 1H), 2.43 (d, J=11.1 Hz, 1H), 2.06-2.25 (m, 2H), 1.95 (m, 1H), 1.68-1.86 (m, 6H), 1.50 (s, 3H), 1.33 (s, 3H), 1.27 (m, 3H), 1.19 (s, 3H), 1.13 (s, 3H), 1.10-1.61 (m, 6H), 0.90 (d, J=5.9 Hz, 3H), 0.74 (d, J=6.6 Hz, 3H).

T67: To a solution of compound 9 (196.3 mg, 0.40 mmol) in CH₂C12 (5 mL) at room temperature was added XeF₂ (81 mg, 0.48 mmol) in a PTFE vial. The mixture was stirred at room temperature for 16 h, then transferred to a separatory funnel with the help of CH₂Cl₂ (10 mL). The mixture was washed with saturated aqueous NaHCO₃ (10 mL). The aqueous phase was separated and extracted with CH₂Cl₂ (2×10 mL). The combined organic extracts were dried over Na₂SO₄, filtered, and concentrated under reduced pressure. The residue was purified by column chromatography (silica gel, eluting with 0-25% acetone in hexanes) to give compound T67 (20.6 mg, 11% yield) as a yellow solid and compound 105 (70 mg, 36% yield) as a yellow solid. T67: m/z=466 (M+1); ¹H NMR (400 MHz, CDCl₃) δ 8.07 (s, 1H), 6.14 (s, 1H), 3.23 (t, J=3.3 Hz, 1H), 2.57 (m, 1H), 1.52 (s, 3H), 1.37 (s, 3H), 1.27 (s, 3H), 1.20 (s, 3H), 1.12 (s, 3H), 1.02-2.12 (m, 15H), 0.89 (d, J=6.3 Hz, 3H), 0.75 (d, J=6.6 Hz, 3H). Compound 105: m/z=490 (M+1).

Compound 106: Compound 18 (222 mg, 0.465 mmol) was dissolved in acetic acid (2.3 mL). NaOAc (76 mg, 0.93 mmol) and peracetic acid (39 wt. % in acetic acid, 158 μL, 0.929 mmol) were added sequentially at room temperature. The mixture was stirred at room temperature under nitrogen for 16 h, and then was cooled to 0° C. 10% aqueous Na₂SO₃ (20 mL) was added. The mixture was stirred at ambient temperature for 20 min. The precipitated white solid was collected by filtration; and was washed with water (30 mL). The wet cake was dissolved in EtOAc (30 mL). The mixture was washed with water (20 mL). The organic extract was dried with MgSO₄, filtered and concentrated. The residue was purified by column chromatography (silica gel, eluting with 0-40% EtOAc in hexanes) to give partially purified compound 106 (180 mg, 79% yield) as a white solid, which was used in the next step without further purification. m/z=448 (M-OCHO).

Compound 107: To a mixture of partially purified compound 106 (178 mg, <0.361 mmol) in MeOH (3.6 mL) was added sodium methoxide solution (25 wt. % in MeOH, 165 μL, 0.721 mmol) at room temperature under nitrogen. The mixture was stirred at 55° C. for 2 h. After cooled to room temperature, the mixture was treated with 10% aqueous NaH₂PO₄ (15 mL) and water (15 mL); and was stirred at room temperature for 10 min. The precipitated white solid was collected by filtration; and was washed with water (30 mL). The wet cake was dissolved in EtOAc (30 mL). The mixture was washed with water (20 mL). The organic extract was dried with MgSO₄, filtered and concentrated. The residue was purified by column chromatography (silica gel, eluting with 0-40% EtOAc in hexanes) to give compound 107 (116 mg, 69% yield) as a white solid. m/z=448 (M-OH).

T68: Compound 107 (116 mg, 0.25 mmol) was dissolved in DMF (0.7 mL). The mixture was cooled to 0° C. A solution of 1,3-dibromo-5,5-dimethylhydantoin (36 mg, 0.13 mmol) in DMF (0.6 mL) was added. The mixture was stirred at 0° C. for 2 h. Pyridine (81 IL, 1.00 mmol) was added. The mixture was stirred at 55° C. for 5-6 h. The mixture was cooled to room temperature; diluted with EtOAc (30 mL); and washed sequentially with 10% aqueous Na₂SO₃ (15 mL), 1 N aqueous HCl (15 mL) and water (15 mL). The organic extract was dried with MgSO₄, filtered and concentrated. The residue was purified by column chromatography (silica gel, eluting with 0-60% EtOAc in hexanes) to give compound T68 (91 mg, 79% yield) as a white solid. m/z=446 (M-OH); ¹H NMR (400 MHz, CDCl₃) 8.07 (s, 1H), 6.13 (s, 1H), 3.56 (d, J=3.9 Hz, 1H), 2.25 (m, 1H), 1.92-2.12 (m, 2H), 1.71-1.89 (m, 5H), 1.51 (s, 3H), 1.38 (s, 3H), 1.27 (s, 3H), 1.20 (s, 3H), 1.10 (s, 3H), 0.98-1.67 (m, 8H), 0.89 (d, J=5.6 Hz, 3H), 0.74 (d, J=6.6 Hz, 3H).

Compound 10: To the solution of compound 9 (100 mg, 0.203 mmol) in CH₂Cl₂ (2 mL) at 0° C. under nitrogen was added oxalyl chloride (53 μL, 0.61 mmol) and DMF (1 drop) sequentially. The mixture was stirred at room temperature for 3 h, and then was concentrated. The residue was dissolved in toluene (2×10 mL) and concentrated under reduced pressure to give crude compound 10 as a brown solid, which was used in the next step without further purification.

Compound 108: Compound 10 (all from the last step, <0.203 mmol) was dissolved in 2-methyltetrahydrofuran (2 mL) and cooled to 0° C. under nitrogen. A suspension of acteylhydrazide (25 mg, 0.30 mmol) in CH₂Cl₂ (2.5 mL) was added. The mixture was stirred at room temperature for 30 min. Additional amount of acteylhydrazide (50 mg, 0.60 mmol) was added as a solid. The mixture was stirred at room temperature for 15 h. The mixture was diluted with EtOAc (40 mL) and washed with water (3×15 mL). The combined aqueous washes were extracted with EtOAc (20 mL), which was washed with water (3×10 mL). The combined organic extracts were dried with MgSO₄, filtered and concentrated. The residue was purified by column chromatography (silica gel, eluting with 0-100% EtOAc in hexanes) to give compound 108 (35 mg, 31% yield from compound 9) as a white solid. m/z=548 (M+1).

T69: The solution of compound 108 (35 mg, 0.064 mmol) in toluene (3 mL) was heated at reflux with Dean-Stark apparatus removal water for 1.5 h. The mixture was cooled to room temperature; diluted with EtOAc (30 mL); and washed with saturated aqueous NaHCO₃(10 mL) and water (10 mL). The organic extract was dried with MgSO₄, filtered and concentrated. The residue was purified by column chromatography (silica gel, eluting with 0-100% EtOAc in hexanes) to give compound T69 (27 mg, 82% yield) as a white solid. m/z=530 (M+1); ¹H NMR (400 MHz, CDCl₃) 8.02 (s, 1H), 6.09 (s, 1H), 3.00 (dd, J=11.3, 3.0 Hz, 1H), 2.50 (s, 3H), 2.19-2.35 (m, 3H), 2.05 (m, 1H), 1.91 (td, J=13.2, 4.5 Hz, 1H), 1.44 (s, 3H), 1.26 (s, 3H), 1.18-1.82 (m, 11H), 1.17 (s, 3H), 1.16 (s, 3H), 1.07 (s, 3H), 0.96 (d, J=6.0 Hz, 3H), 0.79 (d, J=6.6 Hz, 3H).

T70: To the solution of compound 10 (63 mg, 0.125 mmol) in CH₂Cl₂ (1 mL) at 0° C. under nitrogen was add azetidine (17 μL, 0.25 mmol). The mixture was stirred at room temperature for 1 h; and then was diluted with EtOAc (30 mL). The mixture was washed with water (3×10 mL). The organic extract was dried with MgSO₄; filtered; and concentrated. The residue was purified by column chromatography (silica gel, eluting with 0-60% acetone in hexanes) to give compound T70 (19 mg, 29% yield) as a light yellow solid. m/z=531 (M+1); ¹H NMR (400 MHz, CDCl₃) δ 8.05 (s, 1H), 6.12 (s, 1H), 4.31-4.72 (m, 2H), 3.80-4.20 (m, 2H), 2.84 (dd, J=10.9, 3.5 Hz, 1H), 2.67 (d, J=3.5 Hz, 1H), 2.24 (m, 2H), 1.49 (s, 3H), 1.27 (s, 3H), 1.26 (s, 3H), 1.19 (s, 3H), 1.13 (s, 3H), 1.09-2.00 (m, 15H), 0.91 (d, J=6.4 Hz, 3H), 0.74 (d, J=6.6 Hz, 3H).

T71: To the solution of compound 10 (63 mg, 0.125 mmol) in CH₂Cl₂ (1 mL) at 0° C. under nitrogen was add pyrrolidine (21 μL, 0.26 mmol). The mixture was stirred at room temperature for 30 min; and then was diluted with EtOAc (30 mL). The mixture was washed with water (3×10 mL). The organic extract was dried with MgSO₄; filtered; and concentrated. The residue was purified by column chromatography (silica gel, eluting with 0-100% EtOAc in hexanes) to give compound T71 (56 mg, 82% yield) as a light yellow solid. m/z=545 (M+1); ¹H NMR (400 MHz, CDCl₃) δ 8.05 (s, 1H), 6.10 (s, 1H), 3.85 (bs, 1H), 3.55 (bs, 3H), 3.21 (d, J=11.9 Hz, 1H), 2.55 (s, 1H), 1.47 (s, 3H), 1.26 (s, 3H), 1.23 (s, 3H), 1.18 (s, 3H), 1.14 (s, 3H), 1.10-2.11 (m, 19H), 0.93 (d, J=6.3 Hz, 3H), 0.76 (d, J=6.5 Hz, 3H).

T72: To a mixture of compound 10 (32 mg, 0.063 mmol) in THF (1 mL) and water (0.1 mL) at room temperature under nitrogen was added Et₃N (26 μL, 0.19 mmol) and hydroxylamine hydrochloride (8.7 mg, 0.13 mmol) sequentially. The mixture was stirred at room temperature for 16 h; and then was diluted with EtOAc (30 mL). The mixture was washed with water (3×10 mL). The organic extract was dried with MgSO₄; filtered; and concentrated. The residue was purified by column chromatography (silica gel, eluting with 0-85% acetone in hexanes) to give compound T72 (17 mg, 53% yield) as alight yellow solid. m/z=507 (M+1); ¹H NMR (400 MHz, CDCl₃) δ 8.84 (bs, 1H), 8.16 (s, 1H), 7.09 (bs, 1H), 6.20 (s, 1H), 2.64-2.74 (m, 2H), 2.02 (m, 1H), 1.69-1.92 (m, 8H), 1.49 (s, 3H), 1.27 (s, 3H), 1.24 (s, 3H), 1.19 (s, 3H), 1.15-1.63 (m, 6H), 1.13 (s, 3H), 0.92 (d, J=6.1 Hz, 3H), 0.74 (d, J=6.6 Hz, 3H).

T73: To a mixture of compound 10 (32 mg, 0.063 mmol) in THF (1 mL) and water (0.1 mL) at room temperature under nitrogen was added Et₃N (26 μL, 0.19 mmol) and methoxyamine hydrochloride (10 mg, 0.13 mmol) sequentially. The mixture was stirred at room temperature for 16 h; and then was diluted with EtOAc (30 mL). The mixture was washed with water (3×10 mL). The organic extract was dried with MgSO₄; filtered; and concentrated. The residue was purified by column chromatography (silica gel, eluting with 0-60% acetone in hexanes) to give compound T73 (19 mg, 58% yield) as a light yellow solid. m/z=521 (M+1); ¹H NMR (400 MHz, d6-DMSO) δ 11.17 (s, 1H), 8.67 (s, 1H), 6.39 (s, 1H), 3.53 (s, 3H), 2.75 (m, 1H), 2.63 (d, J=3.6 Hz, 1H), 1.42 (s, 3H), 1.18 (s, 3H), 1.14 (s, 3H), 1.08 (s, 3H), 1.05 (s, 3H), 1.01-1.99 (m, 15H), 0.85 (d, J=6.3 Hz, 3H), 0.65 (d, J=6.5 Hz, 3H).

Compound 109: To a solution of compound 85 (100 mg, 0.21 mmol) in MeOH (4 mL) was added Et₃N (36 μL, 0.26 mmol) and methyl acrylate (78 μL, 0.86 mmol) at room temperature. The reaction was heated at 50° C. for overnight. After that, additional amount of Et₃N (36 μL, 0.26 mmol) and methyl acrylate (78 μL, 0.86 mmol) was added. The reaction was stirred at 50° C. for another 3 days. The mixture was concentrated on rotovap. The residue was diluted with EtOAc (20 mL) and 10% aqueous NaH₂PO₄ (10 mL). The organic extract was separated and washed with H₂O (2×20 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give compound 109 (93 mg, 78% yield). m/z=551 (M+1).

Compound 110: To compound 109 (93 mg, 0.17 mmol) was added HCl (4 M in 1,4-dioxane (2 mL, 8 mmol) and 3 drops of water at room temperature. The reaction stirred run at room temperature for overnight. MeCN (1 mL) and HCl (6 M aqueous, 1 mL) were added, and the reaction was stirred at room temperature for overnight. Additional amount of HCl (6 M aqueous, 2 mL) was added, and the reaction was stirred at room temperature for overnight.

Compound 109 was completely consumed. The reaction was cooled down to 0° C. Saturated aqueous NaHCO₃(30 mL) and 10% aqueous NaH₂PO₄ (20 mL) were added to adjust pH to 6-7. The mixture was extracted with EtOAc (30 mL). The aqueous phase was separated and extracted with EtOAc (2×20 mL). The combined organic extracts were washed with brine (30 mL), dried with Na₂SO₄, filtered, and concentrated to give crude compound 110 (91 mg), which was used in the next step without further purification. m/z=537 (M+1).

Compound 111: To a solution of compound 110 (91 mg, 0.17 mmol) in CH₂Cl₂ (3 mL) at 0° C. was added Et₃N (71 μL, 0.51 mmol) and phosphorus oxychloride (24 μL, 0.25 mmol). The reaction was stirred at room temperature for 3 h, and then was diluted with EtOAc (20 mL) and quenched with saturated aqueous NaHCO₃(10 mL). The organic extract was separated and washed with H₂O (2×10 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give compound 111 (75 mg, 85% yield). m/z=519 (M+1).

Compound 112: To a solution of compound 111 (75 mg, 0.14 mmol) in MeOH (3 mL) was added K₂CO₃ (60 mg, 0.43 mmol) at room temperature. The reaction was run at room temperature for overnight, and then was diluted with EtOAc (20 mL) and 10% aqueous NaH₂PO₄ (10 mL). The organic extract was separated and washed with H₂O (2×20 mL). The aqueous phase was extracted with EtOAc (20 mL). The combined organic extracts were washed with brine (20 mL), dried with Na₂SO₄, filtered, and concentrated to give crude compound 112(37 mg, 49%), which was carried to the next step without further purification. m/z=519 (M+1).

T74: Compound 112 (35 mg, 0.067 mmol) was dissolved in DMF (3 mL) and cooled to 0° C. under nitrogen. 1,3-Dibromo-5,5-dimethylhydantoin (10 mg, 0.034 mmol) was added. The mixture was stirred at 0° C. for 1 h. Pyridine (16 μL, 0.2 mmol) was added. The mixture was heated at 60° C. for 9 h. After cooled to 0° C., the reaction was diluted with EtOAc (20 mL) and H₂O (20 mL). The organic extract was separated and washed with H₂O (2×10 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-50% acetone in hexanes) to give compound T74 (11 mg, 32% yield) as an off-white solid. m/z=517 (M+1); ¹H NMR (400 MHz, CDCl₃) 8.04 (s, 1H), 6.15 (s, 1H), 3.27 (m, 1H), 2.99 (m, 1H), 2.76-2.80 (m, 3H), 2.37 (m, 1H), 2.28 (m, 1H), 1.94-2.07 (m, 2H), 1.69-1.85 (m, 6H), 1.50 (s, 3H), 1.32 (s, 3H), 1.27 (s, 3H), 1.20 (s, 3H), 1.14 (s, 3H), 1.08-1.63 (m, 6H), 0.90 (d, J=6.1 Hz, 3H), 0.74 (d, J=6.6 Hz, 3H).

Compound 113: Compound 32 HCl salt (150 mg, 0.313 mmol) and succinic anhydride (47 mg, 0.47 mmol) were combined and dissolved in 1,4-dioxane (3.5 mL). DMAP (17 mg, 0.14 mmol) was added. The reaction was heated in Biotage microwave synthesizer at 150° C. for 16 h. After cooled to room temperature, the reaction was diluted with EtOAc (20 mL) and washed with H₂O (3×20 mL). The aqueous phase was separated and extracted with EtOAc (20 mL). The combined organic extracts were washed with brine (20 mL), dried with Na₂SO₄, filtered, and concentrated. The residue was purified by column chromatography (silica gel, eluting with 0-100% EtOAc in hexanes) to give compound 113 (74 mg, 42% yield). m/z=561 (M+1).

Compound 114: To a solution of compound 113 (74 mg, 0.13 mmol) in MeOH (3 mL) was added K₂CO₃ (73 mg, 0.53 mmol) at room temperature. The reaction was stirred at room temperature for overnight, and then was diluted with EtOAc (20 mL) and 10% aqueous NaH₂PO₄ (20 mL) was added. The organic extract was separated and washed with H₂O (2×20 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give compound 114 (13 mg, 18% yield). m/z=561 (M+1).

T75: Compound 114 (13 mg, 0.023 mmol) was dissolved in DMF (3 mL) and cooled to 0° C. under nitrogen. 1,3-Dibromo-5,5-dimethylhydantoin (3.3 mg, 0.012 mmol) was added. The mixture was stirred at 0° C. for 1 h. Pyridine (5.6 μL, 0.070 mmol) was added. The mixture was heated at 60° C. for 9 h. After cooled to 0° C., the reaction was diluted with EtOAc (20 mL) and H₂O (20 mL). The organic extract was separated and washed with H₂O (2×10 mL). The aqueous phase was extracted with EtOAc (20 mL). The combined organic extracts were washed with brine (20 mL), dried with Na₂SO₄, filtered, and concentrated. The residue was purified by column chromatography (silica gel, eluting with 0-60% acetone in hexanes) to give compound T75 (12 mg, 93% yield) as an off-white solid. m/z=559 (M+1); ¹H NMR (400 MHz, CDCl₃) δ 8.06 (s, 1H), 6.14 (s, 1H), 3.63 (d, J=13.5 Hz, 1H), 3.51 (d, J=13.5 Hz, 1H), 3.36 (d, J=3.7 Hz, 1H), 2.80-2.66 (m, 4H), 2.21-2.10 (m, 2H), 2.03-1.89 (m, 1H), 1.84-1.76 (m, 4H), 1.49 (s, 3H), 1.45 (s, 3H), 1.25 (s, 3H), 1.18 (s, 3H), 1.12 (s, 3H), 1.00-1.65 (m, 9H), 0.84 (d, J=6.2 Hz, 3H), 0.68 (d, J=6.7 Hz, 3H).

Compound 115: To a mixture of compound 75 (110 g, 0.234 mol) in MeCN (1.1 L) was added tert-butyldimethylsilyl trifluoromethanesulfonate (93 g, 0.35 mol) and 2,6-lutidine (63 g, 0.59 mol). The reaction mixture was stirred at room temperature over night. The precipitated solid was collected by filtration and was dried under vacuum to give compound 115 (108 g, 79% yield) as a white solid. ¹H NMR (400 MHz, CDCl₃) δ 5.24 (t, J=3.7 Hz, 1H), 3.60 (s, 3H), 3.18 (dd, J=11.3, 4.6 Hz, 1H), 2.26-2.18 (m, 1H), 2.00 (td, J=13.3, 4.5 Hz, 1H), 1.93-1.87 (m, 2H), 1.77 (td, J=13.6, 4.7 Hz, 1H), 1.70-1.63 (m, 2H), 1.63-1.52((m, 4H), 1.52-1.42((m, 4H), 1.38-1.25 (m, 4H), 1.07 (s, 3H), 1.06-0.97 (m, 2H), 0.95 (s, 3H), 0.94-0.92 (m, 1H), 0.91 (s, 3H), 0.90 (s, 3H), 0.89 (s, 9H), 0.86 (d, J=6.5 Hz, 3H), 0.74 (s, 3H), 0.74 (s, 3H), 0.72-0.67 (m, 1H), 0.03 (s, 6H).

Compound 116: To a solution of compound 115 (30.0 g, 51.3 mmol) in THF (300 mL) was added DIBAL-H (1 M in hexanes, 257 mL, 257 mmol) dropwise at 0° C. under nitrogen. The reaction was stirred at 0° C. for 30 min and then at room temperature for 2 h. The reaction was cooled to 0° C. Water (200 mL) was added carefully. EtOAc (400 mL) and 10% aqueous Roselle salt (400 mL) were then added. The mixture was stirred until the layers were separated. The organic extract was washed with water (2×500 mL) and brine (500 mL); dried over anhydrous Na₂SO₄; filtered and concentrated to give crude compound 116 (31.0 g, quantitative yield), which was used for the next step without further purification. ¹H NMR (400 MHz, CDCl₃) δ 5.14 (t, J=3.6 Hz, 1H), 3.53 (d, J=10.9 Hz, 1H), 3.22-3.16 (m, 2H), 1.98-1.87 (m, 3H), 1.78 (td, J=13.7, 4.7 Hz, 1H), 1.67-1.41 (m, 9H), 1.41-1.29 (m, 3H), 1.29-1.14 (m, 2H), 1.10 (s, 3H), 1.05-0.99 (m, 1H), 0.98 (s, 3H), 0.97-0.95 (m, 1H), 0.94 (s, 3H), 0.94-0.86 (m, 2H), 0.93 (s, 3H), 0.91 (s, 3H), 0.89 (m, 9H), 0.81 (d, J=5.6 Hz, 3H), 0.75 (s, 3H), 0.74-0.67 (m, 1H), 0.03 (s, 6H).

Compound 117: To a mixture of compound 116 (93 g, 167 mmol) in CH₂Cl₂ (900 ML) was added NMO (42.97 g, 366.8 mmol) and 4 A molecular sieves (186 g). The reaction was stirred at room temperature for 15 min. TPAP (5.86 g, 16.7 mmol) was added. The reaction was stirred at room temperature for 2 h. The mixture was filtered through a pad of celite. The filtrate was washed with 10% aqueous Na₂SO₃ (300 mL). The organic extract was washed with water (2×500 mL) and brine (500 mL); dried over Na₂SO₄; filtered and concentrated. The residue was purified by column chromatography (silica gel, eluting with 5% EtOAc in hexanes) to give compound 117 (61 g, 66% yield). ¹H NMR (400 MHz, CDCl₃) δ 9.33 (d, J=1.3 Hz, 1H), 5.31 (t, J=3.8 Hz, 1H), 3.18 (dd, J=11.1, 4.6 Hz, 1H), 2.05-1.93 (m, 2H), 1.93-1.88 (m, 2H), 1.81 (td, J=13.8, 5.0 Hz, 1H), 1.69-1.51 (m, 6H), 1.50-1.34 (m, 5H), 1.33-1.25 (m, 4H), 1.09 (s, 3H), 1.08-1.02 (m, 1H), 1.01-0.97 (m, 1H), 0.96 (s, 3H), 0.92 (s, 3H), 0.90 (s, 3H), 0.88 (s, 9H), 0.87 (d, J=6.0 Hz, 3H), 0.76 (s, 3H), 0.72 (s, 3H), 0.71-0.66 (m, 1H), 0.03 (s, 6H).

Compound 118: Triethyl phosphonoacetate (121.2 g, 540.6 mmol) was added to a mixture of potassium tert-butoxide (60.6 g, 540.1 mmol) in THF (500 mL) at 0° C. The mixture was stirred at 0° C. for 15 min and was allowed to warm to room temperature. A solution of compound 117 (20 g, 36 mmol) in THF (100 mL) was added. The reaction was stirred at room temperature for 4 h, and then was quenched with water (200 mL). The mixture was extracted with EtOAc (2×200 mL). The combined organic extracts were washed with water (2×500 mL) and brine (500 mL); dried over Na₂SO₄; filtered and concentrated. The residue was combined with the crude product obtained from compound 117 (20 g, 36 mmol) using the same procedure, and was purified by column chromatography (silica gel, eluting with 5% EtOAc in hexanes) to give compound 118 (40 g, 89% yield) as a white solid. ¹H NMR (400 MHz, CDCl₃) δ 6.91 (d, J=16.3 Hz, 1H), 5.69 (d, J=16.3 Hz, 1H), 5.22 (t, J=3.6 Hz, 1H), 4.16 (q, J=7.1 Hz, 2H), 3.18 (dd, J=11.2, 4.6 Hz, 1H), 2.16 (td, J=13.6, 4.4 Hz, 1H), 1.97-1.86 (m, 2H), 1.82-1.68 (m, 2H), 1.66-1.56 (m, 3H), 1.54-1.30 (m, 9H), 1.29-1.18 (m, 5H), 1.07 (s, 3H), 1.02-0.87 (m, 3H), 0.94 (s, 3H), 0.92 (s, 3H), 0.90 (s, 3H), 0.89 (s, 9H), 0.84 (d, J=6.4 Hz, 3H), 0.82 (s, 3H), 0.74 (s, 3H), 0.72-0.65 (m, 1H), 0.03 (s, 6H).

Compound 119: A mixture of compound 118 (10 g, 16 mmol), 10% palladium on carbon (1 g) in MeOH (200 mL) was stirred under hydrogen (balloon) at room temperature overnight.

The catalyst was removed by filtration. The filtrate was concentrated to give crude compound 119 (10 g, quantitative yield) as a white solid, which was used in the next step without further purification. ¹H NMR (400 MHz, CDCl₃) δ 5.13 (t, J=3.6 Hz, 1H), 4.09 (q, J=7.1 Hz, 2H), 3.19 (dd, J=11.2, 4.6 Hz, 1H), 2.29-2.05 (m, 2H), 2.00-1.84 (m, 4H), 1.73 (tt, J=12.8, 6.7 Hz, 1H), 1.67-1.29 (m, 13H), 1.29-1.14 (m, 6H), 1.08 (s, 3H), 1.01 (s, 3H), 0.99-0.95 (m, 2H), 0.94 (s, 3H), 0.93-0.86 (m, 4H), 0.91 (d, J=2.9 Hz, 3H), 0.89 (s, 9H), 0.80 (d, J=6.1 Hz, 3H), 0.75 (s, 3H), 0.74-0.67 (m, 1H), 0.03 (s, 6H).

Compound 120: Compound 119 (39 g, 62 mmol) was added to a solution of TBAF (1 M in THF, 622 mL, 622 mmol) at room temperature. The reaction mixture was stirred at room temperature for 4 h, and then was concentrated. The residue was partitioned between EtOAc (500 mL) and brine (500 mL). The organic extract was dried with Na₂SO₄; filtered and concentrated to give crude compound 120 (33 g) as a white solid, which was used in the next step without further purification. ¹H NMR (400 MHz, CDCl₃) δ 5.13 (t, J=3.6 Hz, 1H), 4.17-4.10 (m, 2H), 3.22 (dd, J=10.8, 5.2 Hz, 1H), 2.26-2.05 (m, 2H), 2.01-1.82((m, 4H), 1.80-1.48 (m, 8H), 1.47-1.29 (m, 6H), 1.28-1.14 (m, 6H), 1.09 (s, 3H), 1.01 (s, 3H), 0.99 (s, 3H), 0.99-0.96 (m, 2H), 0.94 (s, 3H), 0.92 (d, J=6.0 Hz, 3H), 0.90-0.83 (m, 1H), 0.80 (d, J=6.0 Hz, 3H), 0.79 (s, 3H), 0.76-0.70 (m, 1H).

Compound 121: To a solution of compound 120 (33 g, <62 mmol) in CH₂Cl₂ (800 mL) was added Dess-Martin periodinane (54.6 g, 128.7 mmol) in portions. The mixture was stirred at room temperature for 2 h. Saturated aqueous Na₂S₂O₃ (600 mL) was added. The mixture was extracted with CH₂Cl₂ (3×500 mL). The combined organic extracts were washed with water (2.0 L); dried with Na₂SO₄; filtered and concentrated. The residue was purified by column chromatography (silica gel, eluting with 10% EtOAc in petroleum ether) to give compound 121 (22 g, 69% yield from compound 119) as a white solid. ¹H NMR (400 MHz, CDCl₃) δ 5.09 (t, J=3.6 Hz, 1H), 4.10-3.99 (m, 2H), 2.48 (ddd, J=15.8, 11.0, 7.3 Hz, 1H), 2.31 (ddd, J=16.0, 6.9, 3.7 Hz, 1H), 2.19-1.99 (m, 2H), 1.96-1.76 (m, 5H), 1.69 (td, J=13.7, 13.2, 4.5 Hz, 1H), 1.60-1.45 (m, 2H), 1.44-1.22 (m, 10H), 1.22-1.08 (m, 3H), 1.19 (s, 3H), 1.17 (s, 3H), 1.01 (s, 3H), 0.99 (s, 3H), 0.98 (s, 3H), 0.96-0.88 (m, 3H), 0.86 (d, J=5.9 Hz, 3H), 0.84-0.77 (m, 2H), 0.73 (d, J=5.8 Hz, 3H).

Compound 122: To a mixture of compound 121 (22.0 g, 43.1 mmol) in ethyl formate (103 mL, 1.27 mol) at 0° C. was added sodium methoxide (5 M solution in MeOH, 130 mL, 0.65 mol). The reaction mixture was stirred at room temperature for 3 h, and then was cooled to 0° C. HCl (6 N aqueous solution, about 108 mL, 0.65 mmol) was added slowly to adjust pH to 1-2. EtOH (400 mL) and hydroxylamine hydrochloride (4.5 g, 65.0 mmol, 1.5 equiv.) were added. The reaction mixture was stirred at 55° C. for 3 h. The mixture was concentrated. The residue was partitioned between CH₂Cl₂ (2 L) and water (200 mL). The aqueous phase was separated and extracted with CH₂Cl₂ (2×500 mL). The combined organic extracts were washed with water (200 mL) and brine (200 mL); dried with Na₂SO₄; filtered and concentrated. The residue was purified by column chromatography (silica gel, eluting with 0-10% EtOAc in hexanes) to give compound 122 (19.5 g, 85% yield) as a white solid. Compound 122 is a mixture of methyl and ethyl esters. m/z=522, 536 (M+1).

Compound 123: The solution of compound 122 (2.0 g, 3.7 mmol) in CH₂Cl₂ (20 mL) was cooled to −78° C. Ozone was bubbled through the reaction mixture until compound 122 was consumed (about 15 min). The reaction mixture was stirred at room temperature overnight. Dimethyl sulfide (1.86 g, 30 mmol) was then added. The mixture was stirred at room temperature for 1 h, and then concentrated. The residue was purified by silica gel column chromatography (silica gel, eluting with 20% EtOAc in petroleum ether) to give compound 123 (1.3 g, 63% yield) as a white solid. Compound 123 is a mixture of methyl and ethyl esters. m/z=538, 552 (M+1).

Compound 124: To a solution of compound 123 (9.0 g, 16.3 mmol) in CH₃CN (90 mL) was added pyridinium tribromide (6.8 g, 21.3 mmol) at room temperature. The reaction mixture was stirred at 50° C. for 3 h; cooled to room temperature; and quenched with 10% aqueous Na₂SO₃ (100 mL). The mixture was extracted with CH₂Cl₂ (3×100 mL). The combined organic extracts were dried over Na₂SO₄; filtered and concentrated. The residue was purified by column chromatography (silica gel, eluting with 0-20% EtOAc in hexanes) to give compound 124 (6 g, 70% yield) as a white solid. Compound 124 is a mixture of methyl and ethyl esters. m/z=536, 550 (M+1).

Compound 125 and 126: To a suspension of compound 124 (300 mg, 0.55 mmol) in MeOH (5 mL) at room temperature was added sodium methoxide (25 wt. % in MeOH, 624 μL, 2.73 mmol). The mixture was stirred at 55° C. for 6 h, then cooled to room temperature. The mixture was concentrated under reduced pressure. The residue was partitioned between aqueous HCl (1 N, 10 mL) and EtOAc (10 mL). The aqueous phase was extracted with EtOAc (10 mL). The combined organic extracts were washed with water (2×10 mL), brine (10 mL), dried over Na₂SO₄, filtered, and concentrated under reduced pressure to give a mixture of compound 125 and 126 (total 295 mg in approximately 6:1 ratio) as a pale yellow solid, which was used in next step without further purification. Compound 125: m/z=536 (M+1); Compound 126: m/z=522(M+1).

T76 and T77: To a solution of compound 125 and 126 (295 mg, <0.55 mmol) in DMF (1 mL) at 0° C. was added 1,3-dibromo-5,5-dimethylhydantoin (76 mg, 0.27 mmol) under argon atmosphere. The mixture was stirred at 0° C. for 1.5 h, then pyridine (178 μL, 2.3 mmol) was added at 0° C. The resultant mixture was stirred at 55° C. for 4.5 h, then cooled to room temperature. The mixture was partitioned between EtOAc (10 mL) and brine (10 mL). The aqueous phase was separated and extracted with EtOAc (2×10 mL). The combined organic extracts were washed with brine (2×10 mL), dried over Na₂SO₄, filtered, and concentrated under reduced pressure. The residue was purified by column chromatography (silica gel, eluting with 0-60% acetone in hexanes) to give compound T76 (60 mg, 20% yield from compound 124) as a white solid, and compound T77 (155 mg, 53% yield from compound 124) as a white solid. T76: m/z=534 (M+1); H NMR (400 MHz, CDCl₃) δ 8.03 (s, 1H), 6.11 (s, 1H), 3.63 (s, 3H), 2.81 (d, J=3.7 Hz, 1H), 2.26 (t, J=8.3 Hz, 2H), 2.15-2.08 (m, 1H), 1.96 (td, J=13.7, 4.3 Hz, 1H), 1.47 (s, 3H), 1.37 (s, 3H), 1.24 (s, 3H), 1.17 (s, 3H), 1.10 (s, 3H), 0.94-1.88 (m, 16H), 0.85 (d, J=6.4 Hz, 3H), 0.66 (d, J=6.6 Hz, 3H). T77: m/z=520 (M+1); ¹H NMR (400 MHz, CDCl₃) δ 8.09 (s, 1H), 6.18 (s, 1H), 2.84 (d, J=2.8 Hz, 1H), 2.32 (t, J=8.2 Hz, 2H), 2.15 (dd, J=9.6 Hz, 1H), 1.99 (td, J=13.7, 3.6 Hz, 1H), 1.50 (s, 3H), 1.38 (s, 3H), 1.27 (s, 3H), 1.20 (s, 3H), 1.12 (s, 3H), 0.98-1.92 (m, 16H), 0.88 (d, J=6.4 Hz, 3H), 0.69 (d, J=6.5 Hz, 3H).

T77: To a solution of compound T76 (60 mg, 0.11 mmol) in MeCN (2 mL) at room temperature was added aqueous HCl (2 N, 0.11 mL, 0.22 mmol). The mixture was stirred at 65° C. for 16 h, then cooled to room temperature. The mixture was partitioned between EtOAc (10 mL) and water (10 mL). The organic phase was washed with brine (10 mL). The combined aqueous phase was extracted with EtOAc (10 mL). The combined organic extracts were dried over Na₂SO₄, filtered, and concentrated under reduced pressure. The residue was purified by column chromatography (silica gel, eluting with 0-40% acetone in hexane) to give partially purified compound T77 (40 mg, 80% yield) as a yellow solid. m/z=520 (M+1).

Compound T78: To a solution of compound T77 (40 mg, 0.077 mmol) in DMF (3 mL) at room temperature was added methylamine (2 M in THF, 48 μL, 0.096 mmol), Et₃N (32 μL, 0.23 mmol) and HATU (59 mg, 0.15 mmol) sequentially. The mixture was stirred at room temperature for 16 h, then partitioned between brine (10 mL) and EtOAc (10 mL). The aqueous phase was extracted with EtOAc (10 mL). The combined organic extracts were washed with brine (2×5 mL), dried over Na₂SO₄, filtered, and concentrated under reduced pressure. The residue was purified by column chromatography (silica gel, eluting with 0-60% EtOAc in hexanes, then 0-60% acetone in CH₂Cl₂) to give compound T78 (27.5 mg, 67% yield) as a white solid. m/z=533 (M+1); ¹H NMR (400 MHz, CDCl₃) δ 8.27 (s, 1H), 6.25 (s, 1H), 5.81 (bs, 1H), 2.82 (d, J=3.6 Hz, 1H), 2.77 (d, J=4.7 Hz, 3H), 1.72-2.24 (m, 1OH), 1.51 (s, 3H), 1.41 (s, 3H), 1.28 (s, 3H), 1.20 (s, 3H), 1.11 (s, 3H), 0.97-1.67 (m, 1OH), 0.87 (d, J=6.4 Hz, 3H), 0.65 (d, J=6.5 Hz, 3H).

T79: To a solution of partially purified compound T77 (40 mg, <0.077 mmol) in DMF (3 mL) at room temperature was added ethylamine (2 M in THF, 48 μL, 0.096 mmol), Et₃N (32 L, 0.23 mmol) and HATU (59 mg, 0.15 mmol) sequentially. The mixture was stirred at room temperature for 16 h, then partitioned between brine (10 mL) and EtOAc (10 mL). The aqueous phase was extracted with EtOAc (10 mL). The combined organic extracts were washed with brine (2×5 mL), dried over Na₂SO₄, filtered, and concentrated under reduced pressure. The residue was purified by column chromatography (silica gel, 0-60% acetone in hexanes) to give compound T79 (9.3 mg, 22% yield) as a white solid. m/z=547 (M+1); ¹H NMR (400 MHz, CDCl₃) δ 8.11 (s, 1H), 6.16 (s, 1H), 5.51 (bs, 1H), 3.26 (m, 2H), 2.83 (d, J=3.7 Hz, 1H), 1.50 (s, 3H), 1.41 (s, 3H), 1.27 (s, 3H), 1.20 (s, 3H), 1.12 (s, 3H), 0.99-2.19 (m, 23H), 0.88 (d, J=6.3 Hz, 4H), 0.68 (d, J=6.6 Hz, 4H).

T80: To a solution of compound T77 (37.5 mg, 0.072 mmol) in DMF (3 mL) at room temperature was added azetidine (4.9 μL, 0.072 mmol), Et₃N (30 μL, 0.22 mmol) and HATU (55 mg, 0.14 mmol) sequentially. The mixture was stirred at room temperature for 16 h, then partitioned between brine (10 mL) and EtOAc (10 mL). The aqueous phase was extracted with EtOAc (10 mL). The combined organic extracts were washed with brine (2×5 mL), dried over Na₂SO₄, filtered, and concentrated under reduced pressure. The residue was purified by column chromatography (silica gel, eluting with 0-60% acetone in hexanes) to give compound TSO (21.6 mg, 54% yield) as a white solid. m/z=559 (M+1); ¹H NMR (400 MHz, CDCl₃) δ 8.07 (s, 1H), 6.13 (s, 1H), 4.15 (t, J=7.7 Hz, 2H), 3.99 (t, J=7.8 Hz, 2H), 2.85 (d, J=3.6 Hz, 1H), 2.27 (m, 2H), 2.14 (dd, J=2.8, 10.8 Hz, 1H), 1.73-2.06 (m, 9H), 1.50 (s, 3H), 1.41 (s, 3H), 1.26 (s, 3H), 1.19 (s, 3H), 1.12 (s, 3H), 0.98-1.61 (m, 1OH), 0.89 (d, J=6.4 Hz, 3H), 0.69 (d, J=6.6 Hz, 3H).

T81: To a solution of compound T77 (39 mg, 0.075 mmol) in DMF (3 ML) at room temperature was added cyclopropyl amine (6.5 μL, 0.094 mmol), Et₃N (31 μL, 0.23 mmol) and HATU (57 mg, 0.15 mmol) sequentially. The mixture was stirred at room temperature for 16 h, then partitioned between brine (10 mL) and EtOAc (10 mL). The aqueous phase was extracted with EtOAc (10 mL). The combined organic extracts were washed with brine (2×5 mL), dried over Na₂SO₄, filtered, and concentrated under reduced pressure. The residue was purified by column chromatography (silica gel, 0-50% acetone in CH₂Cl₂) to give partially purified product, which was purified again by column chromatography (silica gel, 0-60% acetone in hexanes) to give compound T81 (23 mg, 55% yield) as a white solid. m/z=559 (M+1); ¹H NMR (400 MHz, CDCl₃) δ 8.13 (s, 1H), 6.16 (s, 1H), 5.67 (s, 1H), 2.79 (d, J=3.6 Hz, 1H), 2.65 (m, 1H), 1.48 (s, 3H), 1.38 (s, 3H), 1.24 (s, 3H), 1.17 (s, 3H), 1.08 (s, 3H), 0.95-2.20 (m, 20H), 0.85 (d, J=6.4 Hz, 3H), 0.72 (m, 2H), 0.64 (d, J=6.6 Hz, 3H), 0.44 (m, 2H).

Compound 127: A solution of compound 18 (500 mg, 1.05 mmol), hydroxylamine hydrochloride (95 mg, 1.36 mmol) and NaOAc (155 mg, 1.36 mmol) in EtOH (20 mL) and water (1.25 mL) was stirred at room temperature for 16 h. The reaction mixture was concentrated and azeotroped with toluene (2×20 mL). The residue was partitioned between EtOAc (15 mL) and water (10 mL). The organic phase was washed with water (10 mL). The combined aqueous phase was extracted with EtOAc (10 mL). The combined organic phase was dried over Na₂SO₄, filtered, and concentrated under reduced pressure to give compound 127 (530 mg) as a white solid, which was used in next step without further purification. m/z=493 (M+1).

Compound 128 and 129: To a solution of compound 127(530 mg, <1.05 mmol)in MeCN (5 mL) at −10° C. was added aqueous HCl (12 N, 17.5 μL, 0.21 mmol). Then NCS (140 mg, 1.05 mmol) was added as a solution in MeCN (5 mL) dropwise at −10° C. The resultant mixture was stirred at −10° C. for 45 min, then ammonia (29 wt. % in water, 0.7 mL, 10.5 mmol) was added at -10° C. The reaction mixture was stirred at −10° C. for 4 h, slowly warmed up to room temperature and stirred at room temperature for 18 h. The reaction mixture was concentrated under reduced pressure and partitioned between EtOAc (10 mL) and water (10 mL). The aqueous phase was extracted with EtOAc (10 mL) and the combined organic extracts were washed with water (2×5 mL), dried over Na₂SO₄, filtered and concentrated under reduced pressure. The residue was purified by column chromatography (silica gel, eluting with 0-70% acetone in hexanes) to give compound 128 (45 mg, 8.5% yield from compound 18) as a white solid and compound 129 (293 mg, 57% yield, from compound 18) as a white solid. Compound 128: m/z=508 (M+1); Compound 129: m/z=491 (M+1).

Compound 130: To a solution of compound 128 (42 mg, 0.083 mmol) in AcOH (1 mL) at room temperature was added acetic anhydride (12 μL, 0.12 mmol). The mixture was stirred at room temperature for 60 min, then heated up to 100° C. and stirred at 100° C. for 3 h. The reaction mixture was cooled to room temperature, concentrated under reduced pressure. The residue was dissolved in toluene (2×10 mL) and concentrated. The residue was purified by column chromatography (silica gel, eluting with 0-40% acetone in hexane) to give compound 130 (18 mg, 40% yield) as a white solid. m/z=532 (M+1).

Compound 131: To a solution of compound 130 (18 mg, 0.033 mmol) in MeOH (1 mL) at room temperature was added potassium carbonate (14 mg, 0.10 mmol). The mixture was stirred at room temperature for 16 h, then concentrated under reduced pressure. The residue was partitioned between EtOAc (5 mL) and water (5 mL). The aqueous phase was extracted with EtOAc (2×5 mL). The combined organic extracts were washed with water (2×5 mL), dried over Na₂SO₄, filtered, and concentrated under reduced pressure. The residue was purified by column chromatography (silica gel, eluting with 0-70% acetone in hexanes) to give compound 131 (9.2 mg, 52% yield) as a white solid. m/z=532 (M+1).

T82: To a solution of compound 131 (9.2 mg, 0.017 mmol) in DMF (1 mL) at 0° C. was added 1,3-dibromo-5,5-dimethylhydantoin (2.4 mg, 0.0083 mmol) under argon atmosphere. The mixture was stirred at 0° C. for 1.5 h, then pyridine (5.6 μL, 0.069 mmol) was added at 0° C. The resultant mixture was stirred at 55° C. for 3.5 h, then cooled to room temperature and partitioned between EtOAc (5 mL) and brine (5 mL). The aqueous phase was extracted with EtOAc (2×5 mL). The combined organic extracts were washed with brine (2×5 mL), dried over Na₂SO₄, filtered, and concentrated under reduced pressure. The residue was purified by column chromatography (silica gel, eluting with 0-60% EtOAc in hexanes) to give compound T82 (2.5 mg, 28% yield) as a white solid. m/z=530 (M+1); ¹H NMR (400 MHz, CDCl₃) δ 8.02 (s, 1H), 6.09 (s, 1H), 3.11 (dd, J=11.4, 3.7 Hz, 1H), 2.55 (s, 3H), 2.49 (d, J=3.8 Hz, 1H), 2.23 (m, 1H), 1.43 (s, 3H), 1.25 (s, 3H), 1.17 (s, 3H), 1.16 (s, 3H), 1.13-2.08 (m, 14H), 1.03 (s, 3H), 0.94 (d, J=5.6 Hz, 3H), 0.79 (d, J=6.6 Hz, 3H).

Compound 132: To a suspension of compound 18 (150 mg, 0.31 mmol) in MeOH (5 mL) at room temperature was added K₂CO₃ (130 mg, 0.94 mmol). The resultant mixture was stirred at room temperature for 18 h, then concentrated under reduced pressure. The residue was partitioned between aqueous HCl (1 N, 10 mL) and EtOAc (10 mL). The aqueous phase was extracted with EtOAc (3×10 mL). The combined organic extracts were dried over Na₂SO₄, filtered and concentrated under reduced pressure. The residue was purified by column chromatography (silica gel, eluting with 0-40% acetone in hexanes) to give compound 132 (105 mg, 70% yield) as a white solid. m/z=478 (M+1).

T83: To a solution of compound 132 (105 mg, 0.22 mmol) in DMF (1 mL) at 0° C. was added 1,3-dibromo-5,5-dimethylhydantoin (30 mg, 0.11 mmol) under nitrogen atmosphere. The mixture was stirred at 0° C. for 1.5 h, then pyridine (71 μL, 0.98 mmol) was added at 0° C. The resultant mixture was stirred at 55° C. for 4.5 h, then cooled to room temperature. The mixture was partitioned between EtOAc (10 mL) and brine (10 mL). The aqueous phase was extracted with EtOAc (2×10 mL). The combined organic extracts were washed with brine (2×10 mL), dried over Na₂SO₄, filtered and concentrated under reduced pressure. The residue was purified by column chromatography (silica gel, eluting with 0-60% acetone in hexanes) to give compound T83 (61 mg, 58% yield) as a white solid. m/z=476 (M+1); ¹H NMR (400 MHz, CDCl₃) δ 9.30 (s, 1H), 8.01 (s, 1H), 6.10 (s, 1H), 2.68 (dd, J=11.5, 3.8 Hz, 1H), 2.21 (d, J=3.9 Hz, 1H), 1.46 (s, 3H), 1.23 (s, 3H), 1.18 (s, 3H), 1.16 (s, 3H), 1.10 (s, 3H), 1.00-1.94 (m, 15H), 0.90 (d, J=6.1 Hz, 3H), 0.74 (d, J=6.6 Hz, 3H).

T84: Compound T83 (47 mg, 0.099 mmol), hydroxylamine hydrochloride (8.9 mg, 0.13 mmol) and NaOAc (15 mg, 0.18 mmol) were mixed in EtOH (2 mL) and water (0.1 mL) at room temperature. The mixture was stirred at room temperature for 3 h, then concentrated under reduced pressure. The residue was azeotroped with toluene (20 mL), then purified by column chromatography (silica gel, eluting with 0-40% acetone in hexanes) to give compound T84 (30.8 mg, 64%) as a white solid. m/z=491 (M+1); ¹H NMR (400 MHz, CDCl₃) δ 8.02 (s, 1H), 7.48 (s, 1H), 7.18 (s, 1H), 6.10 (s, 1H), 2.70 (d, J=3.7 Hz, 1H), 2.45-2.36 (m, 1H), 2.05-1.93 (m, 2H), 1.86-1.67 (m, 4H), 1.45 (s, 3H), 1.25 (s, 3H), 1.23 (s, 3H), 1.15 (s, 3H), 1.09 (s, 3H), 1.00-1.63 (m, 9H), 0.88 (d, J=6.3 Hz, 3H), 0.70 (d, J=6.6 Hz, 3H).

T85: To a solution of compound 9 (25 mg, 0.051 mmol) in DMF (1 mL) at room temperature was added methylamine (2 M in THF, 32 μL, 0.064 mmol), Et₃N (21 μL, 0.15 mmol) and HATU (39 mg, 0.10 mmol) sequentially. The resultant mixture was stirred at room temperature for 16 h, then partitioned between brine (5 mL) and EtOAc (5 mL). The aqueous phase was extracted with EtOAc (5 mL). The combined organic extracts were washed with brine (2×5 mL), dried over Na₂SO₄, filtered, and concentrated under reduced pressure. The residue was purified by column chromatography (silica gel, eluting with 0-80% EtOAc in hexanes) to give compound T85 (19.4 mg, 75% yield) as a white solid. m/z=505 (M+1); ¹H NMR (400 MHz, CDCl₃) δ 8.72 (s, 1H), 6.47 (s, 1H), 6.24 (q, J=4.6 Hz, 1H), 2.72 (d, J=4.6 Hz, 3H), 2.72-2.65 (m, 1H), 2.54 (d, J=3.5 Hz, 1H), 1.48 (s, 3H), 1.25 (s, 3H), 1.21 (s, 3H), 1.15 (s, 3H), 1.05 (s, 3H), 1.00-1.98 (m, 15H), 0.85 (d, J=5.9 Hz, 3H), 0.60 (d, J=6.5 Hz, 3H).

T86: To a solution of compound 9 (25 mg, 0.051 mmol) in DMF (1 mL) at room temperature was added cyclopropylamine (4.4 μL, 0.064 mmol), Et₃N (21 μL, 0.15 mmol) and HATU (39 mg, 0.10 mmol) sequentially. The resultant mixture was stirred at room temperature for 16 h, then partitioned between brine (5 mL) and EtOAc (5 mL). The aqueous phase was extracted with EtOAc (5 mL). The combined organic extracts were washed with brine (2×5 mL), dried over Na₂SO₄, filtered, and concentrated under reduced pressure. The residue was purified by column chromatography (silica gel, eluting with 0-70% EtOAc in hexanes) to give compound T86 (20.5 mg, 76% yield) as a white solid. m/z=531 (M+1); ¹H NMR (400 MHz, CDCl₃) δ 8.89 (s, 1H), 6.57 (s, 1H), 6.46 (d, J=3.2 Hz, 1H), 2.63-2.74 (m, 2H), 2.50 (d, J=3.4 Hz, 1H), 2.01 (m, 1H), 1.54 (s, 3H), 1.30 (s, 3H), 1.26 (s, 3H), 1.20 (s, 3H), 1.06 (s, 3H), 1.09-1.91 (m, 14H), 0.87 (d, J=5.7 Hz, 3H), 0.71 (m, 2H), 0.59 (d, J=6.5 Hz, 3H), 0.49 (m, 1H), 0.38 (m, 1H).

T87: To a solution of compound 9 (25 mg, 0.051 mmol) in DMF (1 mL) at room temperature was added 2,2-difluoroethylamine (4.5 μL, 0.064 mmol), Et₃N (21 μL, 0.15 mmol) and HATU (39 mg, 0.10 mmol) sequentially. The resultant mixture was stirred at room temperature for 16 h, then partitioned between brine (5 mL) and EtOAc (5 mL). The aqueous phase was extracted with EtOAc (5 mL). The combined organic extracts were washed with brine (2×5 mL), dried over Na₂SO₄, filtered, and concentrated under reduced pressure. The residue was purified by column chromatography (silica gel, eluting with 0-60% EtOAc in hexanes) to give compound T87 (27.3 mg, 97% yield) as a white solid. m/z=555 (M+1); ¹H NMR (400 MHz, CDCl₃) 8.66 (s, 1H), 6.59 (t, J=6.1 Hz, 1H), 6.46 (s, 1H), 5.83 (tt, J=4.1, 56.4 Hz, 1H), 3.74 (m, 1H), 3.44 (m, 1H), 2.78 (dd, J=11.0, 1.9 Hz, 1H), 2.55 (d, J=3.4 Hz, 1H), 1.52 (s, 3H), 1.29 (s, 3H), 1.23 (s, 3H), 1.19 (s, 3H), 1.11-1.96 (m, 15H), 1.09 (s, 3H), 0.90 (d, J=6.1 Hz, 3H), 0.66 (d, J=6.5 Hz, 3H).

Compound 133: To a mixture of (methoxylmethyl)triphenylphosphonium chloride (30.9 g, 90.1 mmol) in THF (200 mL) was added lithium bis(trimethylsilyl)amide (1 M in THF, 90.1 mL, 90.1 mmol) at 0° C. under nitrogen. The mixture stirred at 0° C. for 30 min and was warmed to room temperature. A solution of compound 117 (10 g, 18.0 mmol) in THF (50 mL) was added. The mixture was stirred at room temperature for 4 h. The reaction was quenched with water (200 mL). The mixture was extracted with EtOAc (2×100 mL). The combined organic extracts were washed with H₂O (2×200 mL) and brine (200 mL); dried over Na₂SO₄; filtered and concentrated. The residue was purified by column chromatography (silica gel, eluting with 5% EtOAc in petroleum ether) to give crude compound 133 (11 g, quantitative yield) as a white solid, which was used in the next step without further purification. ¹H NMR (400 MHz, CDCl₃) δ 5.61 (d, J=7.0 Hz, 1H), 5.14 (t, J=3.7 Hz, 1H), 4.09 (d, J=6.9 Hz, 1H), 3.50 (s, 3H), 3.23-3.15 (m, 1H), 1.98-1.80 (m, 4H), 1.65-1.22 (m, 16H), 1.06 (s, 3H), 0.98-0.86 (m, 4H), 0.93 (s, 3H), 0.92 (s, 3H), 0.91 (s, 3H), 0.90 (s, 3H), 0.89 (s, 9H), 0.80 (d, J=6.5 Hz, 3H), 0.75 (s, 3H), 0.73-0.68 (m, 1H), 0.03 (s, 6H).

Compound 134: A solution of compound 133 (12 g, 20.1 mmol) in HCl (4 M solution in 1,4-dioxane, 100 mL) was stirred at room temperature for 16 h. The mixture was concentrated. The residue was washed with MTBE (100 mL) to give compound 134 (8.2 g, 90% yield) as a white solid. m/z=455 (M+1). ¹H NMR (400 MHz, CDCl₃) δ 9.82 (t, J=3.2 Hz, 1H), 5.19 (t, J=3.7 Hz, 1H), 3.23 (dd, J=10.8, 5.1 Hz, 1H), 2.53 (dd, J=14.7, 3.6 Hz, 1H), 2.17-2.04 (m, 2H), 1.98-1.81 (m, 3H), 1.75-1.32 (m, 13H), 1.31-1.17 (m, 2H), 1.12 (s, 3H), 1.10-1.06 (m, 1H), 1.03 (s, 3H), 1.00 (s, 3H), 1.07-0.97 (m, 1H), 0.97-0.87 (m, 1H), 0.95 (s, 3H), 0.93 (s, 3H), 0.82 (d, J=5.9 HZ, 3H), 0.79 (s, 3H), 0.77-0.70 (m, 1H).

Compound 135: To a solution of compound 134 (7.2 g, 15.8 mmol) in acetone (70 mL) at 0° C. was added Jones reagent (2 M in acetone, 17.4 mL, 34.8 mmol). The reaction mixture was stirred at room temperature for 2 h, and was cooled to 0° C. 10% aqueous Na₂SO₃ solution (50 mL) was added. The mixture was extracted with EtOAc (2×50 mL). The combined organic extracts were washed with water (100 mL) and brine (100 mL); dried over Na₂SO₄; filtered and concentrated. The residue was purified by column chromatography (silica gel, eluting with 0-20% EtOAc in hexanes) to give compound 135 (4.8 g, 65% yield) as a white solid. ¹H NMR (400 MHz, DMSO-d6) δ 5.18-5.13 (m, 1H), 2.56-2.47 (m, 3H), 2.42 (d, J=13.8 Hz, 1H), 2.35-2.22 (m, 2H), 2.05-1.65 (m, 6H), 1.65-1.15 (m, 11H), 1.08 (s, 3H), 1.04-0.93 (m, 1H), 1.01 (s, 9H), 0.97 (s, 3H), 0.90 (s, 3H), 0.93-0.83 (m, 1H), 0.78 (d, J=5.5 Hz, 3H).

Compound 136: To a flame-dried round bottom flask equipped with a stir bar under nitrogen was added compound 135 (5.4 g, 11.5 mmol), K₂CO₃ (3.2 g, 23.0 mol) and DMF (15 mL). Iodomethane (2.5 g, 17.3 mmol) was added. The reaction mixture was stirred at room temperature for 16 h. The reaction mixture was poured into water (50 mL) and stirred for 30 min at room temperature. The precipitated solid was collected by filtration and dried under vacuum to give compound 136 (5.0 g, 90% yield) as a white solid. ¹H NMR (400 MHz, CDCl₃) δ 5.19 (t, J=3.6 Hz, 1H), 3.63 (s, 3H), 2.62-2.49 (m, 2H), 2.38 (ddd, J=15.9, 6.9, 3.7 Hz, 1H), 2.08-1.87 (m, 6H), 1.70-1.19 (m, 14H), 1.11 (s, 3H), 1.10 (s, 3H), 1.08 (s, 3H), 1.07 (s, 3H), 1.06 (s, 3H), 1.05-1.01 (m, 1H), 0.96-0.88 (m, 1H), 0.92 (br s, 3H), 0.81 (d, J=6.0 Hz, 3H).

Compound 137: Compound 136 (4.5 g, 9.3 mmol) was mixed with ethyl formate (20.7 g, 279.6 mmol) and was cooled to 0° C. Sodium methoxide solution (5 M solution in MeOH, 28 mL, 140 mmol) was added. The reaction mixture was stirred at room temperature for 3 h, and was cooled to 0° C. HCl (6 M aqueous solution, 23 mL, 138 mmol) was added slowly to adjust pH to 1-2. EtOH (20 mL) and hydroxylamine hydrochloride (973 mg, 14.0 mmol) were added. The reaction mixture was stirred at 55° C. for 3 h. The mixture was concentrated. The residue was partitioned between CH₂Cl₂ (100 mL) and water (100 mL). The aqueous phase was separated and extracted with CH₂Cl₂ (2×50 mL). The combined organic extracts were washed with water (100 mL) and brine (100 mL); dried with Na₂SO₄; filtered and concentrated. The residue was purified by column chromatography (silica gel, eluting with 0-10% EtOAc in hexanes) to give compound 137 (4.0 g, 85% yield) as a white solid. ¹H NMR (400 MHz, CDCl₃) δ 7.98 (s, 1H), 5.23 (t, J=3.6 Hz, 1H), 3.63 (s, 3H), 2.58 (d, J=13.7 Hz, 1H), 2.47 (d, J=15.1 Hz, 1H), 2.14-1.89 (m, 6H), 1.74 (dd, J=11.6, 6.0 Hz, 1H), 1.70-1.34 (m, 10H), 1.32 (s, 3H), 1.31-1.25 (m, 2H), 1.23 (s, 3H), 1.12 (s, 3H), 1.10-1.04 (m, 1H), 1.08 (s, 3H), 0.97-0.88 (m, 1H), 0.93 (s, 6H), 0.82 (d, J=6.1 Hz, 3H).

Compound 138: A solution of compound 137 (4.1 g, 8.07 mmol) in CH₂Cl₂ (20 mL) was cooled to −78° C. Ozone was bubbled through the reaction mixture until compound 137 was completely consumed (about 15 min). The reaction was stirred at room temperature overnight; quenched with dimethyl sulfide (3.1 g, 50 mmol); stirred at room temperature for another 1 h; and then concentration. The residue was purified by column chromatography (silica gel, eluting with 17% EtOAc in petroleum ether) to give compound 138 (3.0 g, 71% yield) as a white solid. ¹H NMR (400 MHz, CDCl₃) δ 8.00 (s, 1H), 3.62 (s, 3H), 2.51-2.41 (m, 3H), 2.34-2.21 (m, 2H), 2.20-1.94 (m, 5H), 1.86-1.75 (m, 1H), 1.75-1.38 (m, 9H), 1.35 (s, 3H), 1.30 (s, 3H), 1.28-1.20 (m, 2H), 1.25 (s, 3H), 1.19-1.02 (m, 2H), 0.98 (s, 3H), 0.97 (s, 3H), 0.93 (d, J=6.1 Hz, 3H), 0.80 (d, J=6.1 Hz, 3H).

Compound 139: To a solution of compound 138 (3.0 g, 5.73 mmol) in CH₃CN (30 mL) was added pyridinium tribromide (2.4 g, 7.45 mmol) at room temperature. The reaction mixture was stirred at 50° C. for 3 h, and then was cooled to room temperature. 10% aqueous Na₂SO₃ solution (30 mL) was added. The mixture was extracted with CH₂Cl₂(3×50 mL). The combined organic extracts were dried over Na₂SO₄, filtered and concentrated. The residue was purified by column chromatography (silica gel, eluting with 0-20% EtOAc in hexanes) to give compound 139 (2 g, 67% yield) as a white solid. m/z=522 (M+1).

Compound 140: To a mixture of compound 139 (5.0 g, 9.6 mmol) in MeOH (50 mL) was added sodium methoxide (30 wt. % in MeOH, 2.6 g, 14.4 mmol) at room temperature under nitrogen. The mixture was stirred at 55° C. for 2 h, and then was cooled to 0° C. The mixture was diluted with MTBE (200 mL) and treated with 1 N aqueous HCl (100 mL). The organic extract was separated and washed with brine (200 mL); dried over Na₂SO₄; filtered and concentrated. The residue was purified by column chromatography (silica gel, eluting with 0-70% EtOAc in hexanes) to give compound 140 (4.0 g, 80% yield) as a white solid. m/z=522 (M+1).

T88: Compound 140 (4.0 g, 7.7 mmol) was dissolved in DMF (40 mL) and cooled to 0° C. under nitrogen. 1,3-Dibromo-5,5-dimethylhydantoin (1.2 g, 4.2 mmol) was dissolved in DMF (40 mL) and added to the reaction mixture. The mixture was stirred at 0° C. for 1.5 h. Pyridine (1.8 g, 22.8 mmol) was added. The mixture was stirred at 55° C. for 3 h, and then cooled to room temperature. The mixture was diluted with EtOAc (400 mL) and was washed with 5% aqueous Na₂SO₃ (200 mL), 1 N aqueous HCl (200 mL) and brine (200 mL) sequentially. The organic extract was dried with Na₂SO₄, filtered and concentrated. The residue was purified by column chromatography (silica gel, eluting with 0-35% EtOAc in hexanes to compound T88 (2.9 g, 73% yield) as a white solid. m/z=520 (M+1); ¹H NMR (400 MHz, CDCl₃) δ 8.04 (s, 1H), 6.12 (s, 1H), 3.65 (s, 3H), 2.88 (d, J=3.8 Hz, 1H), 2.43 (d, J=12.9 Hz, 1H), 2.33 (d, J=12.8 Hz, 1H), 2.22-2.16 (m, 1H), 1.97 (td, J=13.5, 4.1 Hz, 1H), 1.91-1.71 (m, 6H), 1.49 (s, 3H), 1.37 (s, 3H), 1.25 (s, 3H), 1.18 (s, 3H), 1.11 (s, 3H), 1.00-1.60 (m, 8H), 0.86 (d, J=6.3 Hz, 3H), 0.67 (d, J=6.6 Hz, 3H).

Compound 141: To a mixture of compound 139 (500 mg, 0.958 mmol) in MeOH (10 mL) at room temperature under nitrogen was added sodium methoxide (25 wt. % in MeOH, 1.10 mL, 4.79 mmol) and water (1 mL) sequentially. The mixture was stirred at 60° C. for 16 h, and then was cooled to 0° C. 1 N aqueous HCl (50 mL) was added. The mixture was stirred for 5 min. The precipitated solid was collected by filtration and was washed with water (2×15 mL). The wet cake was dissolved in EtOAc (40 mL) and was washed with water (20 mL). The organic extract was dried with MgSO₄, filtered and concentrated. The residue was purified by column chromatography (silica gel, eluting with 0-60% EtOAc in hexanes) to give compound 141 (297 mg, 61% yield) as a light yellow solid. m/z=508 (M+1).

T89: To a solution of compound 141 (492 mg, 0.969 mmol) in DMF (3 mL) at 0° C. under nitrogen was added the solution of 1,3-dibromo-5,5-dimethylhydantoin (139 mg, 0.485 mmol) in DMF (2 mL). The mixture was stirred at 0° C. for 2 h. Pyridine (314 μL, 3.88 mmol) was added. The mixture was stirred at 60° C. for 5-6 h. The mixture was cooled to room temperature. 1 N aqueous HCl (50 mL) was added. The mixture was stirred at room temperature for 10 min. The precipitated solid was collected by filtration and was washed with water (3×15 mL). The wet cake was dissolved in EtOAc (40 mL) and was washed with water (3×15 mL). The organic extract was dried with MgSO₄, filtered and concentrated. The residue was purified by column chromatography (silica gel, eluting with 0-100% EtOAc in hexanes) to give compound T89 (387 mg, 79% yield) as a yellow solid. m/z=506 (M+1); ¹H NMR (400 MHz, CDCl₃) δ 8.04 (s, 1H), 6.14 (s, 1H), 2.87 (d, J=3.7 Hz, 1H), 2.47 (d, J=12.9 Hz, 1H), 2.36 (d, J=12.9 Hz, 1H), 2.30-2.22 (m, 1H), 2.06-1.94 (m, 1H), 1.49 (s, 3H), 1.38 (s, 3H), 1.25 (s, 3H), 1.18 (s, 3H), 1.12 (s, 3H), 1.00-1.90 (m, 14H), 0.87 (d, J=6.3 Hz, 3H), 0.69 (d, J=6.6 Hz, 3H).

Compound 142: To a solution of compound T89 (61 mg, 0.12 mmol) in CH₂Cl₂ (3 mL) at 0° C. under nitrogen was added oxalyl chloride (32 μL, 0.36 mmol). The mixture was stirred at room temperature for 3.5 h, and then was concentrated. The residue was dissolved in toluene (2×3 mL) and concentrated. The residue was dried under vacuum to give the crude acid chloride (63 mg, quantitative yield) as a yellow solid.

T90: To the solution of compound 142 (21 mg, 0.040 mmol) in CH₂Cl₂ (1 mL) at 0° C. under nitrogen was added ammonia (7 M in MeOH, 14 μL, 0.10 mmol). The mixture was stirred at 0° C. for 15 min. 1 N aqueous HCl (3 mL) was added. The mixture was partitioned between CH₂Cl₂ (15 mL) and water (10 mL). The aqueous layer was separated and extracted with CH₂Cl₂ (2×10 mL). The combined organic extracts were dried with MgSO₄, filtered and concentrated. The residue was purified by column chromatography (silica gel, eluting with 0-80% acetone in hexanes) to give compound 7% (11 mg, 54% yield) as a white solid. m/z=505 (M+1); ¹H NMR (600 MHz, CDCl₃) δ 8.05 (s, 1H), 6.14 (s, 1H), 5.39 (s, 1H), 5.34 (s, 1H), 2.85 (d, J=3.7 Hz, 1H), 2.61 (d, J=13.0 Hz, 1H), 2.21-2.16 (m, 1H), 1.98-1.89 (m, 3H), 1.50 (s, 3H), 1.39 (s, 3H), 1.27 (s, 3H), 1.20 (s, 3H), 1.14 (s, 3H), 1.05-1.86 (m, 13H), 0.90 (d, J=6.5 Hz, 3H), 0.70 (d, J=6.6 Hz, 3H).

T91: To the solution of compound 142 (21 mg, 0.040 mmol) in CH₂Cl₂ (1 mL) at 0° C. under nitrogen was added methylamine (2 M in THF, 50 μL, 0.10 mmol). The mixture was stirred at 0° C. for 15 min. 1 N aqueous HCl (3 mL) was added. The mixture was partitioned between CH₂Cl₂ (15 mL) and water (10 mL). The aqueous layer was separated and extracted with CH₂Cl₂ (2×10 mL). The combined organic extracts were dried with MgSO₄, filtered and concentrated. The residue was purified by column chromatography (silica gel, eluting with 0-50% acetone in hexanes) to give compound T91 (11 mg, 53% yield) as a white solid. m/z=519 (M+1); H NMR (600 MHz, CDCl₃) δ 8.05 (s, 1H), 6.14 (s, 1H), 5.42 (q, J=4.8 Hz, 1H), 2.87 (d, J=3.7 Hz, 1H), 2.81 (d, J=4.8 Hz, 3H), 2.63 (d, J=13.1 Hz, 1H), 2.18-2.13 (m, 1H), 1.98 (td, J=13.3, 4.3 Hz, 1H), 1.91 (td, J=13.4, 4.0 Hz, 1H), 1.72-1.66 (m, 1H), 1.50 (s, 3H), 1.38 (s, 3H), 1.27 (s, 3H), 1.19 (s, 3H), 1.13 (s, 3H), 1.05-1.86 (m, 13H), 0.89 (d, J=6.5 Hz, 3H), 0.70 (d, J=6.6 Hz, 3H).

T92: To the solution of compound 142 (22 mg, 0.042 mmol) in CH₂Cl₂ (1 mL) at 0° C. under nitrogen was added cyclopropylamine (8.6 μL, 0.12 mmol). The mixture was stirred at 0° C. for 15 min, and then was concentrated. The residue was purified by column chromatography (silica gel, eluting with 0-50% acetone in hexanes) to give partially purified product, which was purified again by column chromatography (silica gel, eluting with 0-100% EtOAc in hexanes) to give compound T92 (10 mg, 44% yield) as a white solid. m/z=545 (M+1); ¹H NMR (400 MHz, CDCl₃) δ 8.03 (s, 1H), 6.11 (s, 1H), 5.52 (s, 1H), 2.81 (d, J=3.7 Hz, 1H), 2.67 (dq, J=7.1, 3.5 Hz, 1H), 2.57 (d, J=12.9 Hz, 1H), 2.15-2.07 (m, 1H), 2.01-1.81 (m, 1H), 1.48 (s, 3H), 1.36 (s, 3H), 1.25 (s, 3H), 1.17 (s, 3H), 1.11 (s, 3H), 1.00-1.82 (m, 15H), 0.87 (d, J=6.3 Hz, 3H), 0.76 (d, J=7.1 Hz, 2H), 0.67 (d, J=6.6 Hz, 3H), 0.52-0.43 (m, 2H).

T93: To the solution of compound 142 (21 mg, 0.040 mmol) in CH₂Cl₂ (1 mL) at 0° C. under nitrogen was added azetidine (6.8 μL, 0.10 mmol). The mixture was stirred at 0° C. for 15 min. 1 N aqueous HCl (3 mL) was added. The mixture was partitioned between CH₂Cl₂ (15 mL) and water (10 mL). The aqueous layer was separated and extracted with CH₂Cl₂ (2×10 mL). The combined organic extracts were dried with MgSO₄, filtered and concentrated. The residue was purified by column chromatography (silica gel, eluting with 0-60% acetone in hexanes) to give compound T93 (12 mg, 55% yield) as a white solid. m/z=545 (M+1); ¹H NMR (600 MHz, CDCl₃) δ 8.05 (s, 1H), 6.14 (s, 1H), 4.22-4.10 (m, 2H), 4.03-3.97 (m, 2H), 2.85 (d, J=3.7 Hz, 1H), 2.35 (d, J=13.1 Hz, 1H), 2.26-2.20 (m, 2H), 2.19-2.13 (m, 1H), 1.96 (td, J=12.8, 5.4 Hz, 1H), 1.90 (d, J=13.1 Hz, 1H), 1.67 (dt, J=13.2, 3.5 Hz, 1H), 1.50 (s, 3H), 1.37 (s, 3H), 1.27 (s, 3H), 1.19 (s, 3H), 1.13 (s, 3H), 1.05-1.88 (m, 13H), 0.89 (d, J=6.4 Hz, 3H), 0.70 (d, J=6.6 Hz, 3H).

T94: A mixture of compound T89 (120 mg, 0.237 mmol), ethylamine (2 M in THF, 0.18 mL, 0.36 mmol), N,N-Diisopropylethylamine (92 mg, 0.71 mmol) and HATU (180 mg, 0.473 mmol) in CH₂Cl₂ (4 mL) was stirred at room temperature under nitrogen for 2 h. The reaction mixture was diluted with EtOAc (50 mL) and washed with 1 N aqueous HCl (25 mL) and brine (25 mL). The organic extract was dried over Na₂SO₄, filtered and concentrated. The residue was purified by column chromatography (silica gel, eluting with 0-10% MeOH in CH₂Cl₂) to give compound T94 (60 mg, 47% yield) as a white solid. m/z=533 (M+1); ¹H NMR (400 MHz, CDCl₃) δ 8.07 (s, 1H), 6.15 (s, 1H), 5.59 (m, 1H), 3.32-3.25 (m, 2H), 2.87-2.86 (m, 1H), 2.63-2.59 (m, 1H), 2.17-2.15 (m, 1H), 2.12-1.81 (m, 9H), 1.71-1.61 (m, 1H), 1.59-1.50 (m, 2H), 1.57 (s, 3H), 1.39 (s, 3H), 1.27-1.13 (m, 7H), 1.27 (s, 3H), 1.26 (s, 3H), 1.20 (s, 3H), 0.89 (d, J=6.0 Hz, 3H), 0.69 (d, J=6.4 Hz, 3H).

T89: Compound T88 (200 mg, 0.385 mmol) in DME (12 mL) was treated with HCl (6 M aqueous, 4 mL, 24 mmol). The mixture was heated in Biotage microwave synthesizer at 130° C. for 1.5 h, and then cooled to room temperature. Additional 8 reactions using the same condition were conducted, overall used compound T88 (1.8 g, 3.5 mmol). The reaction mixtures from the nine reactions were combined; diluted with EtOAc (200 mL); and washed with brine (100 mL). The organic extract was dried with Na₂SO₄; filtered and concentrated. The residue was purified by column chromatography (silica gel, eluting with 0-100% EtOAc in hexanes containing 0.5% AcOH) to compound T89 (1.0 g, 50% yield) as a white solid. m/z=506 (M+1).

Compound 143: A mixture of compound T89 (250 mg, 0.494 mmol), acethydrazide (55 mg, 0.74 mmol), Et₃N (100 mg, 0.988 mmol) and DMAP (111 mg, 0.909 mmol) in CH₂Cl₂ (10 mL) was stirred at room temperature under nitrogen for 30 min. N-(3-Dimethylaminopropyl)-N′-ethylcarbodiimide hydrochloride (189 mg, 0.988 mmol) was added at room temperature. The mixture was stirred at room temperature for 15 h; and then was diluted with EtOAc (100 mL). The mixture was washed with 1 N aqueous HCl (50 mL) and brine (50 mL). The organic extract was dried over Na₂SO₄, filtered and concentrated. The residue was purified by column chromatography (silica gel, eluting with 0-10% MeOH in CH₂Cl₂) to give compound 143 (140 mg, 50% yield) as a white solid. m/z=562.1 (M+1). 195: Compound 143 (140 mg, 0.249 mmol) and p-toluenesulfonic acid monohydrate (26 mg, 0.14 mmol) in toluene (8 mL) was heated at reflux with Dean-stark apparatus removal water for 1 h. The mixture was cooled to room temperature; diluted with EtOAc (50 mL); and washed with saturated aqueous NaHCO₃(50 mL), water (50 mL) and brine (50 mL). The organic extract was dried with Na₂SO₄; filtered and concentrated. The residue was purified by column chromatography (silica gel, eluting with 20-100% EtOAc in hexanes) to give compound T95 (40 mg, 30% yield) as a white solid. m/z=544 (M+1); ¹H NMR (400 MHz, CDCl₃) δ 8.07 (s, 1H), 6.17 (s, 1H), 3.03-2.98 (m, 2H), 2.84-2.80 (m, 1H), 2.52 (s, 3H), 2.14-2.10 (m, 1H), 2.03-1.98 (m, 2H), 1.92-1.72 (m, 5H), 1.65-1.50 (m, 3H), 1.53 (s, 3H), 1.45 (s, 3H), 1.35 (s, 3H), 1.34-1.21 (m, 3H), 1.20 (s, 3H), 1.18-1.02 (m, 2H), 1.16 (s, 3H), 0.88 (d, J=6.4 Hz, 3H), 0.69 (d, J=6.8 Hz, 3H).

Compound 144: Compound T89 (50 mg, 0.099 mmol), HATU (75 mg, 0.20 mmol) and N′-hydroxyacetimidamide (15 mg, 0.20 mmol) were dissolved in CH₂Cl₂ (4 mL). The reaction was stirred at room temperature for 10 min. N,N-Diisopropylethylamine (52 μL, 0.30 mmol) was added. The reaction was stirred for another 1 h, and then was cooled to 0° C. The mixture was diluted with EtOAc (10 mL) and washed with water (10 mL). The aqueous phase was separated and extracted with EtOAc (2×20 mL). The combined organic extracts were washed with brine (30 mL); dried with Na₂SO₄; filtered; and concentrated. The residue was purified by column chromatography (silica gel, eluting with 0-80% acetone in hexanes) to give compound 144 (50 mg, 90% yield). m/z=562 (M+1).

T96: In a microwave vial, compound 144 (24 mg, 0.043 mmol) was dissolved in toluene (1 mL) and EtOAc (0.1 mL). The vial was sealed and heated in Biotage microwave synthesizer at 200° C. for 20 min. After cooled to room temperature, the reaction mixture was concentrated. The residue was purified by column chromatography (silica gel, eluting with 0-60% acetone in hexanes) to give compound 96 (4 mg, 20% yield) as a light yellow solid. m/z=544 (M+1); ¹H NMR (600 MHz, CDCl₃) δ 8.06 (s, 1H), 6.16 (s, 1H), 3.07 (d, J=14.0 Hz, 1H), 2.96 (d, J=3.8 Hz, 1H), 2.85 (d, J=13.9 Hz, 1H), 2.40 (s, 3H), 2.20-2.14 (m, 1H), 2.05-1.94 (m, 2H), 1.52 (s, 3H), 1.45 (s, 3H), 1.28 (s, 3H), 1.21 (s, 3H), 1.16 (s, 3H), 1.05-1.86 (m, 13H), 0.88 (d, J=6.4 Hz, 3H), 0.70 (d, J=6.7 Hz, 3H).

Compound 145: To a solution of compound 139 (1.6 g, 3.07 mmol) in THF (30 mL) at 0° C. under nitrogen was added DIBAL-H (1 M in hexane, 24.6 mL, 24.6 mmol) dropwise. The reaction was stirred at 0° C. for 30 min and then at room temperature for 2 h. The reaction was cooled to 0° C., H₂O (100 mL) was added, followed by EtOAc (50 mL) and 10% aqueous Roselle salt (50 mL). The mixture was stirred until the layers were separated. The organic extract was washed with water (2×50 mL) and brine (50 mL), dried with Na₂SO₄, filtered and concentrated to give crude compound 145 (1.6 g), which was used for the next step without further purification. m/z=496 (M+1).

Compound 146: To a mixture of compound 145 (1.6 g, <3.07 mmol) in DME (30 mL) and water (3 mL) was added NBS (820 mg, 4.61 mmol). The mixture was stirred in dark for 30 min. 2% aqueous Na₂SO₃ (80 mL) was added to quench the reaction. The mixture was stirred for 15 min; and was extracted with EtOAc (50 mL). The organic extract was washed with brine (50 mL); dried with Na₂SO₄; filtered and concentrated. The residue was purified by column chromatography (silica gel, eluting with 0-40% EtOAc in hexanes) to give compound 146 (1.0 g, 70% yield from compound 139) as a white solid. m/z=494 (M+1).

Compound 147: To a mixture of compound 146 (1.0 g, 2.0 mmol) in MeOH (10 mL) was added sodium methoxide (30 wt. % in MeOH, 548 mg, 3.04 mmol) at room temperature under nitrogen. The mixture was stirred at 55° C. for 2 h, and then was cooled to 0° C. The mixture was diluted with MTBE (50 mL) and was treated with 1 N aqueous HCl (5 mL). The organic extract was separated; washed with brine (50 mL); dried over anhydrous Na₂SO₄; filtered and concentrated. The residue was purified by column chromatography (silica gel, eluting with 0-70% EtOAc in hexanes) to give compound 147(880 mg, 88% yield) as a white solid. m/z=494 (M+1).

T97: To a mixture of compound 147 (150 mg, 0.303 mmol) in THF (3 mL) was added DDQ (90 mg, 0.40 mmol) at room temperature under nitrogen. After stirring at room temperature for 2 h, the mixture was diluted with EtOAc (50 mL) and was treated with 1 N aqueous HCl (20 mL). The organic extract was separated and washed with brine (50 mL); dried over Na₂SO₄; filtered and concentrated. The residue was purified by column chromatography (silica gel, eluting with 0-50% EtOAc in hexanes) to give compound T97 (40 mg, 27% yield) as a white solid. m/z=492 (M+1); ¹H NMR (400 MHz, CDCl₃) δ 8.06 (s, 1H), 6.15 (s, 1H), 3.80-3.73 (m, 2H), 3.00-2.99 (m, 1H), 2.20-2.16 (m, 1H), 2.01-1.80 (m, 6H), 1.66-1.47 (m, 5H), 1.53 (s, 3H), 1.39 (s, 3H), 1.38-1.28 (m, 2H), 1.27 (s, 3H), 1.26-1.18 (m, 2H), 1.20 (s, 3H), 1.13 (s, 3H), 1.12-1.05 (m, 2H), 0.88 (d, J=6.4 Hz, 3H), 0.68 (m, J=6.8 Hz, 3H).

T98: To the solution of compound T19 (38 mg, 0.070 mmol)in MeCN (0.7 mL) was added hydrogen peroxide (30% aqueous, 21 μL, 0.20 mmol) at room temperature. The mixture was stirred at room temperature for 2 h. Additional amount of hydrogen peroxide (30% aqueous, 100 L, 0.98 mmol) was added. The mixture was stirred at room temperature for an additional 6 h, and then was diluted with EtOAc (30 mL). The mixture was washed with 10% aqueous Na₂SO₃ (10 mL), water (10 mL) and brine (10 mL). The organic extract was dried with MgSO₄, filtered and concentrated. The residue was purified by column chromatography (silica gel, eluting with 0-50% acetone in hexanes) to give compound 78 (23 mg, 59% yield) as a white solid. m/z=561 (M+1); ¹H NMR (400 MHz, CDCl₃) 6.20 (s, 1H), 5.69 (t, J=6.4 Hz, 1H), 4.37 (s, 1H), 3.47 (dd, J=13.9, 7.1 Hz, 1H), 3.24 (dd, J=13.8, 5.9 Hz, 1H), 2.97 (d, J=3.7 Hz, 1H), 2.21 (dd, J=11.2, 3.6 Hz, 1H), 1.97-2.07 (m, 2H), 1.91 (td, J=13.6, 4.4 Hz, 1H), 1.40 (s, 3H), 1.28 (s, 3H), 1.20 (s, 3H), 1.17 (s, 3H), 1.13 (s, 3H), 1.03-1.77 (m, 13H), 0.96 (m, 2H), 0.90 (d, J=6.4 Hz, 3H), 0.76 (m, 2H), 0.72 (d, J=6.7 Hz, 3H).

T99: To the solution of compound T1 (11 mg, 0.021 mmol) in MeCN (0.5 mL) was added hydrogen peroxide (30% aqueous, 100 μL, 0.98 mmol) at room temperature. The mixture was stirred at room temperature for 65 h, and then was diluted with EtOAc (30 mL). The mixture was washed with 10% aqueous Na₂SO₃ (15 mL) and water (10 mL). The organic extract was dried with MgSO₄, filtered and concentrated. The residue was purified by column chromatography (silica gel, eluting with 0-60% EtOAc in hexanes) to give compound T9 (9 mg, 79% yield) as a white solid. m/z=546 (M+1); ¹H NMR (400 MHz, CDCl₃) δ 6.18 (s, 1H), 4.32 (s, 1H), 3.18 (dd, J=11.4, 3.7 Hz, 1H), 2.56 (d, J=3.8 Hz, 1H), 2.36 (s, 3H), 2.27 (td, J=13.8, 4.5 Hz, 1H), 1.98-2.07 (m, 2H), 1.22 (s, 3H), 1.21 (s, 3H), 1.20 (s, 3H), 1.17-1.89 (m, 12H), 1.11 (s, 3H), 0.95 (d, J=4.7 Hz, 6H), 0.95 (s, 3H), 0.81 (d, J=6.6 Hz, 3H).

T100: To a solution of compound T17 (15 mg, 0.027 mmol) in MeCN (0.3 mL) at 0° C. was added hydrogen peroxide (30% aqueous, 11 L, 0.11 mmol). The mixture was stirred at room temperature for 4 h. Additional amount of hydrogen peroxide (30% aqueous, 5 μL, 0.05 mmol) was added. The mixture was stirred at room temperature for another 1 h. The mixture was diluted with EtOAc (20 mL); and was washed sequentially with 10% aqueous Na₂SO₃ (10 mL) and water (10 mL). The organic extract was dried with MgSO₄, filtered and concentrated. The residue was purified by column chromatography (silica gel, eluting with 0-50% acetone in hexanes) to give compound T100 (8 mg, 52% yield) as a white solid. m/z=571 (M+1); ¹H NMR (600 MHz, CDCl₃) δ 6.22 (s, 1H), 5.87 (s, 1H), 4.35 (s, 1H), 2.94 (d, J=3.9 Hz, 1H), 2.56-2.52 (m, 1H), 2.41-2.35 (m, 1H), 2.26-2.21 (m, 1H), 2.09-1.97 (m, 2H), 1.76 (t, J=19.3 Hz, 3H), 1.28 (s, 3H), 1.26 (s, 3H), 1.21 (s, 3H), 1.20 (s, 3H), 1.14 (s, 3H), 1.10-1.80 (m, 11H), 0.91 (d, J=5.8 Hz, 3H), 0.77 (d, J=6.7 Hz, 3H).

Compound 148: Compound 5 (1 g, 1.96 mmol) in MeOH (20 mL) was treated with sodium methoxide solution (25 wt. % in MeOH, 0.9 mL, 3.9 mmol) at room temperature under nitrogen. The mixture was heated at 55° C. for 1.5 h, and then cooled to 0° C. The mixture was treated with 10% aqueous NaH₂PO₄ (20 mL) and extracted with EtOAc (2×20 mL). The combined organic extracts were washed with brine (20 mL), dried with Na₂SO₄, filtered and concentrated to give crude compound 148 (1 g), which was used in the next step without further purification. m/z=510 (M+1).

T101: Compound 148 (1 g, 1.96 mmol) was dissolved in DMF (20 mL) and cooled to 0° C. under nitrogen. 1,3-Dibromo-5,5-dimethylhydantoin (280 mg, 0.98 mmol) in DMF (4 mL) was added dropwise. The mixture was stirred at 0° C. for 1 h. Pyridine (476 μL, 5.88 mmol) was added. The mixture was heated at 60° C. for 6 h. After cooled to 0° C., the reaction was diluted with EtOAc (30 mL) and 10% aqueous NaH₂PO₄ (30 mL) was added. The organic extract was separated and washed with H₂O (2×30 mL). The aqueous phase was extracted with EtOAc (30 mL). The combined organic extracts were washed with brine (40 mL), dried with Na₂SO₄, filtered, and concentrated. The residue was purified by column chromatography (silica gel, eluting with 0-40% EtOAc in hexanes) to give compound T101 (973 mg, 98% yield from compound 5). m/z=508 (M+1); ¹H NMR (400 MHz, CDCl₃) δ 7.69 (s, 1H), 3.71 (s, 3H), 2.86-2.70 (m, 2H), 2.52 (dd, J=15.1, 3.6 Hz, 1H), 1.98 (dd, J=13.7, 3.7 Hz, 1H), 1.92 (d, J=11.0 Hz, 1H), 1.90-1.78 (m, 2H), 1.40 (s, 3H), 1.21 (s, 3H), 1.20 (s, 3H), 1.16 (s, 3H), 0.95 (s, 3H), 0.9-1.75 (m, 13H), 0.77 (d, J=5.9 Hz, 3H), 0.70 (d, J=6.2 Hz, 3H).

Compound 149: To the solution of compound 5 (116 mg, 0.228 mmol) in THF (1.2 mL) at 0° C. under nitrogen was added DIBAL-H (1.0 M solution in toluene, 1.14 mL, 1.14 mmol). The mixture was stirred at 0° C. for 1 h and then at room temperature for 2 h. The mixture was cooled to 0° C.; and was treated with carefully with water (10 mL) and 1 N aqueous HCl (15 mL) sequentially. The mixture was extracted with EtOAc (2×15 mL). The combined organic extracts were washed with water (10 mL); dried with MgSO₄; filtered and concentrated. The residue was purified by column chromatography (silica gel, eluting with 0-100% EtOAc in hexanes) to give compound 149 (94 mg, 85% yield) as a white solid. m/z=482 (M+1).

Compound 150: To the solution of compound 149 (67 mg, 0.14 mmol) in acetone (6.7 mL) at 0° C. under nitrogen was added Jones' reagent (2.0 M in acetone, 0.15 mL, 0.30 mmol). The mixture was stirred at 0° C. for 20 min. i-PrOH was added to quench the reaction. The mixture was concentrated. The residue was partitioned between EtOAc (25 mL) and water (20 mL). The organic extract was separated; washed with water (10 mL); dried with MgSO₄; filtered and concentrated. The residue was purified by column chromatography (silica gel, eluting with 0-100% EtOAc in hexanes) to give compound 150 (53 mg, 79% yield) as a white solid. m/z=480 (M+1).

Compound 151: A mixture of compound 150 (72 mg, 0.15 mmol) and potassium carbonate (83 mg, 0.60 mmol) in MeOH (1.5 mL) was stirred at room temperature under nitrogen overnight. The mixture was treated with 10% aqueousNaH2PO₄ (10 mL) and was extracted with EtOAc (2×20 mL). The combined organic extracts were washed with water (20 mL); dried with MgSO₄; filtered and concentrated. The residue was purified by column chromatography (silica gel, eluting with 0-35% EtOAc in hexanes) to give compound 151 (32 mg, 44% yield) as a white solid. m/z=480 (M+1).

T102: To the solution of compound 151 (32 mg, 0.067 mmol) in DMF (0.5 mL) at 0° C. under nitrogen. 1,3-Dibromo-5,5-dimethylhydantoin (9.5 mg, 0.033 mmol) was dissolved in DMF (0.1 mL) and added to the reaction mixture. The mixture was stirred at 0° C. for 2 h. Pyridine (22 L, 0.27 mmol) was added. The mixture was stirred at 55° C. for 5-6 h. The reaction was complete. The mixture was cooled to room temperature; diluted with EtOAc (30 mL); and washed sequentially with 10% aqueousNa₂SO₃ (15 mL), 1 N aqueousHCl (15 mL) and water (15 mL). The organic extract was dried with MgSO₄, filtered and concentrated. The residue was purified by column chromatography (silica gel, eluting with 0-100% EtOAc in hexanes) to give compound T102 (28 mg, 88% yield) as a white solid. m/z=478 (M+1); ¹H NMR (400 MHz, CDCl₃) 7.81 (s, 1H), 4.71 (dt, J=9.8, 6.2 Hz, 1H), 2.19-2.29 (m, 2H), 2.13 (m, 1H), 2.03 (m, 1H), 1.21 (s, 3H), 1.17 (s, 3H), 1.15 (s, 3H), 1.09 (s, 3H), 1.07 (s, 3H), 0.99 (d, J=5.2 Hz, 3H), 0.94 (s, 3H), 0.87-1.88 (m, 16H).

Compound 152: To a suspension of compound 123(300 mg, 0.54 mmol)in MeOH (5 mL) at room temperature was added sodium methoxide (25 wt. % in MeOH, 622 μL, 2.72 mmol). The mixture was stirred at 55° C. for 6 h, then cooled to room temperature and concentrated under reduced pressure. The residue was partitioned between aqueous HCl (1 N, 10 mL) and EtOAc (10 mL). The aqueous phase was extracted with EtOAc (10 mL). The combined organic extracts were washed with water (2×10 mL) and brine (10 mL), dried over Na₂SO₄, filtered, and concentrated under reduced pressure to give compound 152 (281 mg, 99% yield) as a yellow solid, which was used in next step without further purification. m/z=524 (M+1).

Compound 153: To a solution of compound 152 (281 mg, 0.54 mmol) in DMF (1 mL) at 0° C. was added 1,3-dibromo-5,5-dimethylhydantoin (74 mg, 0.26 mmol) under argon atmosphere. The mixture was stirred at 0° C. for 1.5 h, then pyridine (174 μL, 2.2 mmol) was added at 0° C. The resultant mixture was stirred at 55° C. for 4.5 h, then cooled to room temperature. The mixture was partitioned between EtOAc (10 mL) and brine (10 mL). The aqueous phase was extracted with EtOAc (2×10 mL). The combined organic extracts were washed with brine (2×10 mL), dried over Na₂SO₄, filtered, and concentrated under reduced pressure. The residue was purified by column chromatography (silica gel, eluting with 0-60% acetone in hexanes) to give compound 153 (210 mg, 75% yield) as a white solid. m/z=522 (M+1).

T103: To a solution of compound 153 (38 mg, 0.073 mmol) in DMF (3 mL) at room temperature was added ethylamine (46 μL, 0.092 mmol), Et₃N (31 μL, 0.22 mmol) and HATU (56 mg, 0.15 mmol) sequentially. The mixture was stirred at room temperature for 16 h, then partitioned between brine (10 mL) and EtOAc (10 mL). The aqueous phase was extracted with EtOAc (10 mL). The combined organic extracts were washed with brine (2×5 mL), dried over Na₂SO₄, filtered, and concentrated under reduced pressure. The residue was purified by column chromatography (silica gel, eluting with 0-100% EtOAc in hexanes then 0-60% acetone in hexanes) to give compound T103 (22 mg, 56% yield) as white solid. m/z=549 (M+1); ¹H NMR (400 MHz, CDCl₃) δ 7.63 (s, 1H), 6.09 (t, J=5.7 Hz, 1H), 3.26 (m, 2H), 2.36-2.54 (m, 2H), 2.19-2.36 (m, 3H), 1.97-2.13 (m, 2H), 1.32 (s, 3H), 1.29 (s, 3H), 1.25 (s, 3H), 1.18 (s, 3H), 1.11 (t, J=7.3 Hz, 3H), 0.98 (s, 3H), 0.93 (s, 3H), 0.86-1.91 (m, 17H), 0.82 (d, J=6.1 Hz, 3H).

T104: To a solution of compound 153 (38 mg, 0.073 mmol) in DMF (3 mL) at room temperature was added methylamine (2 M in THF, 46 μL, 0.092 mmol), Et₃N (30 μL, 0.22 mmol) and HATU (55 mg, 0.15 mmol) sequentially. The mixture was stirred at room temperature for 16 h, then partitioned between brine (10 mL) and EtOAc (10 mL). The aqueous phase was extracted with EtOAc (10 mL). The combined organic extracts were washed with brine (2×5 mL), dried over Na₂SO₄, filtered, and concentrated under reduced pressure. The residue was purified by column chromatography (silica gel, eluting with 0-100% EtOAc in hexanes then 0-60% acetone in hexanes) to give compound T104 (24 mg, 62% yield) as white solid. m/z=535 (M+1); ¹H NMR (400 MHz, CDCl₃) δ 7.64 (s, 1H), 6.05 (q, J=4.9 Hz, 1H), 2.79 (d, J=4.8 Hz, 3H), 2.20-2.54 (m, 5H), 2.08 (m, 1H), 1.98 (d, J=11.2 Hz, 1H), 1.31 (s, 3H), 1.29 (s, 3H), 1.25 (s, 3H), 1.18 (s, 3H), 0.98 (s, 3H), 0.93 (s, 3H), 0.87-1.91 (m, 17H), 0.81 (d, J=6.1 Hz, 3H).

T105: To a solution of compound 153 (42 mg, 0.080 mmol) in DMF (3 mL) at room temperature was added azetidine (5.4 μL, 0.080 mmol), Et₃N (34 μL, 0.24 mmol) and HATU (61 mg, 0.16 mmol) sequentially. The mixture was stirred at room temperature for 16 h, then partitioned between brine (10 mL) and EtOAc (10 mL). The aqueous phase was extracted with EtOAc (10 mL). The combined organic extracts were washed with brine (2×5 mL), dried over Na₂SO₄, filtered, and concentrated under reduced pressure. The residue was purified by column chromatography (silica gel, 0-60% acetone in hexanes) to give compound T105 (29.8 mg, 66% yield) as a white solid. m/z=561 (M+1); ¹H NMR (400 MHz, CDCl₃) δ 8.00 (s, 1H), 4.18 (m, 2H), 3.86 (t, J=7.7 Hz, 2H), 2.76 (dd, J=17.2, 6.1 Hz, 1H), 2.56 (dd, J=12.0, 6.1 Hz, 1H), 2.27-2.48 (m, 2H), 2.23 (m, 2H), 1.37 (s, 3H), 1.35 (s, 3H), 1.22 (s, 3H), 1.17 (s, 3H), 1.03 (s, 3H), 0.93 (s, 3H), 0.89-2.14 (m, 19H), 0.80 (d, J=6.1 Hz, 3H).

Compound 154: To a mixture of compound 1 (4.02 g, 8.80 mmol) in DMF (15 mL) at room temperature under nitrogen was added HATU (4.02 g, 10.6 mmol) and Et₃N (2.45 mL, 17.6 mmol) sequentially. The mixture was stirred at room temperature for 1 h. Ammonia (7 M in MeOH, 1.9 mL, 13.3 mmol) was added at room temperature. The mixture was stirred at room temperature for 6 h. Additional amount of ammonia (7 M in MeOH, 0.5 mL, 3.5 mmol) was added. The mixture was stirred at room temperature for overnight. The mixture was diluted with water (50 mL) and stirred for 30 min at room temperature. The precipitated solid was collected by filtration; washed with water (3×50 mL); and was stirred with CH₂Cl₂ (200 mL) and water (100 mL). The aqueous phase was separated and extracted with CH₂Cl₂ (100 mL). The combined organic extracts were washed with water (50 mL); dried with MgSO₄; filtered; and concentrated. The product was mixed with toluene (50 mL); concentrated; and dried under vacuum to give compound 154 (4.01 g, quantitative yield) as a white solid, which was used in the next step without further purification. m/z=456 (M+1).

Compound 155: Compound 154 (4.01 g, 8.80 mmol) was dissolved in THF (74 mL) and cooled to 0° C. under nitrogen. LiAH₄ (2 M in THF, 13.2 mL, 26.4 mmol) was added. The mixture was stirred at room temperature for 10 min and then heated at reflux for 5 h. The mixture was cooled to 0° C. Water (74 mL) was added carefully. 3 N aqueous HCl (70 mL) was then added. The mixture was stirred at ambient temperature for 14 h. The precipitated white solid was collected by filtration. The filtrate was concentrated to remove THF. Additional amount of solid precipitated, which was collected by filtration. The solid was mixed with saturated aqueous NaHCO₃(50 mL), 10% aqueous Na₂CO₃ (50 mL) and CH₂Cl₂ (300 mL). The organic layer was separated; the aqueous phase was extracted was extracted with CH₂Cl₂ (2×150 mL). The combined organic extracts were washed with water (100 mL); dried with MgSO₄; filtered and concentrated to give crude compound 155 (3.24 g, 83%) as a white solid, which was used in the next step without further purification. m/z=442 (M+1).

Compound 156: Compound 155 (2.20 g, 4.98 mmol) was dissolved in THF (50 mL). Water (10 mL), di-tert-butyl dicarbonate (1.36 g, 6.23 mmol) and NaHCO₃(502 mg, 5.98 mmol) were added sequentially at room temperature. The reaction mixture was stirred at room temperature for 4 h. The mixture was combined with the reaction mixture obtained from compound 156 (1.04 g, 2.35 mmol) using the same procedure, and then was concentrated. The residue was partitioned between EtOAc (50 mL) and water (30 mL). The aqueous phase was separated and extracted with CH₂Cl₂ (2×50 mL). The combined organic extracts were washed with water (30 mL); dried with MgSO₄; filtered and concentrated to give compound 157 (3.51 g, 89% yield) as a white solid, which was used in the next step without further purification. m/z=468 (M-C4H₉₀).

Compound 157: Compound 156 (3.583 g, 6.612 mmol) was dissolved in EtOAc (30 mL). DMSO (3.30 mL, 46.5 mmol) was added at room temperature. The mixture was cooled to 0° C. Propylphosphonic anhydride (T3P, 50 wt. % in EtOAc, 5.904 mL, 9.919 mmol) was added. The mixture was stirred at 0° C. for 3.5 h. N,N-Diisopropylethylamine (1.73 mL, 9.92 mmol) was added. The mixture was stirred at room temperature for 1 h. Saturated aqueous NaHCO₃(50 mL) was added. The mixture was stirred at room temperature for 10 min. The mixture was extracted with EtOAc (2×50 mL). The combined organic extracts were washed with saturated aqueous NaHCO₃(50 mL) and water (50 mL); dried with MgSO₄; filtered and concentrated to give compound 157 (3.45 g, 97% yield) as a white solid. m/z=484 (M-C₄H₇).

Compound 158: Compound 157 (3.43 g, 6.35 mmol) was dissolved in ethyl formate (15.4 mL, 191 mmol) and was cooled to 0° C. Sodium methoxide (25 wt. % in MeOH, 14.5 mL, 63.5 mmol) was added. The mixture was stirred at room temperature for 2 h, and then cooled to 0° C. 6 N aqueous HCl (11.6 mL, 69.6 mmol) was added to adjust pH -1. EtOH (34 mL), water (3.4 mL) and hydroxylamine hydrochloride (883 mg, 12.7 mmol) were added sequentially. The mixture was heated at 55° C. for 4 h, and then was cooled to room temperature. Saturated aqueous NaHCO₃(50 mL) was added. The mixture was concentrated. The residue was extracted with EtOAc (3×50 mL). The combined organic extracts were washed with water (50 mL); dried with MgSO₄; filtered and concentrated to give partially Boc deprotected compound 158. The crude product was dissolved in THF (32 mL) and water (6 mL). NaHCO₃(534 mg, 6.35 mmol) and di-tert-butyl dicarbonate (1.39 g, 6.35 mmol) were added. The mixture was stirred at room temperature for 2 h, and then was concentrated. The residue was partitioned between EtOAc (50 mL) and saturated aqueous NaHCO₃(50 mL). The aqueous phase was extracted with EtOAc (50 mL). The combined organic extracts were washed with water (30 mL); dried with MgSO₄; filtered and concentrated. The residue was purified by column chromatography (silica gel, eluting with 0-30% EtOAc in hexanes) to give compound 158 (2.872 g, 80% yield) as a white solid. m/z=565 (M+1).

Compound 159: To a solution of compound 158 (2.912 g, 5.155 mmol) in CH₂Cl₂ (52 mL) was added 3-chloroperbenzoic acid (<77%, 5.199 g, <23.20 mmol) at room temperature. The mixture was stirred at room temperature for 40 h, and then cooled to 0° C. 10% aqueous Na₂SO₃ (100 mL) was added. The mixture was stirred at ambient temperature for 10 min. Saturated aqueous NaHCO₃(50 mL) was added. The mixture was extracted with CH₂Cl₂ (2×50 mL) and EtOAc (50 mL). The combined organic extracts were washed with saturated aqueous NaHCO₃(50 mL) and water (50 mL); dried with MgSO₄; filtered and concentrated.

The crude product was dissolved in CH₂Cl₂ (52 mL) and was cooled to 0° C. Methanesulfonic acid (100 μL, 1.54 mmol) was added. The mixture was stirred at 0° C. for 1 h. The mixture was treated with saturated aqueous NaHCO₃(50 mL); stirred for 5 min; and was extracted with CH₂Cl₂ (2×50 mL) and EtOAc (50 mL). The combined organic extracts were washed with water (50 mL); dried with MgSO₄; filtered and concentrated. The residue was purified by column chromatography (silica gel, eluting with 0-30% EtOAc in hexanes) to give compound 159 (2.02 g, 66% yield) as a white solid. m/z=525 (M-C₄H₇).

Compound 160: Compound 159 (200 mg, 0.344 mmol) in MeOH (6 mL) was treated with sodium methoxide solution (25 wt. % in MeOH, 157 μL, 0.69 mmol) at room temperature under nitrogen. The mixture was heated at 55° C. for 1.5 h, and then cooled to 0° C. The mixture was treated with 10% aqueous NaH₂PO₄ (20 mL) and was extracted with EtOAc (2×20 mL). The combined organic extracts were washed with brine (20 mL); dried with Na₂SO₄; filtered and concentrated to give crude compound 160 (200 mg), which was used in the next step without further purification. m/z=525 (M-C₄H₇).

T106: Compound 160 (200 mg, 0.344 mmol) was dissolved in DMF (6 mL) and was cooled to 0° C. under nitrogen. 1,3-Dibromo-5,5-dimethylhydantoin (49 mg, 0.17 mmol) was dissolved in DMF (1 mL) in a vial. The solution was added to the reaction mixture dropwise. DMF (1 mL) was used rinse the vial and was added to the reaction mixture. The mixture was stirred at 0° C. for 1 h. Pyridine (84 μL, 1.03 mmol) was added. The mixture was heated at 60° C. for 4 h, and then was cooled to 0° C. The mixture was diluted with EtOAc (20 mL) and was washed sequentially with 10% aqueous NaH₂PO₄ (20 mL) and water (2×10 mL). The combined aqueous washes were extracted with EtOAc (20 mL). The combined organic extracts were washed with brine (20 mL); dried with Na₂SO₄; filtered; and concentrated. The residue was purified by column chromatography (silica gel, eluting with 0-60% acetone in hexanes) to give compound T106 (158 mg, 79% yield from 159) as a white solid. m/z=523 (M-C₄H₇); ¹H NMR (400 MHz, CDCl₃) δ 7.62 (s, 1H), 5.54 (dd, J=3.6, 9.2 Hz, 1H), 2.99 (dd, J=13.9, 9.7 Hz, 1H), 2.68 (dd, J=13.9, 4.0 Hz, 1H), 2.33-2.53 (m, 3H), 2.21 (dd, J=12.0, 6.6 Hz, 1H), 1.78-1.93 (m, 4H), 1.45 (s, 9H), 1.32 (s, 3H), 1.29 (s, 3H), 1.25 (s, 3H), 1.18 (s, 3H), 0.98 (s, 3H), 0.93 (s, 3H), 0.90-1.70 (m, 12H), 0.84 (d, J=6.2 Hz, 3H).

### Example 2: Biological Data

Tissue Culture: RAW 264.7, a mouse macrophage cell line, was obtained from American Type Culture Collection (Manassas VA) and maintained in the log phase of growth in Roswell Park Memorial Institute Medium 1640 (RPMI 1640) supplemented with 10% heat inactivated fetal bovine serum and 1% penicillin-streptomycin. Cells were cultured and maintained in a humidified incubator at 37° C. under 5% C02. Cells were sub-cultured every 2-4 days. All cell culture supplies were obtained from Life Technologies (Grand Island, NY) and VWR (Radnor, PA).

Nitric Oxide Suppression Assay. RAW 264.7 cells were plated 1 day in advance of experimental treatments at a concentration of 30,000 cells per well onto Falcon-96 well clear bottom plates (Corning, NY) in a total volume of 200 μL per well using RPMI 1640 supplemented with 0.5% fetal bovine serum and 1% penicillin-streptomycin. The next day, cells were pretreated with compounds serially diluted from 1000×stocks. All compounds were dissolved in dimethyl sulfoxide (DMSO) usually at 10 mM stock solutions. Compounds were subsequently diluted in DMSO and RPMI 1640. Each well received a final concentration of 0.1% DMSO. Cells were pretreated for 2 hours and incubation at 37° C., followed by treatment with 20 ng/mL of interferon gamma (R&D Systems, Minneapolis, MN) per well for 24 hours. The next day, a nitrite standard was serially diluted from 100 μM to 1.6 μM in RPMI 1640. Afterwards, 50 μL of cell culture supernatant was transferred from each well into a new Falcon-96 well clear bottom plate. Nitrite was measured as surrogate for nitric oxide using Promega's Griess Detection Kit #G2930 (Madison, WI) which involves the addition of 50 μL of the provided sulfanilamide solution to each well of the transferred cell culture supernatant and standards, followed by a 10-minute incubation at room temperature. Next, 50 μL of the provided N-1-napthylethylenediamine dihydrochloride (NED) solution was added to the sulfanilamide reaction and incubated for 10 minutes at room temperature in the dark. Afterwards, air bubbles were removed using ethanol vapor and absorbance was measured using a Spectramax M2e plate reader with a wavelength set to 525 nm. Viability was assessed using WST-1 cell proliferation reagent from Roche (Basel, Switzerland). After media was removed for the Nitric Oxide suppression assay, 15 μL of WST-1 reagent was added to each well of cells. Plates were briefly mixed on an orbital shaker and the cells were incubated at 37° C. for 30-90 minutes. Absorbance was measured using a Spectramax M2e plate reader with wavelengths set to 440 nm and 700 nm.

For the ability of compounds to suppress the increase in nitric oxide release caused by interferon gamma, the absolute amount of nitrite that was produced in each well was extrapolated from the nitrite standards using a linear regression fit. All values were then normalized to the DMSO-interferon gamma treated wells and plotted as percent nitric oxide. IC₅₀ values were calculated using Excel and/or GraphPad Prism (San Diego, CA). The data is shown in Table 2.

Effect on Ludferase Reporter Activation. Nrf2 is a transcription factor that binds to the antioxidant response element (ARE) sequence in the promoter regions of its target genes. AREc32 reporter cell line (derived from human breast carcinoma MCF7 cells) was obtained was from CXR Bioscience Limited (Dundee, UK) and cultured in DMEM (low glucose) supplemented with 10% FBS, 1% penicillin/streptomycin, and 0.8 mg/ml Geneticin (G418). This cell line is stably transfected with a luciferase reporter gene under the transcriptional control of eight copies of the rat GSTA2 ARE sequence (5′-GTGACAAAGCA-3′) (Wang et al., 2006). Expression of Firefly luciferase from this reporter plasmid is controlled by binding of Nrf2 to these ARE sequences. Measurement of ARE-dependent luciferase activity allows quantitative assessment of Nrf2 induction. AREc32 cell line has previously been used in studies characterizing different Nrf2 activators (Dinkova-Kostova & Wang, 2011; Roubalová et al., 2016; Roubalová et al, 2017; Wu et al, 2012).

The effect of several compounds disclosed herein on luciferase reporter activation was assessed in the AREc32 reporter cell line (see Table 10 and Table 11). This cell line is derived from human breast carcinoma MCF-7 cells and is stably transfected with a luciferase reporter gene under the transcriptional control of eight copies of the antioxidant response element from the rat Gsta2 gene, an Nrf2 target gene (Frilling et al., 1990). AREc32 cells were plated in black 96-well plates in 200 μL media at 20,000 cells per well. Twenty-four hours after plating, cells were treated with vehicle (DMSO) or test compounds at concentrations ranging from 0.03 to 1000 nM for nineteen hours. Media was removed and 100 μL of 1:1 mixture of the One-Glo Luciferase assay reagent and culture medium was added to each well. After incubation for 5 min at room temperature, the luminescence signal was measured on a PHERAstar plate reader. The EC_{2X} value was determined using Excel and GraphPad Prism software. The fold increase in luminescence signal for cells treated with each concentration of compound relative to cells treated with vehicle was determined and a dose-response curve was generated. The dose-response curve was fit using nonlinear regression analysis and used to extrapolate the EC_{2X} value. The EC_{2X} value is defined as the concentration of test compound required to increase the luminescence signal 2-fold above levels in vehicle-treated samples.

TABLE 2
Nitric Oxide Inhibition and AREc32 EC2X Data

| | | | | Relative Avg |
|---|---|---|---|---|
| | | Avg | Avg | AREc32 |
| | Avg | NO IC₅₀ | AREc32 | EC2X |
| Com- | NO IC₅₀ | Relative to | EC2X | Relative to |
| pound | (nM) | RTA402 | (nM) | RTA402 |
| ID | (Mean ± SD) | (fold) | (Mean ± SD) | (fold) |
| T1 | 3.76 ± 1.35 | 2.53 ± 0.74 | 8.44 ± 1.56 | 0.744 ± 0.121 |
| T2 | 21.3 ± 7.4 | 9.86 ± 0.83 | 35 ± 2 | 3.41 ± 0.20 |
| T3 | 5.1 ± 1.02 | 2.51 ± 0.94 | 6.16 ± 1.02 | 0.598 ± 0.137 |
| T4 | 14.8 ± 2.9 | 7.06 ± 0.83 | 19.9 ± 0.2 | 1.93 ± 0.23 |
| T5 | 1.81 ± 0.331 | 1.24 ± 0.13 | 2.4 ± 0.75 | 0.224 ± 0.196 |
| T6 | 7.66 ± 1.52 | 3.67 ± 1.40 | 11.8 ± 1.9 | 1.03 ± 0.01 |
| T7 | 3.65 ± 1.87 | 1.62 ± 0.54 | 6.16 ± 0.70 | 0.595 ± 0.079 |
| T8 | 3.33 ± 0.68 | 1.59 ± 0.20 | 3.72 ± 0.97 | 0.321 ± 0.059 |
| T9 | 11.3 ± 2.4 | 5.37 ± 0.71 | 19.3 ± 4.5 | 1.84 ± 0.20 |
| T10 | 2.08 ± 0.14 | 1.45 ± 0.28 | 2.4 ± 0.3 | 0.193 ± 0.024 |
| T11 | 4.33 ± 1.45 | 2.10 ± 0.49 | 8.74 ± 2.61 | 0.685 ± 0.050 |
| T12 | 6.75 ± 2.19 | 3.29 ± 0.81 | 9.2 ± 2.7 | 0.724 ± 0.074 |
| T13 | 1.86 ± 0.32 | 1.25 ± 0.21 | 4.95 ± 0.63 | 0.358 ± 0.016 |
| T14 | 2.4 ± 1.2 | 1.27 ± 0.31 | 3.91 ± 0.52 | 0.349 ± 0.018 |
| T15 | 4.2 ± 2.4 | 1.99 ± 0.20 | 5.21 ± 1.11 | 0.447 ± 0.085 |
| T16 | 15 ± 3.7 | 7.63 ± 2.99 | 20.5 ± 6.0 | 1.61 ± 0.084 |
| T17 | 15 ± 5.8 | 5.83 ± 1.26 | 22.9 ± 2.6 | 1.92 ± 0.74 |
| T18 | 23 ± 4.1 | 13.7 ± 5.76 | 27.3 ± 5.8 | 1.97 ± 0.24 |
| T19 | 2.33 ± 1.47 | 0.74 ± 0.14 | 2.27 ± 0.52 | 0.224 ± 0.072 |
| T20 | 12.9 ± 4.6 | 3.72 ± 0.60 | 21.5 ± 4.6 | 1.83 ± 0.41 |
| T21 | 26 ± 7.6 | 10.9 ± 1.6 | 14.7 ± 1.3 | 1.27 ± 0.045 |
| T22 | 3.58 ± 2.16 | 0.94 ± 0.14 | 2.99 ± 1.62 | 0.244 ± 0.080 |
| T23 | 3.07 ± 1.43 | 0.80 ± 0.13 | 1.89 ± 0.91 | 0.166 ± 0.071 |
| T24 | 2.87 ± 1.67 | 0.967 ± 0.21 | 1.49 ± 0.31 | 0.126 ± 0.026 |
| T25 | 5.11 ± 1.69 | 1.49 ± 0.35 | 2.88 ± 0.13 | 0.259 ± 0.036 |
| T26 | 5.12 ± 2.22 | 1.71 ± 0.70 | 2.23 ± 0.29 | 0.177 ± 0.020 |
| T27 | 4.03 ± 1.66 | 1.15 ± 0.40 | 1.62 ± 0.22 | 0.144 ± 0.008 |
| T28 | 3.64 ± 1.69 | 1.15 ± 0.32 | 4.33 ± 1.62 | 0.331 ± 0.101 |
| T29 | 4.21 ± 1.75 | 1.47 ± 0.32 | 3.88 ± 1.19 | 0.317 ± 0.086 |
| T30 | 5.12 ± 1.21 | 1.33 ± 0.30 | 3.29 ± 0.61 | 0.27 ± 0.06 |
| T31 | 7.7 ± 3.1 | 2.25 ± 0.49 | 4.36 ± 0.81 | 0.373 ± 0.071 |
| T32 | 5.34 ± 1.15 | 2.52 ± 1.11 | 4.2 ± 1.3 | 0.399 ± 0.111 |
| T33 | 6.84 ± 1.59 | 2.32 ± 0.42 | 6.93 ± 1 | 0.621 ± 0.137 |
| T35 | 5.57 ± 2 | 1.66 ± 0.32 | 5.05 ± 1.1 | 0.442 ± 0.136 |
| T36 | 5.08 ± 1.51 | 1.25 ± 0.47 | 5.21 ± 1.17 | 0.441 ± 0.075 |
| T37 | 5.76 ± 1.38 | 2.08 ± 0.31 | 6.04 ± 2.15 | 0.486 ± 0.047 |
| T38 | 39.2 ± 4.2 | 9.52 ± 1.82 | 34.5 ± 7.6 | 2.76 ± 0.32 |
| T39 | 10.4 ± 2.6 | 3.38 ± 0.68 | 9.58 ± 2.31 | 0.799 ± 0.095 |
| T40 | 25.8 ± 9.3 | 9.8 ± 5.7 | 16.1 ± 1.5 | 1.2 ± 0.2 |
| T41 | 5.41 ± 2.52 | 1.79 ± 0.77 | 4.73 ± 2.12 | 0.354 ± 0.155 |
| T42 | 38.2 ± 10.0 | 14.80 ± 6.91 | 26.3 ± 6.0 | 2.43 ± 0.39 |
| T43 | 15.9 ± 7.0 | 5.93 ± 3.56 | 10.1 ± 1.0 | 0.8 ± 0.1 |
| T44 | 5.53 ± 1.8 | 2.03 ± 0.67 | 5.73 ± 1.79 | 0.53 ± 0.13 |
| T45 | 8.79 ± 2.72 | 3.22 ± 0.99 | 6.14 ± 1.47 | 0.563 ± 0.065 |
| T46 | 32.6 ± 2.2 | 8.4 ± 2.7 | 24.1 ± 6.7 | 2.06 ± 0.26 |
| T47 | 8.19 ± 2 | 2.70 ± 0.52 | 7.08 ± 1.2 | 0.573 ± 0.139 |
| T48 | 13.8 ± 1.5 | 3.66 ± 1.49 | 7.18 ± 1.2 | 0.602 ± 0.010 |
| T49 | 11.5 ± 2.6 | 3.77 ± 0.33 | 7.97 ± 0.73 | 0.643 ± 0.116 |
| T50 | 9.81 ± 2.79 | 3.18 ± 0.30 | 8.26 ± 1.39 | 0.666 ± 0.148 |
| T51 | 17.4 ± 2.5 | 5.79 ± 0.73 | 15.3 ± 3.6 | 1.2 ± 0.1 |
| T52 | 8.99 ± 1.84 | 3.35 ± 0.65 | 8.74 ± 0.51 | 0.654 ± 0.079 |
| T53 | 2.79 ± 0.59 | 0.82 ± 0.08 | 1.74 ± 0.47 | 0.173 ± 0.054 |
| T54 | 6.42 ± 1.07 | 1.84 ± 0.45 | 4.92 ± 1.14 | 0.403 ± 0.103 |
| T55 | 14.6 ± 3.0 | 5.21 ± 2.77 | 14.4 ± 1.3 | 1.12 ± 0.09 |
| T56 | 90.6 ± 40.6 | 35.5 ± 24.9 | 38.6 ± 7.07 | 2.99 ± 0.23 |
| T57 | 20.1 ± 6.0 | 8.81 ± 0.63 | 34.1 ± 4.98 | 2.73 ± 0.34 |
| T58 | 26 ± 8.8 | 8.67 ± 3.30 | 33.6 ± 3.3 | 2.64 ± 0.47 |
| T59 | 16 ± 5 | 5.03 ± 1.61 | 15.4 ± 1.2 | 1.23 ± 0.26 |
| T60 | 18.2 ± 6.2 | 4.94 ± 1.78 | 8.89 ± 1.13 | 0.791 ± 0.111 |
| T61 | 12.5 ± 7.3 | 4.11 ± 0.26 | 7.53 ± 2.25 | 0.601 ± 0.122 |
| T62 | 23.4 ± 5.4 | 8.76 ± 3.40 | 19.7 ± 2.0 | 1.55 ± 0.28 |
| T63 | 58.4 ± 9.1 | 23.3 ± 10.3 | 76 ± 9.6 | 6.8 ± 1.1 |
| T64 | 35.8 ± 6.1 | 14.80 ± 8.92 | 64 ± 7.6 | 6.07 ± 0.87 |
| T65 | 25.5 ± 4.1 | 7.42 ± 3.56 | 12.9 ± 2.0 | 1.14 ± 0.20 |
| T66 | 11 ± 3.0 | 4.64 ± 0.70 | 9.91 ± 0.25 | 0.857 ± 0.057 |
| T67 | 12.2 ± 3.5 | 3.79 ± 1.16 | 11.5 ± 1.6 | 0.984 ± 0.119 |
| T68 | 7.24 ± 0.77 | 2.70 ± 0.28 | 7.56 ± 0.77 | 0.575 ± 0.141 |
| T69 | 16.9 ± 5.0 | 4.94 ± 1.27 | 8.08 ± 1.98 | 0.673 ± 0.139 |
| T70 | 19.6 ± 6.1 | 8.82 ± 3.07 | 21.6 ± 1.3 | 1.78 ± 0.34 |
| T71 | 19.7 ± 1.8 | 13 ± 2.7 | 29.3 ± 8.1 | 2.95 ± 0.14 |
| T72 | 36.1 ± 13.8 | 20.20 ± 5.71 | 17.2 ± 7 | 1.71 ± 0.33 |
| T73 | 23.1 ± 6.6 | 7.02 ± 1.96 | 7.93 ± 0.82 | 0.681 ± 0.159 |
| T74 | 21 ± 7.6 | 5.91 ± 0.94 | 20 ± 2.0 | 1.67 ± 0.13 |
| T75 | 3.23 ± 0.65 | 1.46 ± 0.46 | 2.47 ± 0.64 | 0.242 ± 0.070 |
| T76 | 14.8 ± 2.8 | 9.13 ± 1.38 | 8.7 ± 0.63 | 1.1 ± 0.3 |
| T77 | 8.75 ± 4.81 | 2.31 ± 0.20 | 3.16 ± 1.11 | 0.263 ± 0.085 |
| T78 | 2.43 ± 0.95 | 0.683 ± 0.093 | 2.33 ± 0.78 | 0.194 ± 0.058 |
| T79 | 4.5 ± 1.84 | 1.25 ± 0.13 | 4.32 ± 0.50 | 0.361 ± 0.028 |
| T80 | 2.76 ± 1.02 | 1.09 ± 0.03 | 3.72 ± 0.14 | 0.311 ± 0.023 |
| T81 | 2.31 ± 0.55 | 0.92 ± 0.27 | 3.1 ± 1.5 | 0.238 ± 0.072 |
| T82 | 5.58 ± 0.32 | 3.69 ± 0.82 | 10 ± 3.0 | 1.01 ± 0.11 |
| T83 | 4.43 ± 2.68 | 2.75 ± 1.67 | 2.37 ± 0.33 | 0.294 ± 0.048 |
| T84 | 7.91 ± 2.32 | 4.07 ± 1.76 | 9.29 ± 1.52 | 1.14 ± 0.22 |
| T85 | 26.8 ± 4.7 | 9.84 ± 1.92 | 19 ± 3.5 | 1.63 ± 0.17 |
| T86 | 28.5 ± 6.5 | 7.09 ± 1.54 | 24.8 ± 6.4 | 2.12 ± 0.16 |
| T87 | 26.1 ± 7.6 | 6.55 ± 2.23 | 23.6 ± 0.9 | 2.0 ± 0.1 |
| T88 | 12.9 ± 5.1 | 6.48 ± 0.96 | 10.7 ± 3.2 | 1.03 ± 0.33 |
| T89 | 14.6 ± 2.2 | 8.03 ± 3.23 | 3.62 ± 1.73 | 0.345 ± 0.161 |
| T90 | 8.74 ± 2.77 | 4.60 ± 2.34 | 3.71 ± 0.99 | 0.455 ± 0.131 |
| T91 | 4.69 ± 1.87 | 2.44 ± 1.39 | 2.96 ± 0.77 | 0.359 ± 0.078 |
| T92 | 5.97 ± 2.71 | 3.68 ± 1.63 | 4.33 ± 0.75 | 0.555 ± 0.181 |
| T93 | 5.69 ± 1.93 | 3.06 ± 1.70 | 4.24 ± 0.66 | 0.525 ± 0.129 |
| T96 | 13.3 ± 3.7 | 7.08 ± 3.49 | 10.6 ± 0.7 | 1.32 ± 0.21 |
| T98 | 15.2 ± 4.3 | 4.45 ± 1.25 | 6.85 ± 0.51 | 0.574 ± 0.052 |
| T99 | 14.8 ± 4.7 | 5.92 ± 0.69 | 10.4 ± 0.8 | 0.864 ± 0.046 |
| T100 | 60.6 ± 0.9 | 28.1 ± 9.62 | 29.4 ± 3.69 | 2.87 ± 0.59 |
| T101 | 102 ± 15 | 67.7 ± 3.62 | 142 ± 29 | 15.8 ± 2.1 |
| T102 | 108 ± 19 | 36.5 ± 16.9 | 338 ± 11 | 31.2 ± 5.3 |
| T103 | 22.1 ± 4.2 | 14.60 ± 0.59 | 43.1 ± 10.4 | 4.52 ± 0.90 |
| T104 | 19.1 ± 4.9 | 12.6 ± 1.9 | 29.5 ± 2.5 | 3.15 ± 0.64 |
| T105 | 52.4 ± 13.8 | 36.7 ± 17 | 103 ± 37 | 10.7 ± 3.3 |
| T106 | 57.4 ± 15.7 | 37.4 ± 3.76 | 265 ± 21 | 28.2 ± 4.5 |
| T107 | 17.7 ± 6.9 | 7.07 ± 1.53 | 12.9 ± 1.5 | 1.17 ± 0.25 |

TABLE 3
Nitric Oxide Inhibition and AREc32 EC2X Data of Ursolic Acid Derivatives and their
Oleanolic Acid Derivative Analogs

| | | Avg | Avg |
|---|---|---|---|
| | | NO | ARE |
| | | IC50 | EC-2X |
| Compound | | (nM) | (nM) |
| ID | Structure | (Mean ± SD) | (Mean ± SD) |
| T2 | 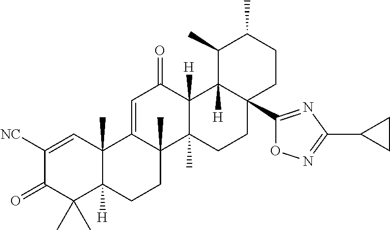 | 21.3 ± 7.4 | 35 ± 2 |
| T4 | 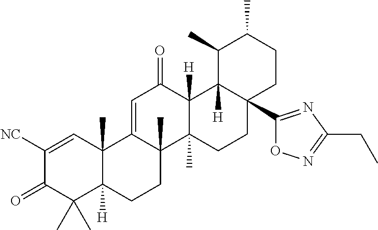 | 14.8 ± 2.9 | 19.9 ± 0.2 |
| T8 | 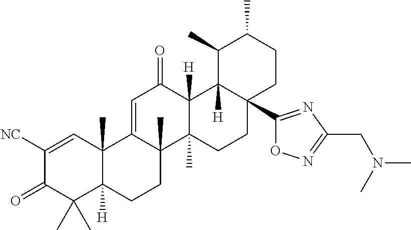 | 3.33 ± 0.68 | 3.72 ± 0.97 |
| T10 | 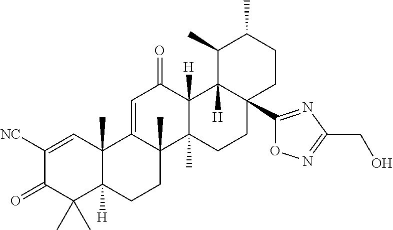 | 2.08 ± 0.14 | 2.4 ± 0.3 |
| T13 | 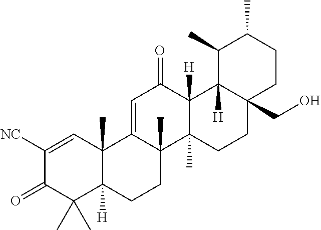 | 1.86 ± 0.32 | 4.95 ± 0.63 |
| T14 | 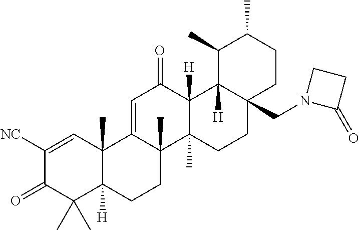 | 2.4 ± 1.2 | 3.91 ± 0.52 |
| T15 | 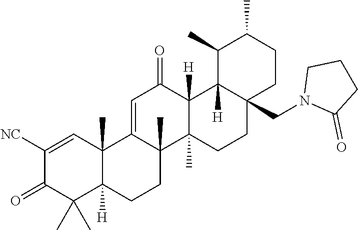 | 4.2 ± 2.4 | 5.21 ± 1.11 |
| T16 | 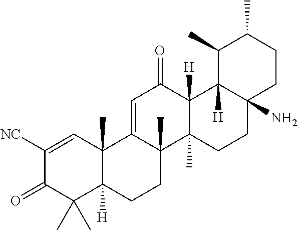 | 15 ± 3.7 | 20.5 ± 6.0 |
| T19 | 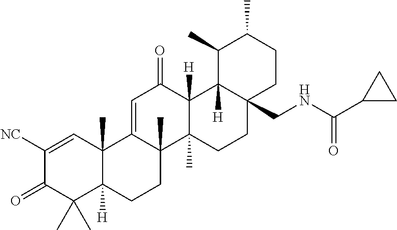 | 2.33 ± 1.47 | 2.27 ± 0.52 |
| T20 | 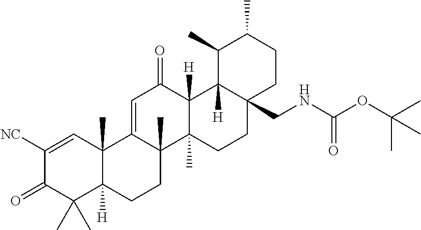 | 12.9 ± 4.6 | 21.5 ± 4.6 |
| T21 | 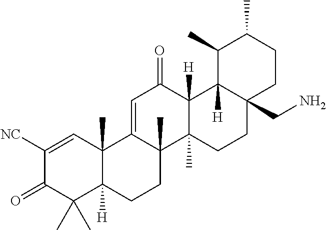 | 26 ± 7.6 | 14.7 ± 1.3 |
| T23 | 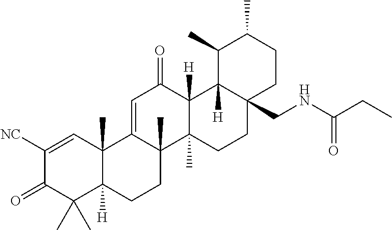 | 3.07 ± 1.43 | 1.89 ± 0.91 |
| T24 | 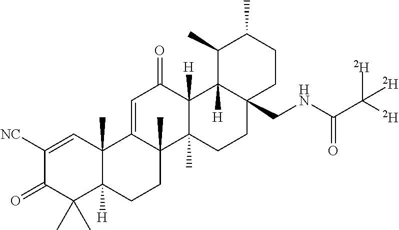 | 2.87 ± 1.67 | 1.49 ± 0.31 |
| T26 | 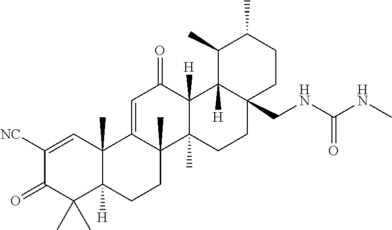 | 5.12 ± 2.22 | 2.23 ± 0.29 |
| T35 | 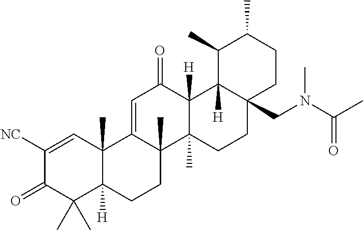 | 5.57 ± 2 | 5.05 ± 1.1 |
| T36 | 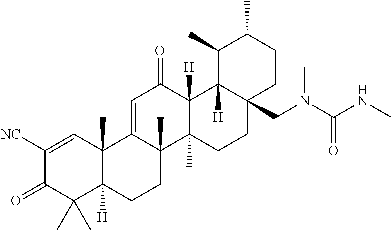 | 5.08 ± 1.51 | 5.21 ± 1.17 |
| T40 | 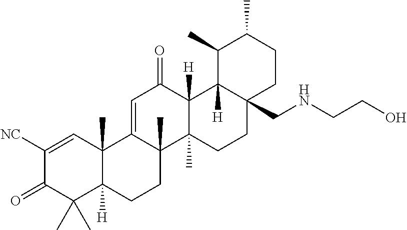 | 25.8 ± 9.3 | 16.1 ± 1.5 |
| T41 | 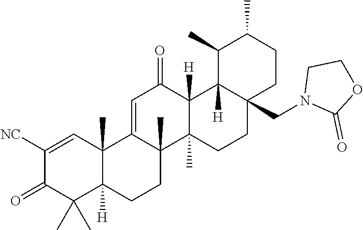 | 5.41 ± 2.52 | 4.73 ± 2.12 |
| T42 | 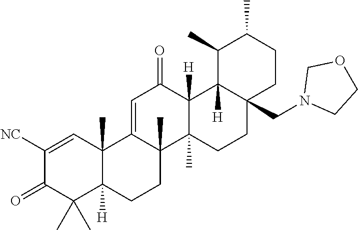 | 38.2 ± 10.0 | 26.3 ± 6.0 |
| T44 | 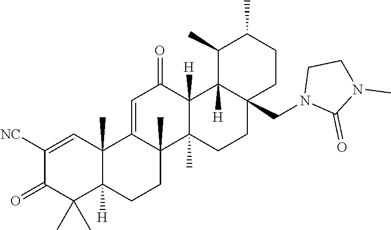 | 5.5 ± 1.8 | 5.73 ± 1.79 |
| T47 | 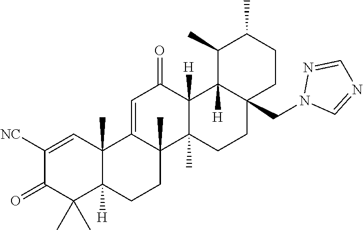 | 8.19 ± 2 | 7.1 ± 1.2 |
| T49 | 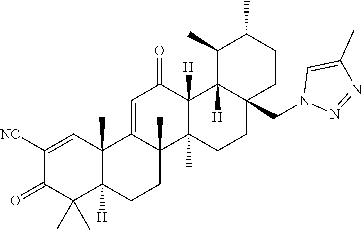 | 11.5 ± 2.6 | 7.97 ± 0.73 |
| T50 | 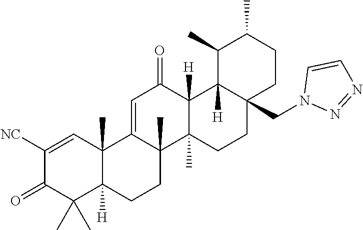 | 9.81 ± 2.79 | 8.26 ± 1.39 |
| T51 | 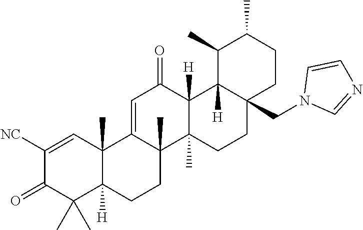 | 17.4 ± 2.5 | 15.3 ± 3.6 |
| T54 | 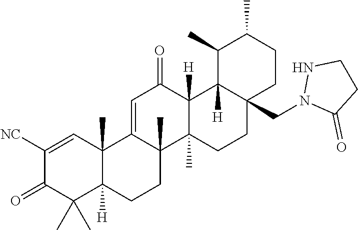 | 6.42 ± 1.07 | 4.92 ± 1.14 |
| T55 | 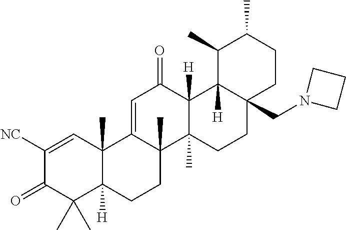 | 14.6 ± 3.0 | 14.4 ± 1.3 |
| T58 | 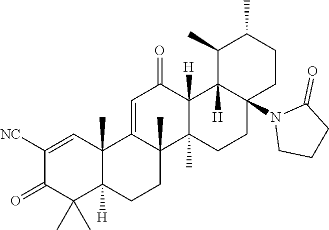 | 26 ± 8.8 | 33.6 ± 3.3 |
| T59 | 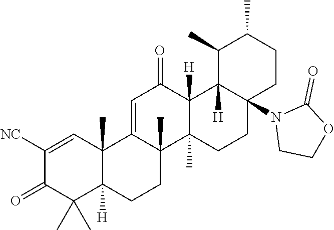 | 16 ± 5 | 15.4 ± 1.2 |
| T62 | 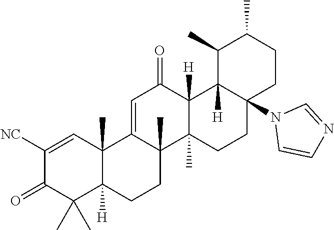 | 23.4 ± 5.4 | 19.7 ± 2.0 |
| T69 | 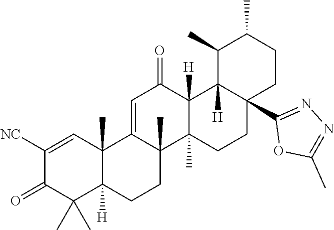 | 16.9 ± 5.0 | 8.08 ± 1.98 |
| T74 | 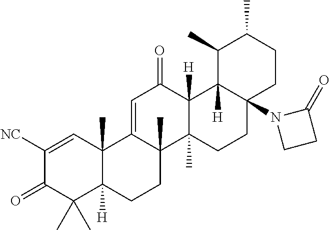 | 21 ± 7.6 | 20 ± 2.0 |
| T82 | 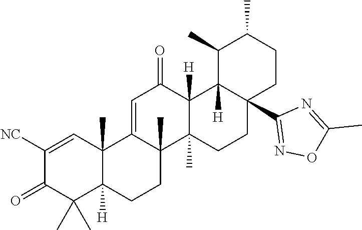 | 5.58 ± 0.32 | 10 ± 3.0 |
| | | Avg | Avg |
| | | NO | ARE |
| Oleanolic | | IC50 | EC-2X |
| analog ID | Structure | (nM) | (nM) |
| CC2 | 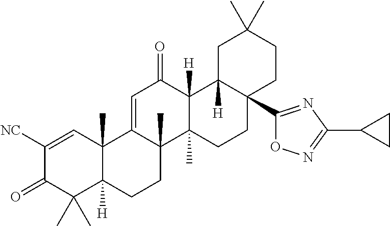 | 4.03 | 43.7 |
| CC4 | 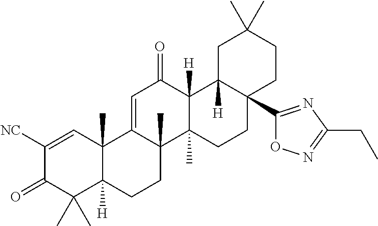 | 5.3 | 21.4 |
| CC8 | 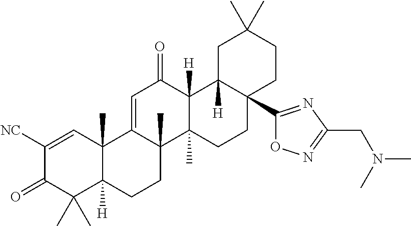 | 3.3 | 3.72 |
| CC10 | 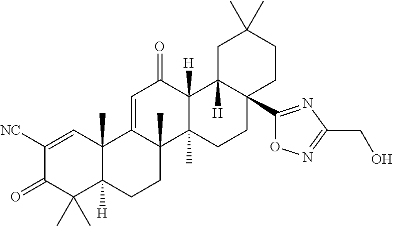 | 2.1 | 3.39 |
| CC13 | 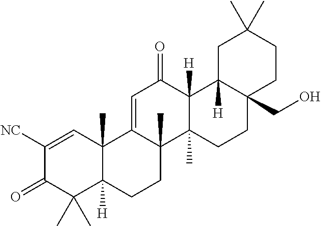 | 0.5 | 8.82 |
| CC14 | 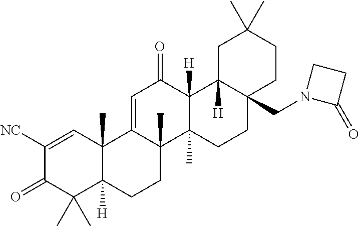 | 1.1 | 5.24 |
| CC15 | 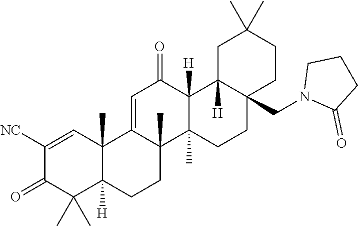 | 1.1 | 6.66 |
| CC16 | 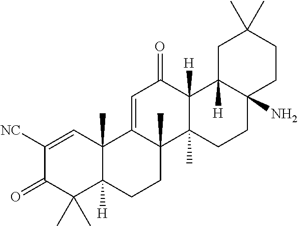 | 3.5 | 27.7 |
| CC19 | 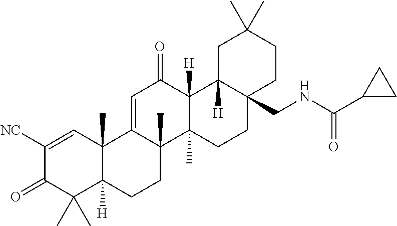 | 0.5 | 3.22 |
| CC20 | 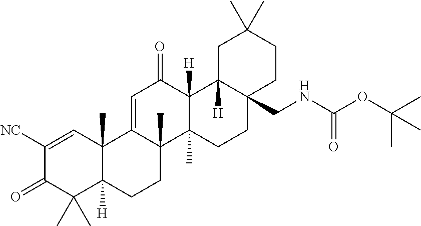 | 5.0 | 21.1 |
| CC21 | 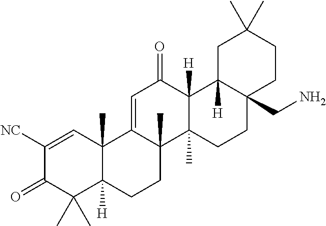 | 3.2 | 15.8 |
| CC23 | 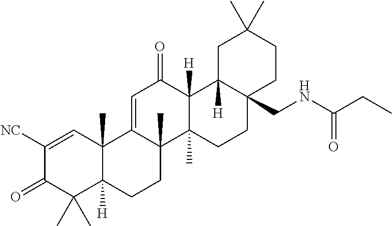 | 0.4 | 2.37 |
| CC24 | 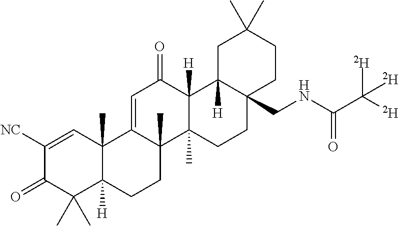 | 0.5 | 2.96 |
| CC26 | 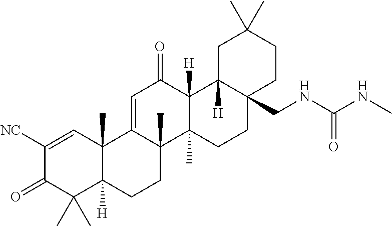 | 0.5 | 2.75 |
| CC35 | 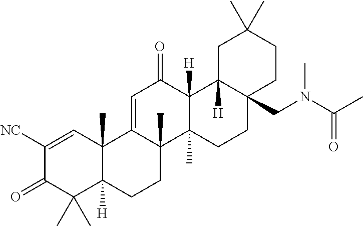 | 0.8 | 6.81 |
| CC36 | 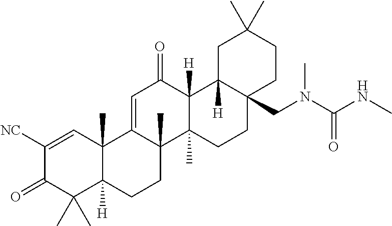 | 0.7 | 7.23 |
| CC40 | 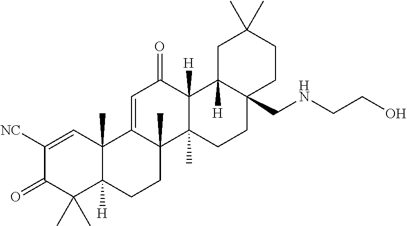 | 7.5 | 21.8 |
| CC41 | 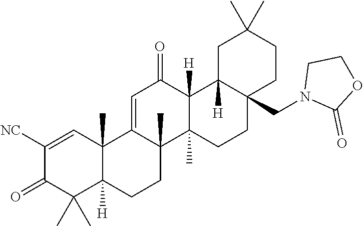 | 1.7 | 5.12 |
| CC42 | 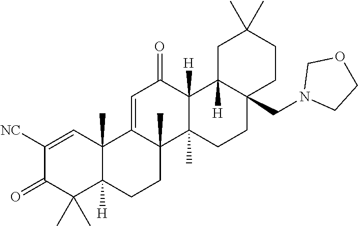 | 17.3 | 122 |
| CC44 | 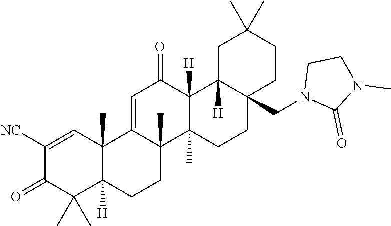 | 1.2 | 6.07 |
| CC47 | 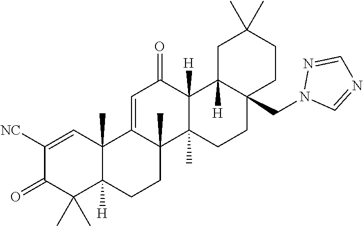 | 1.2 | 10.7 |
| CC49 | 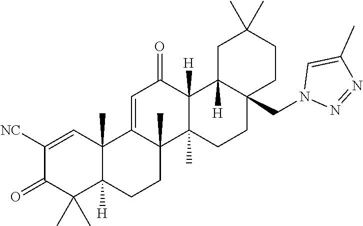 | 2.5 | 13.8 |
| CC50 | 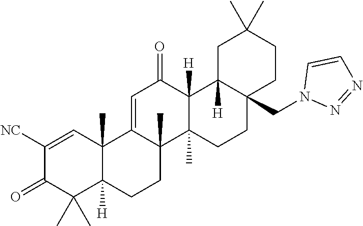 | 1.1 | 9.57 |
| CC51 | 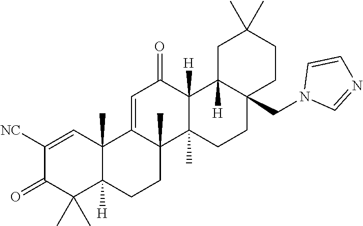 | 3.9 | 18 |
| CC54 | 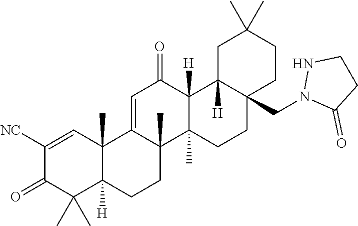 | 8.6 | 11.7 |
| CC55 | 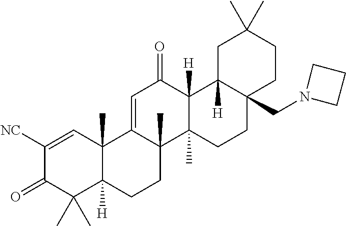 | 2.5 | 22 |
| CC58 | 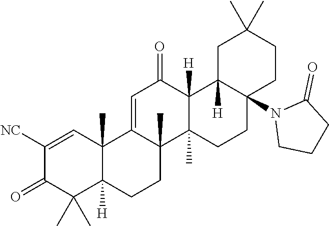 | 3.6 | 37.9 |
| CC59 | 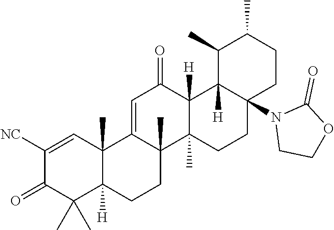 | 2.2 | 23.6 |
| CC62 | 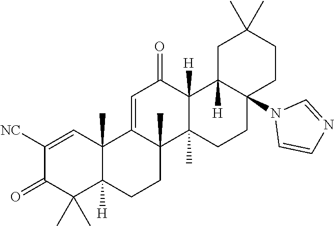 | 3.2 | 26.8 |
| CC69 | 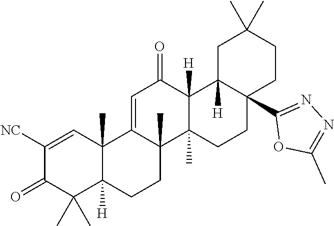 | 2.2 | 9.81 |
| CC74 | 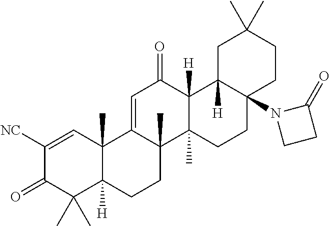 | 4.3 | 36.1 |
| CC82 | 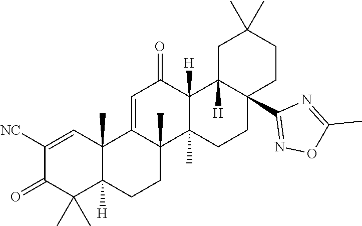 | 3.8 | 22.1 |

All the compounds, formulations, and methods disclosed and claimed herein can be made and executed without undue experimentation in light of the present disclosure. While the compounds, formulations, and methods of this invention have been described in terms of preferred embodiments, it will be apparent to those of skill in the art that variations may be applied to the compounds, formulations, and methods, as well as in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references to the extent that they provide exemplary procedural or other details supplementary to those set forth herein, are specifically incorporated herein by reference.
U.S. Pat. No. 7,915,402
U.S. Pat. No. 7,943,778
U.S. Pat. No. 8,071,632
U.S. Pat. No. 8,124,799
U.S. Pat. No. 8,129,429
U.S. Pat. No. 8,338,618
U.S. Pat. No. 8,993,640
U.S. Pat. No. 9,512,094
U.S. Pat. No. 9,701,709
U.S. Pat. No. 9,889,143
WO 2009/129546
WO 2012/125488
WO 2014/040056
Abraham and Kappas, Free Radical Biol. Med., 39:1-25, 2005.
Ahmad et al. Cancer Res., 68:2920-26, 2008.
Ahmad et al., J. Biol. Chem., 281:35764-9, 2006.
Anderson, Practical Process Research & Development—A Guide for Organic Chemists, 2^{nd} ed., Academic Press, New York, 2012.
Araujo et al., J. Immunol., 171(3):1572-80, 2003.
Bach, Hum. Immunol., 67(6):430-32, 2006.
Chauhan and Chauhan, Pathophysiology, 13(3):171-181 2006.
Dickerson et al., Prog Neuropsychopharmacol Biol. Psychiatry, Mar. 6, 2007.
Dinkova-Kostova et al. Proc. Natl. Acad. Sci. USA, 102(12):4584-89, 2005.
Dinkova-Kostova & Wang. Chem. Biol. Interact., 192(1-2):101-6, 2011.
Dudhgaonkar et al., Eur. J. Pain, 10(7):573-9, 2006.
Favaloro et al., J. Med. Chem., 45:4801-05, 2002.
Forstermann, Biol. Chem., 387:1521, 2006.
Fu et al., Org. Biomol. Chem., 12(28):5192-5200, 2014.
Handbook of Pharmaceutical Salts: Properties, and Use. Stahl and Wermuth Eds., Verlag Helvetica Chimica Acta, 2002.
Hanson et al., BMC Medical Genetics, 6(7), 2005.
Honda et al. Bioorg. Med. Chem. Lett., 12:1027-30, 2002.
Honda et al., Bioorg. Med. Chem. Lett., 16(24):6306-09, 2006.
Honda et al., Bioorg. Med. Chem. Lett., 7:1623-28, 1997.
Honda et al., Bioorg. Med. Chem. Lett., 8(19):2711-14, 1998.
Honda et al., Bioorg. Med. Chem. Lett., 9(24):3429-34, 1999.
Honda et al., J. Med. Chem., 43:4233-46, 2000a.
Honda et al., Org. Biomol. Chem., 1:4384-91, 2003.
Honda, et al., J. Med. Chem., 43:1866-77, 2000b.
Honda, et al., J. Med. Chem., 54(6):1762-78, 2011.
Hong, et al., 2012.
Ishikawa et al., Circulation, 104(15):1831-36, 2001.
Kawakami et al., Brain Dev., 28(4):243-46, 2006.
Kendall-Tackett, Trauma Violence Abuse, 8(2):117-26, 2007.
Kruger et al., J. Pharmacol. Exp. Ther., 319(3):1144-52, 2006.
Lee et al., Glia., 55(7):712-22, 2007.
Lencz et al., Mol. Psychiatry, 12(6):572-80, 2007.
Liby et al., Cancer Res., 65(11):4789-98, 2005.
Liby et al., Mol. Cancer Ther., 6(7):2113-19, 2007b.
Liby et al., Nat. Rev. Cancer, 7(5):357-69, 2007a.
Liu et al., FASEB J., 20(2):207-16, 2006.
Lu et al., J. Clin. Invest., 121(10):4015-29, 2011.
McIver et al., Pain, 120(1-2):161-69, 2005.
Morris et al., J. Mol. Med., 80(2):96-104, 2002.
Morse and Choi, Am. J. Respir. Crit. Care Med., 172(6):660-70, 2005.
Morse and Choi, Am. J. Respir. Crit. Care Med., 27(1):8-16, 2002.
Pall, Med. Hypoth., 69:821-25, 2007.
Pergola et al., N. Engl. J. Med., 365(4):327-36, 2011.
Place et al., Clin. Cancer Res., 9(7):2798-806, 2003.
Rajakariar et al., Proc. Natl. Acad. Sci. USA, 104(52):20979-84, 2007.
Reagan-Shaw et al., FASEB J., 22(3):659-61, 2008
Reisman et al., Arch. Dermatol. Res., 306(5):447-54, 2014.
Ross et al., Am. J. Clin. Pathol., 120(Suppl): S53-71, 2003.
Ross et al., Expert Rev. Mol. Diagn., 3(5):573-85, 2003.
Roubalová et al., Chem. Bio. Interact., 260:58-66, 2016.
Roubalová \acute{ } et al., Fitoterapia, 119:115-20, 2017.
Ruster et al., Scand. J. Rheumatol., 34(6):460-63, 2005.
Sacerdoti et al., Curr Neurovasc Res. 2(2):103-11, 2005.
Salvemini et al., J. Clin. Invest., 93(5):1940-47, 1994.
Sarchielli et al., Cephalalgia. 26(9):1071-79, 2006.
Satoh et al., Proc. Natl. Acad. Sci. USA, 103(3):768-73, 2006.
Schulz et al., Antioxid. Redox. Sig., 10:115, 2008.
Smith, March's Advanced Organic Chemistry: Reactions, Mechanisms, and Structure, 7^{th} Ed., Wiley, 2013.
Suh et al., Cancer Res., 58:717-23, 1998.
Suh et al., Cancer Res., 59(2):336-41, 1999.
Szabo et al., Nature Rev. Drug Disc., 6:662-80, 2007.
Takahashi et al., Cancer Res., 57:1233-37, 1997.
Tanir and Tannebaum, Biochim. Biophys. Acta, 1288: F31-F36, 1996.
Wang et al., Cancer Res., 66:10983-94, 2006.
Wu et al., PloS One, 7(10): e44686, 2012.
Xie T et al., J Biol Chem., 270(12):6894-900, 1995.
Zhou et al., Am. J. Pathol., 166(1):27-37, 2005.

What is claimed is:

1. A compound of the formula: 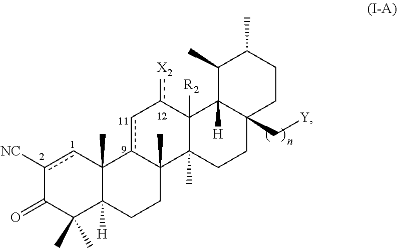
wherein:
the bond between atom 1 and atom 2 is a single bond, a double bond, or an epoxidized double bond;
the bond between atom 9 and atom 11 is a single bond or a double bond;
the bond between atom 12 and X₂ is a single bond or a double bond;
n is -6;
X₂ is oxo, or X₂ is taken together with Y as defined below, provided that when X₂ is oxo, then the bond between atom 12 and X₂ is a double bond, and when X₂ is taken together with Y as defined below, that the bond between atom 12 and X₂ is a single bond
R₂ is hydrogen or hydroxy; or R₂ is taken together with Y as defined below; and
Y is hydrogen, hydroxy, halo, or amino; or
alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups;
arenediyl_{(C≤12)}—R₃, substituted -arenediyl_{(C≤12)}—R₃,
heteroarenediyl_{(C≤12)}—R₃, or substituted -heteroarenediyl_{(C≤12)}—R₃,
wherein:
R₃ is alkyl_{(C≤12)}, cycloalkyl_{(C≤12)}, heterocycloalkyl_{(C≤12)}, or a substituted version of any of these groups; or
alkanediyl_{(C≤12)}—R₄ or substituted -alkanediyl_{(C≤12)}—R₄, wherein:
R₄ is alkoxy_{(C≤12)}, alkylamino_{(C≤2)}, dialkylamino_{(C≤12)}, or a substituted version of any of these groups; or —alkanediyl_{(C≤12)}—C(O)R₅, or substituted -alkanediyl_{(C≤12)}—C(O)R₅,
wherein:
R₅ is hydroxy or amino; or
alkoxy_{(C≤12)}, alkylamino_{(C≤12)}, dialkylamino_{(C≤12)}, cycloalkyl_{(C≤12)}, cycloalkoxy_{(C≤12)}, cycloalkylamino_{(C≤12)}, heterocycloalkyl_{(C≤12)}, or a substituted version of any of these groups;
C(O)R₇, wherein:
R₇ is hydrogen, heterocycloalkyl_{(C≤12)}, substituted heterocycloalkyl_{(C≤12)}, cycloalkylamino_{(C≤12)}, substituted cycloalkylamino_{(C≤12)}, —NHC(NH)-alkyl_{(C≤12)}, or —NHOR₁₃, wherein:
R₁₃ is hydrogen, alkyl_{(C≤12)}, or substituted alkyl_{(C≤12)};
NR₈R₉, wherein:
R₈ is hydrogen, alkyl_{(C≤12)}, or substituted alkyl_{(C≤12)}; and
R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)}, or
CO₂R₁₀, wherein:
R₁₀ is hydrogen, alkyl_{(C≤12)}, cycloalkyl_{(C≤12)}, or a substituted version of any of these groups; or
C(O)R₁₂, wherein:
R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups; or
CH═NOR₁₁, wherein:
R₁₁ is hydrogen, alkyl_{(C≤12)}, or substituted alkyl_{(C≤12)}; or
Y is taken together with R₂ and is —(CH₂)ₘX₁—, wherein:
m is 0-6; and
X₁ is —O—; or
Y is taken together with X₂ and is —(CH₂)oC(O)—, wherein:
is 0-6; and
X₂ is —O—;
or a pharmaceutically acceptable salt thereof.

2. The compound of claim 1, wherein the compound is further defined as: 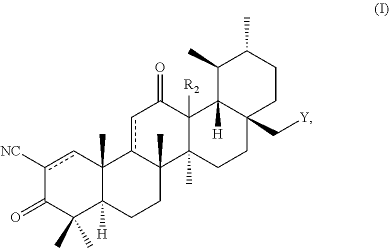
wherein:
the bond between atom 1 and atom 2 is a single bond, a double bond, or an epoxidized double bond;
the bond between atom 9 and atom 11 is a single bond or a double bond;
n is 0-6;
R₂ is hydrogen or hydroxy; or R₂ is taken together with Y as defined below; and
Y is hydrogen, hydroxy, halo, or amino; or
alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups; —arenediyl_{(C≤12)}—R₃, substituted -arenediyl_{(C≤12)}—R₃, —heteroarenediyl_{(C≤12)}—R₃, or substituted -heteroarenediyl_{(C≤12)}—R₃,
wherein:
R₃ is alkyl_{(C≤12)}, cycloalkyl_{(C≤12)}, heterocycloalkyl_{(C≤12)}, or a substituted version of any of these groups; or
-alkanediyl_{(C≤12)}—R₄ or substituted -alkanediyl_{(C≤12)}—R₄, wherein:
R₄ is alkoxy_{(C≤12)}, alkylamino_{(C≤12)}, dialkylamino_{(C≤12)}, or a substituted version of any of these groups; or
alkanediyl_{(C≤12)}—C(O)R₅ or substituted -alkanediyl_{(C≤12)}—C(O)R₃, wherein:
R₅ is hydroxy or amino; or
alkoxy_{(C≤12)}, alkylamino_{(C≤12)}, dialkylamino_{(C≤12)}, cycloalkyl_{(C≤12)}, cycloalkoxy_{(C≤12)}, cycloalkylamino_{(C≤12)}, heterocycloalkyl_{(C≤12)}, or a substituted version of any of these groups; or
C(O)R₇, wherein:
R₇ is hydrogen, heterocycloalkyl_{(C≤12)}, substituted heterocycloalkyl_{(C≤12)}, cycloalkylamino_{(C≤12)}, substituted cycloalkylamino_{(C≤12)}, —NHC(NH)-alkyl_{(C≤12)}, or —NHOR₁₃, wherein:
R₁₃ is hydrogen, alkyl, or substituted alkyl;
NR₈R₉, wherein:
R₈ is hydrogen, alkyl_{(C≤12)}, or substituted alkyl_{(C≤12)}; and
R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)},
CO₂R₁₀, wherein:
R₁₀ is hydrogen, alkyl_{(C≤12)}, cycloalkyl_{(C≤12)}, or a substituted version of any of these groups; or
C(O)R₁₂, wherein:
R₁₂ is hydrogen, amino, alkylamino_{(C≤12)}, dialkylamino_{(C≤12)}, cycloalkylamino_{(C≤12)}, heterocycloalkyl_{(C≤12)} or a substituted version of any of these groups;
CH═NOR₁₁, wherein:
R₁₁ is hydrogen, alkyl_{(C≤12)}, or substituted alkyl_{(C≤12)}; or
Y is taken together with R₂ and is —(CH₂)ₘX₁—, wherein:
m is 0-6; and
X₁ is —O—;
or a pharmaceutically acceptable salt thereof.

3. The compound of either claim 1 or claim 2, wherein the compound is further defined as: 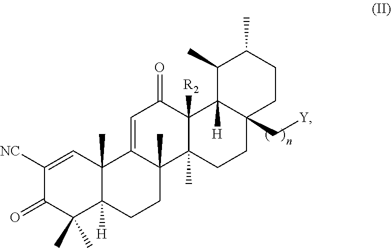
wherein:
n is 0-6;
R₂ is hydrogen or hydroxy; or R₂ is taken together with Y as defined below; and
Y is hydrogen, hydroxy, halo, or amino; or
alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups; or —arenediyl_{(C≤12)}—R₃, substituted -arenediyl_{(C≤12)}—R₃,
heteroarenediyl_{(C≤12)}—R₃, or substituted -heteroarenediyl_{(C≤12)}—R₃,
wherein:
R₃ is alkyl_{(C≤12)}, cycloalkyl_{(C≤12)}, heterocycloalkyl_{(C≤12)}, or a substituted version of any of these groups; or
alkanediyl_{(C≤12)}—R₄ or substituted -alkanediyl_{(C≤12)}—R₄, wherein:
R₄ is alkoxy_{(C≤12)}, alkylamino_{(C≤12)}, dialkylamino_{(C≤12)}, or a substituted version of any of these groups; or
alkanediyl_{(C≤12)}—C(O)R₅ or substituted -alkanediyl_{(C≤12)}—C(O)R₅, wherein:
R₅ is hydroxy or amino; or
alkoxy_{(C≤12)}, alkylamino_{(C≤12)}, dialkylamino_{(C≤12)}, cycloalkyl_{(C≤12)}, cycloalkoxy_{(C≤12)}, cycloalkylamino_{(C≤12)}, heterocycloalkyl_{(C≤12)}, or a substituted version of any of these groups; or
C(O)R₇, wherein:
R₇ is hydrogen, heterocycloalkyl_{(C≤12)}, substituted heterocycloalkyl_{(C≤12)}, cycloalkylamino_{(C≤12)}, substituted cycloalkylamino_{(C≤12)}, —NHC(NH)-alkyl_{(C≤12)}, or —NHOR₁₃, wherein:
R₁₃ is hydrogen, alkyl, or substituted alkyl;
NR₈R₉, wherein:
R₈ is hydrogen, alkyl_{(C≤12)}, or substituted alkyl_{(C≤12)}; and
R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)},
CO₂R₁₀, wherein:
R₁₀ is hydrogen, alkyl_{(C≤12)}, cycloalkyl_{(C≤12)}, or a substituted version of any of these groups; or
C(O)R₁₂, wherein:
R₁₂ is hydrogen, amino, alkylamino_{(C≤12)}, dialkylamino_{(C≤12)}, cycloalkylamino_{(C≤12)}, heterocycloalkyl_{(C≤12)} or a substituted version of any of these groups;
CH═NOR₁₁, wherein:
R₁₁ is hydrogen, alkyl_{(C≤12)}, or substituted alkyl_{(C≤12)}; or
Y is taken together with R₂ and is —(CH₂)ₘX₁—, wherein:
m is 0-6; and
X₁ is -0;
or a pharmaceutically acceptable salt thereof.

4. The compound according to any one of claims 1-3, wherein the compound is further defined as: 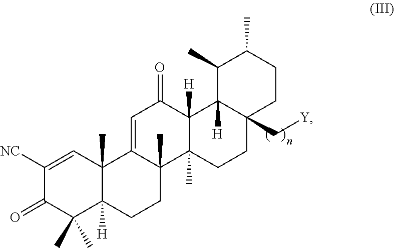
wherein:
n is -6;
Y is hydrogen, hydroxy, halo, or amino; or
alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups; or —arenediyl_{(C≤12)}—R₃, substituted -arenediyl_{(C≤12)}—R₃, —heteroarenediyl_{(C≤12)}—R₃, or substituted -heteroarenediyl_{(C≤12)}—R₃,
wherein:
R₃ is alkyl_{(C≤12)}, cycloalkyl_{(C≤12)}, heterocycloalkyl_{(C≤12)}, or a substituted version of any of these groups; or
-alkanediyl_{(C≤12)}—R₄ or substituted -alkanediyl_{(C≤12)}—R₄, wherein:
R₄ is alkoxy_{(C≤12)}, alkylamino_{(C≤12)}, dialkylamino_{(C≤12)}, or a substituted version of any of these groups; or
alkanediyl_{(C≤12)}—C(O)R₅ or substituted -alkanediyl_{(C≤12)}—C(O)R₅, wherein:
R₅ is hydroxy or amino; or alkoxy_{(C≤12)}, alkylamino_{(C≤12)}, dialkylamino_{(C≤12)}, cycloalkyl_{(C≤12)}, heterocycloalkyl_{(C≤12)}, or a substituted version of any of these groups; or
C(O)R₇, wherein:
R₇ is hydrogen, heterocycloalkyl_{(C≤12)}, substituted heterocycloalkyl_{(C≤12)}, cycloalkylamino_{(C≤12)}, substituted cycloalkylamino_{(C≤12)}, —NHC(NH) -alkyl_{(C≤12)}, or —NHOR₁₃, wherein:
R₁₃ is hydrogen, alkyl, or substituted alkyl;
NR₈R₉, wherein:
R₈ is hydrogen, alkyl_{(C≤12)}, or substituted alkyl_{(C≤12)}; and
R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)},
CO₂R₁₀, wherein:
R₁₀ is hydrogen, alkyl_{(C≤12)}, cycloalkyl_{(C≤12)}, or a substituted version of any of these groups; or
C(O)R₁₂, wherein:
R₁₂ is hydrogen, amino, alkylamino_{(C≤12)}, dialkylamino_{(C≤12)}, cycloalkylamino_{(C≤12)}, heterocycloalkyl_{(C≤12)} or a substituted version of any of these groups;
CH═NOR₁₁, wherein:
R₁₁ is hydrogen, alkyl_{(C≤12)}, or substituted alkyl_{(C≤12)};
or a pharmaceutically acceptable salt thereof.

5. The compound of claim 1 or claim 2, wherein the bond between atom 1 and atom 2 is a double bond or an epoxidized double bond.

6. The compound of either claim 1, claim 2, or claim 5, wherein the bond between atom 1 and atom 2 is a double bond.

7. The compound of either claim 1, claim 2, or claim 5, wherein the bond between atom 1 and atom 2 is an epoxidized double bond.

8. The compound according to either claim 1 or claim 2, wherein the bond between atom 9 and atom 11 is a single bond.

9. The compound according to either claim 1 or claim 2, wherein the bond between atom 9 and atom 11 is a double bond.

10. The compound according to any one of claims 1 or 5—11 wherein X₂ is oxo.

11. The compound according to any one of claims 1-10, wherein n is 0,1, or 2.

12. The compound according to any one of claims 1-11, wherein n is 0.

13. The compound according to any one of claims 1-11, wherein n is 1.

14. The compound according to any one of claims 1-11, wherein n is 2.

15. The compound according to any one of claims 1-14, wherein Y is hydrogen, hydroxy, halo, or amino; or
alkyl_{(C≤12)}, alkenyl_{(C≤12)}, alkynyl_{(C≤12)}, alkylamino_{(C≤12)}, dialkylamino_{(C≤12)}, amido_{(C≤12)}, acyloxy_{(C≤12)}, cycloalkyl_{(C≤12)}, heterocycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups; or
—arenediyl_{(C≤12)}—R₃, substituted -arenediyl_{(C≤12)}—R₃, -heteroarenediyl_{(C≤12)}—R₃, or
substituted -heteroarenediyl_{(C≤12)}—R₃, wherein:
R₃ is alkyl_{(C≤12)}, cycloalkyl_{(C≤12)}, heterocycloalkyl_{(C≤12)}, or a substituted version of any of these groups; or
—alkanediyl_{(C≤12)}—R₄ or substituted -alkanediyl_{(C≤12)}—R₄, wherein:
R₄ is alkoxy_{(C≤12)}, alkylamino_{(C≤12)}, dialkylamino_{(C≤12)}, or a substituted version of any of these groups; or —alkanediyl_{(C≤12)}C(O)—R₅ or substituted -alkanediyl_{(C≤12)}—C(O)R₅, wherein:
R₅ is hydroxy or amino; or
alkoxy_{(C≤12)}, alkylamino_{(C≤12)}, dialkylamino_{(C≤12)}, cycloalkyl_{(C≤12)}, cycloalkoxy_{(C≤12)}, cycloalkylamino_{(C≤12)}, heterocycloalkyl_{(C≤12)}, or a substituted version of any of these groups.

16. The compound according to any one of claims 1-15, wherein Y is amino; or
alkyl_{(C≤12)}, amido_{(C≤12)}, heterocycloalkyl_{(C≤12)}, heteroaryl_{(C≤12)}, or a substituted version of any of these groups; or —heteroarenediyl_{(C≤12)}—R₃ or substituted -heteroarenediyl_{(C≤12)}—R₃, wherein:
R₃ is alkyl_{(C≤12)}, substituted alkyl_{(C≤12)}, cycloalkyl_{(C≤12)}, or substituted cycloalkyl_{(C≤12)}; or
alkanediyl_{(C≤12)}—R₄ or substituted -alkanediyl_{(C≤12)}—R₄, wherein:
R₄ is alkoxy_{(C≤12)} or substituted alkoxy_{(C≤12)}.

17. The compound according to any one of claims 1-16, wherein Y is amino.

18. The compound according to any one of claims 1-16, wherein Y is alkyl_{(C≤12)} or substituted alkyl_{(C≤12)}.

19. The compound according to any one of claims 1-16 and 18, wherein Y is substituted alkyl_{(C≤12)}.

20. The compound according to any one of claims 1-16, 18, and 19, wherein Y is hydroxymethyl, or methylaminomethyl.

21. The compound according to any one of claims 1-16, wherein Y is amido_{(C≤12)} or substituted amido_{(C≤12)}.

22. The compound according to any one of claims 1-16 and 21, wherein Y is amido_{(C≤12)}.

23. The compound according to any one of claims 1-16, 21, and 22, wherein Y is acetamido or propionamido.

24. The compound according to any one of claims 1-16 and 21, wherein Y is substituted amido_{(C≤12)}.

25. The compound according to any one of claims 1-16, 21, and 24, wherein Y is 2,2-difluoropropionamido.

26. The compound according to any one of claims 1-16, wherein Y is heterocycloalkyl_{(C≤12)} or substituted heterocycloalkyl_{(C≤12)}.

27. The compound according to any one of claims 1-16 and 26, wherein Y is heterocycloalkyl_{(C≤12)}.

28. The compound according to any one of claims 1-16, 26, and 27, wherein Y is oxazolidin-3-yl or azetidin-1-yl.

29. The compound according to any one of claims 1-16 and 26, wherein Y is substituted heterocycloalkyl_{(C≤12)}.

30. The compound according to any one of claims 1-16, 26, and 29, wherein Y is selected from the group of: 2-oxooxazolidin-3-yl, 3-methyl-2-oxoimidazolidin-1-yl, 2-oxoimidazolidin-1-yl, 2,5-dioxopyrrolidin-1-yl, methyl 3-oxopyrazolidine-1-carboxylate, 5-oxopyrazolidin-1-yl, 2-oxoazetidin-1-yl and 2-oxopyrrolidin-1-yl.

31. The compound according to any one of claims 1-16, wherein Y is heteroaryl_{(C≤12)} or substituted heteroaryl_{(C≤12)}.

32. The compound according to any one of claims 1-16 and 31, wherein Y is heteroaryl_{(C≤12)}.

33. The compound according to any one of claims 1-16, 31, and 32, wherein Y is 3-methyl-1,2,4-oxadiazol-5-yl, 3-ethyl-1,2,4-oxadiazol-5-yl, 1H-pyrazol-1-yl, 1H-1,2,4-triazol-1-yl, 4-methyl-1H-1,2,3-triazol-1-yl, 1H-tetrazol-1-yl, 1H-1,2,3-triazol-1-yl, 1H-imidazol-1-yl, 5-methyl-1,3,4-oxadiazol-2-yl, or 5-methyl-1,2,4-oxadiazol-3-yl.

34. The compound according to any one of claims 1-16 and 31, wherein Y is substituted heteroaryl_{(C≤12)}.

35. The compound according to any one of claims 1-16, 31, and 34, wherein Y is 4-bromo-1H-pyrazol-1-yl, 3-(2-methoxyethyl)-1,2,4-oxadiazol-5-yl, 3-(methoxymethyl)-1,2,4-oxadiazol-5-yl, 3-(2-hydroxyethyl)-1,2,4-oxadiazol-5-yl, 3-(hydroxymethyl)-1,2,4-oxadiazol-5-yl, 3-((dimethylamino)methyl)-1,2,4-oxadiazol-5-yl, 3-(1-methoxyethyl)-1,2,4-oxadiazol-5-yl, or 3-(fluoromethyl)-1,2,4-oxadiazol-5-yl.

36. The compound according to any one of claims 1-16, wherein Y is —heteroarenediyl_{(C≤12)}—R₃ or substituted -heteroarenediyl_{(C≤12)}—R₃.

37. The compound according to any one of claims 1-16 and 36, wherein Y is —heteroarenediyl_{(C≤12)}—R₃.

38. The compound according to any one of claims 1-16, 36, and 37, wherein Y is a group of the formula:

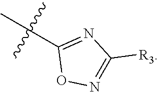

39. The compound according to any one of claims 1-16, 36, and 37, wherein Y is a group of the formula:

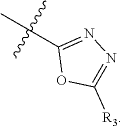

40. The compound according to any one of claims 1-16, 36, and 37, wherein Y is a group of the formula:

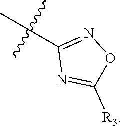

41. The compound according to any one of claims 1-16 and 36—40, wherein R₃ is alkyl_{(C≤12)} or substituted alkyl_{(C≤12)}.

42. The compound according to any one of claims 1-16 and 36—41, wherein R₃ is alkyl_{(C≤12)}.

43. The compound according to any one of claims 1-16 and 36-42, wherein R₃ is methyl or ethyl.

44. The compound according to any one of claims 1-16 and 36-41, wherein R₃ is substituted alkyl_{(C≤12)}.

45. The compound according to any one of claims 1-16, 36-41, and 44, wherein R₃ is 2-methoxyethyl, methoxymethyl, 2-hydroxyethyl, hydroxymethyl, (dimethylamino)methyl, 1-methoxyethyl, or fluoromethyl.

46. The compound according to any one of claims 1-16, 41, and 44, wherein R₃ is polar-substituted alkyl_{(C≤12)}.

47. The compound according to any one of claims 1-16, 41, 44, and 46, wherein R₃ is monopolar-substituted alkyl_{(C≤12)}.

48. The compound according to any one of claims 1-16, 41, 46, and 47 44, wherein R₃ is monoaminoalkyl_{(C≤12)}, monofluoroalkyl_{(C≤12)}, or monohydroxyalkyl_{(C≤12)}.

49. The compound according to any one of claims 1-16, 41, 44, and 46-48, wherein R₃ is monofluoroalkyl_{(C≤12)} or monohydroxyalkyl_{(C≤12)}.

50. The compound according to any one of claims 1-16, 41, 44, and 46-49, wherein R₃ is monohydroxyalkyl_{(C≤12)}.

51. The compound according to any one of claims 1-16, 41, 44, and 46-50, wherein R₃ is 2-hydroxyethyl or hydroxymethyl.

52. The compound according to any one of claims 1-16, 41, 44, and 46-49, wherein R₃ is monofluoroalkyl_{(C≤12)}.

53. The compound according to any one of claims 1-16, 41, 44, and 46-50, wherein R₃ is fluoromethyl.

54. The compound according to any one of claims 1-16 and 36-40, wherein R₃ is cycloalkyl_{(C≤12)} or substituted cycloalkyl_{(C≤12)}.

55. The compound according to any one of claims 1-16 and 36-40, and 54, wherein R₃ is cycloalkyl_{(C≤12)}.

56. The compound according to any one of claims 1-16 and 36-40, 54, and 55, wherein R₃ is cyclopropyl.

57. The compound according to any one of claims 1-16 and 36-40, wherein R₃ is —alkanediyl_{(C≤12)}—R₄ or substituted -alkanediyl_{(C≤12)}—R₄.

58. The compound according to any one of claims 1-16 and 36-40, and 57, wherein R₃ is —methanediyl-R₄.

59. The compound according to any one of claims 1-16 and 36-40, 57, and 58, wherein R₄ is alkoxy_{(C≤12)}.

60. The compound according to any one of claims 1-16 and 36-40, and 57-59, wherein R₄ is t-butoxy.

61. The compound according to any one of claims 1-15, wherein Y is -alkanediyl_{(C≤12)}—C(O)R₅ or substituted -alkanediyl_{(C≤12)}—C(O)R₅, wherein:
R₅ is hydroxy or amino; or
alkoxy_{(C≤12)}, alkylamino_{(C≤12)}, dialkylamino_{(C≤12)}, cycloalkyl_{(C≤12)}, cycloalkoxy_{(C≤12)}, cycloalkylamino_{(C≤12)}, heterocycloalkyl_{(C≤12)}, or a substituted version of any of these groups.

62. The compound according to any one of claims 1-15 and 61, wherein R₅ is hydroxy.

63. The compound according to any one of claims 1-15 and 61, wherein R₅ is amino.

64. The compound according to any one of claims 1-15 and 61, wherein R₅ is alkoxy_{(C≤12)}.

65. The compound according to any one of claims 1-15, 57, or 64, wherein R₅ is methoxy.

66. The compound according to any one of claims 1-15 and 61, wherein R₅ is alkylamino_{(C≤12)}.

67. The compound according to any one of claims 1-15, 61, or 66, wherein R₅ is methylamino or ethylamino.

68. The compound according to any one of claims 1-15, and 61, wherein R₅ is substituted alkylamino_{(C≤12)}.

69. The compound according to any one of claims 1-15, 61, and 68, wherein R₅ is 2,2-difluoroethan-1-amino.

70. The compound according to any one of claims 1-15 and 61, wherein R₅ is cycloalkyl_{(C≤12)} or substituted cycloalkyl_{(C≤12)}.

71. The compound according to any one of claims 1-15, 61, and 70, wherein R₅ is cycloalkyl_{(C≤12)}.

72. The compound according to any one of claims 1-15, 61, 70, and 71, wherein R₅ is cyclopropyl_{(C≤12)}.

73. The compound according to any one of claims 1-15 and 61, wherein R₅ is cycloalkylamino_{(C≤12)}, or substituted cycloalkylamino_{(C≤12)}.

74. The compound according to any one of claims 1-15, 61, and 73, wherein R₅ is cycloalkylamino_{(C≤12)}.

75. The compound according to any one of claims 1-15, 61, 73, and 74, wherein R₅ is cyclopropylamino.

76. The compound according to any one of claims 1-15 and 61, wherein R₅ is heterocycloalkyl_{(C≤12)} or substituted heterocycloalkyl_{(C≤12)}.

77. The compound according to any one of claims 1-15, 61, and 76, wherein R₅ is heterocycloalkyl_{(C≤12)}.

78. The compound according to any one of claims 1-15,61, 76, and 77, wherein R₅ is azetidino or pyrrolidine.

79. The compound according to any one of claims 1-14, wherein Y is:
—C(O)R₇, wherein:
R₇ is hydrogen, heterocycloalkyl_{(C≤12)}, cycloalkylamino_{(C≤12)} or substituted cycloalkylamino_{(C≤12)}, —NHC(NH) -alkyl_{(C≤12)}, or —NHOR₁₃, wherein:
R₁₃ is hydrogen, alkyl, or substituted alkyl;
—NR₈R₉, wherein:
R₈ is hydrogen, alkyl_{(C≤12)}, or substituted alkyl_{(C≤12)}; and
R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)}, or
—CO₂R₁₀, wherein:
R₁₀ is hydrogen, alkyl_{(C≤12)}, or a substituted version of any of these groups; or
—C(O)R₁₂, wherein:
R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups; or
—CH═NOR₁₁, wherein:
R₁₁ is hydrogen, alkyl_{(C≤12)}, or substituted alkyl_{(C≤12)}.

80. The compound according to any one of claims 1-14 and 79, wherein Y is:
—C(O)R₇, wherein:
R₇ is hydrogen, heterocycloalkyl_{(C≤12)}, substituted heterocycloalkyl_{(C≤12)}, cycloalkylamino_{(C≤12)} or substituted cycloalkylamino_{(C≤12)}, —NHC(NH) —alkyl_{(C≤12)}, or —NHOR_{13(C≤12)}, wherein:
R₁₃ is hydrogen, alkyl, or substituted alkyl.

81. The compound according to any one of claims 1-14, 79, and 80, wherein R₇ is hydrogen.

82. The compound according to any one of claims 1-14, 79, and 80, wherein R₇ is cycloalkylamino_{(C≤12)} or substituted cycloalkylamino_{(C≤12)}.

83. The compound according to any one of claims 1-14, 79, 80, and 82, wherein R₇ is cycloalkylamino_{(C≤12)}.

84. The compound according to any one of claims 1-14, 79, 80, 82, and 83, wherein R₇ i: cyclopropylamino.

85. The compound according to any one of claims 1-14, 79, and 80, wherein R₇ is heterocycloalkyl_{(C≤12)} or substituted heterocycloalkyl_{(C≤12)}.

86. The compound according to any one of claims 1-14, 79, 80, and 85, wherein R₇ is heterocycloalkyl_{(C≤12)}.

87. The compound according to any one of claims 1-14, 79, 80, 85, and 86, wherein R₇ is azetidine or pyrrolidine.

88. The compound according to any one of claims 1-14, 79, and 80, wherein R₇ is —NHC(NH)— alkyl_{(C≤12)},

89. The compound according to any one of claims 1-14, 79, 80, and 88, wherein R₇ is —NHC(NH)CH₃.

90. The compound according to any one of claims 1-14, 79, and 80, wherein R₇ is —NHOR_{13(C≤12)}, wherein:
R₁₃ is hydrogen, alkyl_{(C≤12)}, or substituted alkyl_{(C≤12)}.

91. The compound according to any one of claims 1-14, 79, 80, and 90, wherein R₁₃ is hydrogen.

92. The compound according to any one of claims 1-14, 79, 80, and 90, wherein R₁₃ is alkyl_{(C≤12)}.

93. The compound according to any one of claims 1-14, 79, 80, 90, and 92, wherein R₁₃ is methyl.

94. The compound according to any one of claims 1-14 and 79, wherein Y is:
—NR₈R₉, wherein:
R₈ is hydrogen, alkyl_{(C≤12)}, or substituted alkyl_{(C≤12)}; and
R₉ is acyl_{(C≤12)}, substituted acyl_{(C≤12)}, alkylsulfonyl_{(C≤12)}, substituted alkylsulfonyl_{(C≤12)}, cycloalkylsulfonyl_{(C≤12)}, substituted cycloalkylsulfonyl_{(C≤12)}; or —CO₂R₁₀, wherein R₁₀ is hydrogen, alkyl_{(C≤12)}, or substituted alkyl_{(C≤12)}, or a substituted version of any of these groups; or —C(O)R₁₂, wherein:
R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups.

95. The compound according to any one of claims 1-14, 79, and 94, wherein R₈ is hydrogen.

96. The compound according to any one of claims 1-14, 79, and 94, wherein R₈ is alkyl_{(C≤12)} or substituted alkyl_{(C≤12)}.

97. The compound according to any one of claims 1-14, 79, 94, and 96, wherein R₈ is alkyl_{(C≤12)}.

98. The compound according to any one of claims 1-14, 79, 94, 96, and 97, wherein R₈ is methyl.

99. The compound according to any one of claims 1-14, 79, and 94-98, wherein R₉ is acyl_{(C≤12)} or substituted acyl_{(C≤12)}.

100. The compound according to any one of claims 1-14, 79, and 94-99, wherein R₉ is acyl_{(C≤12)}.

101. The compound according to any one of claims 1-14, 79, and 94-100, wherein R₉ is acetyl, methylacetyl, cyclopropanelcarboxyl, or cyclobutanecarboxyl.

102. The compound according to any one of claims 1-14, 79, and 94-99, wherein R₉ is substituted acyl_{(C≤12)}.

103. The compound according to any one of claims 1-14, 79, 94-99, and 102, wherein R₉ is methylaminocarbonyl, difluoroacetyl, or difluoromethylacetyl.

104. The compound according to any one of claims 1-14, 79, and 94-98, wherein R₉ is alkylsulfonyl_{(C≤12)}, substituted alkylsulfonyl_{(C≤12)}, cycloalkylsulfonyl_{(C≤12)}, or substituted cycloalkylsulfonyl_{(C≤12)}.

105. The compound according to any one of claims 1-14, 79, 94-98, and 104, wherein R₉ is alkylsulfonyl_{(C≤12)}.

106. The compound according to any one of claims 1-14, 79, 94-98, 104, and 105, wherein R₉ is methylsulfonyl or ethylsulfonyl.

107. The compound according to any one of claims 1-14, 79, 94-98, and 104, wherein R₉ i: cycloalkylsulfonyl_{(C≤12)}.

108. The compound according to any one of claims 1-14, 79, 94-98, 104, and 107, wherein R₉ is cyclopropylsulfonyl_{(C≤12)}.

109. The compound according to any one of claims 1-14, 79, and 94-98, wherein R₉ is —CO₂R₁₀, wherein R₁₀ is hydrogen, alkyl_{(C≤12)}, or substituted alkyl_{(C≤12)}, or a substituted version of any of these groups.

110. The compound according to any one of claims 1-14, 79, 94-98, and 109, wherein R₁₀ is alkyl_{(C≤12)} or substituted alkyl_{(C≤12)}.

111. The compound according to any one of claims 1-14, 79, 94-98, 109, and 110, wherein R₁₀ is alkyl_{(C≤12)}.

112. The compound according to any one of claims 1-14, 79, 94-98, and 109-111, wherein R₁₀ is methyl or t-butyl.

113. The compound according to any one of claims 1-14, 79, and 94-98, wherein R₉ is —C(O)R₁₂, wherein:
R₁₂ is hydrogen, amino, alkylamino_{(C≤12)}, dialkylamino_{(C≤12)}, cycloalkylamino_{(C≤12)}, heterocycloalkyl_{(C≤12)}, or a substituted version of any of these groups.

114. The compound according to any one of claims 1-14, 79, 94-98, and 113, wherein R₁₂ is hydrogen.

115. The compound according to any one of claims 1-14, 79, 94-98, and 113, wherein R₁₂ is amino.

116. The compound according to any one of claims 1-14 79, 94-98, and 113, wherein R₁₂ is alkylamino_{(C≤12)},or substituted alkylamino_{(C≤12)}.

117. The compound according to any one of claims 1-14 79, 94-98, 113, and 116, wherein R₁₂ is alkylamino_{(C≤12)}.

118. The compound according to any one of claims 1-14 79, 94-98, 113, 116, and 117, wherein R₁₂ is methylamino or ethylamino.

119. The compound according to any one of claims 1-14, 79, 94-98, and 113, wherein R₁₂ is dialkylamino_{(C≤12)},or substituted dialkylamino_{(C≤12)}.

120. The compound according to any one of claims 1-14, 79, 94-98, and 113, wherein R₁₂ is cycloalkylamino_{(C≤12)} or substituted cycloalkylamino_{(C≤12)}.

121. The compound according to any one of claims 1-14, 79, 94-98, 113, and 120, wherein R₁₂ is cycloalkylamino_{(C≤12)}.

122. The compound according to any one of claims 1-14, 79, 94-98, 113, 120, and 121, wherein R₁₂ is cyclopropylamino.

123. The compound according to any one of claims 1-14, 79, 94-98, and 113, wherein R₁₂ is heterocycloalkyl_{(C≤12)} or substituted heterocycloalkyl_{(C≤12)}.

124. The compound according to any one of claims 1-14, 79, 94-98, 113, and 123, wherein R₁₂ is heterocycloalkyl_{(C≤12)}.

125. The compound according to any one of claims 1-14, 79, 94-98, 113, 123 and 124, wherein R₁₂ is azetidine.

126. The compound according to any one of claims 1-14 and 79, wherein Y is: —CH═NOR₁₁, wherein:
R₁₁ is hydrogen, alkyl_{(C≤12)}, or substituted alkyl_{(C≤12)}.

127. The compound according to any one of claims 1-14, 79, and 126, wherein R₁₁ is hydrogen,

128. The compound according to any one of claims 1-14 79, and 126 wherein R₁₁ is alkyl_{(C≤12)} or substituted alkyl_{(C≤12)}.

129. The compound of claim 128, wherein R₁₁ is alkyl_{(C≤12)}.

130. The compound of claim 129, wherein R₁₁ is methyl.

131. The compound according to any one of claims 1-3 and 5-14, wherein Y is taken together with R₂ and is —(CH₂)ₘX₁—, wherein:
m is 0-6; and
X₁ is —O—.

132. The compound of claim 94, wherein Y is taken together with R₂ and is —(CH₂)ₘX₁—, wherein:
m is 1 and
X₁ is —O—.

133. The compound of claim 94, wherein Y is taken together with R₂ and is —(CH₂)ₘX₁—, wherein:
m is 2.
X₁ is —O—.

134. The compound according to any one of claims 1 and 5-14, wherein Y is taken together with X₂ and is —(CH₂)ₒC(O)—, wherein:
is 0-6.

135. The compound according to any one of claims 1 and 5-14, wherein Y is taken together with X₂ and is —(CH₂)ₒC(O)—, wherein:
is 1.

136. The compound according to any one of claims 1-135, wherein the compound is further defined as:

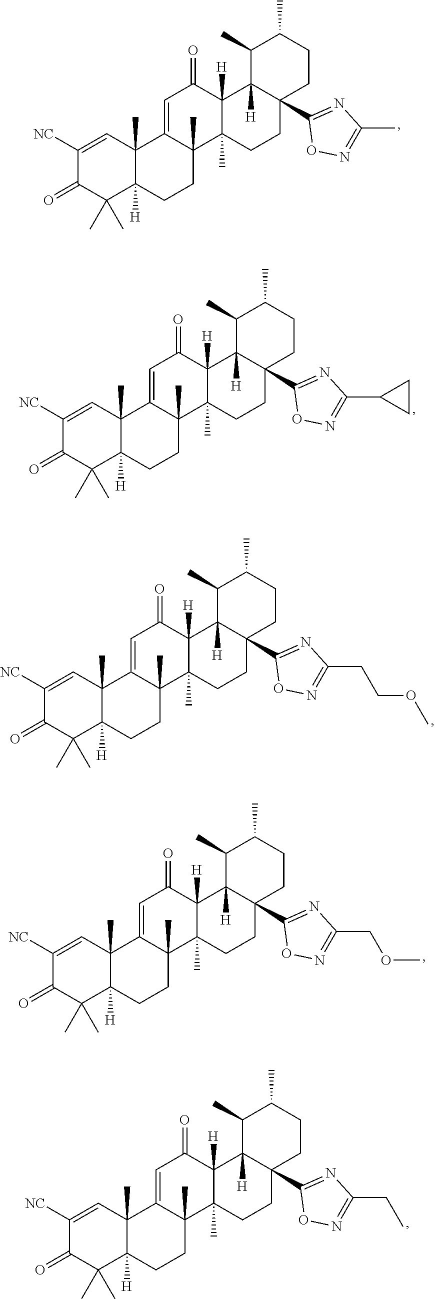

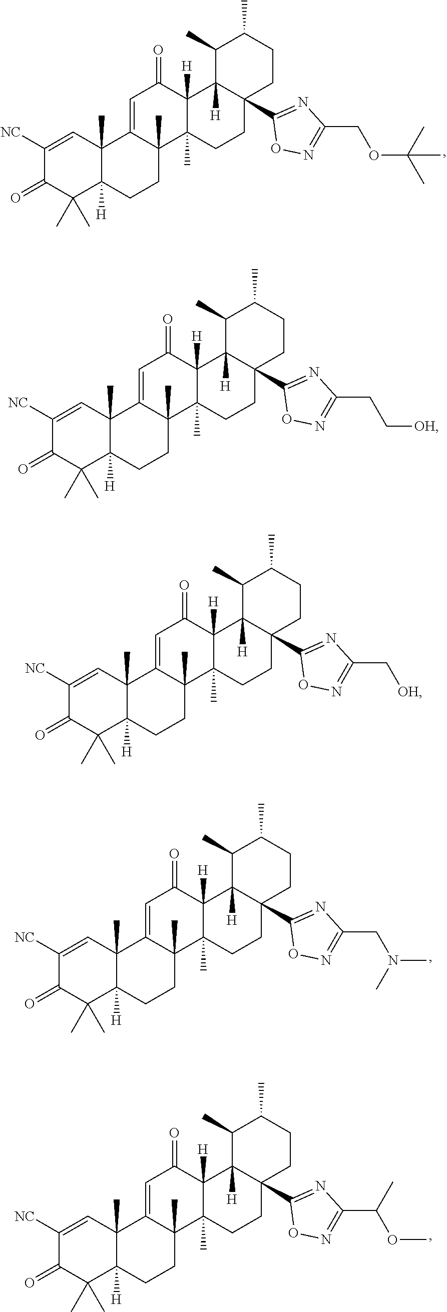

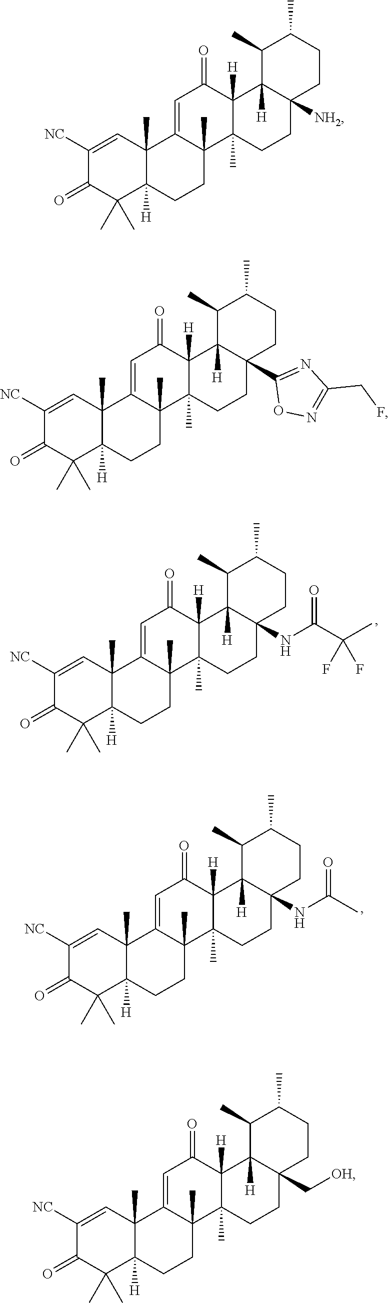

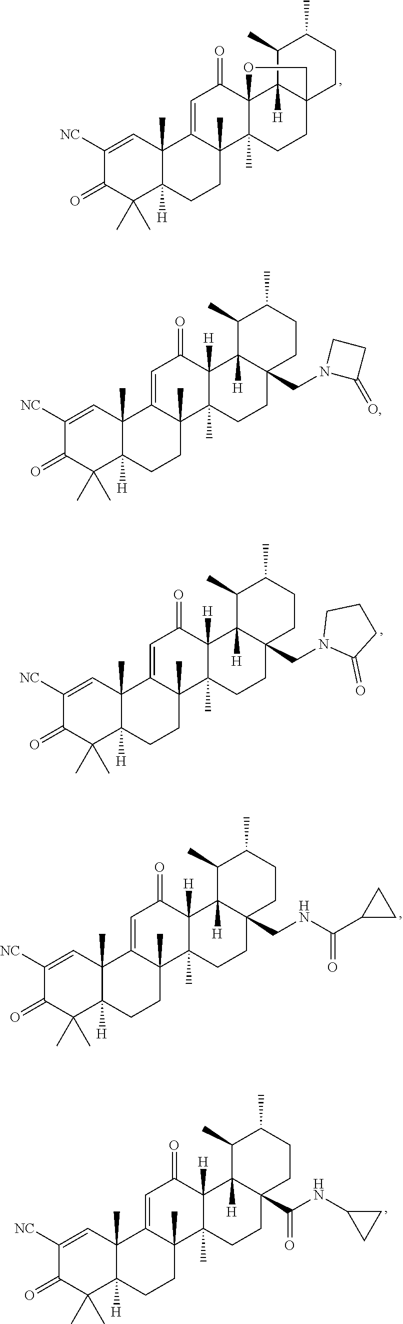

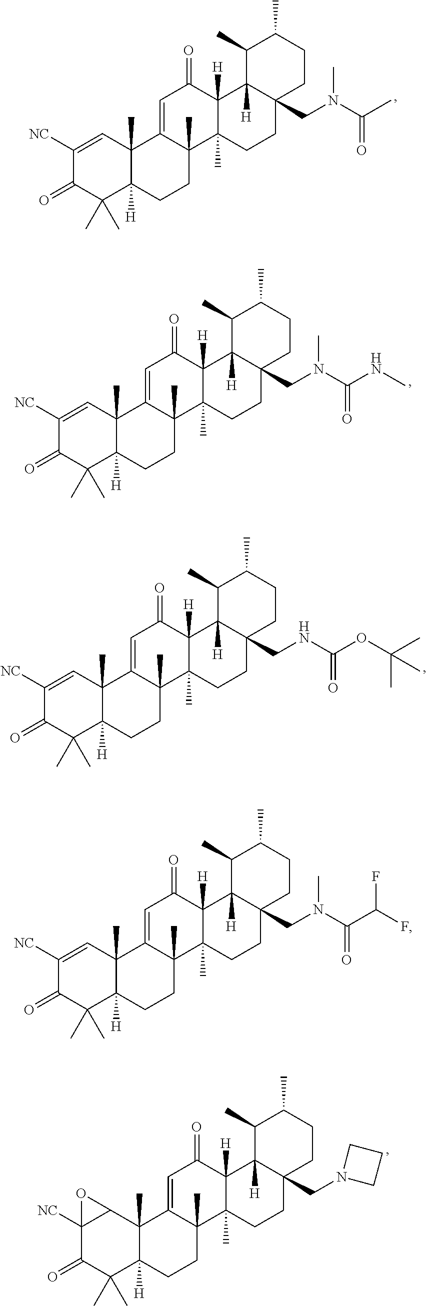

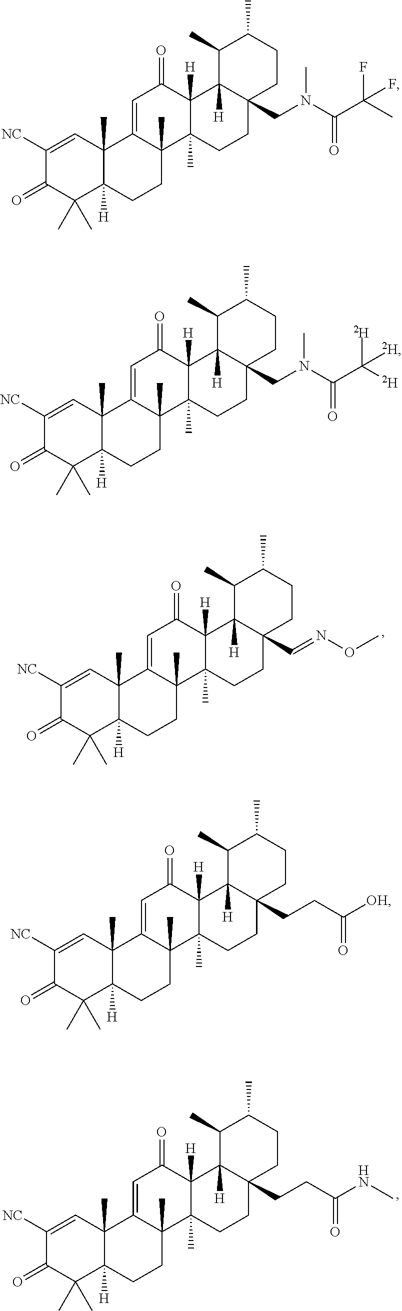

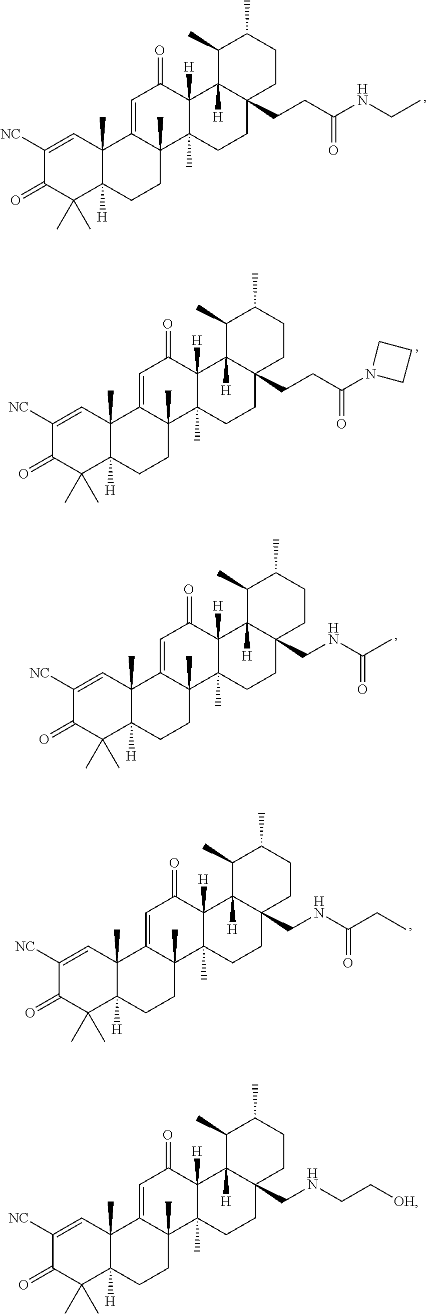

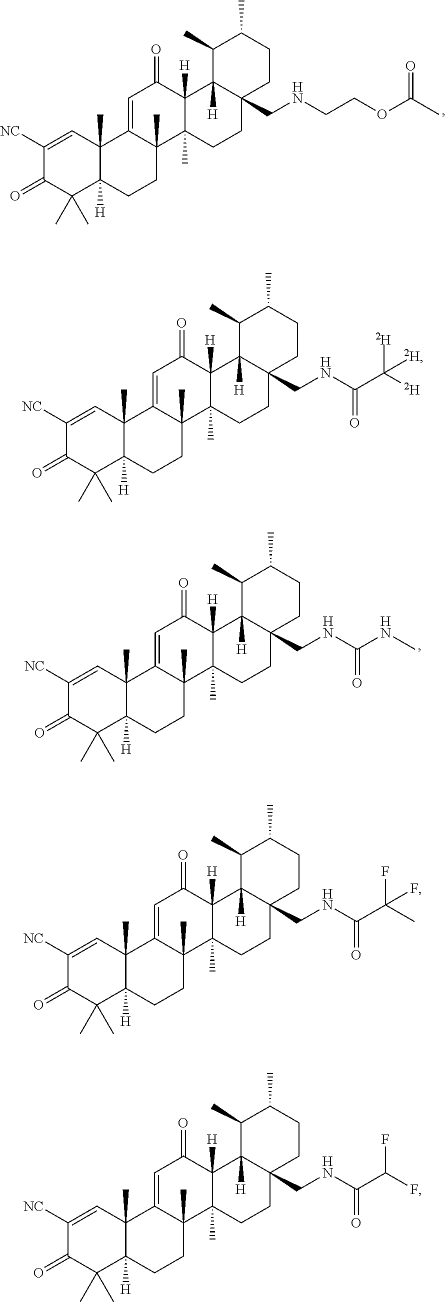

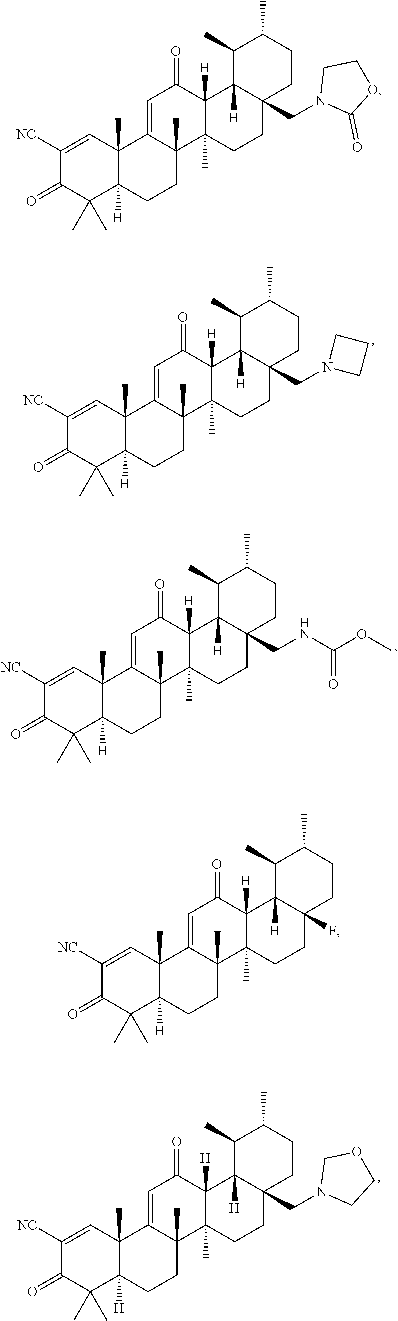

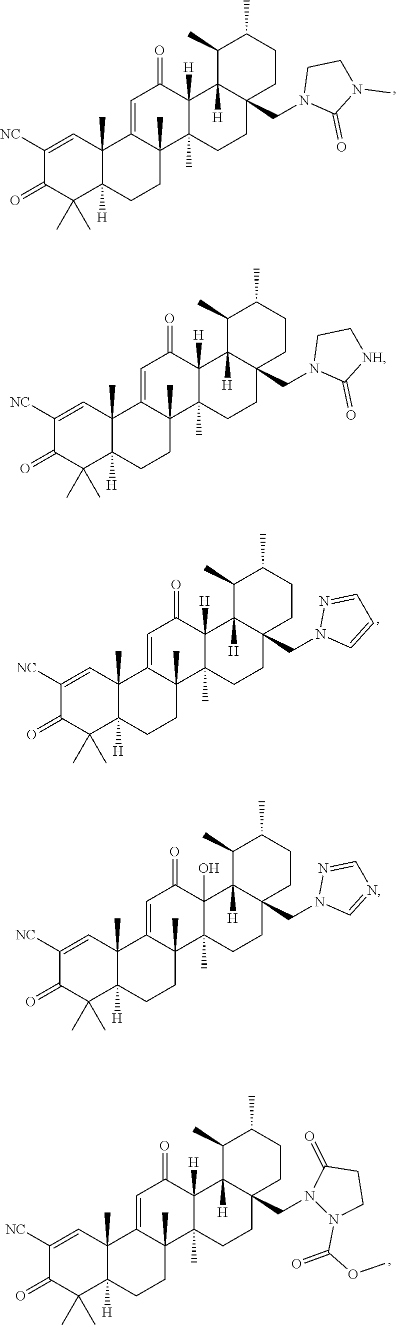

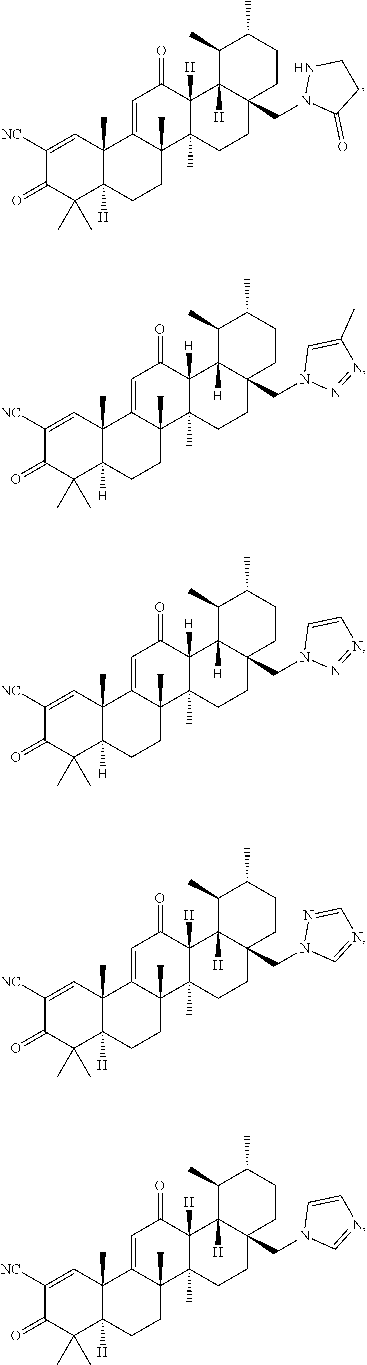

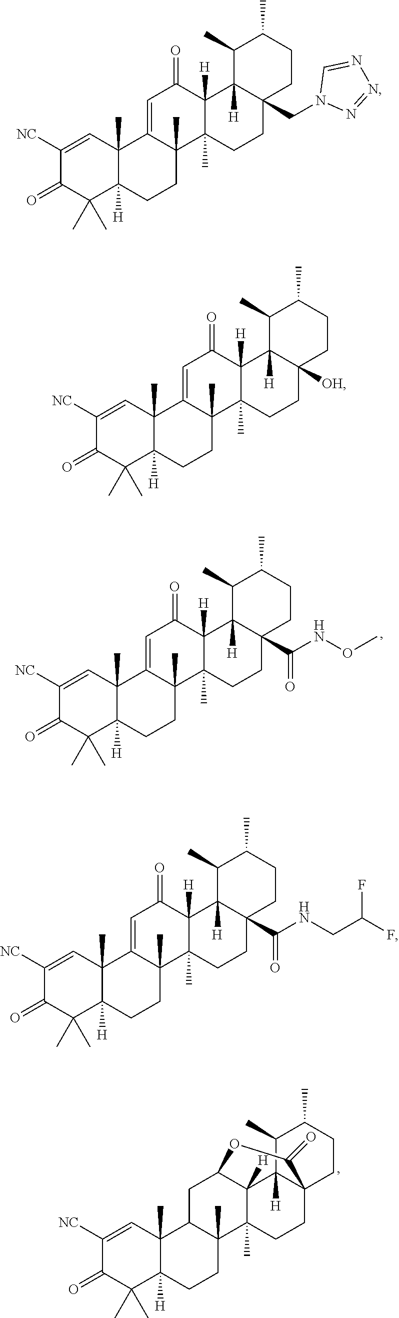

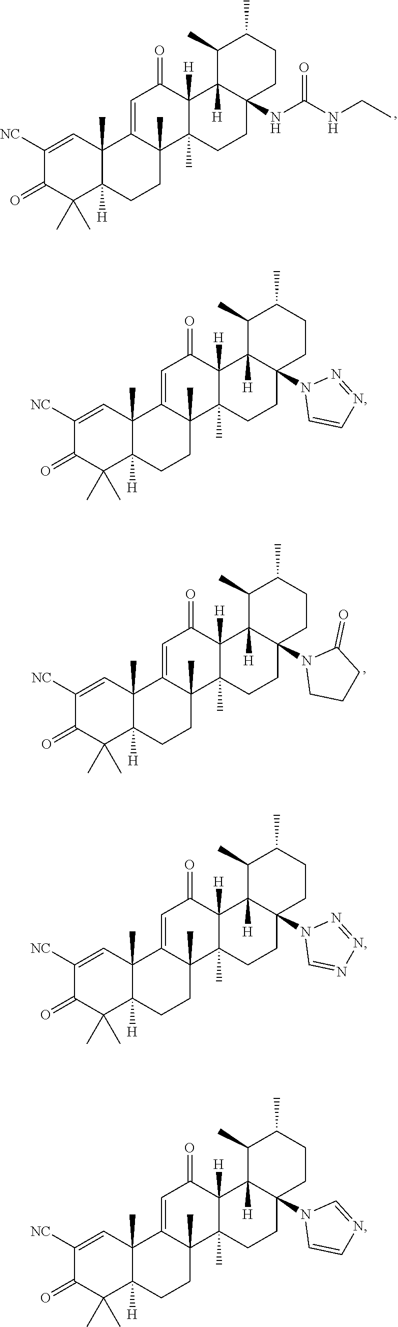

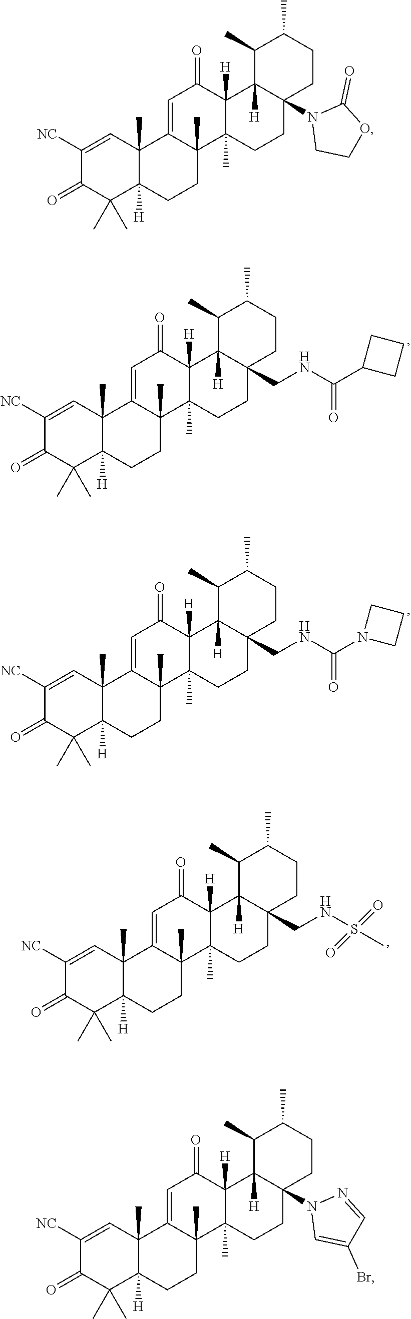

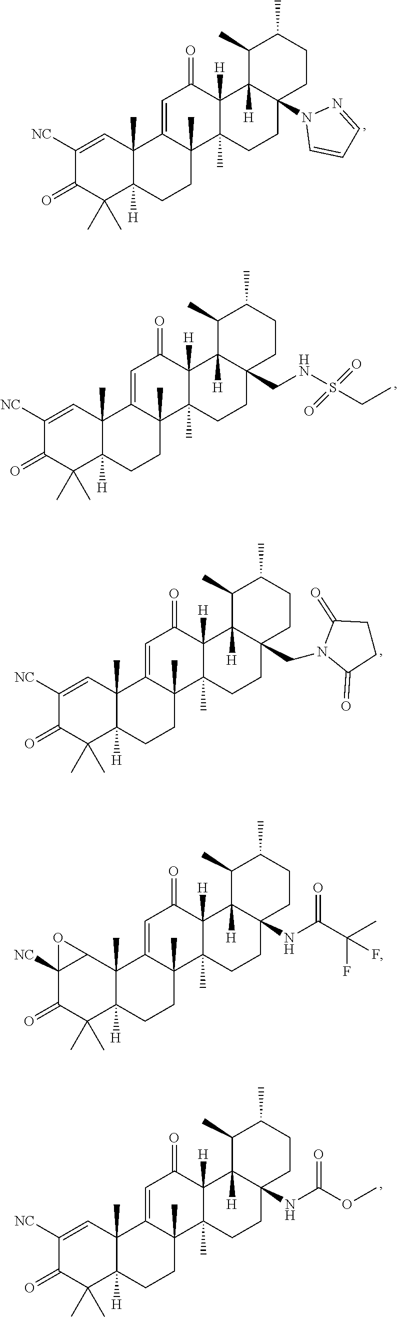

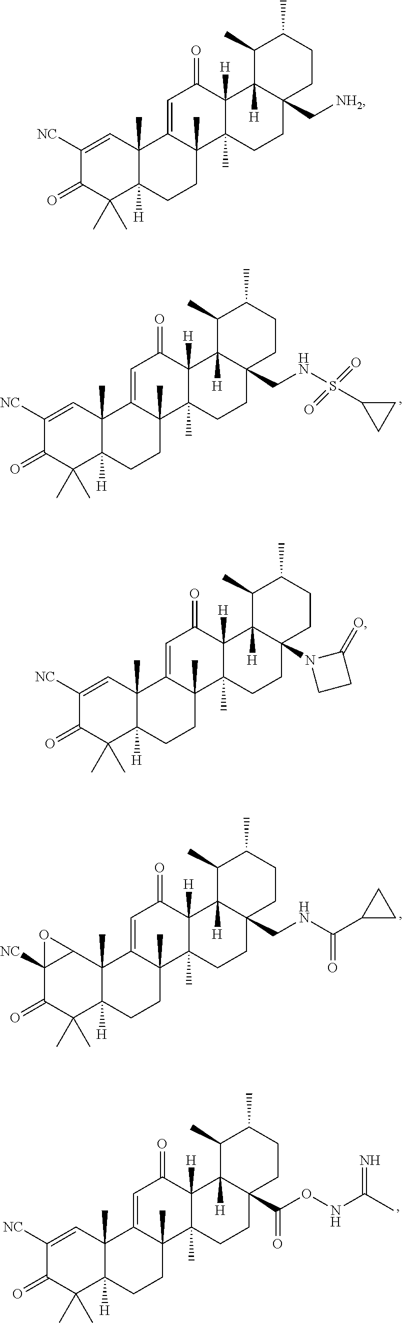

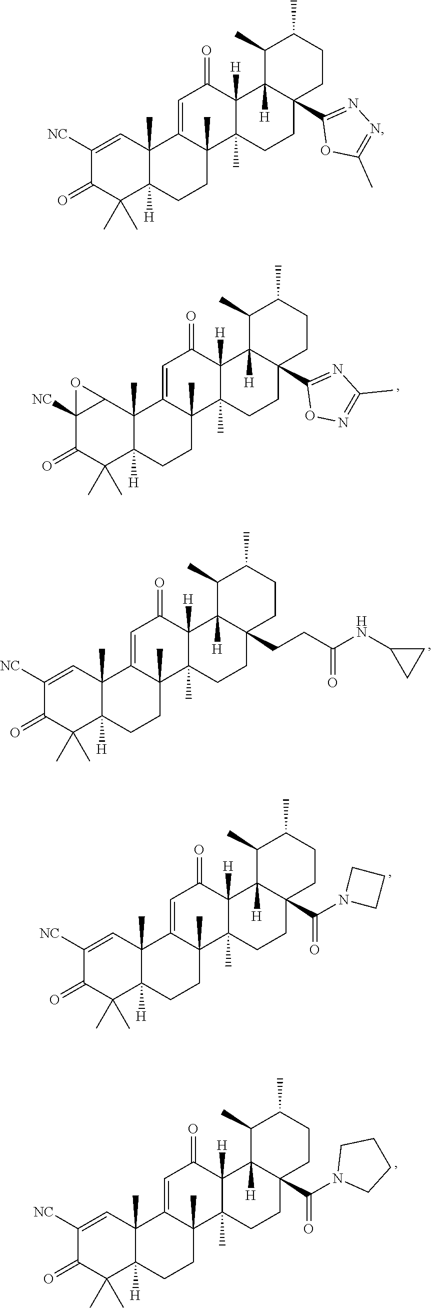

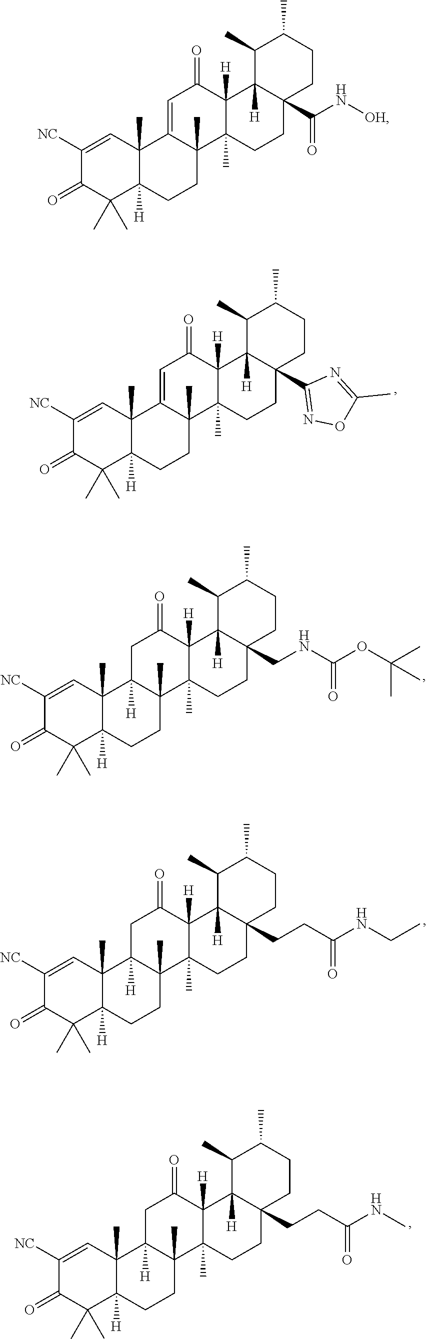

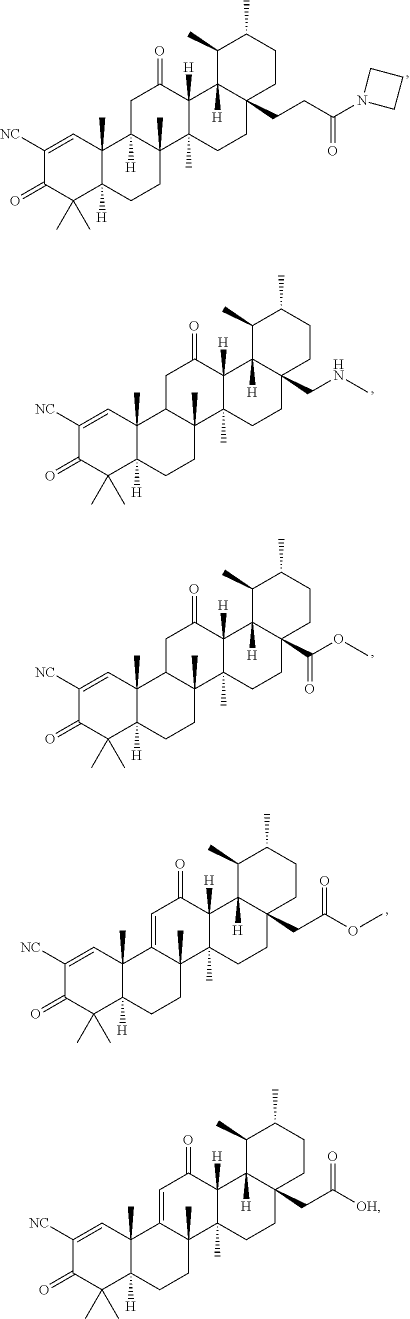

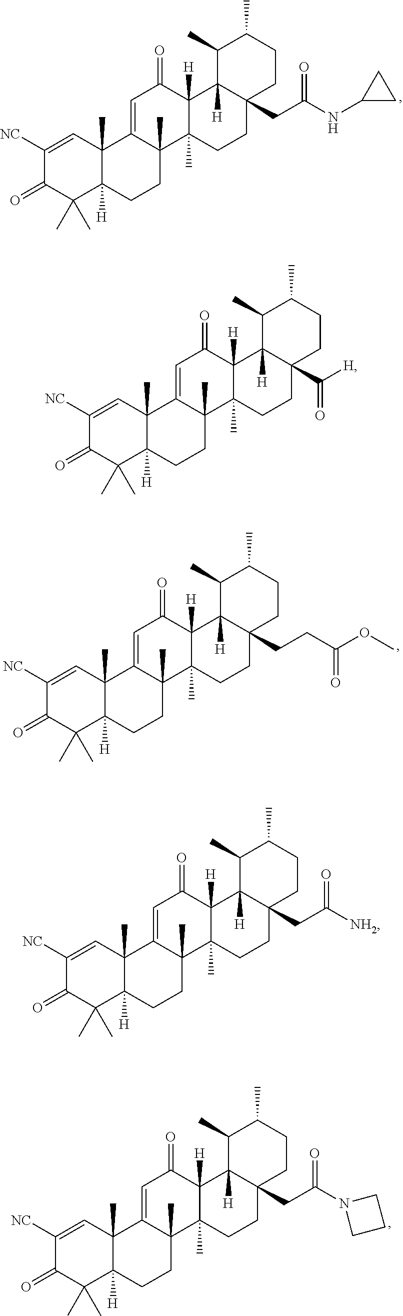

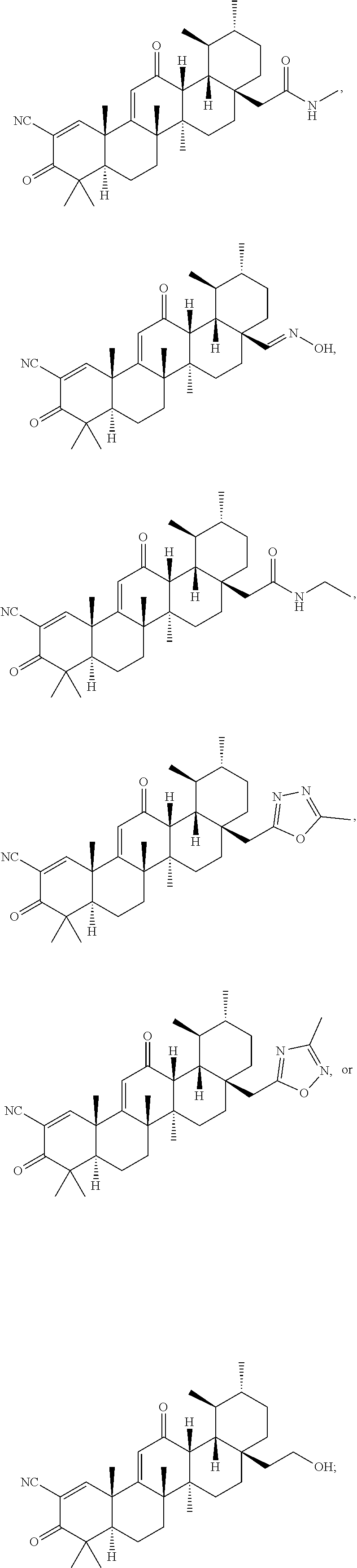

137. The compound of claim 136, wherein the compound is further defined as: 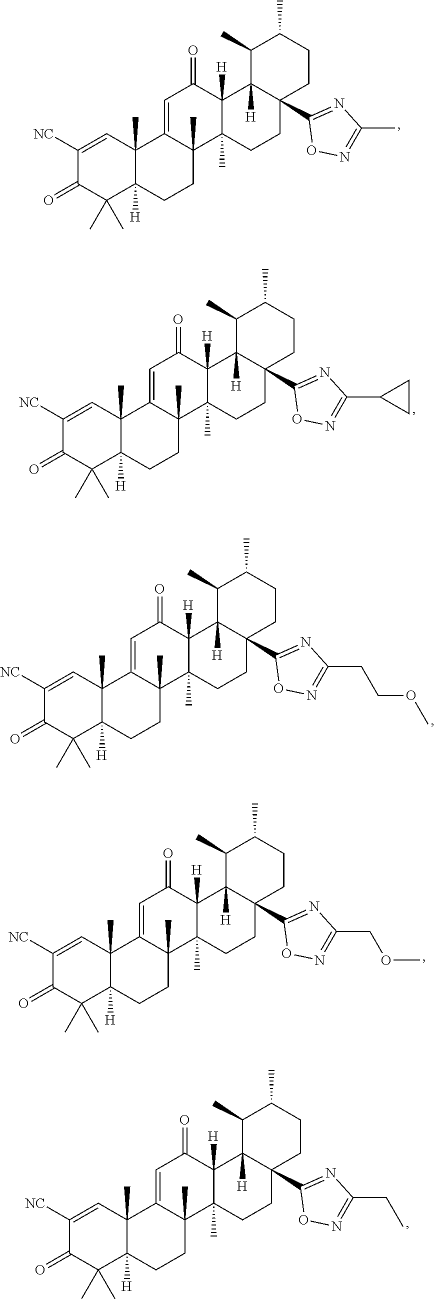 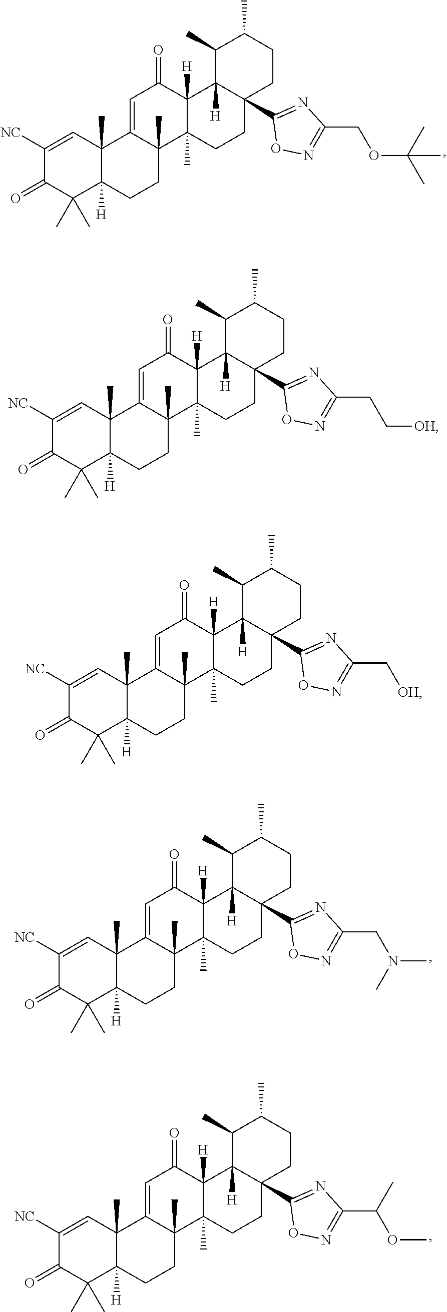 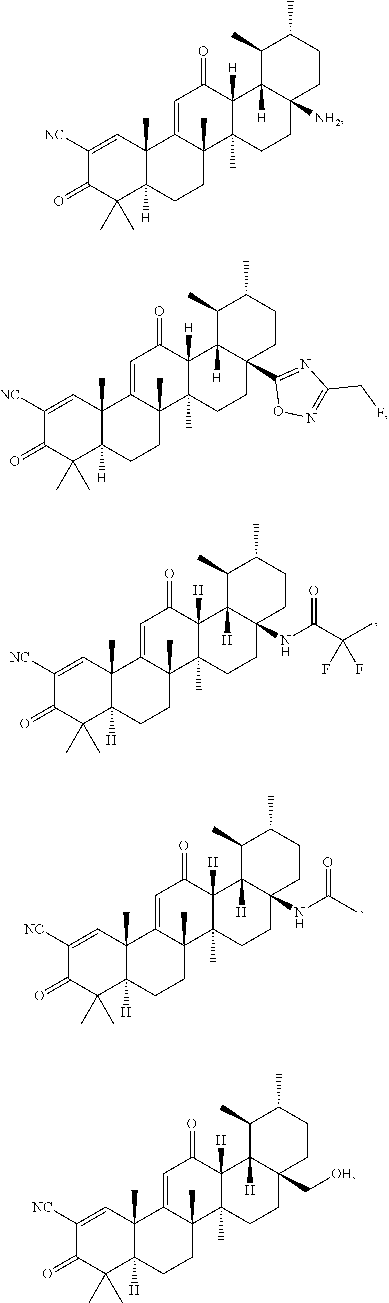 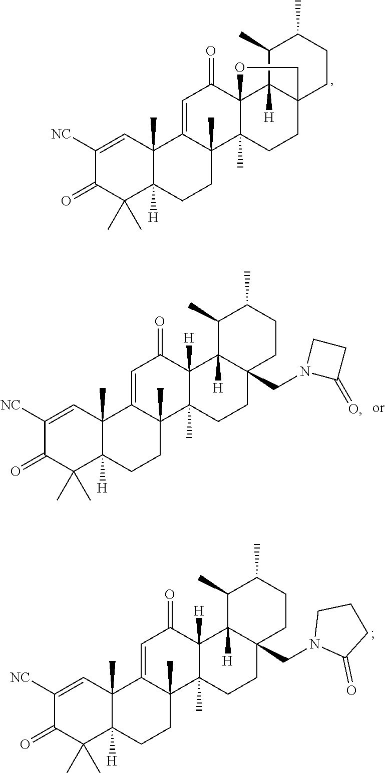
or a pharmaceutically acceptable salt thereof.

138. The compound of claim 136, wherein the compound is further defined as: 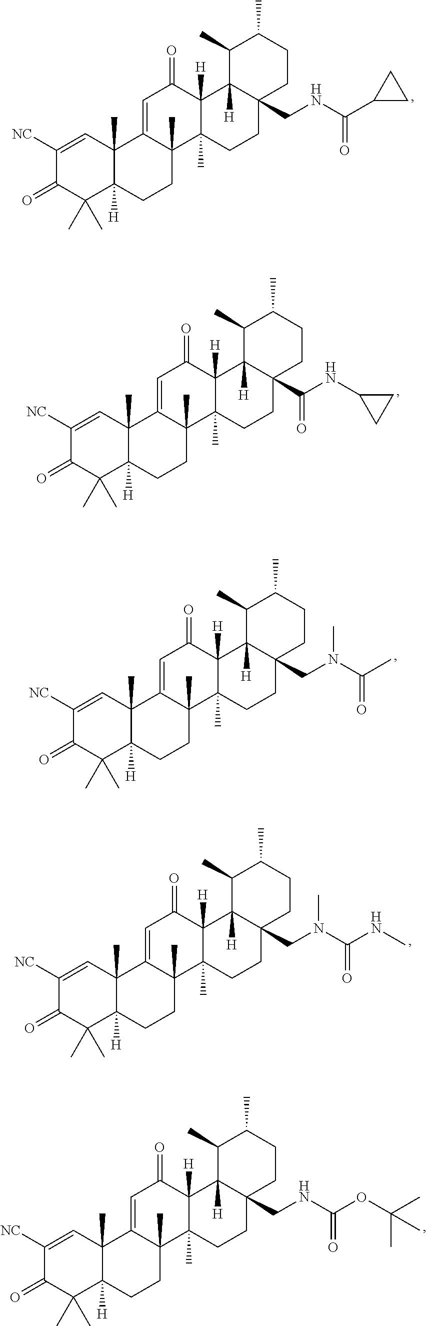 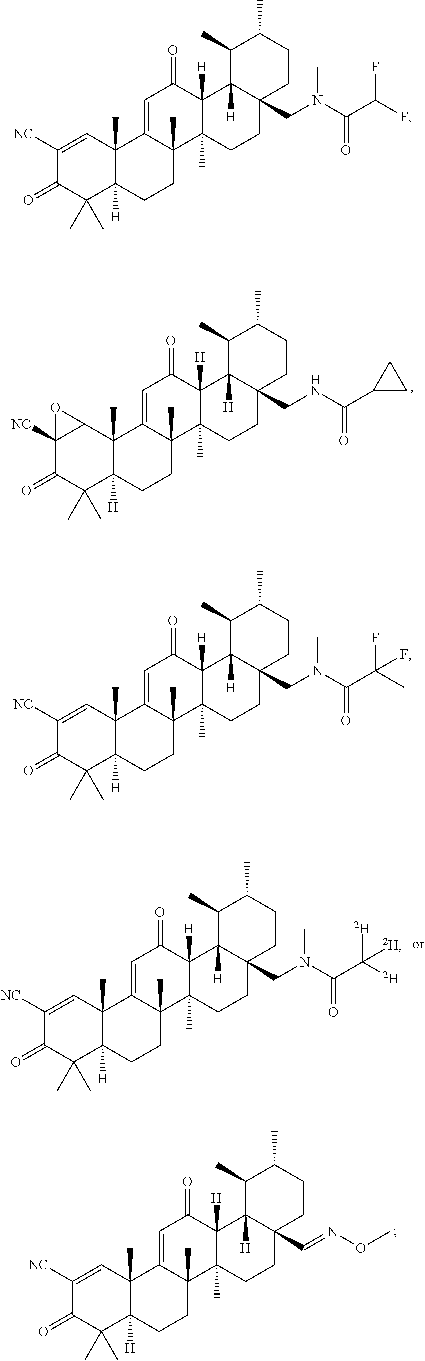
or a pharmaceutically acceptable salt thereof.

139. The compound according to any one of claims 1-135, wherein the compound is further defined as:
(4aR,6aS,6bR,8aS,11R,12S,12aS,12bR,14bS)-4,4,6a,6b,11,12,14b-heptamethyl-8a-(3-methyl-1,2,4-oxadiazol-5-yl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-cyclopropyl-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(2-methoxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(methoxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-ethyl-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(tert-butoxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(2-hydroxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(hydroxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-((dimethylamino)methyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(1-methoxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-amino-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(fluoromethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-2,2-difluoropropanamide;
N-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetamide;
(4aR,6aS,6bR,8aS,11R,12S,12aS,12bR,14bS)-8a-(hydroxymethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aR,6bS,11R,12S,12aR,12bS,14bS)-4,4,6a,6b,11,12,14b-heptamethyl-3,13-dioxo-4,4a,5,6,6a,6b,7,8,10,11,12,12a,13,14b-tetradecahydro-3H,9H-12b,8a-(epoxymethano)picene-2-carbonitrile;
(4aR,6aS,6bR,8aS,11R,12S,12aS,12bR,14bS)-4,4,6a,6b,11,12,14b-heptamethyl-3,13-dioxo-8a-((2-oxoazetidin-1-yl)methyl)-3,4,4a,5,6,6a,6b,7,8,8a,9,10,11,12,12a,12b,13,14b-octadecahydropicene-2-carbonitrile; or
(4aR,6aS,6bR,8aS,11R,12S,12aS,12bR,14bS)-4,4,6a,6b,11,12,14b-heptamethyl-3,13-dioxo-8a-((2-oxopyrrolidin-1-yl)methyl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)cyclopropanecarboxamide;
(1S,2R,4aS,6aR,6bS,8aR,12aS,14aR,14bS)-11-cyano-N-cyclopropyl-1,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxamide;
(4aR,6aS,6bR,8aS,11R,12S,12aS,12bR,14bS)-8a-hydroxy-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-fluoro-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-N-methylacetamide;
1-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-1,3-dimethylurea;
tert-butyl (((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)carbamate;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)acetamide;
1-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-3-methylurea;
(4aR,6aS,6bR,8aS,11R,12S,12aS,12bR,14bS)-8a-((1H-imidazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-((1H-tetrazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-((1H-1,2,3-triazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-((2-oxooxazolidin-3-yl)methyl)-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-((1H-pyrazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-((1H-1,2,4-triazol-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-8a-((3-methyl-2-oxoimidazolidin-1-yl)methyl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(((2-hydroxyethyl)amino)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-8a-(oxazolidin-3-ylmethyl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-8a-((4-methyl-1H-1,2,3-triazol-1-yl)methyl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(azetidin-1-ylmethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)acetamide-2,2,2-d3;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)propionamide;
(4aR,6aS,6bR,8aS,11R,12S,12aS,12bR,14bS)-4,4,6a,6b,11,12,14b-heptamethyl-3,13-dioxo-8a-((5-oxopyrazolidin-1-yl)methyl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-2,2-difluoropropanamide;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-2,2-difluoroacetamide;
(4aR,6aS,6bR,8aS,11R,12S,12aS,12bR,14bS)-4,4,6a,6b,11,12,14b-heptamethyl-3,13-dioxo-8a-((2-oxoimidazolidin-1-yl)methyl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-2,2-difluoro-N-methylacetamide;
2-((((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)amino)ethyl acetate;
(3aR,5aS,5bR,7aS,10R,11S,11aS,11bR,13bS)-7a-(azetidin-1-ylmethyl)-3,3,5a,5b,10,11,13b-heptamethyl-2,12-dioxo-3,3a,4,5,5a,5b,6,7,7a,8,9,10,11,1 1a,1 b,12,13b,13c-octadecahydropiceno[1,2-b]oxirene-1a(2H)-carbonitrile;
methyl (((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)carbamate;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-2,2-difluoro-N-methylpropanamide;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-N-methylacetamide-d3;
(4aR,6aR,6bS,8aS,11R,12S,12aR,14bS)-8a-((1H-1,2,4-triazol-1-yl)methyl)-12b-hydroxy-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
methyl 2-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-3-oxopyrazolidine-1-carboxylate;
(4aR,6aS,6bR,8aS,11R,12S,12aS,12bR,14bS)-8a-((E)-(methoxyimino)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(1S,2R,4aS,6aR,6bS,8aR,12aS,14aR,14bS)-11-cyano-N-methoxy-1,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxamide;
(1S,2R,4aS,6aR,6bS,8aR,12aS,14aR,14bS)- 11-cyano-N-(2,2-difluoroethyl)-1,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxamide;
(4aR,6aR,6bR,8aS,11R,12S,12aS,12bR,13R,14bR)-4,4,6a,6b,11,12,14b-heptamethyl-3,16-dioxo-4,4a,5,6,6a,6b,7,8,9,10,11,12,12a,12b,13,14,14a,14b-octadecahydro-3H-13,8a-(epoxymethano)picene-2-carbonitrile;
1-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-3-ethylurea;
(4aR,6aS,6bR,8aS,11R,12S,12aS,12bR,14bS)-4,4,6a,6b,11,12,14b-heptamethyl-3,13-dioxo-8a-(1H-1,2,3-triazol-1-yl)-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-(2-oxopyrrolidin-1-yl)-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-(1H-tetrazol-1-yl)-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(1H-imidazol-1-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2 carbonitrile;
(4aR,6aS,6bR,8aS,11R,12S,12aS,12bR,14bS)-4,4,6a,6b,11,12,14b-heptamethyl-3,13-dioxo-8a-(2-oxooxazolidin-3-yl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)cyclobutanecarboxamide;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)azetidine-1-carboxamide;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)methanesulfonamide;
(4aR,6aS,6bR,8aS,11R,12S,12aS,12bR,14bS)-8a-(4-bromo-1H-pyrazol-1-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-(1H-pyrazol-1-yl)-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)ethanesulfonamide;
(4aR,6aS,6bR,8aS,11R,12S,12aS,12bR,14bS)-8a-((2,5-dioxopyrrolidin-1-yl)methyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-((1aR,3aR,5aS,5bR,7aS,10R,11S,11aS,11bR,13bS)-1a-cyano-3,3,5a,5b,10,11,13b-heptamethyl-2,12-dioxo-1a,3,3a,4,5,5a,5b,6,7,8,9,10,11,11a,11b,12,13b,13c-octadecahydropiceno[1,2-b]oxiren-7a(2H)-yl)-2,2-difluoropropanamide;
methyl ((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)carbamate;
(4aR,6aS,6bR,8aS,11R,12S,12aS,12bR,14bS)-8a-(aminomethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-(((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)cyclopropanesulfonamide;
(4aR,6aS,6bR,8aS,11R,12S,12aS,12bR,14bS)-4,4,6a,6b,11,12,14b-heptamethyl-3,13-dioxo-8a-(2-oxoazetidin-¹-yl)-3,4,4a,5,6,6a,6b,7,8,8a,9,10,11,12,12a,12b,13,14b-octadecahydropicene-2-carbonitrile;
N-(((1aR,3aR,5aS,5bR,7aS,10R,11S,11aS,11bR,13bS)-1a-cyano-3,3,5a,5b,10,11,13b-heptamethyl-2,12-dioxo-1a,3,3a,4,5,5a,5b,6,7,8,9,10,11,111a,11b,12,13b,13c-octadecahydropiceno[1,2-b]oxiren-7a(2H)-yl)methyl)cyclopropanecarboxamide;
N-(((1S,2R,4aS,6aR,6bS,8aR,12aS,14aR,14bS)-11-cyano-1,2,6a,6b,9,9,12a-heptamethyl-10,14-dioxo-1,2,3,4,4a,5,6,6a,6b,7,8,8a,9,10,12a,14,14a,14b-octadecahydropicene-4a-carbonyl)oxy)acetimidamide;
(4aR,6aS,6bR,8aS,11R,12S,12aS,12bR,14bS)-4,4,6a,6b,11,12,14b-heptamethyl-8a-(5-methyl-1,3,4-oxadiazol-2-yl)-3,13-dioxo-3,4,4a,5,6,6a,6b,7,8,8a,9,10,11,12,12a,12b,13,14b-octadecahydropicene-2-carbonitrile;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)propanoic acid;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-methylpropanamide;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-ethylpropanamide;
(4aR,6aS,6bR,8aR,11R,12S,12aS,12bR,14bS)-8a-(3-(azetidine-1-yl)-3-oxopropyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(1aR,3aR,5aS,5bR,7aS,10R,11S,11aS,11bR,13bS)-3,3,5a,5b,10,11,13b-heptamethyl-7a-(3-methyl-1,2,4-oxadiazol-5-yl)-2,12-dioxo-3,3a,4,5,5a,5b,6,7,7a,8,9,10,11,11a,11b,12,13b,13c-octadecahydropiceno[1,2-b]oxirene-a(2H)-carbonitrile;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-cyclopropylpropanamide;
(4aR,6aS,6bR,8aS,11R,12S,12aS,12bR,14bS)-8a-(azetidine-1-carbonyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-(pyrrolidine-1-carbonyl)-3,4,4a,5,6,6a,6b,7,8,8a,9,10,11,12,12a,12b,13,14b-octadecahydropicene-2-carbonitrile;
(1S,2R,4aS,6aR,6bS,8aR,12aS,14aR,14bS)-11-cyano-N-hydroxy-1,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxamide;
(4aR,6aS,6bR,8aS,11R,12S,12aS,12bR,14bS)-4,4,6a,6b,11,12,14b-heptamethyl-8a-(5-methyl-1,2,4-oxadiazol-3-yl)-3,13-dioxo-3,4,4a,5,6,6a,6b,7,8,8a,9,10,11,12,12a,12b,13,14b-octadecahydropicene-2-carbonitrile;
tert-butyl (((1S,2R,4aS,6aR,6bR,8aR,12aR,14aR,14bS)-11-cyano-1,2,6a,6b,9,9,12a-heptamethyl-10,14-dioxo-1,3,4,5,6,6a,6b,7,8,8a,9,10,12a,12b,13,14,14a,14b-octadecahydropicen-4a(2H)-yl)methyl)carbamate;
3-((1S,2R,4aR,6aR,6bR,8aR,12aR,14aR,14bS)-11-cyano-1,2,6a,6b,9,9,12a-heptamethyl-10,14-dioxo-1,3,4,5,6,6a,6b,7,8,8a,9,10,12a,12b,13,14,14a,14b-octadecahydropicen-4a(2H)-yl)-N-ethylpropanamide;
3-((1S,2R,4aR,6aR,6bR,8aR,12aR,14aR,14bS)-11-cyano-1,2,6a,6b,9,9,12a-heptamethyl-10,14-dioxo-1,3,4,5,6,6a,6b,7,8,8a,9,10,12a,12b,13,14,14a,14b-octadecahydropicen-4a(2H)-yl)-N-methylpropanamide;
(4aR,6aR,6bR,8aR,11R,12S,12aS,12bR,14bR)-8a-(3-(azetidine-1-yl)-³-oxopropyl)-4,4,6a,6b,11,12,14b-heptamethyl-3,13-dioxo-3,4,4a,5,6,6a,6b,7,8,8a,9,10,11,12,12a,12b,13,14,14a,14b-icosahydropicene-2-carbonitrile;
(4aR,6aS,6bR,8aS,11R,12S,12aS,12bR,14bS)-4,4,6a,6b,11,12,14b-heptamethyl-8a-((methylamino)methyl)-3,13-dioxo-3,4,4a,5,6,6a,6b,7,8,8a,9,10,11,12,12a,12b,13,14b-octadecahydropicene-2-carbonitrile;
methyl (1S,2R,4aS,6aR,6bR,8aR,12aR,14aR,14bS)-11-cyano-1,2,6a,6b,9,9,12a-heptamethyl-10,14-dioxo-1,3,4,5,6,6a,6b,7,8,8a,9,10,12a,12b,13,14,14a,14b-octadecahydropicene-4a(2H)-carboxylate;
methyl 2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetate;
2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetic acid;
2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-cyclopropylacetamide;
(4aR,6aS,6bR,8aS,11R,12S,12aS,12bR,14bS)-8a-formyl-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
methyl 3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)propanoate;
2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetamide;
(4aR,6aS,6bR,8aR,11R,12S,12aS,12bR,14bS)-8a-(2-(azetidine-1-yl)-2-oxoethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
2-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-methylacetamide;
methyl 3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)propanoate;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)propanoic acid;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-methylpropanamide;
3-((1S,2R,4aR,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-N-ethylpropanamide; or
(4aR,6aS,6bR,8aR,11R,12S,12aS,12bR,14bS)-8a-(3-(azetidin-1-yl)-3-oxopropyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
or a pharmaceutically acceptable salt thereof.

140. The compound of claim 103, wherein the compound is further defined as:
(4aR,6aS,6bR,8aS,11R,12S,12aS,12bR,14bS)-4,4,6a,6b,11,12,14b-heptamethyl-8a-(3-methyl-1,2,4-oxadiazol-5-yl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-cyclopropyl-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(2-methoxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(methoxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-ethyl-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(tert-butoxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(2-hydroxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(hydroxymethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-((dimethylamino)methyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(1-methoxyethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-amino-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-8a-(3-(fluoromethyl)-1,2,4-oxadiazol-5-yl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
N-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-2,2-difluoropropanamide;
N-((1S,2R,4aS,6aR,6bS,8aR,12aS,14aR,14bS)-11-cyano-1,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)acetamide;
(4aR,6aS,6bR,8aS,11R,12S,12aS,12bR,14bS)-8a-(hydroxymethyl)-4,4,6a,6b,11,12,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,11R,12S,12aS,12bR,14bS)-4,4,6a,6b,11,12,14b-heptamethyl-3,13-dioxo-8a-((2-oxoazetidin-1-yl)methyl)-3,4,4a,5,6,6a,6b,7,8,8a,9,10,11,12,12a,12b,13,14b-octadecahydropicene-2-carbonitrile; or
(4aR,6aS,6bR,8aS,11R,12S,12aS,12bR,14bS)-4,4,6a,6b,11,12,14b-heptamethyl-3,13-dioxo-8a-((2-oxopyrrolidin-1-yl)methyl)-3,4,4a,5,6,6a,6b,7,8,8a,9,10,11,12,12a,12b,13,14b-octadecahydropicene-2-carbonitrile;
or a pharmaceutically acceptable salt thereof.

141. A compound of the formula: 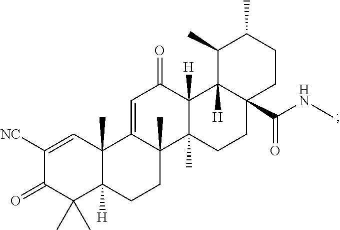
or a pharmaceutically acceptable salt thereof.

142. The compound of claim 141, wherein the compound is further defined as:
(1S,2R,4aS,6aR,6bS,8aR,12aS,14aR,14bS)-11-cyano-N,1,2,6a,6b,9,9,12a-octamethyl-10,14-dioxo-1,3,4,5,6,6a,6b,7,8,8a,9,10,12a,14,14a,14b-hexadecahydropicene-4a(2H)-carboxamide
or a pharmaceutically acceptable salt thereof.

143. A pharmaceutical composition comprising:
(A) a compound according to any one of claims 1-142; and
(B) an excipient.

144. The pharmaceutical composition of claim 143, wherein the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularily, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

145. The pharmaceutical composition of claim 144, wherein the pharmaceutical composition is formulated for oral administration.

146. The pharmaceutical composition of claim 144, wherein the pharmaceutical composition is formulated for administration via injection.

147. The pharmaceutical composition of claim 146, wherein the pharmaceutical composition is formulated for intraarterial administration, intramuscular administration, intraperitoneal administration, or intravenous administration.

148. The pharmaceutical composition of claim 144, wherein the pharmaceutical composition is formulated for administration topically.

149. The pharmaceutical composition of claim 148, wherein the pharmaceutical composition is formulated for topical administration to the skin or to the eye.

150. The pharmaceutical composition according to any one of claims 143-149, wherein the pharmaceutical composition is formulated as a unit dose.

151. A method of treating or preventing a disease or disorder in a patient in need thereof comprising administering to the patient a pharmaceutically effective amount of a compound or composition according to any one of claims 1-150.

152. The method of claim 151, wherein the patient is a mammal.

153. The method of claim 152, wherein the patient is a human.

154. The method of claim 151, wherein the disease or disorder is a condition associated with inflammation and/or oxidative stress.

155. The method of claim 151, wherein the disease or disorder is cancer.

156. The method of claim 151, wherein the disease or disorder is a cardiovascular disease.

157. The method of claim 156, wherein the cardiovascular disease is atherosclerosis.

158. The method of claim 151, wherein the disease or disorder is an autoimmune disease.

159. The method of claim 158, wherein the autoimmune disease is Crohn's disease, rheumatoid arthritis, lupus, or psoriasis.

160. The method of claim 151, wherein the disease or disorder is a neurodegenerative disease.

161. The method of claim 160, wherein the neurodegenerative disease is Alzheimer's disease, Parkinson's disease, amyotrophic lateral sclerosis, or Huntington's disease.

162. The method of claim 151, wherein the disease or disorder is chronic kidney disease, diabetes, mucositis, inflammatory bowel disease, dermatitis, sepsis, ischemia-reperfusion injury (including complications from sickle cell anemia), influenza, osteoarthritis, osteoporosis, pancreatitis, asthma, chronic obstructive pulmonary disease, cystic fibrosis, idiopathic pulmonary fibrosis, multiple sclerosis, muscular dystrophy, cachexia, or graft-versus-host disease.

163. The method of claim 151, wherein the disease or disorder is an eye disease.

164. The method of claim 163, wherein the eye disease is uveitis, glaucoma, macular degeneration, or retinopathy.

165. The method of claim 151, wherein the disease or disorder is neuropsychiatric.

166. The method of claim 165, wherein the neuropsychiatric disease or disorder is schizophrenia, depression, bipolar disorder, epilepsy, post-traumatic stress disorder, attention deficit disorder, autism, or anorexia nervosa.

167. The method of claim 151, wherein the disease or disorder is associated with mitochondrial dysfunction.

168. The method of claim 167, wherein the disease or disorder associated with mitochondrial dysfunction is Friedreich's ataxia.

169. The method of claim 151, wherein the disease or disorder is chronic pain.

170. The method of claim 151, wherein the disease or disorder is neuropathic pain.

171. A method of inhibiting nitric oxide production comprising administering to a patient in need thereof an amount of a compound or composition of claims 1-170 sufficient to cause inhibition of IFN-γ-induced nitric oxide production in one or more cells of the patient.


SEQUENCE LISTING (1 sequence)

SEQ ID NO: 1 - DNA, 11 bp, Artificial Sequence
  Synthetic primer
gtgacaaagca